SEQUENCE LISTING (12 sequences)

SEQ ID NO: 1 - protein, 1241 aa, Bat severe acute respiratory syndrome coronavirus Rm1/2004
MKVLIFALLFSLAKAQEGCGIISRKPQPKMEKVSSSRRGVYYNDDIFRSDVLHLTQDYFLPFDSNLTQYFSLNIDSNKYTYFDNPILDFGDGVYFAATEKSNVIRGWIFGSSFDNTTQSAIIVNNSTHIIIRVCNFNLCKEPMYTVSKGTQQSSWVYQSAFNCTYDRVEKSFQLDTAPKTGNFKDLREYVFKNKGGFLRVYQTYTAVNLPRGFPAGFSVLRPILKLPFGINITSYRVVMTMFSQFNSNFLPESAAYYVGNLKYTTFMLSFNENGTITDAVDCSQNPLAELKCTIKNFNVSKGIYQTSNFRVTPTQEVVRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAQQDQGQYYYRSYRKEKLKPFERDLSSDENGVYTLSTYDFYPSIPVEYQATRVVVLSFELLNAPATVCGPKLSTQLVKNQCVNFNFNGLRGTGVLTTSSKRFQSFQQFGRDTSDFTDSVRDPQTLEILDISPCSFGGVSVITPGTNASSEVAVLYQDVNCTDVPTSIHADQLTPAWRVYSTGVNVFQTQAGCLIGAEHVNASYECDIPIGAGICASYHTASVLRSTGQKSIVAYTMSLGAENSIAYANNSIAIPTNFSISVTTEVMPVSIAKTSVDCTMYICGDSLECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGSALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVAGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 221 aa, Bat severe acute respiratory syndrome coronavirus Rm1/2004
MTDNGTITVEELKQLLEQWNLVIGFIFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTILTRPLLESELVIGAVIIRGHLRMAGHSLGRCDIKDLPKEITVATSRTLSYYKLGASQRVGNDSGFAAYNRYRIGNYKLNTDHSGSNDNIALLVQ

SEQ ID NO: 4 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 5 - protein, 76 aa, Bat severe acute respiratory syndrome coronavirus Rm1/2004
MYSFVSEETGTLIVNSVLLFFAFVVFLLVTLAILTALRLCAYCCNIVNVSLVKPTVYVYSRVKNLNSSEGVPDLLV

SEQ ID NO: 6 - protein, 74 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLL

SEQ ID NO: 7 - protein, 420 aa, Bat severe acute respiratory syndrome coronavirus Rm1/2004
MSDNGPQNQRSAPRITFGGPSDSTDNNQDGGRSGARPKQRRPQGLPNNTASWFTALTQHGKEELRFPRGQGVPINTNSGKDDQIGYYRRATRRVRGGDGKMKELSPRWYFYYLGTGPEASLPYGANKEGIVWVATEGALNTPKDHIGTRNPNNNAAIVLQLPQGTTLPKGFYAEGSRGGSQASSRSSSRSRGNSRNSTPGSSRGNSPARMASGSGETALALLLLDRLNQLESKVSGKGQQQQGQTVTKKSAAEASKKPRQKRTATKSYNVTQAFGRRGPEQTQGNFGDQDLIRQGTDYKYWPQIAQFAPSASAFFGMSRIGMEVTPLGTWLTYHGAIKLDDKDPQFKDNVILLNKHIDAYKTFPPTEPKKDKKKKTDEAQPLPQRKKQPTVTLLPAADMDDFSRQLQNSMSGASADSTQA

SEQ ID NO: 8 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 9 - protein, 508 aa, Human immunodeficiency virus 1
MGARASVLSGGKLDKWEKIRLRPGGKKTYQLKHIVWASRELERFAVNPGLLETGGGCKQILVQLQPSLQTGSEELKSLYNAVATLYCVHQGIEVRDTKEALDKIEEEQNKSKKKAQQAAADTGNSSQVSQNYPIVQNLQGQMVHQAISPRTLNAWVKVIEEKAFSPEVIPMFSALSEGATPQDLNTMLNTVGGHQAAMQMLKETINEEAAEWDRLHPAHAGPNAPGQMREPRGSDIAGTTSTLQEQIGWMTSNPPVPVGEIYKRWIILGLNKIVRMYSPVSILDIRQGPKEPFRDYVDRFYKTLRAEQASQDVKNWMTETLLVQNANPDCKTILKALGPAATLEEMMTACQGVGGPSHKARILAEAMSQVTSPANIMMQRGNFRNQRKTIKCFNCGKEGHLARHCRAPRKKGCWKCGREGHQMKDCTERQANFLGKIWPSHKGRPGNFLQSRPEPTAPPEESFRFGEETTTPPQKQEPLPSQKQETIDKDLYPLASLKSLFGNDPSLQ

SEQ ID NO: 10 - protein, 679 aa, Human immunodeficiency virus 1
MRVKGIRKNYQHWWRWGIMLLGMWMICSAAEKLWVTVYYGVPVWKEATTTLFCASDAKAYDTEVHNVWATHACVPIDPNPQEVFLENVTENFNMWKNNMVEQMHEDIISLWDQSLKPCVKLTPLCVTLKCTDLNDTNTNNSSTSENNTNPTISGGEGMGEGEMKNCSFNVTTNIRDKVQKEYALFYKLDIIPIDNTSYALRHCNTSVITQACPKVSFEPIPIHYCAPAGFAILQCNDKKFNGTGPCSNVSTVQCTHGIRPVVSTQLLLNGSLAEEEIVLRSENFTNNAKTIIVQLNASVEINCTRLNNNTRKSIRIGPGSTFYATGAIIGDIRQAHCNISREKWNDTLKQLVIKLGEQFGNSNIIVFKQSSGGDPEIVMHSFICGGEFFYCNTTQLSNSTWQRSDGTWNRTGGLNETKENITLPCRIKQIINRWQEVGKAMYAPPISGLIRCSSNITGLLLTRDGGNENNGTNGTETFRPEGGNMKDNWRSKLYKYKVVRIEPLGIAPTRARRRVVQREKRAVTFGALFLGFLGAAGSTMGCTSMTLTVQARLLLSGIVQQQNNLLRAIEAQQHLLQLTVWGIKQLQARVLAVERYLRDQQLLGIWGCSGKLICTTNVPWNTSWSNKSENEIWDNMTWMEWDREINNYTNLIYDLLEKSQNQQEKNEQELLELDKWASL

SEQ ID NO: 11 - protein, 1003 aa, Human immunodeficiency virus 1
FFRENLALLQGEARELSSEQARANSPTCRELQVRGRDSSPLLEAGAEGKGAISLSFPQITLWQRPLVTVRIGEQLIEALLDTGADDTVLEDINLPGKWKPKMIGGIGGFIKVRQYDQIHIEICGKKAIGTVLVGPTPVNIIGRNMLTQIGCTLNLPISPIETVPVKLKPGMDGPRVKQWPLTEEKIKALTEICTEMEKEGKISKIGPENPYNTPIFAIKKKDSTKWRKLVDFRELNKRTQDFWEVQLGIPHPAGLKKKKSVTILDVGDAYFSVPLDKEFRKYTAFTIPSINNETPGTRYQYNVLPQGWKGSPAIFQSSMTRILEPFRKQNPEIVIYQYMDDLYVGSDLEIGQHRVKIEELREHLLKWGFTTPDKKHQKEPPFLWMGYELHPDKWTVQPIELPDKESWTVNDIQKLVGKLNWASQIYPGIKVRQLCKLLRGARALTDIVPLTAEAELELAENREILKEPVHGVYYDPSKDLIAEIQKQGHGQWTYQIYQEPHKNLKTGKYAKIKSAHTNDVKQLTEAVQKIAMESIVVWGKTPKFRLPIQKETWETWWTEYWQATWIPDWEFVNTPPLVKLWYQLETEPIVGAETFYVDGAANRETKKGKAGYVTDRGRQRVITLSETTNQKTELQAINLALQDSGSEVNIVTDSQYALGIIQAQPDKSESELVNQIIEQLIKKEKVYLSWVPAHKGIGGNEQVDKLVSSGIRKVLFLDGIDKAQEEHEKYHNNWRAMANDFNLPPIVAKEIVASCDKCQLKGEAMHGQVDCSPGIWQIDCTHLEGKIIIVAVHVASGYIEAEVIPAETGQETAYFILKLAGRWPVKVIHTDNGSNFTSAAVKAACWWANVTQEFGIPYNPQSQGVVESMNKELKKIIGQVRDQAEHLKTAVQMAVFIHNFKRKGGIGGYSAGERIIDIIASDIQTKELQKQIIKIQNFRVYYRDSRDPIWKGPAKLLWKGEGAVVIQDNSEIKVVPRRKAKIIRDYGKQMAGDDCVAGRQDED

SEQ ID NO: 12 - protein, 99 aa, Human immunodeficiency virus 1
PQITLWQRPLVTIKVEGQVKEALLDTGADDTVLEDMNLPGRWKPKMIGGIGGFIKVRQYDQITLEICGKKAIGTVLVGPTPVNIIGRNMLTQLGCTLNF

(19) United States
(12) Patent Application Publication
(10) Pub. No.: US 2022/0249651 A1
(43) Pub. Date: Aug. 11, 2022

(54) UNIVERSAL VACCINES AGAINST IMMUNOGENS OF PATHOGENIC ORGANISMS THAT PROVIDE ORGANISM-SPECIFIC AND CROSS-GROUP PROTECTION

(71) Applicant: AEGLE BIOTECH, Coral Springs, FL (US)

(72) Inventors: Beverly W. Lubit, Kinnelon, NJ (US); Lilit Garibyan, Newton, MA (US); Helen Hao, Piscataway, NJ (US); Sean O'Connell, Budd Lake, NJ (US)

(73) Assignee: AEGLE BIOTECH, Coral Springs, FL (US)

(21) Appl. No.: 17/589,484

(22) Filed: Jan. 31, 2022

Related U.S. Application Data
(60) Provisional application No. 63/144,068, filed on Feb. 1, 2021.

(51) Int. Cl. A61K 39/145; A61P 31/16; C07K 14/005; C12N 15/86; C12N 7/00

(52) U.S. Cl. CPC A61K 39/145; A61P 31/16; A61K 2039/543; C12N 15/86; C12N 7/00; C07K 14/005

(57) ABSTRACT

The present disclosure provides, in part, a priming and boosting vector-based platform to develop vaccines against viral pathogens that is tailored to elicit a broad T cell response targeting conserved viral epitopes while including helper T cell (T_{H}) epitopes and an adjuvant to achieve a balanced immune response consisting of both cellular immunity, coupled with a broad neutralizing antibody response in the design of a candidate universal vaccine to HIV or a human coronavirus, e.g., SARS-CoV-2. The universal vaccines are prepared against an immunogen of an infectious pathogenic virus comprising at least one nucleic acid polynucleotide comprising an open reading frame encoding at least one polypeptide antigen or an immunogenic fragment thereof, wherein the polypeptide antigen, or the immunogenic fragment thereof, comprises a conserved internal protein that is enriched in T cell recognition antigens. The effectiveness of the priming and boosting platform is tested in humanized mouse models: a transgenic mouse model that expresses the hACE2 gene under the control of the human cytokeratin 18 promoter and a humanized mouse model comprising a fully functional human immune system.

### RELATED APPLICATIONS

This application claims the benefit of U.S. Provisional Patent Application No. 63/144,068, filed Feb. 1, 2021, which is a continuation-in-part of U.S. patent application Ser. No. 16/737,546, filed on Jan. 8, 2020, which is a continuation-in-part of International Patent Application No. PCT/CN2018/105020, filed with the Chinese National Intellectual Property Administration Receiving Office on Sep. 11, 2018.

### FIELD OF THE INVENTION

The described invention relates generally to universal vaccines against immunogens of pathogenic organisms that provide organism-specific and cross-group protection.

### SEQUENCE LISTING

The instant application contains a Sequence Listing which has been submitted electronically in ASCII format and is hereby incorporated by reference in its entirety. Said ASCII copy, created on Jan. 27, 2022, is named 130189-00419 SL.txt and is 54,746 bytes in size.

### BACKGROUND

Infectious existing and emerging pathogens continue to cause significant morbidity and mortality worldwide. In 1990 alone, an estimated 16 million people died from infections. Despite the numerous new therapeutic products that have become available since then, in 2010 the number of deaths caused by infections had fallen only to 15 million. The majority of these deaths were caused by just a few pathogens: among the 1400 or so recognized human pathogens and parasites, the majority of deaths were caused by respiratory illness, diarrhea, HIV/AIDS, TB, malaria, meningitis, pertussis, measles, hepatitis B, and sexually transmitted diseases (STDs) (Dye C. After 2015: infectious diseases in a new era of health and development. (2014) Philosophical Transactions of the Royal Society of London. Series B, Biological Sciences, 369(1645), 20130426. doi:10.1098/rstb.2013.0426). Certain diseases are considered particularly important, e.g., because they had a 100% lethality rate when they emerged, for example, HIV/AIDS; or because the infectious viral agent causes disease beyond the principal person of infection, for example the emergence of birth defects from infection with zika virus.

Therapeutic products used to fight pathogens include preventative immunizations, such as vaccines, and post-infection therapeutics, such as anti-bacterials and anti-virals. Vaccines are therapeutics composed of one or a few specific antigens of the causative microbial agent or the microbial or viral body with its whole set of antigens that induce an immune response in the receiving individual and/or a cellular response in the pathogen itself (Cassone, A., & Rappuoli, R. (2010). Universal vaccines: shifting to one for many. Bio. 1(1), e00042-10. doi:10.1128/mBio.00042-10). Vaccines protect by inducing effector mechanisms capable of rapidly controlling replicating pathogens or inactivating their toxic components.

Generally speaking, immune responses are initiated by an encounter between an individual and a foreign substance, e.g., an infectious microorganism. The infected individual rapidly responds with both a humoral immune response with the production of antibody molecules specific for the antigenic determinants/epitopes of the immunogen, and a cell mediated immune response with the expansion and differentiation of antigen-specific regulatory and effector T-lymphocytes, including cells that produce cytokines and killer T cells, capable of lysing infected cells. Primary immunization with a given microorganism evokes antibodies and T cells that are specific for the antigenic determinants/epitopes found on that microorganism; these usually fail to recognize or recognize only poorly antigenic determinants expressed by unrelated microbes (Paul, W. E., “Chapter 1: The immune system: an introduction,” Fundamental Immunology, 4th Edition, Ed. Paul, W. E., Lippincott-Raven Publishers, Philadelphia, (1999), at p. 102).

As a consequence of this initial response, the immunized individual develops a state of immunologic memory. If the same or a closely related microorganism is encountered again, a secondary response ensues. This secondary response generally consists of an antibody response that is more rapid, greater in magnitude and composed of antibodies that bind to the antigen with greater affinity and that are more effective in clearing the microbe from the body, and a similarly enhanced and often more effective T-cell response. However, immune responses against infectious agents do not always lead to elimination of the pathogen (Paul, W. E., “Chapter 1: The immune system: an introduction,” Fundamental Immunology, 4th Edition, Ed. Paul, W. E., Lippincott-Raven Publishers, Philadelphia, (1999), at p. 102).

The human immune system is a complex arrangement of cells and molecules that maintain immune homeostasis to preserve the integrity of the organism by elimination of all elements judged to be dangerous. Responses in the immune system may generally be divided into two arms, referred to as “innate immunity” and “adaptive immunity.” The two arms of immunity do not operate independently of each other, but rather work together to elicit effective immune responses.

The innate arm of the immune system is a nonspecific fast response to pathogens that is predominantly responsible for an initial inflammatory response via a number of soluble factors, including the complement system and the chemokine/cytokine system; and a number of specialized cell types, including mast cells, macrophages, dendritic cells (DCs), and natural killer cells (NKs).

The adaptive immune arm involves a specific, delayed and longer-lasting response by various types of cells that create long-term immunological memory against a specific antigen. It can be further subdivided into cellular and humoral branches, the former largely mediated by T cells and the latter by B cells. This arm further encompasses cell lineage members of the adaptive arm that have effector functions in the innate arm, thereby bridging the gap between the innate and adaptive immune response.

Generally speaking, vaccination educates both innate and adaptive immune systems in order to boost adaptive T and B cell memory responses and provide rapid protection against subsequent infection with related viruses. (Li, G. et al, “Memory T Cells in Flavivirus Vaccination”, Vaccines (2018) 6: 73).

### Vaccines and Vaccination

While vaccination provides a cost effective measure to prevent disease and to control outbreaks of infection at herd level, vaccines currently on the market have significant shortcomings and even failures.

The first vaccine developed was one in which the wild-type disease or the wild-type version of a related disease was “killed” and delivered. While such vaccines were known to work, they carried a significant risk of severe disease or even death in the recipient.

The second type of vaccine developed was attenuated vaccines. This vaccine was based on material obtained from infected rabbit brain attenuated by drying, an uncertain process; vaccines prepared in this way frequently caused serious side effects. Attenuated vaccines are mostly now based on inactivated virus grown in tissue culture. Rabies was the first virus attenuated in a laboratory to create a human vaccine. Acquisition of the ability to grow viruses in tissue culture for an extended period led to the development of attenuated vaccines against measles, poliomyelitis, rubella, influenza, rotavirus, tuberculosis and typhoid. Because the vaccine components are alive, they can spread to non-vaccinated subjects, extending the impact of vaccination to the community at large (See generally, Greenwood B. The contribution of vaccination to global health: past, present and future. (2014). Philosophical transactions of the Royal Society of London. Series B, Biological sciences, 369(1645), 20130433. doi:10.1098/rstb.2013.0433).

Live attenuated virus vaccines are a favored vaccination strategy, in part due to their previous success with the yellow fever virus vaccine, YF-17D, in the 1930s. (Ghaffar, K. A. et al, “Fast Tracks and Roadblocks for Zika Vaccines,” Vaccines (2018) 6, 77; doi: 10.3390/vaccines040077). A single dose of YF-17D vaccine, for example, is able to induce high titers of neutralizing antibody (nAb) which confer protection on at least 95% of recipients (Id., citing Barrett A. D., Teuwen D. E. Curr. Opin. Immunol. (2009) 21: 308-313. doi: 10.1016/j.coi.2009.05.018; Bonaldo, M C et al., Hum. Vaccin. Immunother. (2014) 10: 1256-1265. doi: 10.4161/hv.28117). This strategy has been employed with many other diseases, including polio, measles and mumps (Id., citing Plitnick L. M. Chapter 9—Global Regulatory Guidelines for Vaccines. In: Plitnick L. M., Herzyk D. J., editors. Nonclinical Development of Novel Biologics, Biosimilars, Vaccines and Specialty Biologics. Academic Press; San Diego, Calif., USA: (2013). pp. 225-241). Moreover, the production of attenuated vaccines is cost effective and fairly simple in comparison to other vaccine strategies.

While a live attenuated vaccine has the advantage of being able to elicit immune responses with a single dose, drawbacks include its limited use in immunocompromised or pregnant patients due to the risk of adverse effects. Indeed, because these vaccines contain live virus, mutations may occur in the attenuated vaccine strain with a reversion to virulence, as seen with oral polio vaccine, which causes paralysis in about one in two million recipients. Further, they may cause significant illness in subjects with impaired immunity, as has been seen with the anti-tuberculosis vaccine Bacille Calmette Guerin (BCG) when given to immunodeficient patients, including those with human immunodeficiency virus (HIV) infection.

Next, researchers developed killed vaccines where the pathogens were killed and then used. These vaccines were usually poorly immunogenic and often caused significant side effects, so that whole-cell vaccines have largely given way to subunit vaccines, among other types of vaccines. (See generally, Greenwood B. The contribution of vaccination to global health: past, present and future. (2014). Philosophical transactions of the Royal Society of London. Series B, Biological sciences, 369(1645), 20130433. doi:10.1098/rstb.2013.0433). Subunit vaccines comprise a fragment of a pathogen, i.e. a protein, or peptides (Ghaffar, K. A. et al, “Fast Tracks and Roadblocks for Zika Vaccines,” Vaccines (2018) 6, 77; doi: 10.3390/vaccines040077). While subunit vaccines are generally a safer choice, because they tend to be less immunogenic, an adjuvant and/or multiple doses are required.

The use of mRNA vaccines is a relatively new trend that has gained popularity (Ghaffar, K. A. et al, “Fast Tracks and Roadblocks for Zika Vaccines,” (2018) Vaccines 6, 77; doi: 10.3390/vaccines040077 citing Plitnick L. M. Chapter 9—Global Regulatory Guidelines for Vaccines. In: Plitnick L. M., Herzyk D. J., editors. Nonclinical Development of Novel Biologics, Biosimilars, Vaccines and Specialty Biologics. Academic Press; San Diego, Calif., USA: (2013). pp. 225-241). As the minimal genetic construct, mRNA contains only the elements required for expression of the specific encoded protein region. In addition, mRNA is incapable of interacting with the genome, but instead acts only as a transient carrier of information. Other advantages for its use as a vaccine platform include its safety profile (Id. citing Lundstrom, K., Futre Sci. OA (2018) 4: F50300 doi: 10.4155/fsoa-2017-0151). However, one of the disadvantages of utilizing mRNA as an approach to vaccine design is its rapid degradation by ribonucleases.

DNA vaccines are one of the earliest vaccine platforms to be proposed for human clinical trials following the ZIKV outbreak (Id). The use of genetically engineered DNA plasmids encoding various antigens to induce both humoral and cellular responses also has been explored against various infectious diseases caused by parasites (Id. citing Cherif, M S et al, Vacine (2011) 29: 9038-9050; Cheng, P C et al., PLoS Neg. Trop Dis. (2016) 10: e00044594; doi: 10.1371/journal.pntd.0004459), bacteria (Id., citing Li, X. et al., Clin. Vaccine Immunol. 2012; 19:723-730. doi: 10.1128/CVI.05700-11; Albrecht, M T, et al., Med. Microbiol. (2012) 65: 505-509 doi: 10.1111/j.1574-695X.2012.00974.x). and other viruses (Id., citing Donnelly, J J et al., Nature Med. (1995) 1: 583-597 doi: 10.1038/nm0695-583; Porter, K R et al., Vaccine (2012) 30: 36-341 doi: 10.1016/j.vaccine.2011.10.085).

Adenovirus vectors whereby the vector expresses an unknown antigenic protein have been well studied for gene and cancer therapy and vaccines (Id). Apart from its extensive safety profile, the advantages of utilizing an adenovirus vector are that it is relatively stable, easy to attain high titers and able to infect multiple cell lines which attributes to its potency. Even though recombinant adenoviral vectors are widely used today thanks to its high transduction efficiency and transgene expression, there is likelihood for pre-existing immunity against the vector, because most of the population has been exposed to adenovirus (Id). This has been proven detrimental in a human immunodeficiency virus (HIV-1) phase IIb vaccine trial in which the vector-based vaccines provided favorable conditions for HIV-1 replication (Id., citing Smaill, F. et al., Sci. Transl. Med. (2013) 5: 205ra134. doi: 10.1126/scitranslmed.3006843).

Developing the next generation of vaccines will be increasingly challenging, as many of the organisms to which they are targeted have complex structures and life cycles (e.g., the malaria parasite), or are very effective at outwitting the human immune response through antigenic diversity (e.g., HIV and influenza viruses). Development of new vaccines against other important infectious disease targets such as dengue or novel coronaviruses should theoretically be easier using established technologies, but the modest efficacy of a recently tested dengue vaccine emphasizes that challenges remain even in the development of more conventional vaccines (Greenwood B. The contribution of vaccination to global health: past, present and future. (2014). Philosophical transactions of the Royal Society of London. Series B, Biological Sciences, 369(1645), 20130433. doi:10.1098/rstb.2013.0433).

As a result, other vaccination strategies are being developed in an attempt to overcome the above addressed failures.

Exemplary infectious agents that afflict current human populations around the globe include the following.

### Viruses

General Principles A virus is a very small, infectious, obligate intracellular parasite comprising a genome of either DNA or RNA.

Viruses depend on contact with a compatible host cell for replication. Viral replication begins with attachment of a virus to a host cell, which is followed by entry of the viral genome into the host. Within an appropriate host cell, the viral genome is replicated and directs the synthesis, by cellular systems, of other virion components. Progeny virions are formed by de novo assembly from newly synthesized components within the host cells. A progeny virion assembled during the infectious cycle is the vehicle for transmission of the viral genome to the next host cell or organism, where its disassembly leads to the beginning of the next infectious cycle. To initiate an infection in an individual host, sufficient virus must be available to initiate infection, the cells at the site of infection must be susceptible and permissive for the virus, and the local host antiviral defense systems must be absent or at least initially ineffective. A severe virus infection attacks the host on multiple fronts. Some host defenses may be overcome passively by an overwhelming inoculum of virus. In addition, many viruses have evolved active mechanisms for bypassing or disarming host defenses.

The two primary patterns of infection are acute infections and persistent infections. In acute infections, some viruses rapidly kill the cell while producing a burst of new infectious particles (cytopathic viruses), while others infect cells and actively produce infectious particle without causing immediate host cell death (noncytopathic viruses). In persistent infections (e.g., latent infections, slow, abortive and transforming infections), some viruses infect, but neither kill the cell nor produce any viral progeny. Replicated viruses remain inert unless they attach to the surface of another compatible host cell. [Principles of Virology. Flint, S J, Enquist L W Q, Krug, R M, Racaniello, V R, Skalka, A M, Eds. (2000) ASM Press, Washington, D.C., Chapter 15, pp. 519-551]

To initiate an infection in an individual host, sufficient virus must be available to initiate infection, the cells at the site of infection must be susceptible and permissive for the virus, and the local host antiviral defense systems must be absent or at least initially ineffective. Common sites of viral entry include the mucosal linings of the respiratory, alimentary and urogenital tracts, the outer surface of the eye (conjunctival membranes or cornea), and the skin. Following replication at the site of entry, virus particles can remain localized or can spread to other tissues. Local replication in the respiratory tract is characteristic of influenza virus, parainfluenza virus, rhinovirus and respiratory CoV; replication of rotaviruses and enteric corona- and adenoviruses is restricted to the alimentary tract, and replication of some papillomaviruses is confined to the skin. Local spread of the infection in the epithelium occurs when newly released viruses infects adjacent cells. An infection that spreads beyond the primary site of infection is said to be disseminated. If many organs become infected, the infection is described as systemic.

A severe virus infection attacks the host on multiple fronts. Some host defenses may be overcome passively by an overwhelming inoculum of virus. Directional release of virus particles from polarized cells at the musosal surface can avoid local host defenses and facilitate spread. For example, since virus particles released from the basolateral surfaces of polarized epithelial cells have been moved away from the defenses of the luminal surface, their release provides access to the underlying tissues and may facilitate systemic spread. Viruses that escape from local defenses to produce a disseminated infection often do so by entering the bloodstream (hematogenous spread). In addition, many viruses have evolved active mechanisms for bypassing or disarming host defenses.

### I) Retroviridae Viruses

### A) Overview of the Retroviridae Family

The family Retroviridae is a large diverse group of enveloped RNA viruses which include the following genera: Alpharetrovirus, Betaretrovirus, Deltaretrovirus, Epsilonretrovirus, Gammaretrovirus, Lentivirus, and Spumavirus. Retroviridae viruses include members of the Lentivirus genus, which are complex retroviruses that include human pathogens such as Human Immunodeficiency Virus (HIV) and Equine Infectious Anemia (EIAV), and feline immunodeficiency virus. Other Retrovirdae virus families include Human T-Lymphotrophic viruses (HTLV), and Hepadnaviridae which encompasses Hepatitis B (HVB). Members of Retroviridae are characterized by the ability to transcribe their RNA genome into linear double-stranded DNA during their replication cycle with a reverse transcriptase enzyme. During the replication cycle, viral dsDNA is usually integrated into the host genome as a DNA provirus which can remain silent (i.e., latent) or become transcriptionally active to produce virions (Fermin, Gustavo, and Paula Tennant. Viruses: Molecular Biology, Host Interactions and Applications to Biotechnology, edited by Jerome E. Foster, Elsevier Science & Technology (2018). ProQuest Ebook Central, https://ebookcentral.proquest.com/lib/jhu/detail.action?docID=5322098).

Many lentiviral viruses may be characterized by a long latency period and progressive infection in which the virus evades the immune response of the host. Lentiviruses insert genetic information into the deoxyribonucleic acid (DNA) of the host cell and have the unique ability to replicate in dividing and non-dividing cells.

All replication-competent retroviruses, including HIV, contain the following three genes: gag (group antigen, encoding the core and matrix proteins, p24 and p17), pol (polymerase, encoding the enzymatic proteins, reverse transcriptase, RNAase, protease and integrase), and env (encoding the envelope and transmembrane glycoproteins, gp 120 and gp 41) (Welles, L. and Yarchoan, R., In Antimicrobial Therapy and Vaccines. Yu, V L, Merigan, Jr, T C and Barriere, S L Eds, Williams & Wilkins, Baltimore, (2005), pgs.1264-1287). They share the presence of antigens, the ability to replicate, and a viral envelope.

### i) Human Immunodeficiency Virus (HIV)

Human Immunodeficiency Virus (HIV) is a complex retrovirus which may be transmitted to humans from primates and between humans through the exchange of fluid, such as semen, vaginal and anal mucus, blood, and breast milk, through cuts, openings or mucous membranes of the human body. HIV is a rapidly mutating and recombining RNA virus that exhibits considerable genetic diversity with nine subtypes within just the major group of HIV Type I (HIV-1), rapid turnover rates, and persistency. HIV-1 can further be classified into 4 viral groups, or isolates: M, N, O, and P. The other major group of HIV is HIV Type 2 (HIV-2) (Moss, J. (2013) “HIV/AIDS Review” Radiologic Technology, Vol. 84, No. 3, 247-267). The majority of HIV/AIDS related deaths are concentrated in South Africa, however, the HIV infection is seen globally, including in sub-Saharan Africa, the United States, Europe, and Asia (Id.).

### B) Structure-Based Functional Analyses of Retroviridae Viruses

The mature HIV particle is round, measuring approximately 100 nm in diameter, with an outer lipid membrane as its envelope. The envelope contains 72 knobs, composed of trimers of the Env proteins. The trimers of gp120 surface protein (SU) are anchored to the membrane by the trimers of the transmembrane protein gp41 (TM). Conformation-dependent neutralizing epitopes are found on the gp120 protein. These are present on the native protein but are only partially expressed on the unfolded denatured protein. The viral envelope is composed of a lipid bi-layer and, in mature virus particles, the envelope proteins SU and TM. It covers the symmetrical outer capsid membrane, which is formed by the matrix protein (MA, p17). The conical capsid is assembled from the inner capsid protein p24 (CA). Depending on the section plane, the capsid appears as a cone, a ring or an ellipse. The tapered pole of the capsid is attached to the outer capsid membrane. Two identical molecules of viral genomic RNA are located inside the capsid and several molecules of the viral enzymes RT/RNase H and IN bound to the nucleic acid. Also present in virus particles are oligopeptides that are generated after release from the cell during the maturation of virions by proteolytic processing of the precursor proteins (p55, p160). (German Advisory Committee Blood (Arbeitskreis Blut), Subgroup ‘Assessment of Pathogens Transmissible by Blood’. (2016) “Human Immunodeficiency Virus (HIV).” Transfusion medicine and hemotherapy: offizielles Organ der Deutschen Gesellschaft fur Transfusionsmedizin and Immunhamatologie, Vol. 43, 3: 203-22. doi:10.1159/000445852)

C) Immune Response to Infection with Retroviruses

During the first stage of infection, known as the primary infection stage, the immune system of the infected person begins responding to the virus by generating HIV antibodies (in response to HIV antigens, a process known as seroconversion) and cytotoxic lymphocytes. Following seroconversion, a clinically asymptomatic period normally follows initial HIV infection, where levels of HIV in the peripheral blood decrease but function highly in the lymph nodes to destroy CD4 lymphocytes. The immune system becomes progressively damaged as the patient's immune system deteriorates from excessive damage to tissues and lymph nodes, viral mutation and increased destruction and reduced replacement of T cells (see Moss, J. (2013) “HIV/AIDS Review” Radiologic Technology, Vol. 84, No. 3, 247-267). In the second stage, laboratory results indicate 14% to 29% CD4+ T cells per μL of blood and mild symptoms are perceived. In the third stage, CD4+ T cell count is below 14% and advanced symptoms are seen. By the fourth stage, acquired immunodeficiency syndrome has developed and severe symptoms are seen. (Id.).

A substantial reduction in the number of T cells seriously weakens the immune system. As CD4 lymphocyte counts decrease to fewer than 200 cells/4, of blood, symptomatic HIV infection can be triggered by the emergence of certain opportunistic infections that the immune system would normally prevent. Examples include pneumonia, diarrhea, eye infections, and meningitis. HIV patients are also susceptible to cancers and illnesses, for example Kaposi sarcoma, non-Hodgkin lymphoma, central nervous system lymphoma, HIV encephalopathy, progressive multifocal leukoencephalophathy, lymphoid interstitial pneumonia, and HIV wasting syndrome. (Id.).

HIV initially infects CD4+CCR5+ T cells. The virus then spreads via the blood from the mucosal-associated lymphoid tissue to other lymphoid tissue, especially in the gut associated lymphoid tissue where it can replicate liberally. See Id. In acute HIV-1 infection, memory CD4+ T cells are massively depleted from the lymphoid system, particularly in the gut involving both direct targeting by the virus and bystander activation-induced cell death (See Mattapallil J J, Douek D C, Hill B, Nishimura Y, Martin M, Roederer M (2005) Massive infection and loss of memory CD4⁺ T cells in multiple tissues during acute SIV infection. Nature 434: 1093-1097; see also Douek D C, Brenchley J M, Betts M R, Ambrozak D R, Hill B J, Okamoto Y, Casazza J P, Kuruppu J, Kunstman K, Wolinsky S, et al. (2002) HIV preferentially infects HIV-specific CD4⁺ T cells. Nature 417: 95-98). This applies to all memory CD4+ T-cell populations but those specific for HIV may be preferentially infected and destroyed (See Douek D C, Roederer M, Koup R A (2009) Emerging concepts in the immunopathogenesis of AIDS. Annu Rev Med 60: 471-484). However, the percentage of HIV-specific CD4+ T cells that are infected, even in the presence of high level viremia, is typically only a few percent or less, suggesting that the majority of these cells somehow escape infection despite being activated at a time of very high viremia.

Despite initial and persistent damage to CD4+ T cells, and a lack of detectable HIV-specific CD4+ T helper cells, the magnitude and breadth of CD8+ T-cell responses to HIV in infected humans were found to be robust, with direct effector function of such a magnitude that it could be readily detected in freshly isolated lymphocytes from peripheral blood and bronchoalveolar lavage in persons with AIDS. (Walker, B and McMichael, A. “The T-Cell Response to HIV. (2012 November)” Cold Spring Harb Perspect Med. 2(11): a007054; citing Murray, H W et al. (1984) “Impaired production of lymphokines and immune (γ) interferon in the acquired immunodeficiency syndrome.” N. Engl. J. Med. 310: 883-889; Lane, H C et al. (1985) “Qualitative analysis of immune function in patients with the acquired immunodeficiency syndrome; Evidence for a selective defect in soluble antigen recognition,” N. Engl. J. Med. 313: 79-84). Acute phase CD8+ T-cell responses occur in the setting of acute phase proteins and proinflammatory cytokines. The initial response is narrowly directed, predominantly at epitopes in Env and Nef, regions that are among the most variable in the virus. The breadth of responses increases over time, as do the number of HLA alleles that are involved in recognition of infected cells. Immunization studies in animal models indicate that the CD8+ T-cell compartment has enormous expansion capacity, without affecting the size of the naïve CD4+, CD8+, or B-cell populations, and while preserving memory CD8+ T-cell populations to other pathogens. HIV-specific CD8+ T-cell responses remain detectable throughout the course of disease, and are actually broader and higher in persons with progressive infection than in those with controlled infection. (Id., citing (Vezys, V. et al. (2009) “Memory CD8 T cell compartment grows in size with immunological experience,” Nature 457: 196-199; Pereyra F. et al. (2008) “Genetic and immunologic heterogeneity among persons who control HIV infection in the absence of therapy,” J. Infect. Dis. 197: 563-571).

Specificity of responses during the chronic phase of infection repeatedly suggests that Gag targeting is associated with lower viral load. (Id., citing Edwards, B H et al. (2002) “Magnitude of functional CD8+ T-cell responses to the gag protein of human immunodeficiency virus type 1 correlates inversely with viral load in plasma. J Virol 76: 2298-2305; Zuniga, R. et al. (2006) Relative dominance of Gag p24—specific cytotoxic T lymphocytes is associated with human immunodeficiency virus control. J Virol 80: 3122-3125; Kiepiela et al. (2007) “CD8+ T-cell responses to different HIV proteins have discordant associations with viral load.” Nat Med 13: 46-53). In a large study of persons with clade C virus infection, the broader the Gag-specific response, the lower the viral load, and somewhat paradoxically, the broader the Env-specific response, the higher the viral load. (Id.; citing Kiepiela et al. (2007) “CD8+ T-cell responses to different HIV proteins have discordant associations with viral load.” Nat Med 13: 46-53; Ngumbela, K C et al. (2008) “Targeting of a CD8 T cell env epitope presented by HLA-B*5802 is associated with markers of HIV disease progression and lack of selection pressure.” AIDS Res Hum Retroviruses 24: 72-82).

Generally, in those infected with HIV-1, the T-cell responses are dominated by CD8+ T cells. These are much stronger than CD4+ T-cell responses which are damaged by the virus. (Id., citing (Ramduth, D et al. (2005) Differential immunogenicity of HIV-1 clade C proteins in eliciting CD8+ and CD4+ cell responses. J Infect Dis 192: 1588-1596). In murine models in which CD4+ T cells are depleted either with antibody infusion or genetically, CD8+ T-cell responses are greatly impaired. (Id., citing (Janssen, E M et al. (2003) CD4+ T cells are required for secondary expansion and memory in CD8+ T lymphocytes. Nature 421: 852-856; Shedlock, D J and Shen, H (2003) Requirement for CD4 T cell help in generating functional CD8 T cell memory. Science 300: 337-339; Sun, J C and Bevan, M J (2003) Defective CD8 T cell memory following acute infection without CD4 T cell help. Science 300: 339-342). On antigen stimulation, they expand rapidly to exhaustion and their IL-2-dependent progression to long term memory populations is abrogated. (Id., citing (Kamimura, D and Bevan, M J (2007) Naive CD8+ T cells differentiate into protective memory-like cells after IL-2 anti IL-2 complex treatment in vivo. J Exp Med 204: 1803-1812). In HIV-1 infection CD4+ T cells, though greatly depleted, are not entirely absent, but abnormalities in the development of CD8+ T-cell responses could be consistent with partial loss of CD4+ T-cell help, or impaired function of what cells remain (Id., citing (Pitcher, C J et al. (1999) HIV-1-specific CD4+ T cells are detectable in most individuals with active HIV-1 infection, but decline with prolonged viral suppression. Nat Med 5: 518-525).

Cross-sectional data in chronically infected persons indicate a link between strong CD4+ T-cell responses and effective CD8+ T-cell responses. (Id., citing (Kalams, S A et al. (1999) Association between virus-specific cytotoxic T-lymphocyte and helper responses in human immunodeficiency virus type 1 infection. J Virol 73: 6715-6720). Recent data implicate CD4+ T cells that make IL-21 as particularly important in maintaining CD8+ responses. (Id., citing (Chevalier M F, Jülg B, Pyo A, Flanders M, Ranasinghe S, Soghoian D Z, Kwon D S, Rychert J, Lian J, Muller M I, et al. HIV-1-specific interleukin-21⁺ CD4⁺ T cell responses contribute to durable viral control through the modulation of HIV-specific CD8⁺ T cell function. (2011) J Virol 85: 733-741; Williams L D, Bansal A, Sabbaj S, Heath S L, Song W, Tang J, Zajac A J, Goepfert P A Interleukin-21-producing HIV-1-specific CD8 T cells are preferentially seen in elite controllers. (2011) J Virol 85: 2316-2324)). While early studies showed a lack of CD4+ T-cell responses, it has been reported that when patients were treated very early with antiretroviral drugs, strong CD4+ T-cell responses to HIV antigens could be rescued.

Antiviral CD8+ T cells were first identified as T cells that mediate lysis of virus-infected cells and are often referred to as cytotoxic T lymphocytes (Id., citing Plata F., et al. (1975) Primary and secondary in vitro generation of cytolytic T lymphocytes in the murine sarcoma virus system. Eur J Immunol 5: 227-233). Although most antigen-specific CD8+ T cells have this activity, they can use other effector mechanisms in addition. These include production of interferon-γ, IL-2, TNF-α, MIP-1α (renamed CCL3), MIP-1β (CCL4), and RANTES (CCL5). However, this effector function may not always be present and may take several days to appear. In contrast memory CD8+ T cells respond rapidly producing interferon-γ within a few hours. (Id., citing Lalvani, A. et al. (1997) Rapid effector function in CD8+ memory T cells. J Exp Med 186: 859-865). Production of lytic granules requires a bit longer but once activated, effector memory CD8+ T cells can release perforin and granzymes within minutes. (Id., citing Barber, D L et al. (2003) Cutting edge: Rapid in vivo killing by memory CD8 T cells. J Immunol 171: 27-31). The delay in activating lytic functions in memory T cells probably protects the body from autoimmune attack when the T cell receptor (TCR) encounters weakly binding self antigens.

Although lytic potential may still be essential during chronic infection, once viral set point is established, the other functions of CD8+ T cells may become more important, although lytic potential may still be essential. (Id., citing (Betts, M R and Harari, A (2008) Phenotype and function of protective T cell immune responses in HIV. Curr Opin HIV AIDS 3: 349-355). In patients who control virus well, the T cells are more quiescent than in acute infection. Many studies have shown that T cells in those who control HIV-1 well are polyfunctional, showing not only cytolytic potential but also have the capacity to produce cytokines and chemokines, although it is not clear whether this is cause or effect. (Id., citing (Betts, M R and Harari, A (2008) Phenotype and function of protective T cell immune responses in HIV. Curr Opin HIV AIDS 3: 349-355). Prolonged antigen stimulation in the absence of excessive activation and exhaustion, as occurs in slow progressors, could favor expression of multiple functions. Production of IL-2 may be important in the long term persistence of CD8+ T cells and can be provided by the CD8+ T cell itself or by CD4+ T cells, which survive much better in those whose disease progresses slowly. (Id.; citing (Rosenberg, E S et al. (1997) Vigorous HIV-1-specific CD4+ T cell responses associated with control of viremia. Science 278: 1447-1450; Zimmerli, S C et al. (2005) HIV-1-specific IFN-γ/IL-2-secreting CD8 T cells support CD4-independent proliferation of HIV-1-specific CD8 T cells. Proc Natl Acad Sci 102: 7239-7244). Similar observations have been made in HIV-2 infection in which elite controllers are relatively common. (Id.; citing (Duvall, M G et al. (2008) Polyfunctional T cell responses are a hallmark of HIV-2 infection. Eur J Immunol 38: 350-363). These findings are entirely consistent with data in CD4+ T-cell-depleted mice that show the importance of IL-2 in the maintenance of long-term CD8+ T-cell memory. (Id.; citing Williams M A, Tyznik A J, Bevan M J (2006) Interleukin-2 signals during priming are required for secondary expansion of CD8⁺ memory T cells. Nature 441: 890-893)).

Evidence shows that CD8+ T cells are vital in controlling early HIV infection. Studies of human tissue samples have revealed that T_{RM} are generated in response to HIV infection in multiple locations, including the gastrointestinal tract and the female reproductive tract. Furthermore, individuals who appeared to naturally control infection had T_{RM} that were capable of producing the highest polyfunctional immune responses when compared to individuals who did not. (Muruganandah, V., Sathkumara, H. D., Navarro, S., & Kupz, A. (2018). A Systematic Review: The Role of Resident Memory T Cells in Infectious Diseases and Their Relevance for Vaccine Development. Frontiers in Immunology, 9, 1574. doi:10.3389/fimmu.2018.01574). However, the T_{RM} population within the HIV-specific CD8+ T cell compartment in individuals who controlled infection was under-represented when compared to individuals who were viremic. (Id.)

Similar to other infections in various sites, CD8+ T_{RM} in the context of HIV can be subdivided into two subsets based on the expression of CD103 (also called human mucosal lymphocyte antigen 1, alpha E beta 7 Analysis of the ectocervical epithelium and menstrual blood revealed that HIV-infected women were more likely to have CD103—T_{RM} when compared to healthy individuals. This reduced expression of CD103 may be explained by the HIV-induced depletion of CD4+ T cells which appear to be vital in providing help to CD8+ T cells for up-regulating CD103. The CD103—populations of the ectocervix resided closer to the basement membrane of the epithelium when compared to their CD103+ counterparts. The CD103+ population from infected individuals appears to express higher levels of PD-1. In a separate study, adipose PD-1+ CD4+ T_{RM}, appeared to remain relatively inactive during HIV infection and may serve as a reservoir for HIV. As such chronically activated T_{RM} and T_{RM} exposed to immunomodulated environments (such as the adipose tissue) may be unable to elicit a full effector response, favoring the progression of HIV infection. (Muruganandah, V., Sathkumara, H. D., Navarro, S., & Kupz, A. (2018). A Systematic Review: The Role of Resident Memory T Cells in Infectious Diseases and Their Relevance for Vaccine Development. Frontiers in Immunology, 9, 1574. doi:10.3389/fimmu.2018.01574).

It also appears that HIV has the ability to disrupt CCR5-mediated CD8+ T cell migration into the cervical mucosa, thereby impairing the development of T_{RM} populations. Human studies suggest that T_{RM}, especially CD8+ T_{RM}, play an important role in combating HIV infection. In a Simian Immunodeficiency Virus model of rhesus macaques, intravenous administration of SIVmac239Δnef generated a population of CD8+ T_{RM} in vaginal tissue and the gut that participated in protection. In a murine model, a mucosal vaccination strategy in which intranasal administration of an influenza-vector expressing the HIV-1 Gag protein p24 followed by an intravaginal booster induced CD8+ T_{RM} in the vagina. Antigen stimulation of these CD8+ T_{RM} resulted in the recruitment of B cells, natural killer cells, and CD4+ T cells. While the recruitment of innate and adaptive immune cells may be beneficial in early viral clearance, the recruitment of CD4+ T cells may be detrimental in the context of HIV as they are the target for HIV. Hence, incidental recruitment of CD4+ T cells to sites of HIV entry (female reproductive tract and rectum) by prime and pull vaccination strategies may unintentionally increase susceptibility to infection. (Muruganandah, V., Sathkumara, H. D., Navarro, S., & Kupz, A. (2018). A Systematic Review: The Role of Resident Memory T Cells in Infectious Diseases and Their Relevance for Vaccine Development. Frontiers in Immunology, 9, 1574. doi:10.3389/fimmu.2018.01574).

### D) Retroviridae Viruses Vaccine Development

Vaccine development for retroviruses, lentiviruses, and specifically HIV have been largely unsuccessful. Both antibodies and cytotoxic T lymphocytes are produced upon infection with HIV (Seabright, G. E. et al., (2019) “Protein and Glycan Mimicry in HIV Vaccine Design,” J. MOI/Biol. 431 (12): 2223-2247). However, there have been some protective immune responses to vaccines that invoke T cell-mediated immunity, polyfunctional antibody responses, antibody-dependent cellular cytotoxicity, and broadly neutralizing antibodies (bNab) (MacGregor, R. et al. (2002) “T-cell responses induced in normal volunteers immunized with a DNA-based vaccine containing HIV-1 env and rev.” AIDS 16, 2137-2143).

Despite the growing understanding of human immunodeficiency virus (HIV) disease pathogenesis and the structure of its key antigenic targets, efforts to develop effective vaccines against HIV continue to fail.

The role of T cells in mediating a cure has been previously shown in studies of elite controllers. These patients maintain undetectable levels of HIV, which has been associated with a significantly increased breadth of Gag-specific CD8+ T cell response, when compared to chronic progressors and individuals with ART suppressed HIV. However, DNA vaccines that invoke T cell mediated immunity developed for HIV still lack the ability to induce long-term immune responses. For example, a DNA vaccine that encoded env and rev was shown to induce CD4+ T cell responses and poorly induce CD8+ T cell responses (MacGregor, R. et al. (2002) “T-cell responses induced in normal volunteers immunized with a DNA-based vaccine containing HIV-1 env and rev.” AIDS 16, 2137-2143). Similar results were seen in DNA vaccines that encode gag and pol genes (Tavel, J. A., et al., (2007) “Safety and Immunogenicity of a Gag-Pol candidate HIV-1 DNA vaccine administered by a need-free device in HIV-1-seronegative subjects.” J. AIDS 44, 601-605).

### A) Coronaviridae

Coronaviruses (CoVs), a large family of single-stranded RNA viruses, can infect a wide variety of animals, causing respiratory, enteric, hepatic and neurological diseases [Yin, Y., Wunderink, R G, Respirology (2018) 23 (2): 130-37, citing Weiss, S R, Leibowitz, I L, Coronavirus pathogenesis. Adv. Virus Res. (2011) 81: 85-164]. Human coronaviruses, which were considered to be relatively harmless respiratory pathogens in the past, have now received worldwide attention as important pathogens in respiratory tract infection.

As the largest known RNA viruses, CoVs are further divided into four genera: alpha-, beta-, gamma- and delta-groups; the beta group is further composed of A, B, C and D subgroups. [Xia, S. et al. Sci. Adv. (2019) 5: eaav4580].

CoVs are enveloped with a non-segmented, positive sense, single strand RNA, with size ranging from 26,000 to 37,000 bases; this is the largest known genome among RNA viruses [Yang, Y. et al., J. Autoimmunity (2020) doi.org/10.1016/j.jaut.2020.102434, citing Weiss, S R et al. Microbiol. Mol. Biol. Rev. (2005) 69 (4): 635-64]. The viral RNA encodes structural proteins, and genes interspersed within the structural genes, some of which play important roles in viral pathogenesis [Yang, Y. et al., J. Autoimmunity (2020) doi.org/10.1016/j.jaut.2020.102434, citing Fehr, A R, Perlman, S. Methods Mol. Biol. (2015) 1282: 1-23; Zhao, L. et al. Cell Host Microbe (2012) 11(6): 607-16]. The spike protein (S) is responsible for receptor binding and subsequent viral entry into host cells; it consists of S1 and S2 subunits. The membrane (M) and envelope (E) proteins play important roles in viral assembly; the E protein is required for pathogenesis [Id., citing DeDiego, M L, et al. J. Virol. (2007) 81(4): 1701-13; Nieto-Torres, J L et al. PLoS Pathog. (2014) 10(5): e1004077]. The nucleocapsid (N) protein contains two domains, both of which can bind virus RNA genomes via different mechanisms, and is necessary for RNA synthesis and packaging the encapsulated genome into virions. [Yang, Y. et al., J. Autoimmunity (2020) doi.org/10.1016/j.jaut.2020.102434., citing Fehr, A R, Perlman, S. Methods Mol. Biol. (2015) 1282: 1-23; Song, Z. et al. Viruses (2019) 11(1): 59; Chang, C K et al., J. Biomed. Sci. (2006) 13(1): 59-72; Hurst, K R, et al. J. Virol. (2009) 83 (14): 7221-34]. The N protein also is an antagonist of interferon and viral encoded repressor (VSR) of RNA interference (RNAi), which benefits viral replication [Id., citing Cui, L. et al. J. Virol. (2015) 89 (17): 9029-43].

Before December 2019, six coronavirus species had been identified to infect humans and cause disease. Among them, infections caused by H-CoV-229E and HCoV-NL63 in the alpha group, HCoV-OC43 and HCoV-HKU1 in beta subgroup A are frequently mild, mostly causing common cold symptoms [Xu, X. et al. Eur. J. Nuclear Medicine & Molec. Imaging (2020) doi.org/10.1007/s00259-020-04735-9, citing Su, S. et al. Trends Microbiol. (2016) 24: 490-502]. The other two species, severe acute respiratory syndrome coronavirus (SARS-CoV) in beta subgroup B and Middle East respiratory syndrome coronavirus (MERS-CoV) in beta subgroup C, have a different pathogenicity and have caused fatal illness [Id., citing Cui, J. et al. Nat. Rev. Microbiol. (2019) 17: 181-92]. Human-to-human transmission of SARS-CoV and MERS-CoV occurs mainly through nosocomial transmission: from 43.5-100% of MERS patients in individual outbreaks were linked to hospitals, [Id., citing Hunter, I C et al. Transmission of Middle East respiratory syndrome coronavirus infections in healthcare settings, Abu Dhabi. Emerg. Infect. Dis. (2016) 22: 647-56. Osong Public Health Res. Perspect. (2015) 6: 269-78], which was similar in SARS patients. [Anderson, R M et al. Philos. Trans. R. Soc. Lond. B. Biol. Sci. (2004) 359: 1091-105]. A study from the Republic of Korea revealed that index patients who transmitted to others had more non-isolated days in the hospital, body temperature of ≥38.5° C. and pulmonary infiltration of ≥3 lung zones. [Id., citing Kang, C K, et al. J. Korean Med. Sci. (2017) 32: 744-49]. Transmission between family members occurred in only 13-21% of MERS cases and 22-39% of SARS cases. [Id., citing Kang, C K, et al. J. Korean Med. Sci. (2017) 32: 744-49]. Another Korean study suggested that transmission of MERS from an asymptomatic patient is rare. [Id., citing Moon, S Y, Son, J S. Clin. Infect. Dis. (2017) 64: 1457-58]. In contrast to SARS-CoV and MERS-CoV, direct human-to-human transmission was not reported for the other four HCoVs. [Id., citing Woo, P C et al. Hong Kong Med. J. (2008) 15 (Suppl. 9): 46-47].

Severe Acute Respiratory Syndrome Coronavirus 2 (SARS-CoV-2) is the seventh member of the coronaviruses that infects humans [Zhu, N. et al. N. Engl. J. Med. (2020) 382: 727-33].

### B) SARSCoV

SARS CoV (or SARSCoV-1) is an enveloped RNA virus that replicates with transcription of discontinuous nested messenger RNA (mRNA). The reservoir for the virus is thought to be civet cats, a nocturnal mammal considered a delicacy in southern China. Horseshoe bats may also be a reservoir. The incubation period is 2 to 7 days before symptom onset, and peak viral shedding in respiratory secretions occurs relatively late, between 6 and 11 days. The virus is spread through respiratory secretion shedding and via contact with fomites (meaning inanimate objects that can become contaminated with infectious agents and serve as a mechanism for transfer between hosts). Airborne transmission, particularly on international flights, contributed to superspreader outbreak phenomena. Several epidemiological studies using logistic regression showed that older age and underlying comorbid conditions (diabetes, chronic obstructive pulmonary disease, hepatitis B infection, cancer, and cardiac disease) were associated with worse outcomes including ICU admission, mechanical ventilation, and death. [Huang, E-S, Johnson, R A, Nature Medicine (2000) 6: 863-64], citing Peiris, J S, et al. Lancet (2003) 361 (9366): 1319-25; Booth C M, et al. JAMA (2003) 289 (21): 2801-97].

Postmortem pathology studies and various in vitro and in vivo model systems of SARS CoV infection suggest that the virus enters through the respiratory tract and binds ACE2 in the alveolar epithelium. Infection is followed by serological evidence of increased ACE activity and decreased ACE2 activity. The signaling pathways that are activated by binding the SARS CoV to ACE2 and the subsequent downstream cytokine elaboration appear to share common features with other mechanisms of acute lung injury (ALI) and result in a pathological phenotype indistinguishable from other mechanisms of lung injury. [Hendrickson, C M, Matthay, M A Semin. Respir. Crit. Care Med. (2013) 34: 475-86]

### C) MERS-CoV

The CoV that causes Middle East Respiratory Syndromes (MERS-CoV) is enzootic (meaning a disease that regularly affects animals in a particular district or at a particular season) in dromedary camels across the Arabian Peninsula and in parts of Africa, causing mild upper respiratory tract illness in its camel reservoir and sporadic, but relatively rare human infections. The first known cases of MERS occurred in 2012 in Jordan. Precisely how the virus transmits to humans remains unknown but close and lengthy exposure appears to be a requirement. The majority of human cases occurred in the Kingdom of Saudi Arabia. [McKay, I M & Arden, K E. Virology J. (2015) 12: 222]

In humans, MERS is mostly a lower respiratory tract (LRT) disease involving fever, cough, breathing difficulties and pneumonia that may progress to acute respiratory distress syndrome (ARDS), multiorgan failure and death in 20% to 40% of those infected. However, MERS-CoV has also been detected in mild and influenza-like illnesses and in those with no signs or symptoms. Older males most obviously suffer severe disease and MERS patients often have comorbidities. Compared to SARS, MERS progresses more rapidly to respiratory failure and acute kidney injury (it also has an affinity for growth in kidney cells under laboratory conditions), is more frequently reported in patients with underlying disease and is more often fatal. Most human cases of MERS have been linked to lapses in infection prevention and control in healthcare settings, with approximately 20% of all virus detections reported among healthcare workers and higher exposures in those with occupations that bring them into close contact with camels. Serosurveys found widespread evidence of past infection in adult camels and limited past exposure among humans.

Studies have established that the mean incubation period for MERS is five to six days, ranging from two to 16 days, with 13 to 14 days between when illness begins in one person and subsequently spreads to another [Id., citing Assiri, A. et al. Lancet Infect. Dis. (2013) 13: 752-61; Memish, Z A et al. N. Engl. J. Med. (2013) 368: 2487-94; Assiri, A. et al. N. Engl. J. Med. (2013) 369: 407-16; Ki, M. Epidemiol. Heath (2015) 37 doi: 10.4178/epih/e2015033]. Among those with progressive illness, the median time to death is 11 to 13 days, ranging from five to 27 days [Id., citing Assiri, A. et al. N. Engl. J. Med. (2013) 369: 407-16; Ki, M. Epidemiol. Heath (2015) 37 doi: 10.4178/epih/e2015033]. Fever and gastrointestinal symptoms may be early symptoms indicating the onset of disease, after which symptoms decline, only to be followed by a more severe systemic and respiratory syndrome [Id., citing Kraaij-Dirkzwager, M. et al. Euro Surveill. (2014) 19: 20817-7; Mailles, A. et al. EuroSurveill. (2013) 18: 24].

Transmission of MERS-CoV is defined as sporadic (not sustained), intra-familial, often healthcare associated, inefficient and requiring close and prolonged contact [Id., citing Memish, Z A et al. N. Engl. J. Med. (2013) 368: 2487-94; Drosten, C. et al. N. Eng. J. Med. (2014) 371: 838-35; Puzelli, S. et al. EuroSurveill. (2013) 18 (34): 1; Omrani, A S et al. Int. J. Infect. Dis. (2013) 17: e668-e672; Health Protection Agency UKNCIT. Euro Surveill. (2013) 18: 20427; Memish, Z A et al. Int. J. Infect. Dis. (2014) 29: 307-8; Drosten, C. et al. Clin. Infect. Dis. (2015) 60: 367-77]. In a household study, 14 of 280 (5%) contacts of 26 MERS-CoV positive index patients were RNA or antibody positive; the rate of general transmission, even in outbreaks, is around 3% [Id., citing Drosten, C. et al. N. Engl. J. Med. (2014) 371: 828-35]. The majority of human cases of MERS-CoV, even when numbers appear to increase suddenly, do not readily transmit to more than one other human so localized epidemics of MERS-CoV are not self-sustaining [Id., citing Busch, C T & Oraby, T. Lancet (2013) 382: 662-64; Breban, R. et al. Lancet (2013) 382: 694-99; Cauchemez, S. et al. Lancet Infect. Dis. (2014) 14: 50-56]. Among all humans reported to be infected, nearly 40% died. [Id.]

### D) SARSCoV-2

The coronavirus-19 disease (COVID-19) pandemic caused by SARS-CoV-2 has exceeded 11 million cases worldwide, and caused more than 500,000 deaths in 216 countries [Kuri-Cervantes, L. et al. Sci. Immunol. (2020) 10.1126/sciimmuol.abd7114]. Due to efficient person-to-person transmission, the SARS-CoV-2 pandemic is still evolving. True aerosols drive asymptomatic transmission. The extent of the disease, its epidemiology, pathophysiology and clinical manifestations are being documented on an ongoing basis [Guan w. et al. N. Engl. J. Med. (2020) 382: 1708-20; Yang, Y. et al., J. Autoimmunity (2020) doi.org/10.1016/j aut.2020.102434].

Infected patients predominantly present with fever, cough, and radiological ground glass lung opacities, which resemble SARS-CoV and MERS-CoV infections [Huang, C. et al. Lancet (2020) 395: 497-506]. The absence of fever in COVID-19 is more frequent than in SARS-CoV (1%) and MERS-CoV infection (2%), so afebrile patients may be missed if the surveillance case definition focuses on fever detection. [Guan, W., et al. New Engl. J. Med. (2020) 382: 1708-2]. The incubation period is generally 3-7 days, the longest not more than 14 days. [Pan, Y. et al. European Radiol. (2020) doi.org/10.1007/s00330-020-06731-x]. Lung imaging manifests earlier than clinical symptoms, so imaging examination is vital in preclinical screening. [Pan, Y. et al. European Radiol. (2020) doi.org/10.1007/s00330-020-06731-x]. Some patients with SARS-CoV-2 infection are asymptomatic, while in severe cases, ARDS, septic shock, difficult to correct metabolic acidosis and coagulation dysfunction develop rapidly [Pan, Y. et al. European Radiol. (2020) doi.org/10.1007/s00330-020-06731-x]. Most patients who have died from the virus had other chronic medical conditions, were elderly, or were immunocompromised. [Yang, Y. et al., J. Autoimmunity (2020) doi.org/10.1016/j.jaut.2020.102434].

Although SARS-CoV-2 and SARS-CoV have certain similarities in their biological, epidemiological and pathological characteristics, there are some important differences. First, the most fundamental difference between the two viruses lies in their gene sequence. Second, there are more confirmed cases, suspected cases and deaths from SARS-CoV-2 infection, but the mortality rate is lower than that of SARS. Third, most cases are out-of-hospital infections. [Yang, Y. et al., J. Autoimmunity (2020) doi.org/10.1016/j.jaut.2020.102434]

SARS-CoV preferentially infects alveolar type II cells compared to type I cells [Mason, RJ. Eur. Respiratory J. (2020) 55: 2000607, citing Mossel, E C et al. Virology (2008) 372: 127-35; Weinheimer, V K, et al. J. Infect. Dis. (2012) 206: 1685-94]. Normally, alveolar type II cells are the precursor cells for alveolar type I cells. The infected alveolar units tend to be peripheral and subpleural [Id., citing Wu, J. et al. Invest. Radiol. (2020) doi.org/10.1097/RLI.0000000000000670; Zhang, S. et al. Eur. J. Respir. J. (2020) In press]. SARS-CoV propagates within type II cells, large number of viral particles are released, and the cells undergo apoptosis and die [Id., citing Qian Z. et al. Am. J. Respir. Cell Mol. Biol. (2013) 48: 742-48]. The released viral particles then infect type II cells in adjacent units.

### E) Structure-Based Functional Analysis

Cell entry of CoVs depends on binding of the viral spike (S) proteins to cellular receptors and on S protein priming by host cell proteases, which entails S protein cleavage at the S1/S2 and the S2′ site and allows fusion of viral and cellular membranes, a process driven by the S2 subunit. [Hoffmann, M. et al. Cell (2020) 181 (2): 271-80].

SARS-CoV can use the endosomal cysteine proteases cathepsin B and L (CatB/L) [Id., citing Simmons, G., et al. Proc. Natl. Acad. Sci. USA (2005) 102: 11876-881] and the transmembrane serine protease TMPRSS2 [Id., citing Glowacka, I. et al. J. Virol. (2011) 85: 4122-34, Matsuyama, S. et al. J. Virol. (2010) 84: 12658-664, Shulla, K. et al. J. Virol. (2011) 85: 873-82] for S protein priming in cell lines, and inhibition of both proteases is required for robust blockade of viral entry [Id., citing Kawase, M et al. J. Virol. (2012) 86: 6537-45]. However, only TMPRSS2 activity is essential for viral spread and pathogenesis in the infected host whereas CatB/L activity is dispensable [Id., citing Iwata-Yoshikawa, N. et al. J. Virol. (2019) 93: 10.1128/JVL01815-18, Shirato, K. et al. Virology (2016) 91: 10.1128/JVL01387-16, Shirato, K. et al. Virology (2018) 517: 9-15, Zhou, P et al. Antiviral Res. (2015) 116: 76-84].

SARSCoV-2 uses the SARS-CoV receptor ACE2 to gain entry into host cells and the serine protease TMPRSS2 for S protein priming. [Hoffman, M. et al. Cell (2020) 181 (2): 271-80] One mechanism for SARS-CoV-2 entry occurs when the spike protein on the surface of SARS-CoV-2 binds to an ACE2 receptor followed by cleavage at two cut sites (“priming”) that causes a conformational change allowing for viral and host membrane fusion. [Shrimp, J H et al. ACS Pharmacol. Trans. Sci. (2020) 3(5): 997-1007].

Angiotensin converting enzyme 2 (ACE2) and dipeptidyl peptidase 4 (DPP4) are known host receptors for SARS-CoV and MERS-CoV respectively [Yang, Y. et al., J. Autoimmunity (2020) doi.org/10.1016/j.jaut.2020.102434, citing Kuhn, J H, et al. Cell Mol. Life Sci. (2004) 61 (21): 2738-43; Raj, V S, et al. Nature (2013) 495 (7440): 251-54].

ACE2 is not only highly expressed in lung AT2 cells, esophagus upper and stratified epithelial cells, but also in absorptive enterocytes from the ileum and colon [Yang, Y. et al., J. Autoimmunity (2020) doi.org/10.1016/j.jaut.2020.102434., citing Zhang, H. et al. bioRxiv (2020) 2020.01.30.927806]. Thus, although the respiratory system is a primary target of SARS-CoV-2, bioinformatics analysis of single-cell transcriptosome datasets of lung, esophagus, gastric, ileum and colon tissue reveal that the digestive system is also a potential route of entry for COVID-19; Cardiovascular complications are rapidly emerging as a key threat in COVID-19. [Varga, Z. et al. The Lancet (2020) doi.org/10.1016/S0140-6736(20)30937-5] Endothelial cell involvement across vascular beds of different organs has been demonstrated in a series of patients with COVID-19. [Varga, Z. et al. The Lancet (2020) doi.org/10.1016/S0140-6736(20)30937-5].

The renin angiotensin system (RAS) is a central regulator of renal and cardiovascular function. Classically, it consists of angiotensin converting enzyme (ACE), its product, angiotensin (Ang) II and receptors for Ang II, angiotensin Type 1 (AT1) and angiotensin type 2 (AT2) receptors. RAS further includes ACE2, a monocarboxypeptidase that generates Ang-(1-7) from Ang II. Angiotensin-(1-7) is an endogenous ligand for the G protein-coupled receptor Mas; Mas therefore mediates the biological actions of Ang-(1-7) [Singh, N. et al. Am J. Physiol. Heart Circ. Hysiol. (2015) 309 (10): H1697-H1707, citing Santos, R A et al. Proc. Nat. Acad. Sci. USA (2003) 100: 8258-63]. Ang II produces hypertensive, pro-oxidative, hypertrophic and pro-fibrotic effects in the cardiovascular system. Ang-(1-7) elicits counter-regulatory effects on the ACE/AngII pathway by reducing vasodilatory, antihypertensive, antihypertrophic, antifibrotic and antithrombotic effects [Id., citing Ferreira, A J, et al. Hypertension (2010) 55: 207-13; Jusuf, D. et al. Eur. J. Pharmacol. (2008) 585: 303-12].

It has been reported that even though the expression of hACE2 in T cells is very low, SARS-CoV-2 can infect T cells through receptor-dependent, S protein-mediated membrane fusion; similar to MERS-CoV, SARS-CoV-2 infection of T cells is abortive [Wang, X. et al. Cellular & Mol. Immunol. (2020) doi.org/10.1038/s41423-020-0424-9.]

Dipeptidyl peptidase 4 (DPP4, also known as CD26), the receptor for MERS-CoV, [Anderson, R M et al. Philos. Trans. R. Soc. Lond. B. Biol. Sci. (2004) 359: 1091-105], citing Meyerholz, D K et al. Am. J. Pathol. (2016) 186: 78-86] is a multifunctional cell-surface protein widely expressed on epithelial cells in kidney, small intestine, liver and prostate and on activated leukocytes. DPP4 is expressed in the upper respiratory tract epithelium of camels. [Id., citing Widagdo, W. et al. J. Vir. (2016) 90: 4838-42]. In the human respiratory tract, DPP4 is mainly expressed in alveoli rather than the nasal cavity or conducting airways. [Id., citing Meyerholz, D K et al. Am. J. Pathol. (2016) 186: 78-86] DPP4 is a key factor in the activation of T cells and immune response costimulatory signals in T cells, which could indicate the virus' ability to manipulate the host immune system. [Id., citing Boonacker, E., Van Noorden, C J. Eur. J. Cell Biol. (2003) 82: 53-73].

Human aminopeptidase N (CD13), a cell-surface metalloprotease on intestinal, lung and kidney epithelial cells, has been identified as the receptor for HCoV-229E. [Id., citing Yeager, C L et al. Nature (1992) 357: 420-22].

The receptor for HCoV-OC43 is 9-O-acetylated sialic acid. The receptor for HCoV-HKU1 has not been identified.

F) Immune Response to Infection with CoVs

Extensive global efforts to map the determinants of immune protection against SARS-CoV-2 are ongoing.

### Innate and Adaptive Cell Populations

In one clinical study, a comprehensive analysis of the overall immunologic state of 42 individuals with different trajectories of SARS-CoV-2 infection and COVID-19 (moderate, severe (requiring oxygen at a flow rat higher than 6 L per min. or by an advanced oxygen delivery device) and recovered) was compared with 12 healthy donors using whole blood investigated immunological perturbations and activation occurring in circulating lymphocytes and major granulocyte populations, modulation of the B cell repertoire and its associations with the establishment of a SARS-CoV-2-specific humoral response, and activation of T cells relative to disease severity. Median number of days since onset of symptoms to disease progression in donors with severe COVID-19 was 9; individuals with moderate disease also reported a median of 9 days since onset of symptoms. [Kuri-Cervantes, L. et al. Sci. Immunol. (2020) 10.1126/sciimmunol.abd7114].

As measured by complete blood counts (CBC), the numbers of WBCs and PMNs were elevated above normal in all COVID-19+ patients, and were significantly higher in donors with severe over moderate disease. An expansion in the proportion of both neutrophil and eosinophil populations in severe compared to healthy donors was observed. The neutrophil frequency also differed significantly between moderate and severe disease, but did not show increased activation of cycling. Decreased expression of CD15 in neutrophils was seen between healthy donors and severe patients, but not in eosinophils. Although no significant differences were found in the immature granulocyte frequencies between healthy donors and COVID-19+ individuals, the proportion of immature granulocytes in moderate and severe COVID-19+ donors correlated inversely with the time since onset of symptoms. Monocyte blood counts were also higher than normal values in COVID-19+ donors. The total proportion of monocytes (CD14+ HLA-DR+) and monocyte subsets (CD14+ CD16+) was similar across groups. Donors with severe COVID-19 had lower frequencies of dendritic cells compared to moderate disease and healthy donors, but not with recovered individuals in both conventional (CD11c+ CD1231o/−) and plasmacytoid (CD11c-CD123+) dendritic cell subsets. [Id.]

Consistent with previous reports [Id., citing Chen, N., et al. Lancet (2020) 395: 507-13; Ruan, Q. et al. Intensive Care Med. (2020) 46: 846-48; Henry, B. M. Lancet Respir. Med. (2020) 8: e24; Tan, L. et al. Signal Transduct. Target Ther. (2020) 5:33], a relative decrease in the percentages of all lymphocyte subsets was seen. Severe COVID-19+ individuals had significantly lower proportions of T cells as well as total CD4+ and CD8+ T cells. Lower frequencies of CD8+ MAIT cells, innate lymphoid cells (ILCs, meaning the innate counterparts of T lymphocytes, which lack adaptive antigen receptors generated by the recombination of genetic elements), and natural killer (NK) cells [meaning group I innate lymphocytes] compared to healthy donors was seen. There were no significant differences in the frequencies of these cell subsets between health donors and moderate or recovered COVID-19+ individuals. Within the NK cell lineage, a drastic decrease in the frequencies of both CD56^{bright}CD16− and CD56^{dim}CD16+ NK cells in severe COVID-19 vs. healthy donors was seen. Decreased expression of CD16 on neutrophils, monocytes and immature granulocytes and decreased expression of HLA-DR in monocytes has been associated with sepsis and sepsis outcome. [Id., citing Giamarellos-Bouroulis, E J et al. Cell Host Microbe (2020) 27: 992-1000; Davenport, E E et al. Lancet Respir. Med. (2016) 4: 259-71; Faivre, V. et al. PLoS One (2016) 11: e0164489; Monneret, G. e al. Intensive Care Med. (2006) 32: 1175-83]. In the recovered group, the proportion of T cells, CD8+ MAIT cells, ILCs and NK cells were comparable to healthy donors. The proportions of circulatory follicular helper CD4+ T cells [cT_{FH} cells] and regulatory CD4+ T cells were similar across all groups. There was a negative correlation with the frequency of central memory T (T_{CM}) cells and days since the onset of symptoms; differences in CD4+ and CD8+ memory T cell subsets between groups was not observed.

B cell plasmablasts (meaning a precursor of a plasma cell that develops from a B lymphocyte in reaction to a specific antigen) were significantly expanded in severe COVID-19+ donors compared to healthy donors. There was an unusually high proportion of large clones comprising the majority of the circulating antibody repertoire. Expanded plasmablasts were not found in recovered COVID-19+ donors. The frequency of plasmablasts in individuals with severe COVID-19 did not correlate with age, days since onset of symptoms, the presence of morbidities, APACHE III score (meaning a measure used to assess illness severity of patients admitted to an ICU and to compare risk-adjusted outcomes between ICUs) or frequency of CD4+ cT_{FH} cells. The levels of total IgG in plasma and serum were equivalent across the groups, despite the heightened plasmablast response in severe COVID-19.

The levels of SARS-CoV-2 spike receptor binding domain (RBD)-specific IgM and IgG were significantly higher in the severe and recovered COVID-19+ individuals. There was a positive association between the levels of spike RBD-specific IgM and IgG and time since onset of symptoms in the moderate and severe groups, indicating the development of a strong SARS-CoV-2 specific humoral response. In the memory B cell population, an increased proportion of CD21−CD27− cells in moderate and severe disease, with a parallel decrease in CD21+CD27+ B cells was observed. CD21 (complement receptor type 2, CR2), a co-receptor of the B cell receptor, was also significantly down-regulated in severe COVID-19 patients. Activation, binding of complement or toll like receptor stimulation are known to decrease cell surface expression of CD21, and could lead to impaired B cell responses [Id., citing Charles, E D, et al. Blood (2011) 117: 5425-37; Illingworth, J. et al. J. Immunol. (2013) 190: 1038-47; Visentini, M. et al. Blood (2011) 118: 3440-41, author reply 3442; Weiss, G E et al. J. Immunol. (2009) 183: 2176-82; Gies, V. et al. JCI Insight (2018) 3: e96795].

T cell activation is typically observed during acute viral infections [Id., citing Kahan, S M et al. Virology (2015) 479-80: 180-93; Fenwick, C. et al. Immunol. Rev. (2019) 292: 149-63; Dias, C N S, et al. Immunology (2018) 155: 499-504], and increased activation of both CD4+ and CD8+ T cells in severe COVID-19 was observed. However, T cell activation was very heterogeneous across severe CoV patients, being equivalent to baseline in some, while reaching up to about 25% of memory CD8+ cells in others. This heterogeneity is relatively unusual compared to the symptomatic phase in other acute infections in humans, such as human immunodeficiency virus (HIV), Epstein-Barr virus (EBV), human cytomegalovirus (HCMV), hepatitis B virus (HBV) and Ebola, where activation is uniformly detectable but to varying and sometimes much higher degrees. [Id., citing Ndhlovu, Z M et al., Immunity (2015) 43: 591-604; Demers, K R, et al. PLoS Pathog. (2016) 12: e1005805; Agrati, C. et al. Cell Death Dis. (2016) 7: e2164; Sandalova, E. et al. PLoS Pathog. (2010) 6: e1001051]. There was a marked reduction in the frequency of CD161+CD8+ T cells in donors with severe COVID-19; this subset is composed primarily of mucosal-associated invariant T cells (MAIT) [Id., citing Walker, L J et al. Blood (2012) 119: 422-33], and a small subset of IL-17 secreting cells (Tc17) [Id., citing Billerbeck, E. et al. Proc. Natl Acad. Sci. USA (2010) 107: 3006-11]. Since MAIT cells are primarily γδT cells with limited diversity present in the mucosal immune system that respond to bacterially derived folate derivatives presented by the nonclassical MHC class 1b molecule MR1, it has been suggested that the reduction of CD161+/MAIT CD8+ cells in peripheral blood could be indicative of sequestration in the lungs, potentially exacerbating tissue inflammation. [Kuri-Cervantes, L. et al. Sci. Immunol. (2020) 10.1126/sciimmunol.abd7114].

### G) Antibody Response

In SARS-CoV-1 and SARS-CoV-2 infection, N is highly immunogenic, with N-specific humoral immune responses arising concurrently with S-specific humoral immunity. [Atyeo, C. et al. Immunity (2020) 53: 1-9, citing Liu, W. et al. J. Clin. Microbiol. (2020) 58: e00461-20: Shi, Y. et al. J. Clin. Virol. (2004) 31: 66-68; Timani, K A et al. J. Clin. Virol. (2004) 30: 309-12]. However, immunization of hamsters with a vector expressing N offered no protection against SARS-CoV-2 challenge despite a strong anti-N response, whereas immunization with the same vector expressing S protected hamsters against challenge. [Id., citing Buchholz, U J et al. Proc. Natl. Acad. Sci USA (2004) 101: 9804-9]. It is estimated that 100 copies of S and 1000 copies of N are incorporated into each virion [Id., citing Bar-On, Y M, et al. eLife (2020) 9:e57309].

In one clinical study, SARS-CoV-2 specific humoral responses were profiled in a cohort of 22 hospitalized individuals to determine whether identifiable antibody functional profiles across SARS-CoV-2 antigen specificities evolve early following infection and track differentially with disease outcome. [Atyeo, C. et al. Immunity (2020) 53: 1-9]. Two cross-sectional sample sets of SARS-CoV-2 infected individuals were assembled at the time of hospital admission to begin to comprehensively profile the evolution of the early SARSCoV-2 S-specific response and to define antibody features that are predictive of disease outcome. The data point to antigen-specific and antibody-effector differences early in infection that differ by clinical trajectory.

Nightingale rose plots revealed that deceased individuals exhibited a more N-focused humoral immune response compared with the S-centric response elicited among convalescents. In particular, higher S-specific antibody-dependent complement deposition (ADCD), antibody-dependent neutrophil phagocytosis (ADNP), antibody-dependent cellular phagocytosis (ADCP) and enhanced IgG1, IgA1, and IgM responses were observed among survivors. In contrast, S-specific NK cell-activating responses were enriched in the deceased. The S-derived receptor-binding domain (RBD)-specific antibody-mediated natural killer (NK) cell degranulation (NKD) and ADNP, both driven by the related FCγ receptors FcγR3A and FcγR3B, respectively, trended towards increases among individuals who died, with the exception of RBD-specific monocyte phagocytosis, which was enriched among individuals who survived.

Although not significant, individuals who died exhibited correlated isotype or subclass responses with immunocyte and neutrophil phagocytosis but negative and generally poorer correlations of NK cell-activating and complement-recruiting antibody response with all other functions, suggesting that individuals who die develop a functionally biased humoral immune responses. Although IgG1 responses were associated with all functions across the individuals who later died, diversified isotype and subclass responses were largely inversely correlated with antibody-dependent complement deposition (ADCD) and NK cell functions.

The study reported that as few as 5 features were sufficient to drive separation across the subjects. Features enriched among individuals who later died included N-specific IgM and IgA2, which were linked to a large number of additional N- and RBD-specific poorly functional antibody features. N-specific complement activity (ADCD), IgM and IgA titers were enriched in individuals who died. Conversely S-specific IgM titers, enriched in convalescent individuals, were correlated with functional S-specific IgG3 responses, RBD-specific IgM, and S-specific monocyte and neutrophil phagocytosis. Moreover, S-specific IgA1 responses, also enriched among convalescents, were linked to RBD-specific complement activation (ADCD) and S-, RBD- and N-specific FcγR2A binding, which is the Fcγ receptor involved in phagocytosis. These data suggested the potential importance of a less N-focused but more functional S-specific phagocytic response as an early correlate of recovery from infection.

Although S-specific antibodies able to recruit NK cell activity were expanded in individuals who died, coordination of NK cell and phagocytic activity was enriched among convalescents, which suggested the potential importance of synergy between innate immune effector functions. NK cells have been implicated both in protection [Id., citing, L L et al. Cell (2016) 167: 433-43.e.14; Jegaskanda, S. et al. J. Infect. Dis. (2016) 214: 945-52; van Erp, E A et al. Front. Immunol. (2019) 10: 548] and in pathology [Id., citing Cong, J. & Wei, H. Front. Immunol. (2019) 10: 1416].

Among the comorbidities of age, gender, viral load and days from symptom onset, age was the second major driver of variation in antibody profiles. Fc-Fcγ receptor (FcγR) interactions drive pleiotropic effector functions that limit viral replication and provide potent antiviral protection [Bournazos, S. et al. Nature Revs. Immunol. (2020) doi.org/10.1038/s41577-020-00420-0]; implicating age-associated defects in Fc variation that may contribute to altered antiviral immunity to SARS-CoV-2.

In another clinical study, the anti-SARS-CoV-2 antibody response was examined over a 115 day period in serum (n=496) and saliva (n=90) samples from acute and convalescent patients with laboratory-diagnosed COVID-19, compared to controls. IgG, IgA and IgM responses to the S protein (full length trimer) and its receptor binding domain (RBD) were profiled by ELISAs. Antigen-specific IgG in both serum and saliva was detected by 16-30 days post-symptom onset (PSO) and did not drastically decline in their relative levels as late as 100-115 days PSO. In contrast, antigen-specific IgM and IgA were rapidly induced but subsequently declined. In serum, neutralizing antibodies reached their maximum by 31-45 days PSO, and slowly declined up to 105 days, when there was a more pronounced drop [Isho, B. et al. (2020) doi.org/10.1101/2020.08.01.20166553].

### CD4+ T Cells and CD8+ T Cell Populations

### CD4+ T Cells

The canonical function of CD4+ T cells is the provision of help for B cells in germinal center formation, isotype switching and affinity maturation of antibody responses. [Swain, S L et al. Nat. Rev. Immunol. (2012) 12(2): 136-48]. Follicular helper T (T_{FH}) cells, a specialized subset of CD4+ T cells, provide help to B cells through both cell-cell interactions (most notably CD40L-CD40 interactions) and the release of cytokines. cT_{FH} are important for the generation of long-lasting and specific humoral protection against viral infections. [Elizadi, S R et al. (2020) doi.org/10.1101/2020.07.07.191007] The generation of neutralizing antibodies is a crucial component of protection against many viral pathogens and the goal of most vaccine strategies.

### CD4+ T Cell-Mediated Help for CD8+ T Cells

No helper subset for CD8+ T cells analogous to T_{FH} cells has been identified to date. The mechanisms by which CD4+ T cells promote CD8+ T cell effector and memory responses are less well understood than B cell help, although, as in B cell help, CD40L−CD40 interactions between CD4+ T cells and antigen presenting cells (APCs) are crucial. [Id.] It is not clear whether different T_{H} cell subsets have distinct roles in helping CD8+ T cells.

In addition, CD4+ T cells facilitate the development of functional, pathogen-specific memory CD8+ T cells that can respond following re-infection [Id., citing Shedlock, DJ & Shen, H. Science (2003) 300: 337-39; Sun, J C & Bevan, M J. Science (2003) 300: 339-42; Janssen, E M, et al. Nature (2003) 421: 852-6; Northrop, J K et al. J. Immunol. (2006) 177: 1062-9]. One mechanism by which CD4+ T cells appear to promote this process involves the downregulation of TNF-related apoptosis-inducing ligand (TRAIL) expression on responding CD8+ T cells. It is thought that CD8+ T cells that are helped by CD4+ T cells downregulate TRAIL expression and become less susceptible [Id., citing Janssen, E M et al. Nature (2005) 434: 88-93], or have delayed susceptibility [Id., citing Badovinac, V P et al. J. Immunol. (2006) 177: 999-1006], to TRAIL-mediated apoptosis. In contrast, CD8+ T cells that have not been helped undergo enhanced TRAIL-mediated apoptosis following antigen re-exposure. CD4+ T cell-mediated help also controls the expression of other molecules. For example, CD4+ T cells downregulate the expression of programmed cell death protein 1 (PD1) on CD8+ T cells, which can enhance the function of pathogen-specific memory CD8+ T cells [Id., citing Sacks, JA & Bevan, MJ. J. Immunol. (2008) 180: 4570-76; Fuse, S. et al. J. Immunol. (2009) 182: 4244-4254; Intlekofer, A M et al. J. Exp. Med. (2007) 204: 2015-21]. For example, paracrine IL-2 produced by CD4+ T cells during the initial priming of CD8+ T cells in lymphocytic choriomeningitis virus (LCMV) infection dramatically improves the CD8+ T cell recall response potential [Id., citing Williams, M A et al. Nature (2006) 441: 890-3]. Furthermore, CD4+ T cells have been shown to upregulate the expression of CD25 (also known as IL-2Ra) on CD8+ T cells during infection with vaccinia virus or VSV [Id., citing Obarr, J J et al. Proc. Natl. Acad. Sci. USA (2010) 107: 193-8]. At later stages of the response, CD4+ T cells produce additional cytokines, such as IL-21, which appears to be a crucial signal for downregulating TRAIL expression on CD8+ T cells responding to vaccinia virus [Id., citing Barker, B R et al. Eur. J. Immunol. (2010) 40: 3085-96]. Finally, evidence suggests that direct ligation of CD40 on naive CD8+ T cells by CD40L on CD4+ T cells can enhance the generation of memory CD8+ T cells [Id., citing Bourgeois, C. et al. Science (2002) 297: 2060-63].

CD4+ T cells seem to be particularly important for maintaining memory CD8+ T cell populations [Id., citing Sun, J C et al. Nature Immunol. (2004) 5: 927-33], and the presence of CD4+ T cells during priming may influence the homing pattern and, ultimately, the tissue distribution of memory CD8+ T cells [Id., citing Azadniv, M. et al. PLoS One (2011) 6: e16222].

### Functional Subsets of CD8+ T Cells

CD8 T cells are critical for mediating clearance following many acute viral infections in the lung. [Schmidt, M E & Varga, SM. Front. Immunol. (2018) doi.org/10.3389/fimmu.2018.00678]. Much of what is known about functional subsets of CD8+ T cells in the context of viral infection has been discovered in the context of HIV infection.

CD8+ T cells are a subpopulation of T cells that have a relevant role in host defense mainly against viruses and tumor cells [Gonzalez, S M. et al. Front. Immunol. (2017) doi.org/10.3389/fimmu.2017.00936]. Effector T cell differentiation occurs when naïve CD8+ T cells are activated by antigen-presenting cells (APCs), specifically DCs, that present endogenous peptides in the context of class I MHC molecules. In addition, they require the interaction with co-stimulatory molecules, such as CD80/86, and signaling through cytokines. Although such signaling is usually provided by DCs and activated CD4+ T cells [Id., citing Obar, J J & Lefrancois, L. Ann. NY Acad. Sci. (2010) 1183 (1): 251-66; Ridge, J P et al. Nature (1998) 393 (6684): 474-8], some studies have indicated little or no requirement for additional signaling coming from the CD4+ compartment, at least under certain circumstances [Id., citing Wang, B. et al. J. Immunol. (2001) 167(3): 1283-9; Johnson, A J, J. Virol. (1999) 73 (5): 3702-8].

Once naïve-specific CD8+ T cells are activated, the effector response requires clonal expansion and formation of primary effector cells capable of recognizing peptides from virally infected or tumor cells, leading to direct killing of antigen-bearing cells through perforins, granzymes, and Fas/FasL interaction [Id., citing Obar, J J & Lefrancois, L. Ann. NY Acad. Sci. (2010) 1183 (1): 251-66; Wherry, E J & Ahmed, R. J. Virol. (2004) 78 (11): 5535-45; Kalams, SA & Walker, BD. J. Exp. Med. (1998) 188 (12): 2199-2204; Wong, P. & Pamer, E G. Annu. Rev. Immunol. (2003) 21: 29-70]. In addition, release of cytokines with antimicrobial action, such as TNF-α and IFN-γ [Id., citing Guidotti, L G et al. Proc. Natl. Acad. Sci. USA (1994) 91 (9): 3764-8], and chemokines, such as MIP-1a/(3 and RANTES [Yang, 00 et al. J. Virol. (1997) 71 (4): 3120-8] contribute to clearance of altered cells.

Antigen concentration and persistence play an important role in the differentiation of different subsets of T cells. Although a brief exposure to an antigen presented by APCs can trigger activation, expansion, and differentiation of naïve CD8+ T cells into effector T cells, prolonged exposure to the antigen is usually required to generate an efficient effector response and memory CD8+ T cells [Id., citing Obar, J J & Lefrancois, L. Ann. NY Acad. Sci. (2010) 1183 (1): 251-66; van Stipdonk, M J et al. Nat. Immunol. (2001) 2(5): 423-9]. After resolution of an infection, a phase of T cell contraction is induced as a mechanism of immune regulation, during which most of the effector specific-CD8+ T cells die by apoptosis and some survivor cells (5-10%) are preserved as long-lived memory cells [Id., citing Obar, J J & Lefrancois, L. Ann. NY Acad. Sci. (2010) 1183 (1): 251-66; Wherry, E J & Ahmed, R. J. Virol. (2004) 78 (110: 5535-45).

Despite a CD8+ T cell effector response, the successful eradication of the pathogen is not always guaranteed. Chronic infections such as HIV are characterized by antigen persistence that induces terminally differentiated effector CD8+ T cells over the memory phenotypes, and ultimately immune exhaustion and activation-induced cell death [Id., citing Cox, M A et al. Virology (2013) 435 (1): 157-69]. Indeed, late phases of HIV infection are associated with progressive reduction of CD8+ T cells, lower effector functions, and inability to respond to HIV and other pathogens or tumor cells [Id., citing Klein, M R et al. J. Exp. Med. (1995) 181 (4): 1365-72; Zhang, J-Y, et al. Blood (2007) 109 (11): 4671-8; Yamamoto, T. et al. Blood (2011) 117 (198): 4805-15].

Once activated, CD8+ T cells may differentiate into several functional phenotypes. Initially, they acquire an effector phenotype that will result in high numbers of terminally differentiated effector cells (85-90% of activated CD8+ T cells) that is characterized by high cytotoxic ability and production of cytokines; nonetheless, as the pattern of produced cytokines is variable among CD8+ T cells, they can be classified into different subsets of effector cells: (i) CD8+ T cells with a Th1-like cytokine pattern (Tc1), which produce IFN-γ and TNF-α and exhibit a high cytotoxic function and (ii) CD8+ T cells with a Th2-like cytokine pattern (Tc2), which produce IL-4, IL-5, IL-6, and IL-10 and have lower cytotoxic ability than Tc1 cells. [Id.] In addition, some cells produce both Th1 and Th2 cytokines; naïve CD8+ T cells exhibit a strong preference to differentiate into Tc1 cells [Id., citing Mosmann, T R et al. Semin. Immunol. (1997) 9 (2): 87-92]. An additional pattern recently described and less studied is Tc17 that produces IL-17 but no granzyme B, and has a low capacity to perform lysis in vitro; these cells exhibit some functional plasticity, since they can produce IFN-γ while losing the expression of IL-17 [Id., citing; Yen, H-R et al. J. Immunol. (2009) 183 (11): 7161-8].

Once the pathogen is eradicated, some cells acquire a memory phenotype that persists even in the absence of the antigen [Gonzalez, S M. et al. Front. Immunol. (2017) doi.org/10.3389/fimmu.2017.00936], citing Lau, L L et al. Nature (1994) 369 (6482): 648-52; Murali-Krishna, K., et al. Science (1999) 286 (5443): 1377-81]. These memory CD8+ T cells are characterized by their capacity of self-renewal, reside in lymphoid and non-lymphoid tissues awaiting a second encounter with the specific antigen, and recall effector functions after this encounter [Id., citing Youngblood, B. et al. F1000 Prime Rep. (2015) 7: 38]. They are distinguished from naïve and effector CD8+ T cells that express the marker CD45RA, by the loss of this marker and the gain of CD45RO after differentiation [Id., citing Sallusto, F. et al. Annu. Rev. Immunol. (2004) 22: 745-63]. These memory CD8+ T cells have been classified into the following subsets based on differences in the degrees of effector functions, proliferative capacity, and tissue homing properties: (i) central memory T cells (T_{CM}) restricted mainly to lymphoid tissues because of their expression of the lymphoid homing molecules CD62L and CCR7, which are also considered as the source for self-renewal of the pool of memory cells, generating a second wave of effector T cells; (ii) effector memory cells (T_{EFF}) that provide a first line of defense against infections through immediate effector functions and are present in circulation and non-lymphoid tissues due to the low expression of the lymphoid homing molecules CD62L and CCR7 [Id., citing Wherry, E J & Ahmed, R. J. Virol. (2004) 78 (11): 5535-45; Youngblood, B. et al. F1000 Prime Rep. (2015) 7: 38]; and (iii) a subset of tissue-resident memory cells (T_{RM}) that are located at sites of pathogen entry. The protective CD8+ T cell response is achieved through the collective function of all these effector and memory subsets [Id., citing Youngblood, B. et al. F1000 Prime Rep. (2015) 7: 38]. In addition to the effector and memory subsets, these CD8+ T cells can also be classified according to the level of activation. Based on the expression of the activation markers HLA-DR and CD38, four phenotypes of cells have been identified: (i) HLA-DR+CD38+; (ii) HLA-DR+CD38−; (iii) HLA-DR−CD38+; and (iv) HLA-DR−CD38−. [Gonzalez, S M. et al. Front. Immunol. (2017) doi.org/10.3389/fimmu.2017.00936]]

A subset of effector memory T cells re-expresses CD45RA (termed TEMRA) after antigenic stimulation with unknown molecular characteristics and functions. CD4 TEMRA cells have been implicated in protective immunity against pathogens such as dengue virus (DENV). [Tian, Y. et al. Nature Communic. (2017) 8: 1473]

The co-expression of HLA-DR and CD38 define the classical activation phenotype of CD8+ T cells. Several studies indicate that this population exhibits high effector functions, such as proliferation, cytotoxicity, and cytokine production, as well as higher susceptibility to cell death after their function have been accomplished [Gonzalez, S M. et al. Front. Immunol. (2017) doi.org/10.3389/fimmu.2017.00936, citing Miller, J D et al. Immunity (2008) 28 (5): 710-22; Lindgren, T. et al. J. Virol. (2011) 85 (19): 10252-60]. This subpopulation of activated specific CD8+ T cells performs an important function during acute viral infections, contributing to viral control [Id., citing Ndhlovu, Z M et al. Immunity (2015) 43 (3): 591-604]; however, the maintenance of this activation state observed during chronic viral infections is related to the subsequent loss of their functional abilities, to increased expression of inhibitory molecules related to immune exhaustion, and to activation-induced cell death [Id., citing Sachdeva, M. et al. J. Acquir. Immune Defic. Syndr. (2010) 54 (5): 447-54]. Some studies have focused on the subpopulation HLA-DR+CD38−CD8+ T cells, particularly in the context of chronic viral infection [Id., citing Saez-Cirion, A. et al. Proc. Nat. Acad. Sci. USA (2007) 104 (16): 6776-81; Hua, S. et al. PLoS One (2014) 9 (7): e101920]. This phenotype has been related to a controlled activated phenotype because of its low expression of the proliferation marker Ki-67 and of additional activation markers, such as CD69, CD25, CD71, and CD40. Indeed, the expression of these markers is similar to the one observed in resting HLA-DR−CD38−CD8+ T cells and lower than the one expressed by HLA-DR+CD38+CD8+ T cells. Despite this lower activation, HLA-DR+CD38−CD8+ T cells exhibit a higher functional response, an increased survival rate and a greater ability to suppress viral replication compared to cells expressing both activation markers [Id., citing Saez-Cirion, A. et al. Proc. Nat. Acad. Sci. USA (2007) 104 (16): 6776-81; Hua, S. et al. PLoS One (2014) 9 (7): e101920]. This particular activation phenotype seems to be induced by a higher avidity in the recognition of viral epitopes in the presence of low peptide concentrations [Id., citing Hua, S. et al. PLoS One (2014) 9 (7): e101920].

In addition to these described populations, some CD8+ T cells, which exhibit the ability to suppress T helper activity and induce anergy, are called regulatory CD8+ T cells [Id., citing Liu, Z. et al. Int. Immunol. (1998) 10 (6): 775-83; Colovai, A I et al; Transplantation (2000) 69 (7): 1304-10; Lu, L & Cantor, H. Cell Mol. Immunol. (2008) 5(6): 401-6]; however, they are not well characterized. Some reports indicate that these cells are related to a memory phenotype since they are CD45RA negative; in addition, they express the CD122 marker but neither the CD25 nor the FoxP3 markers, and their regulatory function seems to be mainly achieved by IL-10 production. [Id., citing Li, S. et al. Cell Mol. Immunol. (2014) 11(4): 326-31].

Although multiple models have been proposed to account for the formation of different T cell subsets, phenotypic, functional and molecular studies are most consistent with a linear progressive model beginning with TN cells, and then proceeding in the order T_{SCM} cells, T_{CM} cells, T_{EM} cells, to ultimately terminate with T_{EFF} cells. T_{SCM} cells, which are antigen-experienced cells, possess stem cell-like attributes to a greater extent than any other memory lymphocyte population. Like T_{SCM} cells, T_{CM} and T_{EM} cells can also undergo self-renewal; however, the capacity to form diverse progeny is progressively restricted so that only T_{SCM} cells are capable of generating all three memory subsets and T_{EFF} cells; T_{CM} cells can give rise to T_{CM}M, T_{EM} and T_{EFF} cells; and T_{EM} cells can only produce themselves and T_{EFF} cells. [Gattinoni, L. et al. Nature Reviews/Cancer (2012) 12: 671-84].

Another clinical study investigated the relationship between immune responses and COVID-19 disease trajectory in 125 hospitalized COVID-19 patients. [Mathew, D. et al. Science (2020) 10.1126/science.abc8511]. High dimensional flow cytometry was used to perform deep immune profiling of individual B and T cell populations, with temporal analysis of immune changes during infection; this profiling was combined with extensive clinical data to understand the relationships between immune responses to SARS-CoV2 and disease severity.

First, a defining feature of COVID-19 disease in these hospitalized patients was heterogeneity of the immune response. Many COVID-19 patients displayed robust CD8 T cell and/or CD4 T cell activation and proliferation and plasmablast responses, although a considerable subgroup of patients (about 20%) had minimal detectable response compared to controls. Even within those patients who mounted detectable T and B cell responses during COVID-19 disease, the immune characteristics of this response were heterogeneous.

Three immunotypes in these hospitalized COVID-19 patients were identified:

1) patients with robust activation and proliferation of CD4 T cells, relative lack of cT_{FH}, together with modest activation of TEMRA-like as well as highly activated or exhausted CD8 T cells and a signature of T-bet+ plasmablasts;

2) Tbet^{bright} effector-like CD8 T cell responses, less robust CD4 T cell responses, and Ki67+ plasmablasts and memory B cells; and

(3) an immunotype largely lacking detectable lymphocyte response to infection, suggesting a failure of immune activation.

Immunotype 1, comprised of robust CD4 T cell activation, paucity of cT_{FH} with proliferating effector/exhausted CD8 T cells and T-bet+ plasmablast involvement was connected to more severe disease. Immunotype 2 was characterized by more traditional effector CD8 T cell subsets, less CD4 T cell activation and proliferating plasmablast and memory B cells. Immunotype 3, in which minimal lymphocyte activation response was observed, may represent about 20% of COVID-19 patients.

Even when corrected for differences in age and race, these relationships were preserved, indicating that these immunotypes may reflect fundamental differences in the ways patients respond to SARSCoV-2 infection.

Second, there was a robust plasmablast response. Although blood plasmablast frequencies typically are correlated with blood activated cT_{FH} responses [Id., citing Herati, R S et al. Sci. Immunol. (2017) 2: eaag2152], in COVID-19 patients, the relationship between plasmablast and activated cT_{FH} in these patients was weak.

The durability of the strong T and B cell activation and proliferation in some patients was a striking feature of this study, with the stability of the CD8 and CD4 T cell activation and plasmablast responses suggesting a prolonged period of peak immune responses at the time of hospitalization or perhaps a failure to appropriately down-regulate responses in some patients, which fits with an overaggressive immune response and/or “cytokine storm” in this subset of patients. [Id., citing More, J B & June, C H. Science (2020) 368: 473-74]. Lymphopenia reportedly was preferential for CD8 T cells, and the frequency of the KI67+ or CD38+HLA-DR+ CD8 and CD4 T cell responses in COVID-19 patients was reportedly similar in magnitude to other acute viral infections or live attenuated vaccines in humans. [Id., citing Miller, J D, et al. Immunity (2008) 28: 710-22; Akondy, R S, et al. Nature (2017) 552: 362-67; Wilkinson, T M e al. Nat. Med. (2012) 18: 274-80].

Another clinical study reported that in 15 convalescent subjects after mildly symptomatic COVID-19 infection, a sustained enrichment of RBD-specific memory B cells and S-specific CD4+cT_{FH} in CoV2+ individuals was maintained to at least 3 months post-symptom onset. The proliferative capacity of sorted spike-specific T_{CM} (CD45-CCR7+) and T_{EM} (CD45RA-CCR7-) was determined in vitro following culture with autologous CD14+ monocytes and recombinant spike protein. T_{CM} from CoV2+ individuals displayed significant proliferation frequencies compared to healthy controls samples, although substantial proliferative responses by T_{EM} cells were observed in some CoV2+ individuals. Spike specific T_{CM}, and potentially T_{EM} therefore were maintained throughout the study and had the ability to proliferate and repopulate the memory pool upon antigen reencounter. [Rodda, L B et al. (2020) doi.org/10.1101/2020.08.11.20171843]

In another clinical study, SARS-CoV-2 specific CD4+ and CD8+ T cells were characterized in outcome-defined cohorts of donors (n=206) from Sweden and the functional and phenotypic landscape of SARS-CoV-2 specific T cell responses in unexposed individuals, exposed family members, and individuals with acute or convalescent COVID-19 were mapped. [Sekine, T. et al. Cell (2020) doi.org/10.1016/j.cell.2020.08.017]. As reported, absolute numbers and relative frequencies of CD4+ and CD8+ T cells were unphysiologically low in patients with acute moderate or severe COVID-19. Overlapping peptides spanning the immunogenic domains of the SARS-CoV-2 spike, membrane and nucleocapsid proteins were used to stimulate PBMCs from patients with acute moderate or severe COVID-19. A vast majority of responding CD4+ and CD8+ T cells displayed an activated/cycling (CD38+HLA-DR+ Ki67+PD-1+) phenotype. Using HLA class I tetramers as probes to detect CD8+ T cells specific for predicted optimal epitopes from SARS-CoV-2, a vast majority of tetramer+ CD8+ T cells in the acute phase of infection, but not during convalescence, displayed an activated/cycling phenotype. In general, early SARS-CoV-2-specific CD8+ T cell populations were characterized by the expression of immune activation molecules (CD38, HLA-DR, Ki-67), inhibitory receptors (PD-1, TIM-3) and cytotoxic molecules (granzyme B, perforin, whereas convalescent phase SARS-CoV-2-specific CD8+ T cell populations were skewed towards an early differentiated memory (CCR7+ CD127+ CD45RA-+ TCF1+) phenotype. Virus-specific memory T cells have been shown to persist for many years after infection with SARS-CoV-1 [Id., citing Le Bert, N. et al. Nature doi.org/10.1038/s41586-020-2550-z; Tang, F. et al. J. Immunol. (2011) 186: 7264-8; Yang, L T et al. Clin. Immunol. (2006) 120: 171-78]. Similar memory T cell responses directed against the N protein and S proteins were detected in some individuals lacking detectable circulating antibodies specific for SARSCoV-2. The expression frequencies of CCR7 and CD45RA among SARS-CoV-2-specific CD8+ T cells were positively correlated with the number of symptom-free days after infection, whereas the expression frequency of granzyme B among SARS-CoV-2-specific CD8+ T cells was inversely correlated with the number of symptom free days after infection. Time from exposure therefore was associated with the emergence of stem-like memory SARS-CoV-2-specific CD8+ cells.

### Cytokine Profiles in COVID Patients

Immune responses against pathogens are divided roughly into three types. [Kuri-Cervantes, L. et al. Sci. Immunol. 10.1126/sciimmuol.abd7114 (2020), citing Annunziato, F. et al. J. Allergy Clin. Immunol. (2015) 135: 626-35; Iwasaki, A. & Medzhitov, R. Nat. Immunol. (2015) 16: 343-53; O'Shea, J J & Paul W E. Science (2010) 327: 1098-1102]

Type 1 immunity, characterized by T-bet dependent responses and IFN-γ, is generated against intracellular pathogens, including viruses. Differential expression of the Th1 cell transcription factor T bet and a closely related T-box family transcription factor particularly in CD8+ T cells, Eomesodermin (Eomes), facilitates the cooperative maintenance of the pool of antiviral CD8+ T cells during chronic viral infection. [Paley, M A et a., Science (2012) 338: 1220-125]. During chronic infections, T-bet is reduced in virus-specific CD8+ T cells; this reduction correlates with T cell dysfunction. In contrast, Eomes mRNA expression is up-regulated in exhausted CD8+ T cells during chronic infection. [Id.] In type 1 immunity, pathogen clearance is mediated through effector cells, including innate lymphoid cell 1 (ILC1), natural killer (NK) cells, cytotoxic T lymphocytes and Th1 cells. Th1 cells are a lineage of CD4+ effector T cells that promotes cell-mediated immune responses and are required for host defense against intracellular viral and bacterial pathogens. Th1 cells secrete IFN-gamma, IL-2, IL-10, and TNF-alpha/beta. IL-12 and IFN-γ make naive CD4+ T cells highly express T-bet and STAT4 and differentiate to Th1 cells. (Zhang, Y. et al. Adv. Exp. Med. Bio. (2014) 841: 15-44)/

Type 2 immunity, which relies on the GATA-3 transcription factor, mediates anti-helminth defense through effector molecules including, IL-4, IL-5, IL-13, and IgE designed to expel these pathogens through the concerted action of epithelial cells, mast cells, eosinophils and basophils. GATA-3 is a member of the GATA family of conserved zinc-finger transcription factors, several of which are involved in hematopoiesis. As master regulator of Th2 lineage commitment, GATA-3 acts either as a transcriptional activator or repressor through direct action at many critical loci encoding cytokines, cytokine receptors, signaling molecules as well as transcription factors that are involved in the regulation of T(h)1 and T(h)2 differentiation [Jenner, R G et al., Proc Natl Acad Sci USA. (2009) 106(42):17876-81]. Th2 cells are a lineage of CD4+ effector T cells that secrete IL-4, IL-5, IL-9, IL-13, and IL-17E/IL-25. These cells are required for humoral or antibody-mediated immunity and play an important role in coordinating the immune response to large extracellular pathogens. IL-4 makes naive CD4+ T cells highly express STATE and GATA3 and differentiate to Th2 cells. [Zhang, Y. et al. Adv. Exp. Med. Bio. (2014) 841: 15-44]. For example, GATA3 regulates the expression of Th2 lineage specific cytokine genes such as IL5 and represses the Th1 lineage specific genes IL-12 receptor β2 and STAT4 as well as neutralizing RUNX3 function through protein-protein interaction. Mice lacking Gata3 produce IFN-gamma rather than Th2 cytokines (IL5 and IL13) in response to infection [Zhu, J et al., Nat Immunol. (2004)5(11):1157-65]. GATA3 acts in mutual opposition to the transcription factor T-bet, as T-bet promotes whereas GATA3 represses Fut7 transcription [Hwang, E S et al., Science. (2005) 21; 307(5708):430-3]. It also acts with Tbx21 to regulate cell lineage-specific expression of lymphocyte homing receptors and cytokine in both Th1 and Th2 lymphocyte subsets [Chen, G Y et al., Proc Natl Acad Sci USA. (2006) 103(45):16894-9].

Type 3 immunity is orchestrated by the RORγt-induced cytokines IL-17, IL-22 secreted by ILC3 and Th17 cells. Th17 cells are a CD4+ T-cell subset characterized by production of interleukin-17 (IL-17). IL-17 is a highly inflammatory cytokine with robust effects on stromal cells in many tissues, resulting in production of inflammatory cytokines and recruitment of leukocytes, especially neutrophils, thus creating a link between innate and adaptive immunity. [Tesmer, L A, et al., Immunol. Rev. (2008) 223: 87-113]. Type 3 immunity is mounted against fungi and extracellular bacteria to elicit neutrophil-dependent clearance. [Kuri-Cervantes, L. et al. Sci. Immunol. 10.1126/sciimmuol.abd7114 (2020)]

### Clinical Studies Regarding Cytokine Expression

In one clinical study, immune responses in 113 COVID-19 patients with moderate (non-ICU) and severe (ICU) disease were serially analyzed. PBMCs and plasma samples were analyzed by flow cytometry and ELISA to quantify leukocytes and soluble mediators, respectively. Marked changes in COVID-19 patients compared to uninfected healthcare workers was revealed. COVID-19 patients presented with marked reductions in T cell number and frequency in both CD4+ and CD8+ T cells, even after normalization for age as a possible confounder. Increases in monocytes and low density neutrophils and eosinophils was observed, which correlated with the severity of disease. Increased activation of T cells and a reduction in HLA-DR expression by circulating monocytes was observed. [Lucas, C. et al. Nature (2020) doi 10.1038/s41586-020-2588-y]

With regard to key differences between moderate and severe patients in cytokine expression, chemokines and additional immune markers, a “core COVID-19 signature” shared by both moderate and severe groups of patients was defined by the following inflammatory cytokines that positively correlated with each other: IL-1α, IL-1β, IL-17A, IL12p70, and IFN-α. In severe patients an additional inflammatory cluster defined by thrombopoietin (TPO), IL-33, IL-16, IL-21, IL-23, eotaxin and eotaxin 3 was observed. [Id.] Most of the cytokines linked to cytokine release syndrome (CRS), an acute systemic inflammatory syndrome characterized by fever and multiple organ dysfunction, e.g., IL-1α, IL-1β, IL-6, IL-10, IL-18 and TNF-α, showed increased positive associations in severe patients. These data highlight the broad inflammatory changes involving concomitant release of type-1, type-2, and type-3 cytokines in severe COVID-19 patients. [Id.]

Additional correlations between cytokines emerged in patients with severe disease following day 10, and temporal analyses of PBMCs and soluble proteins in plasma, either by linear regression or grouped intervals supported distinct courses of disease. IFN-α levels were sustained at higher levels in severe patients while they declined in moderate patients. Plasma IFN-λ, levels increased during the first week of symptom onset in ICU patients and remained elevated in later phases. Inflammasome-induced cytokines, such as IL-1β and IL-18 were also elevated in severe patients compared to patients with moderate disease at most time points analyzed. An inflammasome is a pro-inflammatory protein complex that is formed after stimulation of intracellular NO-like receptors; production of an active caspase in the complex processes cytokine proproteins into active cytokines. Consistently, IL-1 receptor antagonist (IL-1Ra), induced by IL-R signaling as a negative feedback regulator, also showed increased levels in ICU patients from day 10 of disease onset. [Id.]

Broad elevation of type 1, type 2, and type 3 signatures were identified in severe cases of COVID-19. (Id).

With regard to type 1 immunity, an increased number of monocytes was observed at approximately 14 days from symptom onset in severe but not in moderate COVID-19 patients. IL12, a key inducer of type 1 immunity and an innate cytokine, displayed a similar pattern to IFN-γ, increasing over time in severe patients, but steadily declining in moderate patients. By intracellular cytokine staining, CD4+ and CD8+ T cells from patients with moderate disease secreted amounts of IFN-γ comparable to those from severe patients. These data suggested that secretion of IFN-γ by non-T cells (ILC1, NK cells) or non-circulating T cells in tissues were the primary contributors to the enhanced levels observed in severe patients. (Id.)

With regard to Type 2 immunity, type 2 immune markers continued to increase in severe patients over time. Eosinophils and eotaxin-2 increased in severe patients and remained higher than levels measured in moderate patients. Type-2 innate immune cytokines, including thymic stromal lymphopoietin (TSLP) and IL-33, did not exhibit significant differences between severe and moderate patients. Hallmark type-2 cytokines, including IL-5 and IL-13, were enhanced in patients with severe over moderate disease. In contrast, although IL-4 was not significantly different, IL-4, like IL-5 and IL-13, exhibited an upward trend over the course of disease in severe patients. IgE levels also were significantly higher in severe patients and continued to increase during the disease course. (Id.)

IL-6 linked to CRS was significantly elevated in severe patients. Circulating neutrophils did not show a significant increase in the longitudinal analysis, although hallmarks of type 3 responses were observed in severe patients, including increased plasm IL-17a and IL-22, as well as IL-17 secretion by circulating CD4 T cells. (Id.)

Regardless of whether the patients exhibited moderate or severe disease, viral load significantly correlated with the levels of IFN-α, IFN-γ, and tumor necrosis factor-related apoptosis-inducing ligand (TRAIL), a member of the TNF cytokine family expressed on the cell surface of some cells, such as NK cells, that induces cell death in target cells by ligation of the death receptors DR4 and DR5. (Id.)

Three main clusters with correlation to distinct disease outcome emerged when patients' baseline measurements were collected before 12 days from symptom onset. Four distinct immune signatures emerged. Signature A contained several stromal growth factors, including EGF, PDGF, and VEG F, which are mediators of wound healing and tissue repair, as well as IL-7, a critical growth factor for lymphocytes. Signature B consisted of eotaxin 3, IL-33, thymic stromal lymphopoietin (TSLP) along with IL-21, IL-23, and IL-17F, thus representing Type 2 & 3 immune effectors. Signature C comprised mixtures of all immunotypes, including type 1 cytokines (IFN-γ, IL12p70, IL-15, IL-2, TNF-α), type-2 (IL-4, IL-5, IL-13) and type 3 (IL-1α, IL-1β, IL-17A, IL-17E, IL-22). Signature D contained a number of chemokines involved in leukocyte trafficking, including CCL1, CCL2, CCL5, CCL8, CCL15, CCL21, CCL22, CCL27, XCCL9, CXCL10, CXCL13, and SDF1. (Id.)

Cluster 1 comprised primarily patients with moderate disease who experienced low occurrences of coagulopathy, shorter lengths of hospital stay and no mortality. This cluster was characterized by low levels of inflammatory markers and similar or increased levels of Signature A parameters containing tissue reparative growth factors. Clusters 2 and 3 were characterized by the rise in inflammatory markers; patients in these clusters had higher incidence of coagulopathy and mortality, which was more pronounced in cluster 3. Cluster 2 showed higher levels of markers in signatures C and D, but lower expression of markers in Signature B, C and D than in Cluster 3. Cluster 3 displayed heightened expression of markers in signatures B, C and D than other clusters. Cluster 3 showed particular enrichment in expression of markers in signature B and markers linked to coagulopathy. Plasma inflammatory markers before the first 12 days of symptom onset strongly associated with severe disease outcomes. (Id).

In another clinical study, blood from convalescent donors in California was collected in acid citrate dextrose tubes and stored at room temperature prior to processing for PBMC isolation and plasma collection. Donors were asked to provide proof of positive testing for SARS-CoV-2, and screened for clinical history and or epidemiological risk factors. Clinical features consistent with COVID-19 included subjective or measured fever, signs or symptoms of lower respiratory tract illness (e.g., cough or dyspnea). Epidemiologic risk factors included close contact with a laboratory-confirmed case of SARS-CoV-2 within 14 days of symptom onset or a history of travel to an area with a high rate of COVID-19 cases within 14 days of symptom onset. Disease severity was defined as mild, moderate, severe or critical based on a modified version of the WHO interim guidance [WHO Ref. WHO/2019-nCoV/clinical/2020.4). Mild disease was defined as an uncomplicated upper respiratory tract infection with potential nonspecific symptoms (e.g., fatigue, fever, cough with or without sputum production, anorexia, malaise, myalgia, sore throat, dyspnea, nasal congestion, headache; rarely diarrhea, nausea and vomiting) that did not require hospitalization. Moderate disease was defined as the presence of lower respiratory tract disease or pneumonia without the need for supplemental oxygen, without signs of severe pneumonia or a URI requiring hospitalization (including observation admission status). Severe disease was defined as severe lower respiratory tract infection or pneumonia with fever plus any one of the following: tachypnea (respiratory rate >30 breaths per min), respiratory distress or oxygen saturation less than 93% on room air. Critical disease was defined as the need for ICU admission or the presence of ARDS, sepsis or septic shock, as defined in the WHO guidance document. Convalescent donors were screened for symptoms prior to scheduling blood draws, and had to be symptom-free and approximately 3 weeks out from symptom onset at the time of the initial blood draw. 70% of donors experienced mild illness. [Grifoni, A. et al. Cell (2020) 181: 1489-1501].

In another clinical study using HLA class I and II predicted peptide megapools, a technique whereby numerous epitopes are solubilized, pooled, and relyophilized to avoid cell toxicity problems [Id., citing Carrasco ProS. et al. J. Immunol. Res. (2015) 763461], circulating SARS-CoV-2 specific CD8+ and CD4+ T cells were identified in about 70% and 100% of COVID convalescent patients, respectively. CD4+ T cell responses to the virus spike protein, the main target of most vaccine efforts, were robust and correlated with the magnitude of anti-SARS-CoV-2 spike RBD IgG and IgA titers. The M, spike and N proteins each accounted for 11% to 27% of the total CD4+ response, with additional responses commonly targeting nsp3, nsp4, ORF3a, and ORF8, among others. These results were reported to demonstrate a pattern of robust and diverse SARS-CoV-2 specific CD4+ T cell reactivity in convalescing COVID-19 cases that correlated largely with predicted viral protein abundance in infected cells. [Grifoni, A. et al. Cell (2020) 181: 1489-1501].

For CD8+ T cells, Spike protein accounted for about 26% of the reactivity and N for about 12%. Significant reactivity in COVID-19 recovered subjects was derived from other antigens, such as M (22%), nsp6 (15%), ORF8 (10%) and ORF3a. [Grifoni, A. et al. Cell (2020) 181: 1489-1501].]

SARS-CoV-2 reactive CD4+ T cells were detected in about 40-60% of unexposed individuals, suggesting cross-reactive T cell recognition between circulating “common cold” coronaviruses and SARS-CoV-2. SARSCoV-2 cross-reactive CD8+ T cells were detected in at least four different donors, with less clear targeting of specific SARS-CoV-2 proteins than was observed for CD4+ T cells, suggesting that CoV CD8+ T cell cross-reactivity exists but is less widespread than CD4+ T cell cross-reactivity.[Grifoni, A. et al. Cell (2020) 181: 1489-1501].

In another clinical study of individuals residing in Singapore, LeBert et al. similarly reported that SARS-CoV-2 NP-specific T cells are part of the T cell repertoire of individuals with a history of SARS infection and are able to robustly expand after encounter with SARS-CoV-2 NP peptides. This finding was interpreted to demonstrate that infection with beta coronaviruses induces multi-specific and long-lasting T cell immunity to SARS-CoV-2 nucleocapsid protein (NP). SARS-CoV-2 specific T cells also were frequently detected in individuals with no history of SARS, COVID-19 or contact with SARS/COVID-19 patients; such SARSCoV-2 T cells in uninfected donors exhibited a different pattern of immunodominance, frequently targeting the ORF-1 coded non-structural proteins NSP7 and NSP13 as well as the NP structural protein. [Le Bert, N. et al. Nature doi. org/10.1038/s41586-020-2550-z].

In another clinical study, PBMCs from ten COVID-19 patients with acute respiratory distress syndrome (ARDS) collected up to three weeks after admission to the ICU were stimulated with megapools of overlapping or prediction based peptides covering the SARSCoV-2 proteome. [Weiskopf, D. et al. Sci. Immunol. 10.1126/sciimmunol.abd2071 (2020), citing Grifoni, A. Cell Host Microbe (2020) 27: 671-80.e2]. Phenotypic analysis of the PBMCs collected 14 days post inclusion via flow cytometry indicated that COVID-19 patients presented with low percentages of CD3+ T cells in peripheral blood, corresponding to the previously reported lymphopenia (12.1±8.7% in COVID-19 vs 44.3±7.1% in healthy controls, p<0.0001. CD4:CD8 ratios were increased in COVID-19 patients when compared to healthy controls (5.5±3.0 in COVID-19 vs. 2.3±0.9 in healthy controls, p=0.0115).

SARS-CoV-2 specific CD4+ and CD8+ T cells were detected in 10/10 and 8/10 COVID-19 patients, respectively. Peptide stimulation of healthy control age-matched PBMC samples collected before the outbreak in most cases resulted in undetectable responses, although some potential cross-reactivity due to infection with ‘common cold’ coronaviruses was observed. The strongest T-cell responses were directed to the spike (S) surface glycoprotein. [Weiskopf, D. et al. Sci. Immunol. 10.1126/sciimmunol.abd2071 (2020)

Phenotyping of CD8+CD69+CD137+ activated T cells showed that these had a mixed phenotype. The majority of virus-specific CD8+ T cells was identified as CCR7-effector memory (T_{EM}) or terminally differentiated effector (T_{EMRA}) [Id., citing Mahnke, Y D et al. Eur. J. Immunol. (2013) 43: 2797-2809]. Both of these CD8+ effector subsets are potent producers of IFNγ, contain preformed perforin granules for immediate antigen-specific cytotoxicity and home efficiently to peripheral lymphoid tissues [Id., citing Sallusto, F. et al. Nature (1999) 401: 708-12; Sallusto, F. et al. Annu. Rev. Immunol. (2004) 22: 745-63].

As for cytokine profiles, antigen-specific production of 13 cytokines in cell culture supernatants from PBMCs was measured after stimulation. SARS-CoV-2 specific T cells predominantly produced effector and Th1 cytokines (IFN-γ, TNF-α, IL-2), although Th2 (IL-5, IL-13, IL-9, IL-10) and Th17 (IL-17A, IL-17F, and IL-22) cytokines also were detected. Not enough COVID-19 ARDS patients were included in this study to correlate specific T cell responses to clinical outcome. [Weiskopf, D. et al. Sci. Immunol. 10.1126/sciimmunol.abd2071 (2020)]

Studies of the kinetics of development of virus-specific humoral and cellular immune responses in COVID-19 ARDS patients showed, by RT-PCR, that SARS-CoV-2 genomes in respiratory tract samples showed a decreasing trend over time, whereas virus-specific serum IgG antibody levels, measured by RBD ELISA, showed a significant increase.

SARS-CoV-2 T cell kinetics studies showed that SARS-CoV-2 specific T cells are present relatively early and increase over time. SARS-CoV-2-specific CD4+ and CD8+ T cells were detected in all patients at multiple time points. For CD4+ T cell responses, the frequencies of virus-specific responder cells increased significantly over time; for CD8+ T cells, this increase was not as apparent. Evidence suggested a direct negative correlation between viral loads and IgG ELISA and viral loads and CD4+ T cells, and a positive correlation between the appearance of IgG antibodies and virus-specific T cells.

Convalescent Plasma

Convalescent plasma has been tested only in small trials without the statistical power to provide firm conclusions of efficacy. The idea is that plasma contains antibodies, some of which might have helped the donor to recover from their infection, and proteins involved in regulating immune responses. However, such plasma varies widely in antibody concentration. As part of the US FDA expanded access program for COVID 19 convalescent plasma, key safety metrics were analyzed in a study of 500 patients; early indicators suggested that transfusion of convalescent plasma is safe in hospitalized patients with COVID-19. [Joyner, M J, et al. J. Clin. Invest. (2020) doi 10.1172/JC1140200]

Vaccine Development

There is great uncertainty about whether adaptive immune responses to SARS-CoV-2 are biologically insufficient, protective or pathogenic, or whether timing, composition or magnitude of the adaptive immune response determines its usefulness. [Grifoni, A. et al. Cell (2020) 181: 1489-1501]. Based on data from SARS patients in 2003-2004, one possibility is that substantial CD4+ T cell, CD8+ T cell, and neutralizing antibody responses develop to SARS CoV-2, all contribute to clearance of the acute infection, and some of the T and B cells are retained long term as immunological memory and protective immunity against SARS-CoV-2 infection. [Id., citing Guo, X., et al. medRxiv. Doi.org/10.1101/2020.02.12.20021386; Li, C K et al J. Immunol. (2008) 181: 5490-5500]. However, it is also possible that substantive adaptive immune responses can fail to occur, and robust protective immunity can fail to develop due to a T cell and/or antibody response of insufficient magnitude or durability, with the neutralizing antibody response being dependent on the CD4+ T cell response. [Id., citing Crotty, S. Immunity (2019) 50: 1132-1148, Zhao, J. et al. Immunity (2016)44: 1379-91].

For example, it has been shown that related, potentially neutralizing monoclonal antibodies that recognize the SARS-CoV-2 receptor binding domain (RBD) are often elicited in SARS-CoV-2 infection. [Weisblum, Y. et al. BioRxiv doi: 10.1101/2020.07.21.214759, citing, e.g., Cao, Y: et al. Cell (2020) 182: 1-12; Chen, X. et al. Cell. & Molec. Immunol. (2020) 17 (6): 647-9; Kreer, C. et al. Cell (2020)]. A significant fraction of COVID-19 convalescents, including some from whom potent neutralizing antibodies have been cloned, exhibit low levels of plasma neutralizing activity [Id., citing Robbiai, D F, et al. Nature (2020); in press; Wu, F. et al. medRxiv (2020); Luchsinger, L L, et al. medRxiv. (2020)], suggesting that natural SARS-CoV-2 infection may often fail to induce sufficient B-cell expansion and maturation to generate high titer neutralizing antibodies.

Using a recombinant chimeric VSV/SARS-CoV-2 reporter virus, it was demonstrated that functional SARS-CoV-2 protein variants with mutations in the receptor binding domain (RBD) and N-terminal domain that confer resistance to monoclonal antibodies or convalescent plasma can be readily selected. [Weisblum, Y. et al. BioRxiv doi: 10.1101/2020.07.21.214759]. Indeed, SARS-CoV-2 S variants that resist commonly elicited neutralizing antibodies are now present at low frequencies in circulating SARS-CoV-2 populations. [Weisblum, Y. et al. BioRxiv doi: 10.1101/2020.07.21.214759]. The emergence of such antibody-resistant SARS-CoV-2 variants might limit the therapeutic usefulness of monoclonal antibodies.

It also has been reported that in a macaque model, some sera from patients who eventually died of SARS-CoV and that displayed faster neutralizing antibody responses to Spike proteins caused severe acute lung injury in productively infected lungs by skewing macrophage responses during the acute phase of infection. [Liu, L. et al. J. Clin. Insight (2019) 4 (40): e123158].

The US Department of Health and Human Services (HHS) launched Operation Warp Speed—a partnership between government and industry—with the goal of delivering 300 million doses of a safe and effective vaccine by January 2021. [O'Callaghan, K P, et al. JAMA (2020) 324 (5): 437-38] This ambitious plan initially focused on 125 potential vaccine candidates, but was rapidly narrowed to 14 candidates in May 2020.

The leading vaccine candidates for COVID-19 all are aimed at inducing neutralizing antibodies directed against the receptor-binding domain (RBD) of the surface spike (S) protein of SARS-CoV-2.

Two of the 5 candidate vaccines are based on mRNA methodology. Moderna, a Massachusetts-based biotechnology company, has developed mRNA-1273, a lipid nanoparticle-encapsulated mRNA vaccine that encodes a full-length, prefusion stabilized spike (S) protein of SARS-CoV-2. [NCT04405076, visited 8/26/20]. Pfizer, in concert with BiBioNTech, a German company, is also developing an mRNA platform focused on lipid nanoparticle-encapsulated mRNA that encodes SARS-CoV-2 spike (S) protein.[NCT04368728, visited 8/26/20]. No vaccines to prevent human disease are commercially available using this strategy, and the complex lipid delivery system is also untested.

Merck Sharp & Dohme is partnering with the International AIDS Vaccine Initiative to develop a replicating recombinant vesicular stomatitis virus (VSV)-vectored platformed vaccine against SARS-CoV-2, using spike (S) protein as an antigenic target. [sciencemag.org/news/2020/05/merck-one-big-pharma-s-biggest-players-reveal s-its-covid-19-vaccine-and-therapy-plans]. Viral vector vaccines, rather than using attenuated versions of the target pathogen, use replication-competent versions of other viruses (the vector) to shuttle antigen-producing genes from the target pathogen to human cells. Merck has had success with this approach in developing an Ebola vaccine (Erbevo™). Merck also is perusing the use of an attenuated measles vector for its vaccine platform.

Two additional strategies involve replication-defective recombinant adenoviral vectors. Johnson & Johnson is the maker of a replication-defective adenovirus type 26 (Ad26) vector that delivers recombinant SARS-CoV-2 spike (S) protein genes to human cells, [https://www.jnj.com/johnson-johnson-announces-acceleration-of-its-covid-19-vaccine-candidate-phase-1-2a-clinical-trial-to-begin-in-second-half-of-july, visited 8/26/20]. AstraZeneca, the manufacturer of a replication-defective simian adenovirus vector (ChAdOx1 nCoV-19) in combination with the Jenner Institute at the University of Oxford, is similarly pursuing a phase 1-2 single blinded study [NCT04324606, visited 8/26/20]. No vaccines to prevent human disease are commercially available using this strategy, and their clinical use has been limited to one licensed vaccine against animal rabies. AstraZeneca also announced dosing of its first participants in a phase I trial of an investigational monoclonal antibody product AZD7442 for preventing and treating COVID19 in up to 48 healthy participants ages 18 to 55 years old in the UK. AZD7442 is a combination of two monoclonal antibodies that were discovered by researchers at Vanderbilt University Medical Center and licensed to AstraZeneca in June. [https://www.fdanews.com/articles/198715-astrazeneca-begins-human-trials-of-covid-19-monoclonal-antibody, Drug Daily Bulletin 17 (165): Aug. 26, 2020].

Vaccination Mediated Protection and its Shortcomings

Vaccine-induced immune effectors are essentially antibodies, produced by B lymphocytes, which are capable of binding specifically to a toxin or a pathogen. Other potential effectors are cytotoxic CD8+ T lymphocytes that may limit the spread of infectious agents by recognizing and killing infected cells or secreting specific antiviral cytokines and CD4+ T-helper (T_{H}) lymphocytes. These T_{H} cells may contribute to protection through cytokine production and provide support to the generation and maintenance of B and CD8+ T-cell responses. Effector CD4+ T_{H} cells were initially subdivided into T-helper 1 (T_{H}1) or T-helper 2 (T_{H}2) subsets depending on their main cytokine production (interferon-γ or interleukin [IL)-4), respectively. T_{H} cells are increasingly shown to include a large number of subsets with distinct cytokine-producing and homing capacities. For example, follicular T-helper (T_{FH}) cells are specially equipped and positioned in the lymph nodes to support potent B-cell activation and differentiation into antibody secreting cells; they were identified as directly controlling antibody responses and mediating adjuvanticity. T-helper 17 (T_{H}17) essentially defend against extracellular bacteria that colonize the skin and mucosa, recruiting neutrophils and promoting local inflammation. These effectors are controlled by regulatory T cells (T_{regs}) involved in maintaining immune tolerance.

Although the nature of a vaccine exerts a direct influence on the type of immune effectors that are elicited, the induction of antigen-specific immune effectors (and/or immune memory cells) by an immunization process does not imply that the resulting antibodies, cells, or cytokines represent surrogates, or even correlates, of vaccine efficacy (Rueckert C, Guzmán C A (2012) Vaccines: From Empirical Development to Rational Design. PLoS Pathog 8(11): e1003001. doi:10.1371/j ournal.ppat.1003001).

The protection provided by current vaccination efforts is largely dependent on the induction of neutralizing antibodies. Antibody-mediated neutralization of viruses is the direct inhibition of viral infectivity resulting from antibody docking to virus particles. Neutralization occurs when the process of virion binding to the cell surface receptors is inhibited, or when the fusion process of virion with cellular endosomal or plasma membranes is disrupted. Neutralizing antibodies precisely target specific antigens. In addition to directly interfering with virus entry into cells, antibodies can further counteract viral infection through their Fc fragments, triggering immune regulatory mechanisms, including ADCC, antibody-dependent cellular phagocytosis (ADCP), and CDCC.

However, neutralizing antibody protection has its limitations. First, there are a number of issues with the process of vaccine development itself, such as animal model unavailability. Second, pathogens present themselves in numerous variants and may undergo mutations to enable immune escape. Third, vaccine induced immunity may not be effective enough to confer long-term immunity. Fourth, despite experimental models, some vaccines may not invoke the desired functional response. Fifth, there are a number of population specific challenges that may alter the immune response to the vaccine. Sixth, there is insufficient information about the mechanisms of protection, as well as the antigens/epitopes required for sufficient activation of the targeted mechanism.

The extreme diversity of human immunodeficiency virus (HIV), a member of the family Retroviridae is a major obstacle to vaccine development, since strains belonging to different subtypes can differ by to 35% in some of their proteins, such as the env proteins. Therefore, while some vaccines may be effective against some virus clades, they may not be effective against other clades (see Hsu, D. et al, (2017) “Progress in HIV vaccine development” Human Vaccines & Immunotherapeutics 13(5): 1018-1030).

In summary then, despite the development of vaccines, morbidity and mortality from pathogens worldwide has not truly decreased. There are no universally accepted strategies and tools to rationally design vaccines, and vaccine development generally is still a tedious and costly empiric process. In many cases, rationally designed vaccines have not been successful, due to insufficient knowledge about the mechanisms of protection. Although the repertoire of immune clearance mechanisms to fight pathogens is known, the specific contributions of different effector mechanisms are well-characterized for only a few pathogens. It is also largely unclear what determines the immunogenicity and selection of particular epitopes among all possible antigenic options offered by a pathogen. For example, it is not known which factors determine dominant or balanced immune responses, and what are the mechanisms leading to long-term protection for each individual pathogen (Dye C. (2014). After 2015: infectious diseases in a new era of health and development. Philosophical transactions of the Royal Society of London. Series B, Biological sciences, 369(1645), 20130426. doi:10.1098/rstb.2013.0426).

Cell mediated reactions depend on direct interactions between specific T cells and target or antigen presenting cells which display the specific antigen in the context of either class I or II MEW molecules.

CD4+ and CD8+ T cell epitopes are present in many viral proteins (Jaye, A.; Herberts, C. A.; Jallow, S.; Atabani, S.; Klein, M. R.; Hoogerhout, P.; Kidd, M.; van Els, C. A.; Whittle, H. C. (2003) Vigorous but short-term gamma interferon T-cell responses against a dominant HLA-A*02-restricted measles virus epitope in patients with measles. J. Virol., 77, 5014-5016); (Ota, M. O.; Ndhlovu, Z.; Oh, S.; Piyasirisilp, S.; Berzofsky, J. A.; Moss, W. J.; Griffin, D. E. (2007) Hemagglutinin protein is a primary target of the measles virus-specific HLA-A2-restricted CD8+ T cell response during measles and after vaccination. J. Infect. Dis. 195, 1799-1807); (Schellens, I. M.; Meiring, H. D.; Hoof, I.; Spijkers, S. N.; Poelen, M. C.; van Gaans-van den Brink, J. A.; Costa, A. I.; Vennema, H.; Ke,smir, C.; van Baarle, D.; et al. (2015) Measles virus epitope presentation by HLA: Novel insights into epitope selection, dominance, and microvariation. Front. Immunol. 6, 546) and MeV-specific cytotoxic CD8+ T lymphocytes, IFN-γ-producing type 1 CD4+ and CD8+ T cells, along with soluble indicators of T cell activation (e.g., β2 microglobulin, cytokines and soluble CD4, CD8 and Fas), are in circulation during the rash phase of disease when infectious virus is being cleared (Lin, W. H.; Kouyos, R. D.; Adams, R. J.; Grenfell, B. T.; Griffin, D. E. (2012) Prolonged persistence of measles virus RNA is characteristic of primary infection dynamics. Proc. Natl. Acad. Sci. USA 109, 14989-14994); (Griffin, D. E.; Moench, T. R.; Johnson, R. T.; Lindo de Soriano, I.; Vaisberg, A. (1986) Peripheral blood mononuclear cells during natural measles virus infection: Cell surface phenotypes and evidence for activation. Clin. Immunol. Immunopathol. 40, 305-312); Griffin, D. E.; Ward, B. J.; Juaregui, E.; Johnson, R. T.; Vaisberg, A. (1992) Immune activation during measles: Beta 2-microglobulin in plasma and cerebrospinal fluid in complicated and uncomplicated disease. J. Infect. Dis. 166, 1170-1173); Griffin, D. E.; Ward, B. J.; Jauregui, E.; Johnson, R. T.; Vaisberg, A. (1990) Immune activation during measles: Interferon-gamma and neopterin in plasma and cerebrospinal fluid in complicated and uncomplicated disease. J. Infect. Dis. 161, 449-453; Griffin, D. E.; Ward, B. J.; Jauregui, E.; Johnson, R. T.; Vaisberg, A. (1989) Immune activation in measles. N. Engl. J. Med. 320, 1667-1672; Ward, B. J.; Johnson, R. T.; Vaisberg, A.; Jauregui, E.; Griffin, D. E. (1990) Spontaneous proliferation of peripheral mononuclear cells in natural measles virus infection: Identification of dividing cells and correlation with mitogen responsiveness. Clin. Immunol. Immunopathol. 55, 315-326; Jaye, A.; Magnusen, A. F.; Sadiq, A. D.; Corrah, T.; Whittle, H. C. (1998) Ex vivo analysis of cytotoxic T lymphocytes to measles antigens during infection and after vaccination in Gambian children. J. Clin. Investig., 102, 1969-1977). Depletion of CD8+ T cells from experimentally infected macaques results in higher and more prolonged viremias (Permar, S. R.; Klumpp, S. A.; Mansfield, K. G.; Kim, W. K.; Gorgone, D. A.; Lifton, M. A.; Williams, K. C.; Schmitz, J. E.; Reimann, K. A.; Axthelm, M. K.; et al. (2003) Role of CD8(+) lymphocytes in control and clearance of measles virus infection of rhesus monkeys. J. Virol. 77, 4396-4400) and CD8+ T cells can control virus spread in vitro (De Vries, R. D.; Yuksel, S.; Osterhaus, A. D.; de Swart, R. L. (2010) Specific CD8(+) T-lymphocytes control dissemination of measles virus. Eur. J. Immunol. 40, 388-395). As CD4+ and CD8+ T cells infiltrate sites of virus replication (Polack, F. P.; Auwaerter, P. G.; Lee, S. H.; Nousari, H. C.; Valsamakis, A.; Leiferman, K. M.; Diwan, A.; Adams, R. J.; Griffin, D. E. (1999) Production of atypical measles in rhesus macaques: Evidence for disease mediated by immune complex formation and eosinophils in the presence of fusion-inhibiting antibody. Nat. Med. 5, 629-634), infectious virus decreases rapidly to undetectable levels, the rash fades and the fever resolves.

While improving humoral immunity to viral infection is the target of many current conventional vaccines, for example influenza vaccines, such vaccines are generally not cross-protective. Moreover, most conserved viral proteins lie within the virus, and thus are out of reach of antibodies. Determining the peptide epitopes that are naturally generated by antigen presenting cells during virus infection would allow for the development of other vaccine formulations (i.e. peptide based) that can induce robust and cross reactive T cell responses.

The present disclosure describes universal vaccines targeting conserved virus T cell epitopes. The present disclosure is based, in part, on the identification of virus-specific CD8+ T cells which have a broad-spectrum anti-viral effect and importantly, can kill cells infected with different viral subtypes. Vaccine efficacy may be increased by inclusion of a Tx epitope; by increasing the size of peptides, forcing them to be presented by professional APCs, and by the use of DC-activating adjuvants.

The described immunogens, and vaccines comprising the same, offer a number of advantages over current vaccine strategies. One such advantage is that the immunogen comprises a highly conserved viral CD8+ T cell epitope that binds to human MHC class I molecules with high affinity, and is capable of inducing a broad spectrum of high levels of virus-specific T cell immunity. The broad immune response can effectively respond to virus escape from the host immune response due to antigenic drift and antigenic shift, and further may confer cross-protection effects across different viral subtypes. The immunization methods described herein employ, in some embodiments, a plurality of different vectors for sequential immunization, and use different inoculation methods to effectively activate a broad-spectrum T cell immune response both locally and systemically to enhance immune response across different viral subtypes. Accordingly, the present disclosure provides compositions and methods to stimulate the immune system more comprehensively, effectively, and permanently through the variety of different vaccine vectors, which are strategically combined to provide broader and more effective virus protection.

Taken together, the composition and methods provided by the disclosure provide advantages over, e.g., prior HIV vaccines, which have shown suboptimal specificity and limited breadth of vaccine-induced T-cell responses. For example, as has been seen in the past, the inclusion of full-length HIV-1 proteins in vaccine immunogens may drive CTL responses towards immunodominant epitopes, but often also have variable and therefore non-protective ‘decoy’ epitopes similar to those elicited by natural HIV-1 infection.

### SUMMARY OF THE INVENTION

According to one aspect, the described invention provides a universal vaccine against an immunogen of an infectious pathogenic organism., According to one aspect, the present disclosure provides a universal vaccine against an immunogen of an infectious pathogenic virus selected from a human Coronaviridae and a human Retroviridae virus comprising a pharmaceutical composition containing: at least one ribonucleic acid (RNA) polynucleotide comprising an open reading frame encoding at least one polypeptide antigen or an immunogenic fragment thereof, wherein the polypeptide antigen, or the immunogenic fragment thereof, comprises a conserved internal protein that is enriched in T cell recognition antigens, optionally helper T cell (TH) epitopes comprising at least one full-length protein that is immunogenic; or overlapping peptides of at least 30 amino acids in length that together cover the conserved internal viral protein; an optional immune response enhancer; and a pharmaceutically acceptable carrier, wherein a cytotoxic T lymphocyte (CTL) epitope consists of peptides of about 7 to about 14 residues in length.

According to some embodiments of the universal vaccine, the Coronaviridae virus is a human coronavirus. According to some embodiments, the optional helper T cell (TH) epitopes comprise at least one full-length protein of a human coronavirus selected from an S protein, an M protein, an E protein, or an N protein, wherein the full length protein is immunogenic; or (b) overlapping peptides of at least 30 amino acids in length that together cover the coronavirus S protein.

According to some embodiments, the immunogen contains at least one conserved protein of a human coronavirus, wherein the conserved protein is a coronavirus spike (S) protein of amino acid sequence SEQ ID NO: 1 or an immunogenic fragment thereof; or the conserved protein is a coronavirus spike (S) protein of amino acid sequence SEQ ID NO: 1 or an immunogenic fragment thereof of an amino acid sequence at least 85% identical to SEQ ID NO: 1; or the conserved protein is an isolated coronavirus S protein 51 subunit or an immunogenic fragment thereof of amino acid sequence SEQ ID NO: 1; or the conserved protein is an isolated coronavirus S protein 51 subunit cleaved by a transmembrane serine protease (TMPRSS2) comprising a receptor binding domain (RBD) or an immunogenic fragment thereof; or the conserved protein is an isolated coronavirus S protein 51 subunit cleaved by a transmembrane serine protease (TMPRSS2) comprising an RBD domain of the isolated 51 subunit enzymatically stripped of at least 5% of its covering of N-linked glycans, O-linked glycans or both by limited digestion; or the conserved protein is an isolated coronavirus S protein S2 subunit or an immunogenic fragment thereof; or the conserved protein is an isolated coronavirus S protein S2 subunit enzymatically stripped of at least 5% of its covering of N-linked glycans by limited digestion, or the conserved protein is a coronavirus membrane (M) protein or an immunogenic fragment thereof of amino acid sequence SEQ ID NO: 3 or 4; or the conserved protein is a coronavirus membrane (M) protein or an immunogenic fragment thereof; of an amino acid sequence at least 85% identical to SEQ ID NO: 3 or 4; or the conserved protein is a coronavirus envelope (E) protein or an immunogenic fragment thereof of amino acid sequence SEQ ID NO: 5 or 6; or the conserved protein is a coronavirus envelope (E) protein or an immunogenic fragment thereof of an amino acid sequence at least 85% identical to SEQ ID NO: 5 or SEQ ID NO: 6; or the conserved protein is a coronavirus nucleocapsid (N) protein or an immunogenic fragment thereof of amino acid sequence SEQ ID NO: 7 or 8; or the conserved protein is a coronavirus nucleocapsid (N) protein or an immunogenic fragment thereof of an amino acid sequence at least 85% identical to SEQ ID NO: 7 or SEQ ID NO: 8, or the conserved protein or immunogenic fragment is a combination thereof.

According to some embodiments, the Retroviridae virus is a human immunodeficiency virus (HIV). According to some embodiments, the immunogen contains at least one conserved protein of a human immunodeficiency virus (HIV), wherein the conserved protein is an HIV conserved capsid protein (gag), or an immunogenic fragment thereof of amino acid sequence SEQ ID NO: 9; or the conserved protein is an HIV conserved capsid protein (gag), or an immunogenic fragment thereof of an amino acid sequence at least 85% identical to SEQ ID NO: 9, or the conserved protein is an HIV conserved envelope protein (env) or an immunogenic fragment thereof of amino acid sequence SEQ ID NO: 10; or the conserved protein is an HIV conserved envelope protein (env) or an immunogenic fragment thereof of an amino acid sequence at least 85% identical to SEQ ID NO: 10, or the conserved protein is an HIV conserved polymerase protein (pol) or an immunogenic fragment thereof of amino acid sequence SEQ ID NO: 11; or the conserved protein is an HIV conserved polymerase protein (pol) or an immunogenic fragment thereof of an amino acid sequence at least 85% identical to SEQ ID NO: 11, or the conserved protein is an HIV conserved protease protein (pro) or an immunogenic fragment thereof of amino acid sequence SEQ ID NO: 12; or the conserved protein is an HIV conserved protease protein (pro) or an immunogenic fragment thereof of an amino acid sequence at least 85% identical to SEQ ID NO: 12, or the conserved protein or immunogenic fragment is a combination thereof.

According to some embodiments, the activated cell populations comprise activated cytotoxic T lymphocytes (CTLs). According to some embodiments, the activated CTLs comprise one or more of an NK cell population, an NKT cell population, an LAK cell population, a CIK cell population, a MAIT cell population, a CD8+ CTL population, or a CD4+ CTL population.

According to some embodiments, the immune enhancer comprises an adjuvant; or the immune enhancer comprises a naked DNA vector encoding a conserved polypeptide antigen or immunogenic fragment thereof comprising about 1 nanogram to about 2000 micrograms of DNA, inclusive; or the immune enhancer comprises both an adjuvant and a naked DNA vector encoding the conserved protein antigen.

According to some embodiments, the adjuvant comprises one or more of alum, aluminum salts, a saponin, an oil-in-water emulsion based on squalene, an unmethyl CpG dinucleotide; monophosphoryl lipid A (MPL) or an aminoalkyl glucosaminide-4-phosphate (AGP) mimetic thereof; 3-de-O-acylated monophosphoryl lipid A (3D-MPL), together with an aluminum salt; a monophosphoryl lipid A and saponin derivative; a polyoxyethylene ether; an anti-CD40 antibody; or GM-CSF.

According to some embodiments, the universal vaccine is prepared by a process comprising: (a) identifying and selecting from a consensus amino acid sequence a highly conserved internal protein of an infectious viral pathogen or an immunogenic fragment thereof enriched in T cell recognition antigens; (b) constructing immunogen sequences of the highly conserved internal proteins in (a); (c) constructing: a DNA vector comprising the immunogen sequences of (b); an adenovirus-based (AdV) vector comprising the immunogen sequences of (b); a replication-competent recombinant vaccinia virus based (VV) vector comprising the immunogen sequences of (b); (d) propagating separately each of the recombinant vectors comprising encoded immunogens in (c) for immunizing a subject in vivo in an amount effective to elicit or stimulate a therapeutic or prophylactic cell mediated immune response against an infection with the infectious pathogen by: priming the fully human immune system by immunizing with the phage DNA vector; boosting the fully human immune system by immunizing with the AdV vector of followed by the VV vector, or the VV vector of followed by the AdV vector of.

According to some embodiments of the universal vaccine prepared by the process, the DNA vector is selected from the group consisting of a Streptomyces phage SV1.0 DNA vector, an attenuated Mycobacterium tuberculosis vector, a Bacillus Calmette Guerin (BCG) vector, a Salmonella species bacterial vector, a Shigella species bacterial vector, the AdV viral vector is selected from the group consisting of Adenovirus (Ad vectors) based on Ad serotype 5 (AdHu5), adeno-associated virus (AAV), AD26 vector chimpanzee adenoviral isolate Y25, AdC68/Sad-V25), ChAd63, AdC68 (SAdV-25), AdC7 (SAdV-24) and AdC6 (SAdV-23), and ChAdOx1; the vaccinia virus viral vector is selected from the group consisting of attenuated vaccinia strains Modified Vaccinia Ankara (MVA), chorioallantois vaccinia virus Ankara (CVA) strain], live vaccinia virus strains WR strain, New York City Board of Health (NYCBH) strain, ACAM2000, Lister strain, LC16 m8, Elstree-BNm, Copenhagen strain, and Tiantan strain (VTT). According to some embodiments of the universal vaccine prepared by the process, the viral pathogen is a human coronavirus. According to some embodiments of the universal vaccine prepared by the process, the conserved protein is a coronavirus spike (S) protein of amino acid sequence SEQ ID NO: 1; or the conserved protein is a coronavirus spike (S) protein of an amino acid sequence at least 85% identical to SEQ ID NO: 1; or the conserved protein is an isolated coronavirus S protein S1 subunit of amino acid sequence SEQ ID NO: 1; or the conserved protein is an isolated coronavirus S protein S1 subunit cleaved by a transmembrane serine protease (TMPRSS2) comprising a receptor binding domain (RBD); or the conserved protein is an isolated coronavirus S protein S1 subunit cleaved by a transmembrane serine protease (TMPRSS2) comprising an RBD domain of an S1 subunit enzymatically stripped of at least 5% of its covering of N-linked glycans, O-linked glycans or both by limited digestion; or the conserved protein is an isolated coronavirus S protein S2 subunit subunit of amino acid sequence SEQ ID NO: 1; or the conserved protein is an isolated coronavirus S protein S2 subunit enzymatically stripped of at least 5% of its covering of N-linked glycans by limited digestion, or the conserved protein is a coronavirus membrane (M) protein of amino acid sequence SEQ ID NO: 3 or 4; or the conserved protein is a coronavirus membrane (M) protein of an amino acid sequence at least 85% identical to SEQ ID NO: 3 or 4; or the conserved protein is a coronavirus envelope (E) protein of amino acid sequence SEQ ID NO: 5 or 6; or the conserved protein is a coronavirus envelope (E) protein of an amino acid sequence at least 85% identical to SEQ ID NO: 5 or SEQ ID NO: 6; or the conserved protein is a coronavirus nucleocapsid (N) protein of amino acid sequence SEQ ID NO: 7 or 8; or the conserved protein is a coronavirus nucleocapsid (N) protein or immunogenic fragment thereof of an amino acid sequence at least 85% identical to SEQ ID NO: 7 or SEQ ID NO: 8, or the conserved protein or immunogenic fragment is a combination thereof.

According to some embodiments of the universal vaccine prepared by the process, the virus is a Retroviridae virus and the Retroviridae virus is a human immunodeficiency virus (HIV). According to some embodiments of the universal vaccine prepared by the process, the conserved protein is an HIV conserved capsid protein (gag) of SEQ ID NO: 9; or the conserved protein is an HIV conserved capsid protein (gag) of an amino acid sequence at least 85% identical to SEQ ID NO: 9, or the conserved protein is an HIV conserved envelope protein (env) of SEQ ID NO: 10; or the conserved protein is an HIV conserved envelope protein (env) of an amino acid sequence at least 85% identical to SEQ ID NO: 10, or the conserved protein is an HIV conserved polymerase protein (pol) of SEQ ID NO: 11; or the conserved protein is an HIV conserved polymerase protein (pol) of an amino acid sequence at least 85% identical to SEQ ID NO: 11, or the conserved protein is an HIV conserved protease protein (pro) of SEQ ID NO: 12; or the conserved protein is an HIV conserved protease protein (pro) of an amino acid sequence at least 85% identical to SEQ ID NO: 12, or the conserved protein or immunogenic fragment is a combination thereof.

According to another aspect, the present disclosure provides an engineered nucleic acid encoding at least one RNA polynucleotide comprising an open reading frame encoding at least one polypeptide antigen or an immunogenic fragment thereof, wherein the polypeptide antigen, or the immunogenic fragment thereof, comprises a conserved internal protein that is enriched in T cell recognition antigens of the universal vaccine.

According to another aspect, the present disclosure provides an expression vector comprising an engineered nucleic acid encoding at least one RNA polynucleotide comprising an open reading frame encoding at least one polypeptide antigen or an immunogenic fragment thereof, wherein the polypeptide antigen, or the immunogenic fragment thereof, comprises a conserved internal protein that is enriched in T cell recognition antigens of the universal vaccine.

According to another aspect, the present disclosure provides a host cell comprising an engineered nucleic acid encoding at least one RNA polynucleotide comprising an open reading frame encoding at least one polypeptide antigen or an immunogenic fragment thereof, wherein the polypeptide antigen, or the immunogenic fragment thereof, comprises a conserved internal protein that is enriched in T cell recognition antigens of the universal vaccine.

According to another aspect, the present disclosure provides a method of inducing an immune response in a subject, the method comprising administering to the subject a universal vaccine against an immunogen of an infectious pathogenic virus selected from a human Coronaviridae or a human Retroviridae virus comprising at least one ribonucleic acid (RNA) polynucleotide comprising an open reading frame encoding at least one antigenic polypeptide or an immunogenic fragment thereof, wherein the antigenic peptide, or the immunogenic fragment thereof, comprises a conserved internal protein that is enriched in T cell recognition antigens, optional helper T cell (TH) epitopes comprising at least one full-length protein that is immunogenic; or overlapping peptides of at least 30 amino acids in length that together cover the conserved internal viral protein; an optional immune response enhancer; and a pharmaceutically acceptable carrier, wherein a T cell recognition antigen consists of peptides of about 7 to about 14 residues in length, and (2) the immune response produced in response to the vaccine comprises one or more of: (i) activation of one or more T cell populations directed to an antigen(s) present in the vaccine; or (ii) neutralization of infectivity of the pathogen; or (iii) an antigen-specific response comprising destruction of the infectious pathogenic organism; lysis of cells infected with the infectious pathogenic organism, or both; compared to a control immunized with the immunogen without the ribonucleic acid (RNA) polynucleotide comprising the open reading frame encoding the at least one polypeptide antigen or the immunogenic fragment thereof.

According to some embodiments of the method, the immune enhancer comprises an adjuvant; or the immune enhancer comprises a naked DNA vector encoding a conserved polypeptide antigen or immunogenic fragment thereof comprising about 1 nanogram to about 2000 micrograms of DNA, inclusive; or the immune enhancer comprises both an adjuvant and a naked DNA vector encoding the conserved protein antigen. According to some embodiments the adjuvant comprises one or more of alum, aluminum salts, a saponin, an oil-in-water emulsion based on squalene, an unmethyl CpG dinucleotide; polyinosinic-polycytidylic acid (poly(I:C); monophosphoryl lipid A (MPL) or an aminoalkyl glucosaminide-4-phosphate (AGP) mimetic thereof; 3-de-O-acylated monophosphoryl lipid A (3D-MPL), together with an aluminum salt; a monophosphoryl lipid A and saponin derivative; a polyoxyethylene ether; an anti-CD40 antibody; or GM-CSF. According to some embodiments, the method further comprises priming the subject with a naked nucleic acid or DNA vector comprising a first immunogen sequence encoding a conserved internal protein that is enriched in T cell recognition antigens; and then boosting the subject with a boosting composition comprising a an attenuated, replication-competent recombinant vaccinia virus based (VV) vector comprising a second immunogen sequence encoding a conserved internal protein that is enriched in CD8+ T cell recognition antigens. According to some embodiments, the DNA vector is selected from the group consisting of a Streptomyces phage SV1.0 DNA vector, an attenuated Mycobacterium tuberculosis vector, a Bacillus Calmette Guerin (BCG) vector, a Salmonella species bacterial vector, a Shigella species bacterial vector, or the AdV viral vector is selected from the group consisting of Adenovirus (Ad vectors) based on Ad serotype 5 (AdHu5), adeno-associated virus (AAV), AD26 vector chimpanzee adenoviral isolate Y25, AdC68/Sad-V25), ChAd63, AdC68 (SAdV-25), AdC7 (SAdV-24) and AdC6 (SAdV-23), and ChAdOx1; or the vaccinia virus viral vector is selected from the group consisting of attenuated vaccinia strains Modified Vaccinia Ankara (MVA), chorioallantois vaccinia virus Ankara (CVA) strain], Live vaccinia virus strains WR strain, New York City Board of Health (NYCBH) strain, ACAM2000, Lister strain, LC16 m8, Elstree-BNm, Copenhagen strain, and Tiantan strain (VTT). According to some embodiments, the naked nucleic acid or the DNA vector encodes one or more conserved protein of a human immunodeficiency virus; and the boosting composition comprises a subunit or protein vaccine comprising one or more conserved proteins of the human immunodeficiency virus. According to some embodiments, the naked nucleic acid or DNA vector encodes a conserved protein of a human coronavirus; and the boosting composition comprises a subunit or protein vaccine comprising one or more conserved proteins of the human coronavirus.

According to some embodiments, the conserved protein of the human coronavirus is one or more of: a human coronavirus (S) protein of amino acid sequence SEQ ID NO: 2 or an immunogenic fragment thereof; or a coronavirus membrane (M) protein or an immunogenic fragment thereof of amino acid sequence SEQ ID NO: 3 or 4; or a coronavirus nucleocapsid (N) protein or an immunogenic fragment thereof of amino acid sequence SEQ ID NO: 7 or 8; or or a coronavirus envelope (E) protein or an immunogenic fragment thereof of amino acid sequence SEQ ID NO: 5 or 6. According to some embodiments, the naked nucleic acid or DNA vector encodes a conserved protein of a human immunodeficiency virus; and the boosting composition comprises a subunit or protein vaccine comprising one or more conserved proteins of the human immunodeficiency virus.

According to some embodiments, the conserved protein of the human immunodeficiency virus is one or more of: an HIV conserved capsid protein (gag) of amino acid sequence SEQ ID NO: 9 or an immunogenic fragment thereof; or an HIV conserved envelope protein (env) or an immunogenic fragment thereof of amino acid sequence SEQ ID NO: 10; or an immunogenic fragment thereof; or an HIV conserved polymerase protein (pol) or an immunogenic fragment thereof of amino acid sequence SEQ ID NO: 11, or an immunogenic fragment thereof; or an HIV conserved protease protein (pro) or an immunogenic fragment thereof of amino acid sequence SEQ ID NO: 12.

According to some embodiments, the method comprises administering the vaccine to the subject by inhalation, insufflation a or by intramuscular injection. According to some embodiments, the mode of administration of the priming dose and the mode of administration of the booster dose are different. According to some embodiments, the subject is a mouse of phenotype NOD-scid γc−/− or BALB/c Rag2−/− γc−/−. According to some embodiments, the method comprises reconstituting the mouse of phenotype NOD-scid γc−/− with human C34+CD133+ cord blood cells injected intracardially as newborns into the NOD-scid γc−/− mouse. According to some embodiments, the method comprises reconstituting the mouse of phenotype BALB/c Rag2−/− γc−/− comprises CD34+ hematopoietic progenitor cells (HPCs) isolated from human fetal liver transferred intrahepatically into newborn BALB/c Rag2−/− γc−/−.

According to another aspect, the present disclosure provides a method for inducing a pan-coronavirus specific cellular immune response in vivo in an animal model comprising a fully human functional immune system comprising: identifying and selecting from a consensus amino acid sequence a plurality of highly conserved coronavirus viral proteins enriched in T cell recognition antigens; constructing concatenated immunogen sequences of the highly conserved coronavirus viral proteins; constructing: a DNA vector, comprising the concatenated immunogen sequences; an adenovirus-based (AdV) vector comprising the concatenated immunogen sequences; an attenuated, replication-competent recombinant vaccinia virus based (VV) vector comprising the concatenated immunogen sequences; propagating separately each of the recombinant vectors comprising encoded immunogens; immunizing the animal model comprising the fully human functional immune system in vivo by: priming the fully human immune system by immunizing with the DNA vector; boosting the fully human immune system by immunizing with the AdV vector of followed by the VV vector, or the VV vector of 3(c) followed by the AdV vector; and after the immunizing, challenging the animal model comprising the immunized fully human functional immune system with a SARS-CoV-1, MERS-CoV, or SARS-CoV-2 virus.

According to some embodiments, the DNA vector is selected from the group consisting of a Streptomyces phage SV1.0 DNA vector, an attenuated Mycobacterium tuberculosis vector, a Bacillus Calmette Guerin (BCG) vector, a Salmonella species bacterial vector, a Shigella species bacterial vector, the AdV viral vector is selected from the group consisting of Adenovirus (Ad vectors) based on Ad serotype 5 (AdHu5), adeno-associated virus (AAV), AD26 vector chimpanzee adenoviral isolate Y25, AdC68/Sad-V25), ChAd63, AdC68 (SAdV-25), AdC7 (SAdV-24) and AdC6 (SAdV-23), and ChAdOx1; the pox virus viral vector is selected from the group consisting of attenuated vaccinia strains Modified Vaccinia Ankara (MVA), chorioallantois vaccinia virus Ankara (CVA) strain], Live vaccinia virus strains WR strain, New York City Board of Health (NYCBH) strain, ACAM2000, Lister strain, LC16 m8, Elstree-BNm, Copenhagen strain, and Tiantan strain (VTT).

According to some embodiments, the step of immunizing further comprising administering a pharmaceutical composition containing helper T cell (TH) epitopes comprising at least one full-length protein that is immunogenic; or overlapping peptides of at least 30 amino acids in length that together cover the conserved internal viral protein; an optional immune response enhancer; and a pharmaceutically acceptable carrier. According to some embodiments, the immune response enhancer is an adjuvant, and the adjuvant comprises one or more of alum, aluminum salts, a saponin, an oil-in-water emulsion based on squalene, an unmethyl CpG dinucleotide; polyinosinic-polycytidylic acid (poly(I:C); monophosphoryl lipid A (MPL) or an aminoalkyl glucosaminide-4-phosphate (AGP) mimetic thereof; 3-de-O-acylated monophosphoryl lipid A (3D-MPL), together with an aluminum salt; a monophosphoryl lipid A and saponin derivative; a polyoxyethylene ether; an anti-CD40 antibody; or GM-CSF. According to some embodiments, the method comprises administering the vaccine to the subject by intradermal injection, intranasally, by insufflation, or by intramuscular injection. According to some embodiments, the mode of administration of the priming dose and the booster dose are different. According to some embodiments, the animal model is a mouse of phenotype NOD-scid γc−/− or BALB/c Rag2−/− γc−/−. According to some embodiments, the method comprises reconstituting the mouse of phenotype NOD-scid γc−/− with human C34+ CD133+ cord blood cells injected intracardially as newborns into the NOD-scid γc−/− mouse. According to some embodiments, the method comprises reconstituting the mouse of phenotype BALB/c Rag2−/− γc−/− comprises CD34+ hematopoietic progenitor cells (HPCs) isolated from human fetal liver transferred intrahepatically into newborn BALB/c Rag2−/− γc−/−. According to some embodiments, the coronavirus specific cellular and humoral immune response in the animal model may be effective to reduce spread of infection in a population of unimmunized reconstituted mice. According to some embodiments, the conserved protein is a coronavirus spike (S) protein of amino acid sequence SEQ ID NO: 1; or the conserved protein is a coronavirus spike (S) protein of an amino acid sequence at least 85% identical to SEQ ID NO: 1; or the conserved protein is an isolated coronavirus S protein S1 subunit of amino acid sequence SEQ ID NO: 1; or the conserved protein is an isolated coronavirus S protein S1 subunit cleaved by a transmembrane serine protease (TMPRSS2) comprising a receptor binding domain (RBD); or the conserved protein is an isolated coronavirus S protein S1 subunit cleaved by a transmembrane serine protease (TMPRSS2) comprising an RBD domain of an S1 subunit enzymatically stripped of at least 5% of its covering of N-linked glycans, O-linked glycans or both by limited digestion; or the conserved protein is an isolated coronavirus S protein S2 subunit subunit of amino acid sequence SEQ ID NO: 1; or the conserved protein is an isolated coronavirus S protein S2 subunit enzymatically stripped of at least 5% of its covering of N-linked glycans by limited digestion, or the conserved protein is a coronavirus membrane (M) protein of amino acid sequence SEQ ID NO: 3 or 4; or the conserved protein is a coronavirus membrane (M) protein of an amino acid sequence at least 85% identical to SEQ ID NO: 3 or 4; or the conserved protein is a coronavirus envelope (E) protein of amino acid sequence SEQ ID NO: 5 or 6; or the conserved protein is a coronavirus envelope (E) protein of an amino acid sequence at least 85% identical to SEQ ID NO: 5 or SEQ ID NO: 6; or the conserved protein is a coronavirus nucleocapsid (N) protein of amino acid sequence SEQ ID NO: 7 or 8; or the conserved protein is a coronavirus nucleocapsid (N) protein or immunogenic fragment thereof of an amino acid sequence at least 85% identical to SEQ ID NO: 7 or SEQ ID NO: 8, or the conserved protein or immunogenic fragment is a combination thereof.

### BRIEF DESCRIPTION OF DRAWINGS

The patent or application file contains at least one drawing executed in color. Copies of this patent or patent application publication with color drawing(s) will be provided by the Office upon request and payment of the necessary fee

FIG. 1 shows structural features of the SARS-CoV-2 spike (S) protein. (A) Ribbon diagram of the homotrimeric S. (B) Side view of the prefusion structure of S with a single RBD inn open (“up) conformation (green). (C) Top view of the prefusion structure of S, with two single RBDs in closed (“down”) conformation (white and gray) and one single RBD in open (“up”) conformation (green). (D) Single monomer of S with the RBD in closed (“down”) conformation (green). (E) Single monomer of S, with the RBD in open (“up”) conformation (green). {Taken from Sternberg, A. & Naujokat, C. Life Sciences (2020) 257: 118056].

### DETAILED DESCRIPTION

### Definitions

Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. All publications mentioned herein are incorporated by reference. Nothing herein is to be construed as an admission that the disclosure is not entitled to antedate such disclosure by virtue of prior disclosure.

As used in the specification and the appended claims, the singular forms “a,” “an” and “the” include plural referents unless the context clearly dictates otherwise.

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. For example, a mature B cell can be activated by an encounter with an antigen that expresses epitopes that are recognized by its cell surface immunoglobulin Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC). The soluble product of an activated B lymphocyte is immmunoglobulins (antibodies). The soluble product of an activated T lymphocyte is lymphokines (meaning cytokines produced by lymphocytes).

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer. According to some embodiments, to A without B (optionally including elements other than B). According to some embodiments, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of” “only one of” or “exactly one of” “Consisting essentially of,” when used in the claims, shall have its ordinary meaning as used in the field of patent law.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. Thus, a polypeptide “comprises” an amino acid sequence when the amino acid sequence might be part of the final amino acid sequence of the polypeptide. Such a polypeptide can have up to several hundred additional amino acids residues (e.g. tag and targeting peptides as mentioned herein). “Consisting essentially of” means excluding other components or steps of any essential significance. Thus, a composition consisting essentially of the recited components would not exclude trace contaminants and pharmaceutically acceptable carriers. A polypeptide “consists essentially of” an amino acid sequence when such an amino acid sequence is present with eventually only a few additional amino acid residues. “Consisting of” means excluding more than trace elements of other components or steps. For example, a polypeptide “consists of” an amino acid sequence when the polypeptide does not contain any amino acids but the recited amino acid sequence.

As used herein, “substantially equal” means within a range known to be correlated to an abnormal or normal range at a given measured metric. For example, if a control sample is from a diseased patient, substantially equal is within an abnormal range. If a control sample is from a patient known not to have the condition being tested, substantially equal is within a normal range for that given metric.

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, preferred materials and methods are described herein.

The terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses.

The terms “activating CD8+ T cells” or “CD8+ T cell activation” as used herein are meant to refer to a process (e.g., a signaling event) causing or resulting in one or more cellular responses of a CD8+ T cell (CTL), selected from: proliferation, differentiation, cytokine secretion, cytotoxic effector molecule release, cytotoxic activity, and expression of activation markers. As used herein, an “activated CD8+ T cell” refers to a CD8+ T cell that has received an activating signal, and thus demonstrates one or more cellular responses, selected from proliferation, differentiation, cytokine secretion, cytotoxic effector molecule release, cytotoxic activity, and expression of activation markers. Suitable assays to measure CD8+ T cell activation are known in the art and are described herein.

The terms “activating an NK cell” or “NK cell activation” as used herein is meant to refer to a process (e.g., a signaling event) causing or resulting in an NK cell being capable of killing cells with deficiencies in MHC class I expression. As used herein, an “activated NK cell” refers to an NK cell that has received an activating signal, and is thus capable of killing cells with deficiencies in MHC class I expression. Suitable assays to measure NK cell activation are known in the art and are described herein.

The term “active immunization” as used herein refers to The term “active immunization” as used herein refers to the production of active immunity, meaning immunity resulting from a naturally acquired infection or intentional vaccination (artificial active immunity).

Acute lung injury (ALI) and acute respiratory distress syndrome (ARDS). ALI and its more severe form, ARDS, are syndromes of acute respiratory failure that result from acute pulmonary edema and inflammation. ALI/ARDS is a cause of acute respiratory failure that develops in patients of all ages from a variety of clinical disorders, including sepsis (pulmonary and nonpulmonary), pneumonia (bacterial, viral, and fungal), aspiration of gastric and oropharyngeal contents, major trauma, and several other clinical disorders, including severe acute pancreatitis, drug over dose, and blood products [Ware, L. and Matthay, M., N Engl J Med, (2000) 342:1334-1349,]. Most patients require assisted ventilation with positive pressure. The primary physiologic abnormalities are severe arterial hypoxemia as well as a marked increase in minute ventilation secondary to a sharp increase in pulmonary dead space fraction. Patients with ALI/ARDS develop protein-rich pulmonary edema resulting from exudation of fluid into the interstitial and airspace compartments of the lung secondary to increased permeability of the barrier. Additional pathologic changes indicate that the mechanisms involved in lung edema are complex and that edema is only one of the pathophysiologic events in ALI/ARDS. One physiologic consequence is a significant decrease in lung compliance that results in an increased work of breathing [Nuckton T. et al., N Engl J Med. (2002) 346:1281-1286,], one of the reasons why assisted ventilation is required to support most patients. It has been reported that soon after onset of respiratory distress from COVID, patients initially retain relatively good compliance despite very poor oxygenation. [Marini, J J and Gattinoni, L., JAMA Insights (2020) doi: 10.1001/jama.2020.6825, citing Grasselli, G. et al., JAMA (2020) doi: 10.1001/jama.2020.5394; Arentz, M. et al. JAMA (2020) doi: 10.1001/jama.2020.4326]. Minute ventilation is characteristically high. Infiltrates are often limited in extent and, initially, are usually characterized by a ground-glass pattern on CT that signifies interstitial rather than alveolar edema. Many patients do not appear overtly dyspneic. These patients can be assigned, in a simplified model, to “type L,” characterized by low lung elastance (high compliance), lower lung weight as estimated by CT scan, and low response to PEEP. {Id., citing Gattinoni, L. et al. Intensive Care Med. (2020) doi: 10.1007/s00134-020-06033-2}. For many patients, the disease may stabilize at this stage without deterioration while others, either because of disease severity and host response or suboptimal management, may transition to a clinical picture more characteristic of typical ARDS. These can be defined as “type H,” with extensive CT consolidations, high elastance (low compliance), higher lung weight, and high PEEP response. Types L and H are the conceptual extremes of a spectrum that includes intermediate stages, in which their characteristics may overlap.

The term “adaptor molecule” as used herein refers to a specialized protein that links protein components of a signaling pathway, thereby aiding intracellular signal transduction.

The terms “adoptive immunity” and “acquired immunity” are used interchangeably to refer to passive cell mediated immunity produced by the transfer of living lymphoid cells from an immune cell source.

The term “adjuvant” as used herein, is meant to refer to a compound that provides the help needed to enhance the immunogenicity of vaccine antigens. There are two distinct reasons to incorporate an adjuvant into a vaccine (1) to increase the magnitude of an adaptive response to a vaccine, based on antibody titer or ability to prevent infection, which facilitates the use of smaller doses of antigen and permits immunization with fewer doses of vaccine; and (2) to achieve qualitative alteration of the immune response, for example, to provide functionally appropriate types of immune response (Th1 cell vs Th2 cell; CD8+ vs. CD4+ T cells, specific antibody isotypes), to increase the generation of immunological memory (e.g., T cell memory) [Cottman, R L et al, Immunity (2010) 33 (4): 492-503, citing Galli, G. et al. (2009) Proc Natl Acad Sci USA. 106:3877-3882; Leroux-Roels, I. et al. (2010) Vaccine. 2010; 28:849-857; Vandepapeliere, P. et al (2008) Vaccine. 26:1375-1386; to increase speed of initial response, which may be critical in a pandemic outbreak of infection [Id., citing Gallli, G. (2009) Proc Natl Acad Sci USA. 106: 3877-3882; Huleatt, J W et al (2007) Vaccine. 25:763-775; Khurana, S. et al (2010) Sci Transl Med. 2:15ra15; and to alter the breadth, specificity or affinity of the response [Id., citing Khurana, S. et al (2010) Sci Transl Med. 2:15ra15; Malherbe, L. et al (2008) Immunity. 2008; 28:698-709. Modification of the immune response includes intensification or broadening the specificity of either or both antibody and cellular immune responses as well as decreasing or suppressing certain antigen-specific immune responses.

The immune system is optimized to generate adaptive responses to microbial antigens delivered to APCs in intimate association with PRR ligands, as would be the case for viral and microbial infections and live attenuated vaccines. Members of nearly all of the PRR families are potential targets for adjuvants. These include TLRs recognizing lipids, lipoproteins, nucleic acids, and proteins; NOD-like receptors (NLR, also defined as “nucleotide-binding domain and leucine-rich repeat containing” receptors) responding to multiple ligands such as peptidoglycan species, flagellin, toxins, and ATP; helicases (RIG-I-like receptors, RLR) triggered by cytoplasmic RNA; and C-type lectin receptors (CLRs) recognizing carbohydrates and lipids [Id., citing Beutler, B A (2009) Blood 113: 1399-1407; Iwasaki A, Medzhitov R. Science (2010) 327:291-295; Kawai T, Akira S. Nat Immunol. (2010) 11:373-384; Takeuchi O, Akira S. Cell. 2010; 140:805-820]. They signal through pathways involving distinct adaptor molecules and intermediates such as MyD88 (an adaptor protein that functions in signaling by all TLR proteins except TLR3), TRIF (an adaptor protein that alone is involved in signaling by TLR-3, and that, when paired with TRAM (an adaptor protein that pairs with TRIF in signaling by TLR-4), functions in signaling by TLR-4, RIP2 (a caspase activation and recruitment (CARD) domain containing serine-threonine kinase that functions in signaling by NOD proteins to activate the NFκB transcription factor), Caspase Recruitment Domain Family Member 9 (CARDS, a signaling adaptor protein involved in the transduction of signals from PRRs, including c-type lectin receptors and intracellular NOD receptors and nuclei acid sensors) and interferon-beta promoter stimulator 1 (IPS-1, an adaptor involved in RIG-I- and Mda5-mediated antiviral immune responses; ‘knockdown’ of IPS-1 by small interfering RNA was demonstrated to block interferon induction by virus infection [Kawai, T. et al. Nat. Immunol. (2005) 6 (10): 981-8] that partially dictate the outcome of receptor-ligand interaction. Two key transcriptional programs involving the transcription factors NF-κB (activated by the stimulation of TLRs and by antigen receptor signaling composed of p50 and p65 subunits), IRF-3, and IRF-7 (members of the IRF family of transcription factors that interact with activated STAT1 and STAT2 to form the complex called ISGF3, which induces transcription of many interferon-stimulated genes (ISGs)) are activated by these signaling circuits, resulting in the induction of genes encoding cytokines, chemokines, and costimulatory molecules that play a key role in priming, expansion, and polarization of immune responses [Cottman, R L et al, Immunity (2010) 33 (4): 492-503, citing O'Neill L A, Bowie A G. Curr Biol. (2010) 20:R328-R333].

Signaling pathways triggered by constituents of damaged or dying host cells can also contribute to the function of adjuvants. This process occurs in part through the inflammasome, a molecular complex that activates caspase 1, which in turn cleaves pro-interleukin-1 β (IL-1β) and pro-IL-18 into their bioactive forms [Id., citing Martinon, F. et al. Annu Rev Immunol. (2009) 27:229-265]. The inflammasome complex is formed upon triggering of nucleotide-binding domain, leucine rich containing proteins (NLRB) such as NLRP3 (NOD-, LRR- and pyrin domain-containing protein 3), which is an intracellular sensor that detects a broad range of microbial motifs, endogenous danger signals and environmental irritants, resulting in the formation and activation of the NLRP3 inflammasome] and NLRC4 (trigger (e.g. cytosolic flagellin), sensor (NAIP), nucleator (NLRC4), adaptor (ASC), and effector (CASP1) [Duncan, J A, Canna, S W. Immunol. Rev. (2018) 28 (1): 115-123]. This can occur through recognition of microbial ligands such as flagellin or through indirect mechanisms such as host lysosomal damage resulting from the phagocytosis of crystalline particles (e.g., alum and uric acid). Necrotic cells release ATP and uric acid, which activate the NLRP3 inflammasome, thereby linking cellular damage to an inflammatory response [Cottman, R L et al, Immunity (2010) 33 (4): 492-503, citing Hornung, V. et al. Nat. Immunol. (2008) 9: 847-56; Iyer, S S et al. Oric, Natl Acad. Sci. USA (2009) 106: 20388-93].

For example, alum and aluminum-salts have long been utilized as adjuvants. [See, e.g., Lindblad (2004) Immunol. Cell. Biol. 82(5):497-505; Gupta and Siber (1995) A Vaccine 13(14):1263-1276; Gupta and Rost (2000) In O'Hagan D, editor Vaccine Adjuvants: Preparation Methods and Research Protocols, ed., Totowa, N.J.: Humana Press Inc. p 65-89; Cox and Coulter, (1997) Vaccine 15(3):248-256]. MF59 (Novartis) and AS03 (GlaxoSmithKline) are both oil-in-water emulsions based on squalene, an oil that is more readily metabolized than the paraffin oil used in Freund's adjuvants. MF59 is licensed in most of Europe for use with seasonal flu vaccines in the elderly, and both are used in approved pandemic flu vaccines. As a result, there are considerable human data comparing flu vaccination with these adjuvants to the same vaccine without adjuvant or with alum [Id., citing Mbow, M L et al. Curr Opin Immunol. (2010) 22:411-416]. These emulsions stimulate stronger antibody responses, permit fewer doses and antigen dose sparing, and generate marked memory responses, with a mixed Th1-Th2 cell phenotype [Id., citing Ott, G. et al. Pharm Biotechnol. (1995) 6:277-296]. MF59 induces substantial local stimulation, recruitment of DCs, granulocytes, and differentiation of monocytes into DCs [Id., citing Seubert A. et al. J. Immunol. (2008) 180:5402-5412], as well as increased uptake of antigen by DCs [Id., citing Dupuis, M. et al., Cell Immunol. (1998) 186: 18-27]. Intramuscular injection of MF59 leads to a pattern of induced genes that is both larger and distinct from that induced by either alum or a TLR9 agonist [Id., citing Mosca, F. et al. Proc Natl Acad Sci USA. (2008) 105:10501-10506]. Others include saponin-based Matrix-M™ [Isconova AB, Uppsala, Sweden; see Bengtsson, K L et al. Expert Review of Vaccines (2011) 4: 401-403], CpG 1018 [Dynavax' vaccine adjuvant for its hepatitis B vaccine, HEPLISAV-B, which strongly favors development of the Th1 subset of helper cells, and targets TLR9, https://www.dynavax.com/science/cpg-1018/, visited Nov. 24, 2020]; AS01 (a liposome-based vaccine adjuvant system containing two immunostimulants: 3-O-desacyl-4′-monophosphoryl lipid A (MPL) and the saponin QS-21; see Didierlaurent, A M et al. Expert Rev. Vaccines (2017) 16 (1): 55-63]. mRNA or viral vectors can act as their own adjuvant.

Exemplary molecular defined strong dendritic cell-activating adjuvants include, without limitation, oligodeoxynucleotide (ODN)-CpG [Zwaveling, S. et al. J. Immunol. (2002) 169: 350-8, citing Sparwasser, T. et al. Eur. J. Immunol. (1998) 28: 2045; Weiner, G J, et al. Proc. Natl Acad. Sci. USA (1997) 94: 10833; Vabulas, R M, et al. J. Immunol. (2000) 164: 237], polyinosinic-polycytidylic acid (poly-IC); monophosphoryl lipid A (MPL) [Id., citing Thompson, H S, et al. Vaccine (1998) 16: 1993; Ulrich, J T, Myers, K R. Pharm. Biotechnol. (1995) 6: 495] or a mimetic thereof (e.g., RC-529 or RC-544, members of the aminoalkyl glucosaminide-4-phosphate family of lipid A mimetics; Persing, D H et al. Trends in Microbiology (2002) 10 (10) Suppl] S32-S37], anti-CD40 antibody (commercially available e.g., from Abcam, Cambridge, Mass.) [Id., citing Diehl, L et al. (1999) Nature Med. (1999) 5: 774] and GM-CSF (Sargramostim™) [Id., citing Disis, M L et al. Blood (1996) 88: 202]. It has been demonstrated that CpG containing DNA and GM-CSF increased DC activation and maturation synergistically [Hartmann, G. et al. Proc. Nat. Acad. Sci. USA (1999) 96: 9305-10].

The term “administration” and its various grammatical forms as it applies to a mammal, cell, tissue, organ, or biological fluid, as used herein is meant to refer without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. “Administration” also encompasses in vitro and ex vivo treatments, e.g., of a cell, by a reagent, diagnostic, binding composition, or by another cell.

The terms “amino acid residue” or “amino acid” or “residue” are used interchangeably herein to refer to an amino acid that is incorporated into a protein, a polypeptide, or a peptide, including, but not limited to, a naturally occurring amino acid and known analogs of natural amino acids that can function in a similar manner as naturally occurring amino acids. The amino acids may be L- or D-amino acids. An amino acid may be replaced by a synthetic amino acid, which is altered so as to increase the half-life of the peptide, increase the potency of the peptide, or increase the bioavailability of the peptide. The single letter designation for amino acids is used predominately herein. Such single letter designations are as follows: A is alanine; C is cysteine; D is aspartic acid; E is glutamic acid; F is phenylalanine; G is glycine; H is histidine; I is isoleucine; K is lysine; L is leucine; M is methionine; N is asparagine; P is proline; Q is glutamine; R is arginine; S is serine; T is threonine; V is valine; W is tryptophan; and Y is tyrosine. The following represents groups of amino acids that are conservative substitutions for one another: 1) Alanine (A), Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

The term “anergy” as used herein refers to a state of lymphocyte nonrespnsiveness to specific antigen induced by an encounter of the lymphocyte with cognate antigen under less than optimal conditions, such as in the absence of costimulation.

Antibody. The term “antibody” as used herein refers to a polypeptide or group of polypeptides comprised of at least one binding domain that is formed from the folding of polypeptide chains having three-dimensional binding spaces with internal surface shapes and charge distributions complementary to the features of an antigenic determinant of an antigen.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface. Human antibodies show two kinds of light chains, κ and λ; individual molecules of immunoglobulin generally are only one or the other.

Human antibodies show two kinds of light chains, κ and λ; individual molecules of immunoglobulin generally are only one or the other. In normal serum, 60% of the molecules have been found to have κ determinants and 30 percent λ. Many other species have been found to show two kinds of light chains, but their proportions vary. For example, in the mouse and rat, λ, chains comprise but a few percent of the total; in the dog and cat, κ chains are very low; the horse does not appear to have any κ chain; rabbits may have 5 to 40% λ, depending on strain and b-locus allotype; and chicken light chains are more homologous to λ, than κ.

In mammals, there are five classes of antibodies, IgA, IgD, IgE, IgG, and IgM, each with its own class of heavy chain—α (for IgA), δ (for IgD), ε (for IgE), γ (for IgG) and μ (for IgM). In addition, there are four subclasses of IgG immunoglobulins (IgG1, IgG2, IgG3, IgG4) having γ1, γ2, γ3, and γ4 heavy chains respectively. In its secreted form, IgM is a pentamer composed of five four-chain units, giving it a total of 10 antigen binding sites. Each pentamer contains one copy of a J chain, which is covalently inserted between two adjacent tail regions.

All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur; this phenomenon gives rise to cross-reactions. Cross reactions are of major importance in understanding the complementarity or specificity of antigen-antibody reactions. Immunological specificity or complementarity makes possible the detection of small amounts of impurities/contaminations among antigens.

An antibody may be an oligoclonal antibody, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques. Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies. Diverse libraries of immunoglobulin heavy (V_{H}) and light (V_{κ} and V_{λ}) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage. The technique of guided selection is based on human immunoglobulin V gene shuffling with rodent immunoglobulin V genes. The method entails (i) shuffling a repertoire of human λ light chains with the heavy chain variable region (V_{H}) domain of a mouse monoclonal antibody reactive with an antigen of interest; (ii) selecting half-human Fabs on that antigen (iii) using the selected λ light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, antibody molecule in the mouse myeloma. An antibody may be from any species. The term antibody also includes binding fragments of the antibodies of the invention; exemplary fragments include Fv, Fab, Fab′, single stranded antibody (svFC), dimeric variable region (Diabody) and di-sulphide stabilized variable region (dsFv). Structural and functional domains can be identified by comparison of the nucleotide and/or amino acid sequence data to public or proprietary sequence databases. For example, computerized comparison methods can be used to identify sequence motifs or predicted protein conformation domains that occur in other proteins of known structure and/or function. Methods to identify protein sequences that fold into a known three-dimensional structure are known. [See, for example, Bowie et al. Science 253:164 (1991), which is incorporated by reference in its entirety].

The term “affinity” as used herein refers to strength of the binding interaction between an epitope and the antibody combining site. It depends on the closeness of the stereochemical fit between the antibody combining site and the epitope, the size of the area of contact between them, and the distribution of charged and hydrophobic groups. The affinity constant (K_{A}) is a measure of how much antibody-antigen complex exists at the point equilibrium is reached, and can be represented as:

$${K}_{A} = \frac{[ Ab - Ag ]}{[ Ab ] [ Ag ]}$$

where [Ab} is the molar concentration of free antibody; [Ag] is the molar concentration of free antigen; and [Ab-Ag] is the molar concentration of the antibody-antigen complexes.

The term “affinity maturation” as used herein refers to an increase in affinity for the specific antigen of the antibodies produced as an adaptive immune response progresses. It is particularly prominent in secondary and subsequent immunizations. Affinity maturation requires the positive selection of germinal center B cells expressing high-affinity variant B cell receptors (BCRs). Receipt and integration of signals through the BCR are involved in positive selection and must be based on the affinity for antigen. After activated B cells interact with T_{FH} cells at the follicle border, they migrate to germinal centers. In the dark zone of the germinal center, somatic hypermutation alters the immunoglobulin V genes. In some B cells, the mutated BCR will have low or no affinity for the antigen, while in other B cells, the mutated BCR affinity may be higher. After exiting the dark zone, the B cells with higher affinity BCRs will capture antigen trapped on follicular dendritic cells (FDCs) and then process and present it on MHC class II molecules. B cells with low-affinity BCRs will fail to capture and present antigen. B cells that present linked antigen epitopes to T_{FH} cells will receive help through CD-40 and IL-21, which promote survival and proliferation. CD40 signaling in B cells is activated by CD40L on T_{FH} cells and increases expression of the survival molecule Bcl-XL, a relative of Bcl-2; this family of intracellular proteins includes members that promote apoptosis (Bax, Bak, and Bok) and members that inhibit apoptosis (Bcl-2, Bcl-W and Bcl-XL). B cells that lack antigen on MHC clss II molecules receive no help and will eventually die. Some of the proliferating B cells undergo repeated cycles of entry to the dark zone, mutation and selection, and other progeny B cells undergo differentiation to either memory B cells or plasma cells. [Janeway's Immunobiology, 9^{th} Ed., Murphy, K. and Weaver, C. eds. Garland Science, New York (2017) at 410-413].

The term “alum” as used herein refers to aluminum hydroxyphosphate, Al(OH)x(PO4)y (AP) and aluminum oxyhydroxide, Al(O)OH (AH), which act as adjuvants when mixed with antigens. Aluminum adjuvants selectively stimulate a Th2 immune response upon injection of mice and a mixed response in human beings. They support activation of CD8 T cells, but these cells do not undergo terminal differentiation to cytotoxic T cells. Adsorption of antigens to aluminum adjuvants enhances the immune response by facilitating phagocytosis and slowing the diffusion of antigens from the injection site which allows time for inflammatory cells to accumulate. Aluminum adjuvants activate dendritic cells via direct and indirect mechanisms. Phagocytosis of aluminum adjuvants followed by disruption of the phagolysosome activates NLRP3-inflammasomes resulting in the release of active IL-1β and IL-18. Aluminum adjuvants also activate dendritic cells by binding to membrane lipid rafts. Injection of aluminum-adjuvanted vaccines causes the release of uric acid, DNA, and ATP from damaged cells which in turn activate dendritic cells. The use of aluminum adjuvant is limited by weak stimulation of cell-mediated immunity, which can be enhanced by addition of other immunomodulatory molecules. The widespread use of aluminum adjuvants can be attributed in part to the excellent safety record based on a 70-year history of use. They cause local inflammation at the injection site, but also reduce the severity of systemic and local reactions by binding biologically active molecules in vaccines. [HogenEsch, H. Front Immunol. (2012) 3: 406].

The term “avidity” as used herein refers to a measure of the total strength of all the associations established between a multivalent ligand (antigen) and a multivalent antibody. It is dependent on affinity of the antibody for the eipitope, valency of both the antibody and the antigen, and structural arrangement of the parts that interact.

Antibody combining site. The antigen combining site, also called the “antigen binding site” or “paratope” is defined by the set of amino acid residues that make contact with the antigen. V_{H} and V_{L} combine by non-covalent association to form the Fv region, which contains the antigen binding or combining site. Each domain contributes three hypervariable loops (HVLs) or CDRs, with CDR-L1, CDR-L2, and CDR-L3 formed by V_{L} and CDR-H1, CDR-H2, and CDR-H3 by V_{H}. In the FV, the two β-sheets and the non-hypervariable loops are referred to as Framework Regions (FRs). CDR-L1 and CDR-H1 HVLs correspond to the residues within the loops connecting β-strands B and C (Gilliland et al., 2012). For CDR-L2 and CDR-H2, the HVLs are formed by the loops connecting β-strands C′ and C″, and for CDR-L3 and CDR-H3, the HVLs are formed by the loops connecting β-strands F and G (Gilliland et al., 2012). Due to the large number of different V-regions that can comprise the Fv, both amino acid sequence and length can vary significantly for the HVLs. [Taken from Therapeutic Antibody Engineering, Stohl, W R and Stohl L M, Eds., Woodhead Publishing Ltd. (2012)]

Antibody-dependent cellular cytotoxicity (ADCC), also called antibody-dependent cell-mediated cytotoxicity, is an immune mechanism through which Fc receptor-bearing effector cells can recognize and kill antibody-coated target cells expressing tumor- or pathogen-derived antigens on their surface. It is mediated by the recruitment of cytotoxic effector cells, such as natural killer (NK) cells, macrophages, and polymorphonuclear leukocytes (PMNs), that express Fc gamma receptors (FcγRs) on their surface.

Antibody-dependent cellular phagocytosis (ADCP) is a potent mechanism of elimination of antibody-coated foreign particles such microbes or tumor cells. Engagement of FcγRIIa and FcγRI expressed on macrophages triggers a signalling cascade leading to the engulfment of the IgG-opsonized particle.

The term “antibody titer” as used herein refers to the concentration of antigen-specific antibodies in an antiserum.

The term “antigen” as used herein, is meant to refer to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 7, 8, 9, 10, 11, 12, 13, 14, or 15 amino acids. The term includes polypeptides which include modifications, such as deletions, additions and substitutions (generally conservative in nature) as compared to a native sequence, as long as the protein maintains the ability to elicit an immunological response, as defined herein. These modifications may be deliberate, as through site-directed mutagenesis, or may be accidental, such as through mutations of hosts which produce the antigens.

The term “antigenic drift” as used herein refers to subtle modification of pathogen antigens through random point mutations; it usually involves surface proteins that would normally be the target of neutralizing antibodies.

The term “antigenic shift” as used herein refers to dramatic modification of viral antigens due to reassortment of genomic segments of two different strains of a virus that simultaneously infect the same individual to generate progeny virions with new combinations of genome segments and thus new proteins.

The term “antigen presentation” as used herein, generally refers to the display of antigen on the surface of a cell, e.g., in the form of peptide fragments bound to WIC molecules.

The term “antigen-presenting cell (APC)” as used herein is meant to refer to a cell that can process and display foreign antigens in association with major histocompatibility complex (WIC) molecules on its surface.

The term “antiserum” as used herein refers to the clear liquid (serum) fraction of clotted blood containing antibodies.

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits IAP proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. Loberg, R D, et al., J. Biol. Chem. 277 (44): 41667-673 (2002). It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

The terms “B lymphocyte” or “B cell” are used interchangeably to refer to a broad class of lymphocytes, which are precursors of antibody-secreting cells, that express clonally diverse cell surface immunoglobulin (Ig) receptors (BCRs) recognizing specific antigenic epitopes. Mammalian B-cell development encompasses a continuum of stages that begin in primary lymphoid tissue (eg, human fetal liver and fetal/adult marrow), with subsequent functional maturation in secondary lymphoid tissue (eg, human lymph nodes and spleen). The functional/protective end point is antibody production by terminally differentiated plasma cells. A mature B cell can be activated by an encounter with an antigen that expresses epitopes that are recognized by its cell surface immunoglobulin (Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”).[LeBien, TW & TF Tedder, B lymphocytes: how they develop and function. Blood (2008) 112 (5): 1570-80].

The term “B cell receptor” or “BCR” as used herein refers to the antigen-receptor complex of B lineage cells, which is composed of a membrane bound Ig (mIg) monomer plus the Igα/Igβ complex required for intracellular signaling.

The term “binding” and its various grammatical forms means a lasting attraction between chemical substances. Binding specificity involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “CD40” as used herein refers to a tumor necrosis factor receptor (TNFR) superfamily member expressed on APCs, such as dendritic cells (DC), B cells, and monocytes as well as many non-immune cells and a wide range of tumors. Interaction with its trimeric ligand CD40 Ligand (CD40L) on activated T helper cells results in APC activation, required for the induction of adaptive immunity. CD40 on B cells and CD40 ligand on activated helper T cells are co-stimulatory molecules whose interaction is required for the proliferation and class switching of antigen activated naïve B cells. CD40 is also expressed by dendritic cells; where the CD40-CD40L interaction provides co-stimulatory signals to naïve T cells.

The term “cell line” as used herein, is meant to refer to a permanently established cell culture developed from a single cell and therefore consisting of cells with a uniform genetic makeup that will proliferate indefinitely.

The term “clade” as used herein refers to related organisms descended from a common ancestor.

The term “class switching”, “isotype switching” or “class switch recombination” as used herein refers to a somatic gene recombination process in activated B cells that replaces one heavy chain constant region with one of a different isotype, switching the isotype of antibodies from IgM to IgG, IgA or IgE. This affects the antibody effector functions but not their antigen specificity.

The term “coding region” as used herein, is meant to refer to that portion of a gene that either naturally or normally codes for the expression product of that gene in its natural genomic environment, i.e., the region coding in vivo for the native expression product of the gene. The coding region can be from a normal, mutated or altered gene, or can even be from a DNA sequence, or gene, wholly synthesized in the laboratory using methods well known to those of skill in the art of DNA synthesis. A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

The term “complement” as used herein refers to a system of over 30 soluble and membrane-bound proteins that act through a tightly regulated cascade of pro-protein cleavage and activation to mediate cell lysis through assembly of the membrane attack complex (MAC) composed of complement components C5b, C6, C7, C8, and C9 in a target cell membrane. Intermediates in the complement cascade play a variety of roles in antigen clearance. The activation of complement can lead to lysis of an antibody-opsonized cell by complement-dependent cytotoxicity (CDC) or complement-dependent cell-mediated cytotoxicity (CDCC) [Meyer, S. et al. MAbs (2014) 6 (5): 1133-44].

The term “component” as used herein, is meant to refer to a constituent part, element or ingredient.

The term “composition” as used herein, is meant to refer to a material formed by a mixture of two or more substances.

As used herein, the term “condition” as used herein, is meant to refer to a variety of health states and is meant to include disorders or diseases caused by any underlying mechanism or disorder.

As used herein, the term “consensus sequence” is used to describe a theoretical representative nucleotide or amino acid sequence in which each nucleotide or amino acid is the one which occurs most frequently at that site in the different sequences which occur in nature. The phrase also refers to an actual sequence which approximates the theoretical consensus. For example, a consensus sequence can be used to represent a known conserved sequence set which is a sequence of amino acids in a polypeptide or of nucleotides in DNA or RNA that is similar across multiple species.

The term “control” as used herein refers to a holding back, a restraint or a curb.

The term “costimulation” as used herein refers to the second signal required for completion of lymphocyte activation and prevention of anergy, which is supplied by engagement of CD28 by CD80 and CD86 (T cells) and of CD40 by CD40 Ligand (B cells).

The term “contact” and its various grammatical forms as used herein, are meant to refer to a state or condition of touching or of immediate or local proximity. Contacting a composition to a target destination may occur by any means of administration known to the skilled artisan.

The term “control elements” as used herein is a generic term for a region of DNA, such as a promoter or enhancer adjacent to (or within) a gene that allows the regulation of gene expression by the binding of transcription factors. Typical “control elements”, include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences; and/or sequence elements controlling an open chromatin structure [see e.g., McCaughan et al. (1995) PNAS USA 92:5431-5435; Kochetov et al (1998) FEBS Letts. 440:351-355].

The terms “CpG oligodeoxynucleotide”, “oligodeoxynucleotide CpG”, ODN-CpG” and “CPG-ODN” are used interchangeably to refer to a synthetic oligodeoxynucleotide containing unmethylated CpG motifs derived from bacterial DNA with immunostimulatory activities. CpG-ODN binds to and activates Toll-like receptor 9 (TLR9) and is taken up into cells by endocytosis; once internalized, it may activate numerous signaling transduction pathways resulting in the release of multiple cytokines. Through activation of TLR9, a CpG ODN can directly stimulate B-lymphocytes, dendritic and NK cells, resulting in an increase in innate immunity and antibody-dependent cell cytotoxicity (ADCC). Additionally, a CpG ODN can indirectly modulate T-cell responses, through the release of cytokines (IL-12 and IFN gamma), to induce a preferential shift to the Th1 (helper) phenotype resulting in enhanced CD8+ cellular cytotoxicity.

As used herein, the term “cross-protection” is used to describe immunity against at least two subgroups, subtypes, strains and/or variants of a virus, bacteria, parasite or other pathogen with a single inoculation with one subgroup, subtype, strain and/or variant thereof.

The term “culture” and its other grammatical forms as used herein, is meant to refer to a process whereby a population of cells is grown and proliferated on a substrate in an artificial medium.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “cytotoxic T lymphocytes” (CTLs) as used herein, is meant to refer to effector CD8+ T cells. Cytotoxic T cells kill by inducing their targets to undergo apoptosis. They induce target cells to undergo programmed cell death via extrinsic and intrinsic pathways.

The term “dendritic cell” or “DC” as used herein, is meant to refer to a diverse population of morphologically similar cell types found in a variety of lymphoid and non-lymphoid tissues that present foreign antigens to T cells, [see Steinman, Ann. Rev. Immunol. 9:271-296 (1991)].

The term “derived from” as used herein, is meant to encompasses any method for receiving, obtaining, or modifying something from a source of origin

The term “detectable marker” encompasses both selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like.

The term “detectable response” as used herein, is meant to refer to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The term “differentiate” and its various grammatical forms as used herein, are meant to refer to the process of development with an increase in the level of organization or complexity of a cell or tissue, accompanied with a more specialized function.

The term “dose” as used herein, is meant to refer to the quantity of a therapeutic substance prescribed to be taken at one time.

The term “dye” (also referred to as “fluorochrome” or “fluorophore”) as used herein refers to a component of a molecule which causes the molecule to be fluorescent. The component is a functional group in the molecule that absorbs energy of a specific wavelength and re-emits energy at a different (but equally specific) wavelength. The amount and wavelength of the emitted energy depend on both the dye and the chemical environment of the dye. Many dyes are known, including, but not limited to, FITC, R-phycoerythrin (PE), PE-Texas Red Tandem, PE-Cy5 Tandem, propidium iodem, EGFP, EYGP, ECF, DsRed, allophycocyanin (APC), PerCp, SYTOX Green, courmarin, Alexa Fluors (350, 430, 488, 532, 546, 555, 568, 594, 633, 647, 660, 680, 700, 750), Cy2, Cy3, Cy3.5, Cy5, Cy5.5, Cy7, Hoechst 33342, DAPI, Hoechst 33258, SYTOX Blue, chromomycin A3, mithramycin, YOYO-1, SYTOX Orange, ethidium bromide, 7-AAD, acridine orange, TOTO-1, TO-PRO-1, thiazole orange, TOTO-3, TO-PRO-3, thiazole orange, propidium iodide (PI), LDS 751, Indo-1, Fluo-3, DCFH, DHR, SNARF, Y66F, Y66H, EBFP, GFPuv, ECFP, GFP, AmCyan1, Y77W, S65A, S65C, S65L, S65T, ZsGreen1, ZsYellow1, DsRed2, DsRed monomer, AsRed2, mRFP1, HcRed1, monochlorobimane, calcein, the DyLight Fluors, cyanine, hydroxycoumarin, aminocoumarin, methoxycoumarin, Cascade Blue, Lucifer Yellow, NBD, PE-Cy5 conjugates, PE-Cy7 conjugates, APC-Cy7 conjugates, Red 613, fluorescein, FluorX, BODIDY-FL, TRITC, X-rhodamine, Lissamine Rhodamine B, Texas Red, TruRed, and derivatives thereof.

The term “effective dose” as used herein, generally refers to that amount of an immunogen comprising an internal conserved protein, or an immunogenic fragment thereof, of an infectious agent or pathogen described herein, or a vaccine comprising the immunogen, sufficient to induce immunity, to control and/or ameliorate an infection or to reduce at least one symptom of an infection and/or to enhance the efficacy of another dose of a immunogen or vaccine comprising the immunogen. An effective dose may refer to the amount of immunogen or vaccine comprising the immunogen sufficient to delay or minimize the onset of an infection. An effective dose may also refer to the amount of immunogen or vaccine comprising the immunogen that provides a therapeutic benefit in the treatment or management of an infection. Further, an effective dose is the amount with respect to an immunogen or vaccine comprising the immunogen of the disclosure alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

The term “effective amount” as used herein, is meant to refer to an amount of immunogen or vaccine comprising the immunogen necessary or sufficient to realize a desired biologic effect. An effective amount of the composition would be the amount that achieves a selected result, and such an amount could be determined as a matter of routine experimentation by a person skilled in the art. For example, an effective amount for preventing, controlling, treating and/or ameliorating an infection could be that amount necessary to cause activation of the immune system, resulting in the development of an antigen specific immune response upon exposure to immunogens or vaccines comprising the immunogen of the disclosure. The term is also synonymous with “sufficient amount.”

The term “effector cell” as used herein refers to a cell that carries out a final response or function. The main effector cells of the immune system, for example, are activated lymphocytes and phagocytes.

The term “enrich” as used herein refers to increasing the proportion of a desired substance, for example, to increase the relative frequency of a subtype of cell compared to its natural frequency in a cell population. Positive selection, negative selection, or both are generally considered necessary to any enrichment scheme. Selection methods include, without limitation, magnetic separation and FACS. Regardless of the specific technology used for enrichment, the specific markers used in the selection process are critical, since developmental stages and activation-specific responses can change a cell's antigenic profile.

The terms “expanding a CD8+ T cell” or “CD8+ T cell expansion” as used herein, are meant to refer to a process wherein a population of CD8+ T cells undergoes a series of cell divisions and thereby increases in cell number. The term “expanded CD8+ T cells” relates to CD8+ T cells obtained through CD8+ T cell expansion. Suitable assays to measure T cell expansion are known in the art and are described herein.

The terms “expanding an NK cell” or “NK cell expansion” as used herein, are meant to refer to a process wherein a population of NK cells undergoes a series of cell divisions and thereby increases in cell number. The term “expanded NK cells” relates to NK cells obtained through NK cell expansion. Suitable assays to measure NK cell expansion are known in the art and are described herein.

The terms “expanding a population of type-I NKT cells” or “type-I NKT cell expansion” are meant to refer to a process wherein a population of type-INKT cells undergoes a series of cell divisions and thereby expands in cell number (for example, by in vitro culture).

The term “express” or “expression” as used herein, is meant to encompass the biosynthesis of mRNA, polypeptide biosynthesis, polypeptide activation, e.g., by post-translational modification, or an activation of expression by changing the subcellular location or by recruitment to chromatin. Expression may be, e.g., increased by a number of approaches, including: increasing the number of genes encoding the polypeptide, increasing the transcription of the gene (such as by placing the gene under the control of a constitutive promoter), increasing the translation of the gene, knock out of a competitive gene, or a combination of these and/or other approaches.

The term “expression vector” as used herein, is meant to refer to a DNA molecule comprising a gene that is expressed in a host cell. Typically, gene expression is placed under the control of certain regulatory elements including, but not limited to, promoters, tissue specific regulatory elements, and enhancers. Such a gene is said to be “operably linked to” the regulatory elements.

The term “Fc receptor” (FcR) as used herein refers to a leukocyte receptor expressed primarily on effector cells that binds to the Fc region of a specific antibody isotype. Their ectodomains bind ligand, the IgG antibody Fc region, and belong to the Ig-superfamily. They include the high affinity IgE receptor FcεRI and the distantly related IgA receptor FcαRI, but the largest group are the IgG receptors or the FcγRs which themselves comprise several groups—FcγRI, the high affinity IgG receptor, the FcγRII family (FcγRIIA, FcγRIIB, FcγRIIC), and the FcγRIII family. FcγRIIA1 is the most widespread and abundant of all FcγR, present on Langerhans cells, platelets and all leukocytes, with the exception of most lymphocytes. FcγRIIA3 is expressed by neutrophils and monocytes, and FcγRIIA2 mRNA is present in platelets, megakaryocytes, and Langerhans cells. The levels of FcγRIIA expression are influenced by cytokine exposure. Interferon (IFN)-γ, interleukin (IL)-3, IL-6, C5a, prostaglandin-E (PGE), and dexamethasone increase expression, but IL-4, tumor necrosis factor (TNF)-α, and TNF-β reduce expression. There are also reports of FcγRII induction on CD4 and CD8 T cells upon mitogen or TCR stimulation. Both FcγRIIA and FcγRIIB are reported to be expressed on activated CD4 T cells. Like other activating-type immunoreceptors, FcγRIIA and FcγRIIC signal via the Immunoreceptor Tyrosine-based Activation Motif (ITAM) pathway, but unlike other activating immunoreceptors, the ITAM is present in its own IgG binding chain. Furthermore, the FcγRIIA ITAM is unusual in that it does not fit the canonical ITAM consensus sequence and includes three additional aspartic residues. The FcγRIIA (also FcγRIIC) and FcγRIIB proteins have opposing cellular functions. FcγRIIA proteins are activating-type Fc receptors. In contrast, FcγRIIB is a key immune checkpoint that modulates the action of activating-type Fc receptors and the antigen receptor of B cells. When expressed, the FcγRIIC proteins retain the activating function of the cytoplasmic tail of FcγRIIA and the binding specificity of FcγRIIB ectodomains. [Anania, J C et al., The human FcγRII(CD32) family of leukocyte FcR in health and disease”. Front. Immunol. (2019) doi.org/10.3389/fimmu.2019.00464].

The term “flow cytometry” as used herein, is meant to refer to a tool for interrogating the phenotype and characteristics of cells. It senses cells or particles as they move in a liquid stream through a laser (light amplification by stimulated emission of radiation)/light beam past a sensing area. The relative light-scattering and color-discriminated fluorescence of the microscopic particles is measured. Flow Analysis and differentiation of the cells is based on size, granularity, and whether the cell is carrying fluorescent molecules in the form of either antibodies or dyes. As the cell passes through the laser beam, light is scattered in all directions, and the light scattered in the forward direction at low angles)(0.5-10° from the axis is proportional to the square of the radius of a sphere and so to the size of the cell or particle. Light may enter the cell; thus, the 90° light (right-angled, side) scatter may be labeled with fluorochrome-linked antibodies or stained with fluorescent membrane, cytoplasmic, or nuclear dyes. Thus, the differentiation of cell types, the presence of membrane receptors and antigens, membrane potential, pH, enzyme activity, and DNA content may be facilitated. Flow cytometers are multiparameter, recording several measurements on each cell; therefore, it is possible to identify a homogeneous subpopulation within a heterogeneous population (Marion G. Macey, Flow cytometry: principles and applications, Humana Press, 2007). Fluorescence-activated cell sorting (FACS), which allows isolation of distinct cell populations too similar in physical characteristics to be separated by size or density, uses fluorescent tags to detect surface proteins that are differentially expressed, allowing fine distinctions to be made among physically homogeneous populations of cells.

The terms “follicular helper T cell” (“T_{FH}”), and “circulatory follicular helper CD4+ T cells” [“cT_{FH}”] as used herein are used interchangeably to refer to a type of effector CD4 T cell that resides in lymphoid follicles and provides help to B cells for antibody production.

The terms “functional equivalent” and “functionally equivalent” are used interchangeably herein to refer to substances, molecules, polynucleotides, proteins, peptides, or polypeptides having similar or identical effects or use.

As used herein, the term “gene” is used broadly to refer to any locatable segment of nucleic acid associated with expression of a given RNA or protein. Thus, genes include regions encoding expressed RNAs (which typically include polypeptide coding sequences) and, often, the regulatory sequences required for their expression. Genes can be obtained from a variety of sources, including cloning from a source of interest or synthesizing from known or predicted sequence information, and may include sequences designed to have specifically desired parameters.

The term “granulocyte-macrophage colony-stimulating factor (GM-CSF) as used herein refers to a cytokine involved in the growth and differentiation of cells of the myeloid lineage, including dendritic cells, monocytes and tissue macrophages, and granulocytes.

The term “granulocytes” as used herein refers to myeloid leukocytes that harbor large intracellular granules containing microbe-destroying hydrolytic enzymes, and includes neutrophils, basophils and eosinophils.

The term “herd immunity” as used herein refers to protection conferred to unvaccinated individuals in a population produced by vaccination of others and reduction in the natural reservoir for infection.

The term “heterosubtypic immunity” (“HSI”) as used herein refers to immunity based on immune recognition of antigens conserved across all viral strains.

The term “heterotypic” as used herein is used to refer to being of a different or unusual type or form (e.g., different subgroup, subtype, strain and/or variant of a virus, bacteria, parasite or other pathogen).

The term “homotypic” as used herein is used to refer to being of the same type or form, e.g., same subgroup, subtype, strain and/or variant of a virus, bacteria, parasite or other pathogen.

The terms “immune response” and “immune-mediated” as used herein, are used interchangeably herein to refer to any functional expression of a subject's immune system, against either foreign or self-antigens, whether the consequences of these reactions are beneficial or harmful to the subject. The term “immunological response” to an antigen or composition as used herein, is meant to refer to the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or γδ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The term “immune phenotype” or “immunotype” as used herein refers to the collective frequency of various immune cell populations and their functional responses to stimuli (cell signaling and antibody responses). [See Kaczorowski, K J et al. Proc. Nat. Acad. Sci. USA (2017) doi/10.1073/pnas.1705065114]

The term “immune system” as used herein refers to the body's system of defenses against disease, which comprises the innate immune system and the adaptive immune system. The innate immune system provides a non-specific first line of defense against pathogens. It comprises physical barriers (e.g. the skin) and both cellular (granulocytes, natural killer cells) and humoral (complement system) defense mechanisms. The reaction of the innate immune system is immediate, but unlike the adaptive immune system, it does not provide permanent immunity against pathogens. The adaptive immune response is the response of the vertebrate immune system to a specific antigen that typically generates immunological memory.

The term “immunodominant epitope” as used herein refers to the epitope against which the majority of antibodies is raised, or to which the majority of T cells responds.

The term “immunological repertoire” refers to the collection of transmembrane antigen-receptor proteins located on the surface of T and B cells. [Benichou, J. et al. Immunology (2011) 135: 183-191)] The combinatorial mechanism that is responsible for encoding the receptors does so by reshuffling the genetic code, with a potential to generate more than 10¹⁸ different T cell receptors (TCRs) in humans [Id., citing Venturi, Y. et al. Nat. Rev. Immunol. (2008) 8: 231-8] and a much more diverse B-cell repertoire. These sequences, in turn, will be transcribed and then translated into protein to be presented on the cell surface. The recombination process that rearranges the gene segments for the construction of the receptors is key to the development of the immune response, and the correct formation of the rearranged receptors is critical to their future binding affinity to antigen.

The term “integrate into the genome” as used herein refers to a recombinant DNA sequence being concomitantly joined to the genomic DNA comprising a host cell's genome.

The term “isolated” as used herein, is meant to refer to material removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. According to some embodiments, an isolated population of a particular cell type refers to greater than 10% pure, greater than 20% pure, greater than 30% pure, greater than 40% pure, greater than 50% pure, greater than 60% pure, greater than 70% pure, greater than 80% pure, greater than 90% pure, or greater than 95% pure.

The term “isotype” as used herein refers to classes of Igs defined on the basis of the amino acid sequences of their constant regions, including IgM, IgD, IgA, IgG and IgE heavy chain isotypes and Igκ and Igλ light chain isotypes.

The term “labeling” as used herein refers to a process of distinguishing a compound, structure, protein, peptide, antibody, cell or cell component by introducing a traceable constituent. Common traceable constituents include, but are not limited to, a fluorescent antibody, a fluorophore, a dye or a fluorescent dye, a stain or a fluorescent stain, a marker, a fluorescent marker, a chemical stain, a differential stain, a differential label, and a radioisotope.

The terms “limited digestion” or “partial digestion” are used interchangeably herein to refer to conditions such as limited time of incubation or limited enzyme concentration that permit only a fraction of the total number of sites in individual molecules to be cleaved.

The term “lymphocyte” as used herein refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens. This commitment, which exists before the first contact of the immune system with a given antigen, is expressed by the presence on the lymphocyte's surface membrane of receptors specific for determinants (epitopes) on the antigen. Each lymphocyte possesses a population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions.

The terms “marker” or “cell surface marker” are used interchangeably herein to refer to an antigenic determinant or epitope found on the surface of a specific type of cell. Cell surface markers can facilitate the characterization of a cell type, its identification, and eventually its isolation. Cell sorting techniques are based on cellular biomarkers where a cell surface marker(s) may be used for either positive selection or negative selection, i.e., for inclusion or exclusion, from a cell population.

The term “mediate” and its various grammatical forms as used herein, are meant to refer to bringing about a result.

The term“memory cells” as used herein refers to B and T lymphocytes generated during a primary immune response that remain in a quiescent state until fully activated by a subsequent exposure to specific antigen (secondary immune response). Memory cells generally are more sensitive than naïve lymphocytes to antigen and respond rapidly on reexposure to the antigen that originally induced them.

The terms “major histocompatibility complex” or “MHC molecule” as used herein, is meant to refer to one of a large family of ubiquitous cell-surface glycoproteins encoded by genes of the major histocompatibility complex (MHC). They bind peptide fragments of foreign antigens and present them to T cells to induce an immune response. Class I MHC molecules, which are encoded by a series of highly polymorphic genes, are present on almost all cell types and present viral peptides on the surface of virus-infected cells, where they are recognized by cytotoxic T cells. In the MHC class I mechanism, foreign peptides are endocytosed for transport within an antigen presenting cell. Then, at least some of the foreign protein is proteolyzed by the cytosolic proteasome to form short peptides, which are transported into the lumen of the endoplasmic reticulum of the antigen presenting cell. There, the foreign peptides are loaded onto MHC class I molecules and transported by vesicles to the cell surface of the antigen presenting cell for recognition by CD8+ cytotoxic T cells. For example, MHC I expression on cancer cells is required for detection and destruction by T-cells, and cytotoxic T lymphocytes (CTLs, CD8+) require tumor antigen presentation on the target cell by MHC Class I molecules to delineate self from non-self. One of the most common means by which tumors evade the host immune response is by downregulation of MHC Class I molecule expression by tumor cells, such that the tumor has low MHCI expression, thereby rendering any endogenous or therapeutic anti-tumor T cell responses ineffective (Haworth et al., Pediatr Blood Cancer. 2015 April; 62(4): 571-576). Most often, the loss of MHC expression on tumor cells is mediated by epigenetic events and transcriptional downregulation of the MHC locus and/or the antigen processing machinery. Lack of a processed peptide antigen leads to decreased MHC expression since empty MHC molecules are not stable on the cell surface.

A class II MHC molecule, which is present on professional antigen presenting cells, presents foreign peptides to helper T cells. Foreign peptides are endocytosed and degraded in the acidic environment of the endosome, which means that the peptides are never present in the cytosol and remain in a subcellular compartment topologically equivalent to the extracellular space. The peptides bind to preassembled MHC class II proteins in a specialized endosomal compartment, and the loaded MHC class II molecule is then transported to the plasma membrane of the antigen presenting cell for presentation to CD4+ helper T cells. (Alberts et al. Molecular Biology of the Cell 4th Ed., Garland Science, New York (2002) p. 1407).

Antigens also can be loaded onto antigen presenting cells by acquisition of MHC class II molecules from the surface of donor cells. This peptide-MHC transfer (cross-dressing”), involves generation of peptide-MHC class II complexes within the donor cell, and their subsequent transfer to recipient antigen presenting cells, which are then able to present the intact, largely unprocessed peptide-MHC class II complexes to helper T cells. (Campana, S. et al., Immunol. Letters (2015) 168(2): 349-54). Endogenous antigens can also be presented by MHC class II when they are degraded through autophagy. (Schmid, D. et al. (2007) Immunity 26(1): 79-92).

The term “modify” and its various grammatical forms as used herein, are meant to refer to a change of the form or qualities of.

The term “modulate” and its various grammatical forms as used herein, are meant to refer to regulating, altering, adapting or adjusting to a certain measure or proportion. Such modulation may be any change, including an undetectable change.

The term “modified” or “modulated” as used herein with respect to an immune response to tumor cells is meant to refer to changing the form or character of the immune response to the tumor cells via one or more recombinant DNA techniques such that the immune cells are able to recognize and kill tumor cells.

“The term “monophosphoryl lipid A (MPL)” as used herein refers to an adjuvant with low toxicity that is derived from the lipopolysaccharide fraction of the cell walls of gram-negative bacteria such as Salmonella minnesota. Its structural formula is shown below.

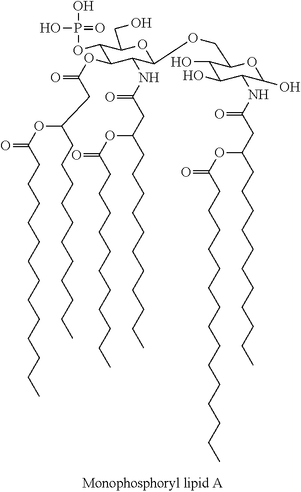 [SMILES: CCCCCCCCCCCCCCCC(=O)OC(CCCCCCCCCCC)CC(=O)NC1C(O)OC(COC2OC(CO)C(OP(=O)(O)O)C(OC(=O)CC(CCCCCCCCCCC)OC(=O)CCCCCCCCCCCCC)C2NC(=O)CC(CCCCCCCCCCC)OC(=O)CCCCCCCCCCC)C(O)C1O]

MPLA, an adjuvant for T cell priming, is a detoxified form of the endotoxin lipopolysaccharide (LPS) [Casella, C R, Mitchell, T C. Cell Mol. Life Sci. (2008) 65 (20): 3231-40]. Both LPS and MPLA require TLR4 for adjuvant function. Stimulation of human monocytes by MPL has been shown to lead to an up-regulation of the costimulatory molecules CD80 and CD86 [Martin, M. et al., Infect. Immun. (2003) 71 (5): 2498-2507]. The degree of Th1-associated immune responses depends on both the type of antigen being given as well as on the route of administration (intravenous vs. intranasal vs. subcutaneous injection); MPLA therefore has a strong but not overwhelming ability to promote Th1 responses. [Casella, C R, Mitchell, T C. Cell Mol. Life Sci. (2008) 65 (20): 3231-40].

The term “natural killer (NK) cells” as used herein is meant to refer to lymphocytes in the same family as T and B cells, classified as group I innate lymphocytes. They have an ability to kill tumor cells without any priming or prior activation, in contrast to cytotoxic T cells, which need priming by antigen presenting cells. NK cells secrete cytokines such as IFNγ and TNFα, which act on other immune cells, like macrophages and dendritic cells, to enhance the immune response. Activating receptors on the NK cell surface recognize molecules expressed on the surface of cancer cells and infected cells and switch on the NK cell. Inhibitory receptors act as a check on NK cell killing. Most normal healthy cells express MHCI receptors, which mark them as “self.” Inhibitory receptors on the surface of the NK cell recognize cognate MHCI, which switches off the NK cell, preventing it from killing. Once the decision is made to kill, the NK cell releases cytotoxic granules containing perforin and granzymes, which leads to lysis of the target cell. Natural killer reactivity, including cytokine secretion and cytotoxicity, is controlled by a balance of several germ-line encoded inhibitory and activating receptors such as killer immunoglobulin-like receptors (KIRs) and natural cytotoxicity receptors (NCRs). The presence of the MHC Class I molecule on target cells serves as one such inhibitory ligand for MHC Class I-specific receptors, the Killer cell Immunoglobulin-like Receptor (KIR), on NK cells. Engagement of KIR receptors blocks NK activation and, paradoxically, preserves their ability to respond to successive encounters by triggering inactivating signals. Therefore, if a KIR is able to sufficiently bind to MHC Class I, this engagement may override the signal for killing and allows the target cell to live. In contrast, if the NK cell is unable to sufficiently bind to MHC Class I on the target cell, killing of the target cell may proceed. Consequently, those tumors which express low MHC Class I and which are thought to be capable of evading a T-cell-mediated attack may be susceptible to an NK cell-mediated immune response instead.

The term “natural killer T cell” or “NKT” as used herein, is meant to refer to invariant natural killer T (iNKT) cells, also known as type-I NKT cells, as well as all subsets of non-invariant (Vα24− and Vα24+) natural killer T cells, which express CD3 and an αβ T cell receptor (TCR) (herein termed “natural killer αβ T cells”) or γδ TCR (herein termed “natural killer γδ T cells”), all of which have demonstrated capacity to respond to non-protein antigens presented by CD1 antigens. The non-invariant NKT cells share in common with type-I NKT cells the expression of surface receptors commonly attributed to natural killer (NK) cells, as well as a TCR of either αβ or γδ TCR gene locus rearrangement/recombination.

The term “invariant natural killer T cell” as used herein, is meant to be used interchangeably with the term “iNKT,” and is meant to refer to a subset of T-cell receptor (TCR)α-expressing cells that express a restricted TCR repertoire that, in humans, is composed of a Vα24-Ja18 TCRα chain, which is, for example, coupled with a Vβ11 TCRβ chain. iNKT is meant to encompass all subsets of CD3+Vα24+type-I NKT cells (CD3+CD4+CD8−Vα24+, CD3+CD4−CD8+Vα24−+, and CD3+CD4−CD8−Vα24+) as well as those cells, which can be confirmed to be type-I NKT cells by gene expression or other immune profiling, but have down-regulated surface expression of Vα24 (CD3+Vα24−). This includes cells which either do or do not express the regulatory transcription factor FOXP3. Unlike conventional T cells, which mostly recognize peptide antigens presented by MEW molecules, iNKT cells recognize glycolipid antigens presented by the non-polymorphic MEW class 1-like CD1d.

The term “neutrophils” or “polymorphonuclear neutrophils (PMNs)” as used herein refers to the most abundant type of white blood cells in mammals, which form an essential part of the innate immune system. They form part of the polymorphonuclear cell family (PMNs) together with basophils and eosinophils. Neutrophils are normally found in the blood stream. During the beginning (acute) phase of inflammation, particularly as a result of bacterial infection and some cancers, neutrophils are one of the first-responders of inflammatory cells to migrate toward the site of inflammation. They migrate through the blood vessels, then through interstitial tissue, following chemical signals such as interleukin-8 (IL-8) and C5a in a process called chemotaxis.

The term “non-expanded” as used herein, is meant to refer to a cell population that has not been grown in culture (in vitro) to increase the number of cells in the cell population.

The term “non-replicating” or “replication-impaired” virus refers to a virus that is not capable of replication to any significant extent in the majority of normal mammalian cells or normal primary human cells.

The term “normal healthy control subject” as used herein refers to a subject having no symptoms or other clinical evidence of a viral infection.

The term “nucleic acid” as used herein, is meant to refer to a deoxyribonucleotide or ribonucleotide polymer in either single- or double-stranded form, and, unless otherwise limited, encompasses known analogues having the essential nature of natural nucleotides in that they hybridize to single-stranded nucleic acids in a manner similar to naturally occurring nucleotides (e.g., peptide nucleic acids). Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). Measuring the effects of base incompatibility by quantifying the rate at which two strands anneal can provide information as to the similarity in base sequence between the two strands being annealed. A nucleic acid that selectively hybridizes undergoes hybridization, under stringent hybridization conditions, of the nucleic acid sequence to a specified nucleic acid target sequence to a detectably greater degree (e.g., at least 2-fold over background) than its hybridization to non-target nucleic acid sequences and to the substantial exclusion of non-target nucleic acids.

The term “nucleotide sequence” as used herein, is meant to refer to a heteropolymer of deoxyribonucleotides. The nucleotide sequence encoding for a particular peptide, oligopeptide, or polypeptide may be naturally occurring or they may be synthetically constructed.

The term “open reading frame” as used herein, is meant to refer to a sequence of nucleotides in a DNA molecule that has the potential to encode a peptide or protein: it starts with a start triplet (ATG), is followed by a string of triplets each of which encodes an amino acid, and ends with a stop triplet (TAA, TAG or TGA).

The phrase “operably linked” as used herein, is meant to refer (1) to a first sequence(s) or domain being positioned sufficiently proximal to a second sequence(s) or domain so that the first sequence(s) or domain can exert influence over the second sequence(s) or domain or a region under control of that second sequence or domain; and (2) to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, are in the same reading frame. According to some embodiments, the phrase “operatively linked” refers to a linkage in which two or more protein domains or polypeptides are ligated or combined via recombinant DNA technology or chemical reaction such that each protein domain or polypeptide of the resulting fusion protein retains its original function.

The term “optimized viral polypeptide” as used herein, is meant to refer to an immunogenic polypeptide that is not a naturally-occurring viral peptide, polypeptide, or protein. Optimized viral polypeptide sequences are initially generated by modifying the amino acid sequence of one or more naturally-occurring viral gene products (e.g., peptides, polypeptides, and proteins) to increase the breadth, intensity, depth, or longevity of the antiviral immune response (e.g., cellular or humoral immune responses) generated upon immunization (e.g., when incorporated into a vaccine of the disclosure) of a mammal (e.g., a human). Thus, the optimized viral polypeptide may correspond to a “parent” viral gene sequence; alternatively, the optimized viral polypeptide may not correspond to a specific “parent” viral gene sequence but may correspond to analogous sequences from various strains or quasispecies of a virus. Modifications to the viral gene sequence that can be included in an optimized viral polypeptide include amino acid additions, substitutions, and deletions. According to some embodiments of the disclosure, the optimized viral polypeptide is the composite or merged amino acid sequence of two or more naturally-occurring viral gene products (e.g., natural or clinical viral isolates) in which each potential epitope (e.g., each contiguous or overlapping amino acid sequence of 5, 6, 7, 8, 9, 10, 11, 12, 13, 14, 15, 16, 17, 18, 19, 20, 21, 22, 23, 24, 25, 26, 27, 28, 29, 30 or more amino acids in length) is analyzed and modified to improve the immunogenicity of the resulting optimized viral polypeptide. Optimized viral polypeptides that correspond to different viral gene products can also be fused to facilitate incorporation in a vaccine of the disclosure. Methods of generating an optimized viral polypeptides are described in, e.g., Fisher et al. (2007) “Polyvalent Vaccine for Optimal Coverage of Potential T-Cell Epitopes in Global HIV-I Variants,” Nat. Med. 13(1): 100-106 and International Patent Application Publication WO 2007/024941, herein incorporated by reference. Once the optimized viral polypeptide sequence is generated, the corresponding polypeptide can be produced or administered by standard techniques as described herein.

The term “overall survival” (OS) as used herein, is meant to refer to the length of time from either the date of diagnosis or the start of treatment for a disease that patients diagnosed with the disease are still alive.

The term “parenteral” and its other grammatical forms as used herein, is meant to refer to administration of a substance occurring in the body other than by the mouth or alimentary canal. For example, the term “parenteral” as used herein refers to introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord or under the arachnoid membrane of the brain), or infusion techniques.

The term “pattern recognition receptors” or “PRRs” as used herein, is meant to refer to receptors that are present at the cell surface to recognize extracellular pathogens; in the endosomes where they sense intracellular invaders, and finally in the cytoplasm. They recognize conserved molecular structures of pathogens, called pathogen associated molecular patterns (PAMPs) specific to the microorganism and essential for its viability. PRRs are divided into four families: toll-like receptors (TLR); nucleotide oligomerization receptors (NLR); C-type leptin receptors (CLR), and RIG-1 like receptors (RLR).

The term “peptide” is used herein to designate a series of amino acid residues, connected one to the other typically by peptide bonds between the alpha-amino and carbonyl groups of the adjacent amino acids. Peptides are typically 9 amino acids in length, but can be as short as 8 amino acids in length, and as long as 14 amino acids in length. A series of amino acids are considered an “oligopeptide” when the amino acid length is greater than about 14 amino acids in length, typically up to about 30 to 40 residues in length. When the amino acid residue length exceeds 40 amino acid residues, the series of amino acid residues is termed a “polypeptide”.

A peptide, oligopeptide, polypeptide, protein, or polynucleotide coding for such a molecule is “immunogenic” and thus an immunogen within the present disclosure if it is capable of inducing an immune response. In the present disclosure, immunogenicity is more specifically defined as the ability to induce a CTL-mediated response. Thus, an immunogen would be a molecule that is capable of inducing an immune response, and in the present disclosure, a molecule capable of inducing a CTL response. An immunogen may have one or more isoforms, sequence variants, or splice variants that have equivalent biological and immunological activity, and are thus also considered for the purposes of this disclosure to be immunogenic equivalents of the original, natural polypeptide.

In accordance with the present disclosure, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:

Percent Identity=100[1−(C/R)]

wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acId.

The terms “peripheral blood mononuclear cells” or “PBMCs” are used interchangeably herein to refer to blood cells having a single round nucleus such as, for example, a lymphocyte or a monocyte.

The term “pharmaceutical composition” as used herein is meant to refer to a composition that is employed to prevent, reduce in intensity, cure or otherwise treat a target condition, syndrome, disorder or disease.

The term “pharmaceutically acceptable carrier” as used herein is meant to refer to any substantially non-toxic carrier conventionally useable for administration of pharmaceuticals in which the isolated polypeptide of the present disclosure will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition.

The term “pharmaceutically acceptable salt” as used herein is meant to refer to those salts which are, within the scope of sound medical judgment, suitable for use in contact with the tissues of humans and lower animals without undue toxicity, irritation, allergic response and the like and are commensurate with a reasonable benefit/risk ratio. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present disclosure or separately by reacting a free base function with a suitable organic acId. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acId. Basic addition salts may be prepared in situ during the final isolation and purification of compounds described within the disclosure by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The term “plasma cell” as used herein refers to terminally differentiated B cells that secrete antibody. They may be short-lived, with no isotype switching or somatic hypermutation, or long lived, meaning they undergo isotype switching and somatic hypermutation.

The term “plasmablasts” as used herein refer to proliferating progeny of an activated B cell. Plasmablasts become plasma cells. Antigen binding to the BCR triggers activation of Src family kinases such as Lyn and Fyn leading to phosphorylation of Igα (CD79a) and Igβ (CD79b), recruitment of Syk kinase and subsequent recruitment and phosphorylation of BLNK, Btk and PLCγ [Luo, W. et al. J. Immunol. (2014) 193(2): 909-20, citing Packard, T A & Cambier, J C. F1000 prime reports (2013) 5: 40]. These events activate the Ras pathway, PKC pathway and calcium flux, eventually triggering the activation of NF-κB, Erk and JNK. These positive signals are normally counterbalanced by negative signals that limit B cell activation and prevent spontaneous B cell proliferation and differentiation to plasma cells [Id., citing Nitschke, L. Curr. Opin. Immunol. (2005) 17: 2990-97]. Negative signals are generated by a series of membrane receptors (CD22, CD72, FcγRIIb, PIR-B, Siglec-G, etc.) that are phosphorylated by Lyn. This allows them to recruit phosphatases such as SHP1 and SHIP1 that reverse phosphorylation of signaling molecules in the BCR pathway and dampen BCR signaling [Id., citing Poe, J C & Tedder, T F, Trends Immunol. (2012) 33: 413-20; Tsubata, T. Infecious disorders drug targets (2012) 12: 181-90; Vang, T. et al. Annu. Rev. Immunol. (2008) 26: 29-55].

The terms “portion,” “segment,” and “fragment,” when used herein in relation to polypeptides, are meant to refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. When used in relation to polynucleotides, such terms refer to the products produced by treatment of said polynucleotides with endonucleases.

The term “prevention” as used herein, is meant to refer to a process of prophylaxis in which an animal, especially a mammal, and most especially a human, is exposed to an immunogen of the present disclosure prior to the induction or onset of the disease process. This could be done where an individual is at high risk for any viral infection based on the living or travel to the virus pandemic areas. Alternatively, the immunogen could be administered to the general population as is frequently done for any infectious diseases. Alternatively, the term “suppression” is often used to describe a condition wherein the disease process has already begun but obvious symptoms of said condition have yet to be realized. Thus, the cells of an individual may have been infected but no outside signs of the disease have yet been clinically recognized. In either case, the term prophylaxis can be applied to encompass both prevention and suppression.

The term “proliferate” and its various grammatical forms as used herein is meant to refer to the process that results in an increase of the number of cells, and is defined by the balance between cell division and cell loss through cell death or differentiation.

The term “protect” or “protection of” a subject from developing a disease or from becoming susceptible to an infection as referred herein means to partially or fully protect a subject. As used herein, to “fully protect” means that a treated subject does not develop a disease or infection caused by an agent such as a virus, bacterium, fungus, protozoa, helminth, and parasites, or caused by a cancer cell. To “partially protect” as used herein means that a certain subset of subjects may be fully protected from developing a disease or infection after treatment, or that the subject does not develop a disease or infection with the same severity as an untreated subject.

The term “protective immune response” or “protective response” as used herein, is meant to refer to an immune response mediated by antibodies against an infectious agent, which is exhibited by a vertebrate (e.g., a human), that prevents or ameliorates an infection or reduces at least one symptom thereof. Vaccines of the present disclosure can stimulate the production of antibodies that, for example, neutralize infectious agents, block infectious agents from entering cells, block replication of said infectious agents, and/or otherwise protect host cells from infection and destruction. The term can also refer to an immune response that is mediated by T-lymphocytes and/or other white blood cells against an infectious agent, exhibited by a vertebrate (e.g., a human), that prevents or ameliorates a viral infection or reduces at least one symptom thereof.

The term “recombinant” as used herein to describe a nucleic acid molecule is meant to refer to a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

The term “recombinant” vector, such as a DNA plasmid, pseudotyped lentiviral or retroviral vector as used herein, is meant to refer to a vector wherein the material (e.g., a nucleic acid or encoded protein) has been artificially or synthetically (non-naturally) altered by human intervention. The alteration can be performed on the material within, or removed from, its natural environment or state. Specifically, e.g., a protein derived from influenza virus is recombinant when it is produced by the expression of a recombinant nucleic acId. For example, a “recombinant nucleic acid” is one that is made by recombining nucleic acids, e.g., during cloning, or other procedures, or by chemical or other mutagenesis; and a “recombinant polypeptide” or “recombinant protein” is a polypeptide or protein which is produced by expression of a recombinant nucleic acId. Some embodiments of a recombinant nucleic acid includes an open reading frame encoding an HA, NA, and/or a protease, and can further include non-coding regulatory sequences, and introns.

The term “reporter gene” (“reporter”) or “assay marker” as used herein is meant to refer to a gene and/or peptide that can be detected, or easily identified and measured. The expression of the reporter may be measured at either the RNA level, or at the protein level. The gene product, which may be detected in an experimental assay protocol, includes, but is not limited to, marker enzymes, antigens, amino acid sequence markers, cellular phenotypic markers, nucleic acid sequence markers, and the like. Researchers may attach a reporter gene to another gene of interest in cell culture, bacteria, animals, or plants. For example, some reporters are selectable markers, or confer characteristics upon on organisms expressing them allowing the organism to be easily identified and assayed. To introduce a reporter gene into an organism, researchers may place the reporter gene and the gene of interest in the same DNA construct to be inserted into the cell or organism. For bacteria or eukaryotic cells in culture, this may be in the form of a plasmid. Commonly used reporter genes may include, but are not limited to, fluorescent proteins, luciferase, beta-galactosidase, and selectable markers, such as chloramphenicol and kanomycin.

The term “selectable marker” as used herein is meant to refer to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like.

The term “seroconversion” as used herein refers to the phase of an infection when antibodies against the infecting agent are first detectable in the blood.

The term “seronegative” as used herein refers to lacking an antibody of a specific type in serum.

The term “seropositive” as used herein refers to containing antibody of a specific type in serum.

The term “somatic hypermutation” as used herein revers to introduction of random point mutations at an unusually high frequency into the V exons of Ig genes, which increases V region variability in Ig proteins.

The term “subject” as used herein is meant to refer to any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

The phrase “subject in need thereof” as used herein is meant to refer to a patient that (i) will be administered a vaccine according to the described disclosure, (ii) is receiving a vaccine according to the described disclosure; or (iii) has received a vaccine according to the described disclosure, unless the context and usage of the phrase indicates otherwise.

The term “stimulate an immune cell” or “stimulating an immune cell” as used herein is meant to refer to a process (e.g., involving a signaling event or stimulus) causing or resulting in a cellular response, such as activation and/or expansion, of an immune cell, e.g. a CD8+ T cell.

The term “substantially identical” as used herein is meant to refer to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). For example, such a sequence is at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

The terms “T lymphocyte” or “T cell” are used interchangeably to refer to cells that mediate a wide range of immunologic functions, including the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines. T cells recognize antigens on the surface of antigen presenting cells (APCs) and mediate their functions by interacting with, and altering, the behavior of these APCs. T cells can also be classified based on their function as helper T cells; T cells involved in inducing cellular immunity; suppressor T cells; and cytotoxic T cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC).

The term “T cell antigen” as used herein is meant to refer to a protein or fragment thereof which can be processed into a peptide that can bind to either Class I MHC, Class II MHC, nonclassical MHC, or CD1 family molecules (collectively antigen presenting molecules), and in this combination can engage a T cell receptor on a T cell.

The term “T cell epitope” as used herein is meant to refer to a short peptide molecule that binds to a class I or II MHC molecule and that is subsequently recognized by a T cell. T cell epitopes that bind to class I MHC molecules are typically 8-14 amino acids in length, and most typically 9 amino acids in length. T cell epitopes that bind to class II MHC molecules are typically 12-20 amino acids in length. In the case of epitopes that bind to class II MHC molecules, the same T cell epitope may share a common core segment, but differ in the length of the carboxy- and amino-terminal flanking sequences due to the fact that ends of the peptide molecule are not buried in the structure of the class II MHC molecule peptide-binding cleft as they are in the class I MHC molecule peptide-binding cleft.

The term “T cell mediated immune response” as used herein is meant to refer to a response that occurs as a result of recognition of a T cell antigen bound to an antigen presenting molecule on the cell surface of an APC, coupled with other interactions between costimulatory molecules on the T cell and APC. This response serves to induce T cell proliferation, migration, and production of effector molecules, including cytokines and other factors that can injure cells.

The term “T cell receptor” (TCR) as used herein, is meant to refer to a complex of integral membrane proteins that participate in the activation of T cells in response to an antigen. The TCR expressed by the majority of T cells consisting of α and β chains. A small group of T cells express receptors made of γ and δ chains. Among the α/β T cells are two sublineages: those that express the coreceptor molecule CD4 (CD4+ cells), and those that express CD8 (CD8+ cells). These cells differ in how they recognize antigen and in their effector and regulatory functions. CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated. The cytokines can be directly toxic to target cells and can mobilize potent inflammatory mechanisms. CD8+ T cells, can develop into cytotoxic T-lymphocytes (CTLs) capable of efficiently lysing target cells that express antigens recognized by the CTLs.

The term “toll-like receptor (TLR)” as used herein refers to innate receptors on macrophages, dendritic cells, and some other cells, that recognize pathogens and their products, such as bacterial lipopolysaccharide (LPS). Recognition stimulates the receptor-bearing cells to produce cytokines that help initiate immune responses. For example, TLR-1 is a cell surface toll-like receptor that acts in a heterodimer with TLR-2 to recognize lipoteichoic acid and bacterial lipoproteins. TLR-2 is a cell surface toll-like receptor that acts in a heterodimer with either TLR-1 or TLR-6 to recognize lipoteichoic acid and bacterial lipoproteins. TLR-4 is a cell surface toll-like receptor that, in conjunction with accessory proteins MD-2 and CD14, recognizes bacterial lipopolysaccharide and lipoteichoic acid. TLR5 is a cell surface toll-like receptor that recognizes the flagellin protein of bacterial flagella. TLR 6 is a cell surface toll-like receptor that acts in a heterodimer with TLR2 to recognize lipoteichoic acid and bacterial lipoproteins. TLR3 is an endosomal toll-like receptor that recognizes double-stranded viral RNA. TLR-7 is an endosomal toll-like receptor that recognizes single-stranded viral RNA. TLR-8 is an endosomal toll-like receptor that recognizes single-stranded viral RNA. TLR-9 is an endosomal toll-like receptor that recognizes DNA containing unmethylated CpG.

The term “topical administration” and “topically applying” as used herein are used interchangeably to refer to delivering a peptide, a nucleic acid, or a vector comprising the peptide or the nucleic acid, onto one or more surfaces of a tissue or cell, including epithelial surfaces. The composition may be applied by pouring, dropping, or spraying, if a liquid; rubbing on, if an ointment, lotion, cream, gel, or the like; dusting, if a powder; spraying, if a liquid or aerosol composition; or by any other appropriate means.

The term “treatment” as used herein is meant to refer to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

The terms “therapeutic amount”, “effective amount”, an “amount effective”, or “pharmaceutically effective amount” of an active agent are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described disclosure. In prophylactic or preventative applications of the described disclosure, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

The term “therapeutic effect” as used herein is meant to refer to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan.

General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The term “therapeutic window” as used herein is meant to refer to a concentration range that provides therapeutic efficacy without unacceptable toxicity. Following administration of a dose of a drug, its effects usually show a characteristic temporal pattern. A lag period is present before the drug concentration exceeds the minimum effective concentration (“MEC”) for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. Accordingly, the duration of a drug's action is determined by the time period over which concentrations exceed the MEC. The therapeutic goal is to obtain and maintain concentrations within the therapeutic window for the desired response with a minimum of toxicity. Drug response below the MEC for the desired effect will be subtherapeutic, whereas for an adverse effect, the probability of toxicity will increase above the MEC. Increasing or decreasing drug dosage shifts the response curve up or down the intensity scale and is used to modulate the drug's effect. Increasing the dose also prolongs a drug's duration of action but at the risk of increasing the likelihood of adverse effects. Accordingly, unless the drug is nontoxic, increasing the dose is not a useful strategy for extending a drug's duration of action.

Instead, another dose of drug should be given to maintain concentrations within the therapeutic window. In general, the lower limit of the therapeutic range of a drug appears to be approximately equal to the drug concentration that produces about half of the greatest possible therapeutic effect, and the upper limit of the therapeutic range is such that no more than about 5% to about 10% of patients will experience a toxic effect. These figures can be highly variable, and some patients may benefit greatly from drug concentrations that exceed the therapeutic range, while others may suffer significant toxicity at much lower values. The therapeutic goal is to maintain steady-state drug levels within the therapeutic window. For most drugs, the actual concentrations associated with this desired range are not and need not be known, and it is sufficient to understand that efficacy and toxicity are generally concentration-dependent, and how drug dosage and frequency of administration affect the drug level. For a small number of drugs where there is a small (two- to three-fold) difference between concentrations resulting in efficacy and toxicity, a plasma-concentration range associated with effective therapy has been defined.

In this case, a target level strategy is reasonable, wherein a desired target steady-state concentration of the drug (usually in plasma) associated with efficacy and minimal toxicity is chosen, and a dosage is computed that is expected to achieve this value. Drug concentrations subsequently are measured and dosage is adjusted if necessary to approximate the target more closely.

In most clinical situations, drugs are administered in a series of repetitive doses or as a continuous infusion to maintain a steady-state concentration of drug associated with the therapeutic window. To maintain the chosen steady-state or target concentration (“maintenance dose”), the rate of drug administration is adjusted such that the rate of input equals the rate of loss. If the clinician chooses the desired concentration of drug in plasma and knows the clearance and bioavailability for that drug in a particular patient, the appropriate dose and dosing interval can be calculated.

The term “vaccinated” as used herein is meant to refer to being treated with a vaccine.

The term “vaccination” as used herein is meant to refer to treatment with a vaccine.

The term “vaccine” as used herein is meant to refer to a formulation which is in a form that is capable of being administered to a vertebrate and which induces a protective immune response sufficient to induce immunity and/or to prevent and/or ameliorate an infection and/or to reduce at least one symptom of an infection and/or to enhance the efficacy of another dose of a formulation. Typically, the vaccine comprises a conventional saline or buffered aqueous solution medium in which the composition of the present disclosure is suspended or dissolved. In this form, the composition of the present disclosure can be used conveniently to prevent, ameliorate, or otherwise treat a viral infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

The term “vaccine therapy” as used herein is meant to refer to a type of treatment that uses a substance or group of substances to stimulate the immune system to destroy a tumor or infectious microorganisms.

The term “variant” or “derivative” with respect to a peptide or DNA sequence as used herein is meant to refer to a non-identical peptide or DNA sequence that is modified from its original sequence. The differences in the sequences may by the result of changes, by design, in sequence or structure. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence. The terms “variant” or “derivative” with respect to cells as used herein refers to a cell line that has been modified from its cell line of origin (e.g. modified to express recombinant DNA sequences).

The term “vector” as used herein, is meant to refer to a DNA construct that contains a promoter operably linked to a downstream gene or coding region (e.g., a cDNA or genomic DNA fragment, which encodes a polypeptide or polypeptide fragment). Introduction of the vector into a recipient cell (e.g., a prokaryotic or eukaryotic cell, e.g., a bacterium, yeast, insect cell, or mammalian cell, depending upon the promoter within the expression vector) or organism (including, e.g., a human) allows the cell to express mRNA encoded by the vector, which is then translated into the encoded optimized viral polypeptide of the disclosure. Vectors for in vitro transcription/translation are well known in the art and are described further herein. A vector may be a genetically engineered plasmid, virus, or artificial chromosome derived from, e.g., a bacteriophage, adenovirus, retrovirus, poxvirus, or herpesvirus.

The term “virus immune escape” or “virus escape” as used herein refers to mechanisms by which viruses evade the immune system of the host.

The term “virus-like particle” or “VLP” as used herein, is meant to refer to a nonreplicating, viral shell. VLPs are generally composed of one or more viral proteins, such as, but not limited to those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art and discussed more fully below. The presence of VLPs following recombinant expression of viral proteins can be detected using conventional techniques known in the art, such as by electron microscopy, biophysical characterization, and the like. See, e.g., Baker et al., Biophys. J. (1991) 60:1445-1456; Hagensee et al., J. Virol. (1994) 68:4503-4505. For example, VLPs can be isolated by density gradient centrifugation and/or identified by characteristic density banding (e.g., Examples). Alternatively, cryoelectron microscopy can be performed on vitrified aqueous samples of the VLP preparation in question, and images recorded under appropriate exposure conditions. Additional methods of VLP purification include but are not limited to chromatographic techniques such as affinity, ion exchange, size exclusion, and reverse phase procedures.

The term “wild-type” as used herein is meant to refer to the typical form of an organism, strain, gene, protein, nucleic acid, or characteristic as it occurs in nature. Wild-type refers to the most common phenotype in the natural population. The terms “wild-type” and “naturally occurring” are used interchangeably.

### EMBODIMENTS

### Compositions

The theory behind the design of a T cell vaccine is that the presence of a strong and immediate CTL response present at the time of viral exposure would, at a minimum, reduce viral loads in infected individuals by reducing acute viremia. However, the induction of a functional antibody response, such as in the form of broadly neutralizing antibodies (NAb), is a means to provide an early and, desirably, protective immune response. Accordingly, as described in more detail throughout the description and Examples herein, the disclosure addresses the need for a balanced immune response consisting of both cellular immunity, coupled with a broad neutralizing antibody response in the design of a candidate universal vaccine to HIV or a CoV, e.g., SARS-CoV-2.

According to one aspect, the disclosure provides an immunogen, or an immunogenic portion thereof, that is designed against an internal CD8+ T cell epitope of an infectious agent as described herein, by using the internal conserved proteins of the infectious agent. As described in more detail throughout the description and Examples herein, consensus amino acid sequences can be deduced for example from viral strains available in the Genbank database, and the amino acid having the highest frequency of occurrence at each position of the amino acid sequence can be used as the consensus amino acid at that site. Thus, the resulting protein sequence constitutes the shared amino acid of each site, and can be analyzed using a publicly available online CD8+ T cell epitope prediction software and at tools.immuneepitope.org/main/tcell/ (available online at syfpeithi.de and described in Singh H, et al. (2003) Bioinformatics; 19: 1009-1014 and Moutaftsi M, et al. (2006) Nat Biotechnol. 24: 817-819, the contents of each of which are incorporated by reference in their entireties herein). The sequences can then be modified following optimized mammalian codon usage.

According to some embodiments, the infectious agent is a virus belongs to a virus family selected from, but not limited to the group consisting of Coronviridae and Retroviridae. According to some embodiments, the virus from the Retroviridae family is from the genus Deltaretrovirus. According to some embodiments, the virus from the Retroviridae family is from the genus Epsilonretrovirus. According to some embodiments, the virus from the Retroviridae family is from the genus Gammaretrovirus. According to some embodiments, the virus from the Retroviridae family is from the genus Lentivirus. According to some embodiments, the virus from the Retroviridae family is from the genus Spumavirus.

Exemplary conserved viral proteins and corresponding RefSeq Nos are shown in Table 1A and Table 1B and Example 1A. It is to be understood that Table 1A and Table 1B are merely exemplary, and any internal conserved protein of any desired virus that is publicly available in GenBank can be used in the methods described herein.

According to some embodiments, nucleic acids are provided that express immunogenic domains rather than the entire protein. These portions (or fragments) may be of any length sufficient to be immunogenic or antigenic. Immunogenicity can be determined using any of the assays described herein. Fragments may be at least four amino acids long, or for example 5, 6, 7, 8, or 9 amino acids long, or may be longer, such as e.g. 10, 11, 12, 13, 14, 15, 16, 17, 18, 19, 20, 30, 40, 50, 60, 70, 80, 90, 100, 200, 300, 400, 500 amino acids long, inclusive, or more, or any length in between. Epitopes that induce a protective immune response to pathogens such as bacteria, viruses, fungi or protozoae may be combined with heterologous gene sequences that encode proteins with immunomodulating activities, such as cytokines, interferon type 1, gamma interferon, colony stimulating factors, and interleukin-1, -2, -4, -5, -6, -12.

Immunogens as described herein comprise polypeptides with amino acid sequences comprising conserved CD8+ T cell epitopes of internal antigens of the infectious agent generated and selected according to the methods described herein, wherein said immunogens facilitate a cytotoxic T lymphocyte (CTL)-mediated immune response against various strains of cells infected with the agent. Also provided by the present disclosure are nucleic acid molecules that encode polypeptides comprising said epitopic peptide, and which can also be used to facilitate an immune response against the infected cells.

The present disclosure provides compositions comprising the polypeptides and nucleic acid molecules that encode the polypeptides described herein whereby the oligopeptides and polypeptides of such immunogens are capable of inducing a CTL response against cells expressing a protein comprising conserved CD8+ T cell epitopes of internal antigens of an infectious agent generated and selected according to the methods described herein, presented in association with Class I MHC protein, which cells are infected with various strains of an infectious agent. Alternatively, the immunogens of the present disclosure can be used to induce a CTL response in vitro. The generated CTL population(s) can then be introduced into a patient with an infection caused by the infectious agent. Alternatively, since lymphocyte activation is manifested by various cellular processes, such as cell proliferation, gene expression and protein secretion, these may be evaluated by various methods. Accordingly, the ability to generate CTLs in vitro in a cell based activation immunoassay can serve as a diagnostic for early pathogen detection.

The immunogens described herein include fragments or portions thereof that maintain the same biological activity as the reference immunogen. Binding of peptides to MHC (major histocompatibility complex) molecules is a prerequisite for a peptide to be an immunogen. Accordingly, in some embodiments, the immunogens, or fragment thereof, described herein, comprise infectious agent-specific CD8+epitopes that bind with high affinity to human MHC class I molecules to elicit a CD8+ T cell response.

### Immunogens

Using the methods described herein to identify and generate universal vaccines targeting conserved T cell epitopes, it should be possible to immunize against a wide spectrum of infectious agents such as, but not limited to, those described below.

According to some embodiments, an immunogen (i.e., a conserved immunogen as described herein), by inducing an immune response, inhibits an infectious disease, e.g., reduces or alleviates a cause or symptom of an infectious disease, or improves a value for a parameter associated with the infectious disease.

According to some embodiments, the immunogens of the disclosure can be chemically synthesized and purified using methods which are well known to the ordinarily skilled artisan. The immunogens can also be synthesized by well-known recombinant DNA techniques. According to some embodiments, the immunogenic peptides and polypeptides of the disclosure are prepared synthetically, or by any means known in the art, including those techniques involving recombinant DNA technology.

According to some embodiments, the coding sequences for peptides contemplated herein can be synthesized on commercially available automated DNA synthesizers or modified to a desired amino acid substitution. The coding sequence can be transformed or transfected into suitable hosts to produce the desired fusion protein.

Genetic modifications including codon optimization, RNA optimization, and/or the addition of a high efficient immunoglobin leader sequence to increase the immunogenicity of constructs are contemplated.

### Viral Immunogens

According to some embodiments, the vaccine of the present disclosure immunizes against a Coronaviridae virus. For example, the Coronaviridae virus is a coronavirus, torovirus, SARs-like coronavirus, and the like.

According to some embodiments, the immunogen of the present disclosure comprises at least one conserved protein of a Coronaviridae virus or an immunogenic fragment thereof. For example, the Coronaviridae virus is a SARS virus. According to some embodiments, new SARS isolates may be identified by a percent homology of 99%, 95%, 92%, 90%, 85%, or 80% homology of the polypeptide sequence encoded by the polynucleotide of specific genomic regions of the new SARS virus to the polypeptide sequence encoded by the polynucleotides of specific regions of a known SARS virus. Table 1A below shows SARS-CoV isolates accessed from GenBank.

TABLE 1A

| | |
|---|---|
| Genome sequences | Accession number |

SARS-CoV isolates

| | |
|---|---|
| Complete | |
| SIN2500 | AY283794 |
| SIN2677 | AY283795 |
| SIN2679 | AY283796 |
| SIN2748 | AY283797 |
| SIN2774 | AY283798 |
| TOR2 | NC_004718 |
| URBANI | AY278741 |
| CUHKU-W1 | AY278554 |
| HKU-39849 | AY278491 |
| SARS-CoV-2 | NC_045512.2 |
| Partial | |
| GZ01 | AY278489 |
| BJ01 | AY278488 |
| BJ02 | AY278487 |
| BJ03 | AY278490 |
| BJ04 | AY279354 |

Coronavirus Isolates

| | |
|---|---|
| MHV strain 2 | AF201929.1 |
| MHV Penn 97-1 | AF208066 |
| MHV | NC_001846 |
| MHV strain ML-10 | AF208067 |
| Bovine CoV | NC_003045.1 |
| BCoV Quebac strain | AF220295.1 |
| AIBV | NC_001451.1 |
| TGV | NC_002306.2 |
| TGV | Z34093.1 |
| HCoV 229E | NC_002645.1 |
| PEDV strain C | AF35311.1 |

SARS-CoV isolates

| | |
|---|---|
| PEDV | NC_003436.1 |
| Rat SDAV | AF207551.1 |
| Porcine HEV | AY078417.1 |

Ruan et al. (The Lancet, vol. 361. May 24, 2003)

TABLE 11
of EP1618127B1, which is reproduced below, shows protein
homologies between SARS and other coronaviruses. The
numbers in reproduced Table 11 indicate percentage of amino
acid identity between SARS proteins and corresponding gene
products of other coronaviruses. More conserved pairs are in bold;
more variable pairs are underlined.

| | Group 1 | | | Group 2 | | Group 3 |
|---|---|---|---|---|---|---|
| Proteins | 229E | TGV | PEDV | MHV | BCoV | AIBV |

REPLICASE REGION

| | | | | | | |
|---|---|---|---|---|---|---|
| leader | <20 | <20 | <20 | 27 | <20 | <20 |
| protein p28 | | | | | | |
| p65 | <20 | 23 | 23 | <20 | 20 | <20 |
| homologue | | | | | | |
| nsp1 | 25.5 | 25.8 | 25.4 | 29 | 30 | 25 |
| (PLP protease) | | | | | | |
| nsp2 | 40.4 | 43.8 | 44.6 | 50 | 48.4 | 41 |
| (3CL prtease) | | | | | | |
| nsp3 | 30 | 27 | 29.4 | 34.2 | 35.5 | 28.5 |
| nsp4 | 38.6 | 42.2 | 39.8 | 47.5 | 46.1 | 37.3 |
| nsp5 | 48.2 | 42.9 | 43.9 | 46.8 | 47.3 | 38.7 |
| n5p6 | 45.1 | 38.9 | 45.1 | 45.1 | 46.9 | 39.8 |
| nsp7 | 53.8 | 54.5 | 56.1 | 56.2 | 55.4 | 58.3 |
| nsp9 | 59.8 | 59.6 | 60 | 67.3 | 66.9 | 62.4 |
| (polymerase0 | | | | | | |
| nsp10 | 60.7 | 62 | 62.3 | 67.2 | 68.6 | 58.9 |
| (helicase) | | | | | | |
| nsp11 | 52.3 | 53.7 | 52.3 | 57.6 | 57.6 | 52 |
| nsp12 | 43.1 | 43 | 45.4 | 45.9 | 45 | 40.2 |
| nsp13 | 56.4 | 54.4 | 55.3 | 63 | 65 | 53.4 |

STRUCTURAL REGION

| | | | | | | |
|---|---|---|---|---|---|---|
| Spike (S) | 28.8 | 31.6* | 30.3 | 31.1 | 31 | 32.7* |
| Envenlope € | 33* | 27.9 | 20 | 23 | 26.5 | 23.2 |
| Membrane | 30.6 | 32.5 | 34.8 | 40.8 | 41.9 | 32.5 |
| glycoprotein | | | | | | |
| (M) | | | | | | |
| Nucleocapsid | 26.9 | 30.1 | 29.5 | 37.3 | 37.4 | 31.5 |
| (N) | | | | | | |

*These three alignments were obtained only on a fragment of the whole protein.

According to some embodiments, the SARS virus is SARS-CoV. According to some embodiments, the SARS virus is SARS-CoV-2. The complete genome of severe acute respiratory syndrome coronavirus 2 (SARS-CoV-2) isolate Wuhan-Hu-1 is set forth at NCBI Reference Sequence: NC_045512.2. The envelope protein E is a very short polypeptide of 76 aa, involved in the morphogenesis of the virion envelope. (Godet et al., Virology 1992; 188(2):666-675). Computer analysis predicts a long transmembrane domain close to the N-terminus and two N-glycosylation sites. As shown in Table 11 of EP1618127B1, the level of amino acid similarity with other coronaviruses is very low and the best homology is with the small envelope protein of the transmissible gastroenteritis virus (TGV).

The matrix glycoprotein (M) is a 221-residue polypeptide with a predicted molecular weight of 25 kDa. Computer analysis predicts a topology consisting of a short amino terminal ectodomain, three transmembrane segments and a carboxyl terminus located at the interior side of the viral envelope. By analogy with the matrix glycoprotein of TGV, that of the avian infective bronchitis virus (AIBV) and that of the hypervirulent MHV-2 strain, the SARS M glycoprotein is N-glycosylated at the N-terminus. SARS M protein shows highest similarity to group 2 viruses, as shown in Table 11 of EP1618127B1.

The nucleocapsid protein N is a 397-residue-long phosphoprotein that interacts with viral genomic RNA to form the nucleocapsid. The level of conservation with other coronaviruses is low, ranging from 26.9% identity with the HCoV-229E to 37.4% identity to the Bovine coronavirus (BcoV), as shown in Table 11 of EP1618127B1.

According to some embodiments, the immunogen of the present disclosure comprises at least one conserved protein of a Coronaviridae virus or an immunogenic fragment thereof, selected from at least one of the following genomic regions: 5′ untranslated region (UTR), leader sequence, ORF1a, ORF1b, nonstructural protein 2 (NS2), hemagglutinin-esterase glycoprotein (HE) (also referred to as E3), spike glycoprotein (S) (also referred to as E2), ORF3a, ORF3b, ORF3x, nonstructural protein 4 (NS4), envelope (small membrane) protein (E) (also referred to as sM), membrane glycoprotein (M) (also referred to as E1), ORF5a, ORF5b, nucleocapsid phosphoprotein (N), ORF7a, ORF7b, intergenic sequences, 3′UTR, or RNA dependent RNA polymerase (pol). According to some embodiments, an immunogen includes at least one protein encoded by any one of these genomic regions. An immunogen of the present disclosure may be a protein or an immunogenic fragment thereof, which is conserved among viruses of the Coronaviridae family.

According to some embodiments, the immunogen of the present disclosure comprises at least one SARS-CoV conserved spike protein (S), membrane protein (M), envelope protein (E), or nucleocapsid protein (N), or an immunogenic fragment thereof.

Several studies have shown that effective cytotoxic lymphocyte priming can be induced by the inclusion of helper T cell (Th) epitopes in peptide vaccines [see, e.g., Zwaveling, S. et al. J. Immunol. (2002) citing Shirai, M, et al. J. Immunol. (1994) 152: 549; Partidos, C D et al. Immunology (1996) 87: 179; Hiranuma, K. et al. J. Gen. Virol. (1999) 80: 18; 7; Bristol, J A, et al. Cell. Immunol. (2000) 205: 73]. Without being limited to any one theory, the effectiveness of these Th cells may lie in their capacity to deliver essential activation signals to professional APCs, such as dendritic cells.

According to some embodiments, the immunogen contains overlapping peptides of at least 30 amino acids in length that together cover the full length sequence of a coronavirus S protein. According to some embodiments, the immunogen contains overlapping peptides of at least 30 amino acids in length that together cover the full length sequence of a coronavirus M protein. According to some embodiments, the immunogen contains overlapping peptides of at least 30 amino acids in length that together cover the full length sequence of a coronavirus E protein. According to some embodiments, the immunogen contains overlapping peptides of at least 30 amino acids in length that together cover the full length sequence of a coronavirus N protein.

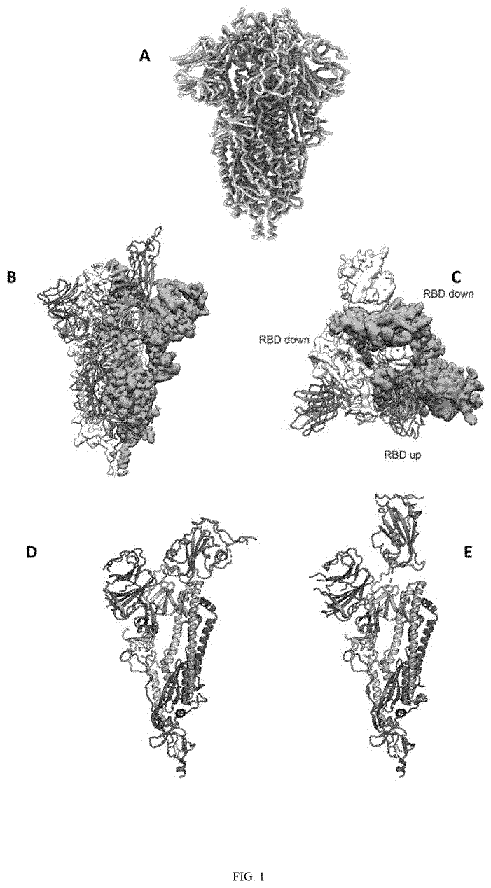

The S protein of coronaviruses exists in a metastable prefusion conformation and comprises two functional subunits for binding to the host cell receptor ACE 2 (S1 subunit) and for fusion of the viral and host cell membranes (S2 subunit). [Sternberg, A. & Naujokat, C. Life Sci. (2020) 257: 118056, citing Walls, A C et al. Cell (2020) 181: 281-92; Kirchdoerfer, R N et al. Nature (2016) 531: 118-21]. The S protein of SARS-CoV is cleaved by a host cell protease, the transmembrane protease/serine subfamily member 2 (TMPRSS2), an airway and alveolar cell serine protease preferentially expressed on epithelial cells of the respiratory tract, such as type II pneumocytes. [Id., citing Matsuyama, S. et al. J. Virol. (2010) 84: 12685-64; Shulla, A. et al. J. Virol. (2011) 85: 873-82; Glowacka, I. et al. J. Virol. (2011) 85: 4122-34]. TMPRSS2-mediated cleavage and priming of SARSCoV S protein is required for binding to ACE2, membrane fusion and cell entry that requires a concerted action of a viral and host cell machinery comprising S protein, TMPRSS2 and ACE2 [Id., citing Matsuyama, S. et al. J. Virol. (2010) 84: 12685-64; Shulla, A. et al. J. Virol. (2011) 85: 873-82; Glowacka, I. et al. J. Virol. (2011) 85: 4122-34]. SARS-CoV-2 also employs TMPRSS2 for priming of its S protein (S) and S-driven cell entry via ACE2 [Id., citing Hoffman, M. et al. Cell (2020) 181: 271-80; Zhou, P. et al. Nature (2020) 579: 270-73; Wrapp, D. et al. Science (2020) 367: 1260-63; Walls, A C et al. Cell (2020) 181: 281-92; Ziegler, C G K et al. Cell (2020) doi.org/10.1016/j.ce11.2020.04.035]. Further, the S1/S2 boundary of SARS-CoV-2 S harbors multiple arginine residues not found in SARS-CoV and SARS-CoV-related S proteins. This boundary constitutes the cleavage site for the subtilisin-like host cell protease furin, which is ubiquitously expressed in humans [Id., citing Wrapp, D. et al. Science (2020) 367: 1260-63; Walls, A C et al. Cell (2020) 181: 281-92; Hoffman, M. et al. Mol. Cell (2020) 78: 1-6]. For ACE2 receptor engagement of SARS-CoV-2, the RBDs located at the apex of 51 undergo hinge-like conformational movements that transiently expose (open status, “up”) or hide (closed status, “down”) the subdomains required for receptor binding, whereby the open status allows for receptor engagement, followed by shedding of 51 and refolding of S2 for membrane fusion. [Sternberg, A. & Naujokat, C. Life Sci. (2020) 257: 118056, citing Wrapp, D. et al. Science (2020) 367: 1260-63; Walls, A C et al. Cell (2020) 181: 281-92]. See FIG. 1. Although the RBDs of the 51 subunit are more exposed on the viral surface than the S2 fusion machinery, the S2 fusion machinery is densely decorated with heterogeneous N-linked glycans protruding from the S2 surface that may interfere with the elicitation of humoral immune responses and the accessibility to neutralizing antibodies. [Id., citing Walls, A C et al. Cell (2020) 181: 281-92]. In addition, the RBDs of 51 also contain N-linked glycans and unexpected O-linked glycans attached to the surface of 51 RBDs that also may interfere with the elicitation of neutralizing antibodies upon immune exposure or vaccination. [Id., citing Shajahan, a. et al. Glycobiology (2020) doi.org/10.1093/glycob/cwaa042 May 4; Vigerust, D J & Shepard, V L. Trends Microbiol. (2007) 15: 211-18]. In convalescent patients, adaptive immunity in SARS-CoV-2 is largely mediated by CD4+ T cells with a T cell receptor repertoire specific for S epitopes, leading to the robust generation of neutralizing IgG, IgM and IgA antibodies against the RBDs and the ectodomain trimer of 51 [Id., citing Grifoni, A. et al. Cell (2020) 181: 1489-1501; Cao, Y. et al. Cell (2020) doi.org/10.1016/j.ce11.2020.05.025].

The S protein has the functions of combining virus and host cell membrane receptors and fusing membranes. According to some embodiments, the S protein determines the host range and specificity of the virus. According to some embodiments, the S protein can realize transmission among different hosts through gene recombination or mutation of a Receptor Binding Region (RBD), and thereby lead to higher lethality rate. According to some embodiments the S protein when used as an immunogen can generate neutralizing antibodies.

According to some embodiments, the immunogen comprises 51, or an immunogenic fragment thereof. According to some embodiments, the immunogen comprises S2, or an immunogenic fragment thereof. According to some embodiments, the immunogen contains overlapping peptides of at least 30 amino acids in length that together cover the full length sequence of a coronavirus S1 subunit. According to some embodiments, the immunogen contains overlapping peptides of at least 30 amino acids in length that together cover the full length sequence of a coronavirus S2 subunit.

According to some embodiments, the immunogen is an isolated coronavirus S protein S1 subunit cleaved by a transmembrane serine protease (TMPRSS2) comprising a receptor binding domain (RBD) or an immunogenic fragment thereof. According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 5% of its covering of N-linked glycans, O-linked glycans, or both by limited digestion. N- and O-glycases are commercially available, for example, from Genovis.com. According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 10% of its covering of N-linked glycans, O-linked glcans, or both by limited digestion. According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 15% of its covering of N-linked glycans, 0-linked glcans, or both by limited digestion. According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 20% of its covering of N-linked glycans, O-linked glcans, or both by limited digestion.

According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 25% of its covering of N-linked glycans, O-linked glcans, or both by limited digestion. According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 30% of its covering of N-linked glycans, O-linked glcans, or both by limited digestion. According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 35% of its covering of N-linked glycans, O-linked glcans, or both by limited digestion. According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 40% of its covering of N-linked glycans, O-linked glcans, or both by limited digestion. According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 45% of its covering of N-linked glycans, O-linked glcans, or both by limited digestion.

According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 50% of its covering of N-linked glycans, O-linked glcans, or both by limited digestion. According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 55% of its covering of N-linked glycans, O-linked glcans, or both by limited digestion. According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 60% of its covering of N-linked glycans, O-linked glcans, or both by limited digestion. According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 65% of its covering of N-linked glycans, O-linked glcans, or both by limited digestion. According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 70% of its covering of N-linked glycans, O-linked glcans, or both by limited digestion.

According to some embodiments, the immunogen is an isolated coronavirus S protein S1 subunit cleaved by a transmembrane serine protease (TMPRSS2) comprising a receptor binding domain (RBD) of the isolated S1 subunit enzymatically stripped of at least 75% of its covering of N-linked glycans, O-linked glcans, or both by limited digestion.

The coronavirus spike proteins (and other similar surface viral proteins) are thought to undergo a conformational change upon receptor binding to the target cell membrane. One or more of the hydrophobic Membrane Fusion peptides are thought to become exposed and inserted into the target membrane as a result of this conformational change. The free energy released upon subsequent refolding of the spike protein to its most stable conformation is believed to play a role in the merger of the viral and cellular membranes.

According to some embodiments, the spike (S) protein comprises SEQ ID NO: 1, shown below.

MKVLIFALLFSLAKAQEGCGIISRKPQPKMEKVSSSRRGVYYNDDIFRS
DVLHLTQDYFLPFDSNLTQYFSLNIDSNKYTYFDNPILDFGDGVYFAAT
EKSNVIRGWIFGSSFDNTTQSAIIVNNSTHIIIRVCNFNLCKEPMYTVS
KGTQQSSWVYQSAFNCTYDRVEKSFQLDTAPKTGNFKDLREYVFKNKGG
FLRVYQTYTAVNLPRGFPAGFSVLRPILKLPFGINITSYRVVMTMFSQF
NSNFLPESAAYYVGNLKYTTFMLSFNENGTITDAVDCSQNPLAELKCTI
KNFNVSKGIYQTSNFRVTPTQEVVRFPNITNRCPFDKVFNASRFPNVYA
WERTKISDCVADYTVLYNSTSFSTFKCYGVSPSKLIDLCFTSVYADTFL
IRSSEVRQVAPGETGVIADYNYKLPDDFTGCVIAWNTAQQDQGQYYYRS
YRKEKLKPFERDLSSDENGVYTLSTYDFYPSIPVEYQATRVVVLSFELL
NAPATVCGPKLSTQLVKNQCVNFNFNGLRGTGVLTTSSKRFQSFQQFGR
DTSDFTDSVRDPQTLEILDISPCSFGGVSVITPGTNASSEVAVLYQDVN
CTDVPTSIHADQLTPAWRVYSTGVNVFQTQAGCLIGAEHVNASYECDIP
IGAGICASYHTASVLRSTGQKSIVAYTMSLGAENSIAYANNSIAIPTNF
SISVTTEVMPVSIAKTSVDCTMYICGDSLECSNLLLQYGSFCTQLNRAL
TGIAIEQDKNTQEVFAQVKQMYKTPAIKDFGGFNFSQILPDPSKPTKRS
FIEDLLFNKVTLADAGFMKQYGECLGDISARDLICAQKFNGLTVLPPLL
TDEMIAAYTAALVSGTATAGWTFGAGSALQIPFAMQMAYRFNGIGVTQN
VLYENQKQIANQFNKAISQIQESLTTTSTALGKLQDVVNQNAQALNTLV
KQLSSNFGAISSVLNDILSRLDKVEAEVQIDRLITGRLQSLQTYVTQQL
IRAAEIRASANLAATKMSECVLGQSKRVDFCGKGYHLMSFPQAAPHGVV
FLHVTYVPSQERNFTTAPAICHEGKAYFPREGVFVSNGTSWFITQRNFY
SPQIITTDNTFVAGNCDVVIGIINNTVYDPLQPELDSFKEELDKYFKNH
TSPDVDLGDISGINASVVNIQKEIDRLNEVAKNLNESLIDLQELGKYEQ
YIKWPWYVWLGFIAGLIAIVMVTILLCCMTSCCSCLKGACSCGSCCKFD
EDDSEPVLKGVKLHYT

According to some embodiments, the spike (S) protein comprises SEQ ID NO: 2, shown below.

MFVFLVLLPLVSSQCVNLTTRTQLPPAYTNSFTRGVYYPDKVFR
SSVLHSTQDLFLPFFSNVTWFHAIHVSGTNGTKRFDNPVLPFNDGVYFA
STEKSNIIRGWIFGTTLDSKTQSLLIVNNATNVVIKVCEFQFCNDPFLG
VYYHKNNKSWMESEFRVYSSANNCTFEYVSQPFLMDLEGKQGNFKNLRE
FVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAV
DCALDPLSETKCTLKSFTVEKGIYQTSNFRVQPTESIVRFPNITNLCPF
GEVFNATRFASVYAWNRKRISNCVADYSVLYNSASFSTFKCYGVSPTKL
NDLCFTNVYADSFVIRGDEVRQIAPGQTGKIADYNYKLPDDFTGCVIAW
NSNNLDSKVGGNYNYLYRLFRKSNLKPFERDISTEIYQAGSTPCNGVEG
FNCYFPLQSYGFQPTNGVGYQPYRVVVLSFELLHAPATVCGPKKSTNLV
KNKCVNFNFNGLTGTGVLTESNKKFLPFQQFGRDIADTTDAVRDPQTLE
ILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSP
RRARSVASQSIIAYTMSLGAENSVAYSNNSIAIPTNFTISVTTEILPVS
MTKTSVDCTMYICGDSTECSNLLLQYGSFCTQLNRALTGIAVEQDKNTQ
EVFAQVKQIYKTPPIKDFGGFNFSQILPDPSKPSKRSFIEDLLFNKVTL
ADAGFIKQYGDCLGDIAARDLICAQKFNGLTVLPPLLTDEMIAQYTSAL
LAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISS
VLNDILSRLDKVEAEVQIDRLITGRLQSLQTYVTQQLIRAAEIRASANL
AATKMSECVLGQSKRVDFCGKGYHLMSFPQSAPHGVVFLHVTYVPAQEK
NFTTAPAICHDGKAHFPREGVFVSNGTHWFVTQRNFYEPQIITTDNTFV
SGNCDVVIGIVNNTVYDPLQPELDSFKEELDKYFKNHTSPDVDLGDISG
INASVVNIQKEIDRLNEVAKNLNESLIDLQELGKYEQYIKWPWYIWLGF
IAGLIAIVMVTIMLCCMTSCCSCLKGCCSCGSCCKFDEDDSEPVLKGVK
LHYT

According to some embodiments, at least one conserved protein of a Coronaviridae virus is a S protein, or fragment thereof, comprising an amino acid sequence at least 85% identical to SEQ ID NO: 1 or SEQ ID NO: 2. According to some embodiments, at least one conserved protein of a Coronaviridae virus is a S protein, or fragment thereof, comprising an amino acid sequence at least 90% identical to SEQ ID NO: 1 or SEQ ID NO: 2. According to some embodiments, at least one conserved protein of a Coronaviridae virus is a S protein, or fragment thereof, comprising an amino acid sequence at least 95% identical to SEQ ID NO: 1 or SEQ ID NO: 2. According to some embodiments, at least one conserved protein of a Coronaviridae virus is a S protein, or fragment thereof, comprising an amino acid sequence at least 98% identical to SEQ ID NO: 1 or SEQ ID NO: 2. According to some embodiments, at least one conserved protein of a Coronaviridae virus is a S protein, or fragment thereof, comprising an amino acid sequence at least 99% identical to SEQ ID NO: 1 or SEQ ID NO: 2. According to some embodiments, at least one conserved protein of a Coronaviridae virus is a S protein, that consists of SEQ ID NO: 1 or SEQ ID NO: 2.

According to some embodiments, the membrane (M) protein comprises SEQ ID NO: 3, shown below:

MTDNGTITVEELKQLLEQWNLVIGFIFLAWIMLLQFAYSNRNRFLYIIK
LVFLWLLWPVTLACFVLAAVYRINWVTGGIAIAMACIVGLMWLSYFVAS
FRLFARTRSMWSFNPETNILLNVPLRGTILTRPLLESELVIGAVIIRGH
LRMAGHSLGRCDIKDLPKEITVATSRTLSYYKLGASQRVGNDSGFAAYN
RYRIGNYKLNTDHSGSNDNIALLVQ

According to some embodiments, the membrane (M) protein comprises SEQ ID NO: 4, shown below.

MADSNGTITVEELKKLLEQWNLVIGFLFLTWICLLQFAYANRNR
FLYIIKLIFLWLLWPVTLACFVLAAVYRINWITGGIAIAMACLVGLMWL
SYFIASFRLFARTRSMWSFNPETNILLNVPLHGTILTRPLLESELVIGA
VILRGHLRIAGHHLGRCDIKDLPKEITVATSRTLSYYKLGASQRVAGDS
GFAAYSRYRIGNYKLNTDHSSSSDNIALLVQ

According to some embodiments, at least one conserved protein of a Coronaviridae virus is a M protein, or fragment thereof, comprising an amino acid sequence at least 85% identical to SEQ ID NO: 3 or SEQ ID NO: 4. According to some embodiments, at least one conserved protein of a Coronaviridae virus is a M protein, or fragment thereof, comprising an amino acid sequence at least 90% identical to SEQ ID NO: 3 or SEQ ID NO: 4. According to some embodiments, at least one conserved protein of a Coronaviridae virus is a M protein, or fragment thereof, comprising an amino acid sequence at least 95% identical to SEQ ID NO: 3 or SEQ ID NO: 4. According to some embodiments, at least one conserved protein of a Coronaviridae virus is a M protein, or fragment thereof, comprising an amino acid sequence at least 98% identical to SEQ ID NO: 3 or SEQ ID NO: 4. According to some embodiments, at least one conserved protein of a Coronaviridae virus is a M protein, or fragment thereof, comprising an amino acid sequence at least 99% identical to SEQ ID NO: 3 or SEQ ID NO: 4. According to some embodiments, at least one conserved protein of a Coronaviridae virus is a M protein, that consists of SEQ ID NO: 3 or SEQ ID NO: 4.

According to some embodiments, the envelope (E) protein comprises SEQ ID NO: 5, shown below.

MYSFVSEETGTLIVNSVLLFFAFVVFLLVTLAILTALRLCAYCCNIVNV
SLVKPTVYVYSRVKNLNSSEGVPDLLV

According to some embodiments, the envelope (E) protein comprises SEQ ID NO: 6, shown below.

MYSFVSEETGTLIVNSVLLFLAFVVFLLVTLAILTALRLCAYCC
NIVNVSLVKPSFYVYSRVKNLNSSRVPDLL

According to some embodiments, at least one conserved protein of a Coronaviridae virus is an E protein, or fragment thereof, comprising an amino acid sequence at least 85% identical to SEQ ID NO: 5 or SEQ ID NO: 6. According to some embodiments, at least one conserved protein of a Coronaviridae virus is an E protein, or fragment thereof, comprising an amino acid sequence at least 90% identical to SEQ ID NO: 5 or SEQ ID NO: 6. According to some embodiments, at least one conserved protein of a Coronaviridae virus is an E protein, or fragment thereof, comprising an amino acid sequence at least 95% identical to SEQ ID NO: 5 or SEQ ID NO: 6. According to some embodiments, at least one conserved protein of a Coronaviridae virus is an E protein, or fragment thereof, comprising an amino acid sequence at least 98% identical to SEQ ID NO: 5 or SEQ ID NO: 6. According to some embodiments, at least one conserved protein of a Coronaviridae virus is an E protein, or fragment thereof, comprising an amino acid sequence at least 99% identical to SEQ ID NO: 5 or SEQ ID NO: 6. According to some embodiments, at least one conserved protein of a Coronaviridae virus is an E protein, that consists of SEQ ID NO: 5 or SEQ ID NO: 6.

According to some embodiments, the nucleocapsid (N) protein comprises SEQ ID NO: 7, shown below.

MSDNGPQNQRSAPRITFGGPSDSTDNNQDGGRSGARPKQRRPQGLPNNT
ASWFTALTQHGKEELRFPRGQGVPINTNSGKDDQIGYYRRATRRVRGGD
GKMKELSPRWYFYYLGTGPEASLPYGANKEGIVWVATEGALNTPKDHIG
TRNPNNNAAIVLQLPQGTTLPKGFYAEGSRGGSQASSRSSSRSRGNSRN
STPGSSRGNSPARMASGSGETALALLLLDRLNQLESKVSGKGQQQQGQT
VTKKSAAEASKKPRQKRTATKSYNVTQAFGRRGPEQTQGNFGDQDLIRQ
GTDYKYWPQIAQFAPSASAFFGMSRIGMEVTPLGTWLTYHGAIKLDDKD
PQFKDNVILLNKHIDAYKTFPPTEPKKDKKKKTDEAQPLPQRKKQPTVT
LLPAADMDDFSRQLQNSMSGASADSTQA

According to some embodiments, the nucleocapsid (N) protein comprises SEQ ID NO: 8, shown below.

MSDNGPQNQRNAPRITFGGPSDSTGSNQNGERSGARSKQRRPQG
LPNNTASWFTALTQHGKEDLKFPRGQGVPINTNSSPDDQIGYYRRATRR
IRGGDGKMKDLSPRWYFYYLGTGPEAGLPYGANKDGIIWVATEGALNTP
KDHIGTRNPANNAAIVLQLPQGTTLPKGFYAEGSRGGSQASSRSSSRSR
NSSRNSTPGSSRGTSPARMAGNGGDAALALLLLDRLNQLESKMSGKGQQ
QQGQTVTKKSAAEASKKPRQKRTATKAYNVTQAFGRRGPEQTQGNFGDQ
ELIRQGTDYKHWPQIAQFAPSASAFFGMSRIGMEVTPSGTWLTYTGAIK
LDDKDPNFKDQVILLNKHIDAYKTFPPTEPKKDKKKKADETQALPQRQK
KQQTVTLLPAADLDDFSKQLQQSMSSADSTQA

According to some embodiments, at least one conserved protein of a Coronaviridae virus is a N protein, or fragment thereof, comprising an amino acid sequence at least 85% identical to SEQ ID NO: 7 or SEQ ID NO: 8. According to some embodiments, at least one conserved protein of a Coronaviridae virus is a N protein, or fragment thereof, comprising an amino acid sequence at least 90% identical to SEQ ID NO: 7 or SEQ ID NO: 8. According to some embodiments, at least one conserved protein of a Coronaviridae virus is a N protein, or fragment thereof, comprising an amino acid sequence at least 95% identical to SEQ ID NO: 7 or SEQ ID NO: 8. According to some embodiments, at least one conserved protein of a Coronaviridae virus is a N protein, or fragment thereof, comprising an amino acid sequence at least 98% identical to SEQ ID NO: 7 or SEQ ID NO: 8. According to some embodiments, at least one conserved protein of a Coronaviridae virus is a N protein, or fragment thereof, comprising an amino acid sequence at least 99% identical to SEQ ID NO: 7 or SEQ ID NO: 8. According to some embodiments, at least one conserved protein of a Coronaviridae virus is a N protein, that consists of SEQ ID NO: 7 or SEQ ID NO: 8.

As discussed herein, according to some embodiments, the conserved proteins of the present disclosure are represented as conserved sequences comprising pathogen antigen sequences, which are selected by achieving the highest degree of protection that avoids the concomitant administration of neutralizing antibody epitopes. Furthermore, such sequences must be able to withstand the high mutation rates and new genetic variants that are resistant to immune responses generated against earlier pathogen subtypes and subvert the immune response generated by altered peptide-ligand phenomena as described herein. Therefore, according to some embodiments, the conserved protein sequences utilized herein will be generated to overcome pathogen genomic variation. Moreover, according to some embodiments, the conserved protein sequences utilized herein will also be generated to achieve cross-protection against pathogens with multiple strains, variants, groups (clades, serotypes or subtypes), and against related pathogenic species, related pathogenic genera, and/or related pathogenic families.

According to some embodiments, the vaccine of the present disclosure immunizes against a Retroviridae virus. For example, the Retroviridae virus is a Lentivirus, a Retrovirus, a Spumaretrovirinae virus, and the like. For example, the Lentivirus is human immunodeficiency virus 1 and human immunodeficiency virus 2. For example, the Retrovirus is mammalian type B retroviruses, mammalian type C retroviruses, avian type C retroviruses, type D retrovirus group, BLV-HTLV retroviruses, and the like.

According to some embodiments, the immunogen of the present disclosure comprises at least one conserved protein of a Retroviridae virus or an immunogenic fragment thereof. For example, the retrovirus is the human immunodeficiency virus. According to some embodiments, the immunogen of the present disclosure comprises at least one human immunodeficiency virus conserved capsid protein (gag), envelope protein (env), polymerase protein (pol), or protease protein (pro), or an immunogenic fragment thereof. According to some embodiments, the immunogen contains overlapping peptides of at least 30 amino acids in length that together cover the full length sequence of a Retroviridae viral protein.

According to some embodiments, the human immunodeficiency virus conserved capsid protein (gag) comprises SEQ ID NO: 9, shown below.

MGARASVLSGGKLDKWEKIRLRPGGKKTYQLKHIVWASRELERFAVNPG
LLETGGGCKQILVQLQPSLQTGSEELKSLYNAVATLYCVHQGIEVRDTK
EALDKIEEEQNKSKKKAQQAAADTGNSSQVSQNYPIVQNLQGQMVHQAI
SPRTLNAWVKVIEEKAFSPEVIPMFSALSEGATPQDLNTMLNTVGGHQA
AMQMLKETINEEAAEWDRLHPAHAGPNAPGQMREPRGSDIAGTTSTLQE
QIGWMTSNPPVPVGEIYKRWIILGLNKIVRMYSPVSILDIRQGPKEPER
DYVDREYKTLRAEQASQDVKNWMTETLLVQNANPDCKTILKALGPAATL
EEMMTACQGVGGPSHKARILAEAMSQVTSPANIMMQRGNFRNQRKTIKC
FNCGKEGHLARHCRAPRKKGCWKCGREGHQMKDCTERQANFLGKIWPSH
KGRPGNFLQSRPEPTAPPEESERFGEETTTPPQKQEPLPSQKQETIDKD
LYPLASLKSLFGNDPSLQ

According to some embodiments, at least one conserved protein of a Retroviridae virus is a human immunodeficiency virus conserved capsid protein (gag), or fragment thereof, comprising an amino acid sequence at least 85% identical to SEQ ID NO: 9. According to some embodiments, at least one conserved protein of a Retroviridae virus is a human immunodeficiency virus conserved capsid protein (gag), or fragment thereof, comprising an amino acid sequence at least 90% identical to SEQ ID NO: 9. According to some embodiments, at least one conserved protein of a Retroviridae virus is a human immunodeficiency virus conserved capsid protein (gag), or fragment thereof, comprising an amino acid sequence at least 95% identical to SEQ ID NO: 9. According to some embodiments, at least one conserved protein of a Retroviridae virus is a human immunodeficiency virus conserved capsid protein (gag), or fragment thereof, comprising an amino acid sequence at least 98% identical to SEQ ID NO: 9. According to some embodiments, at least one conserved protein of a Retroviridae virus is a human immunodeficiency virus conserved capsid protein (gag), or fragment thereof, comprising an amino acid sequence at least 99% identical to SEQ ID NO: 9. According to some embodiments, at least one conserved protein of a Retroviridae virus is a human immunodeficiency virus conserved capsid protein (gag), that consists of SEQ ID NO: 9.

According to some embodiments, the human immunodeficiency virus conserved envelope protein (env) comprises SEQ ID NO: 10, shown below.

MRVKGIRKNYQHWWRWGIMLLGMWMICSAAEKLWVTVYYGVPVWKEATT
TLFCASDAKAYDTEVHNVWATHACVPIDPNPQEVFLENVTENFNMWKNN
MVEQMHEDIISLWDQSLKPCVKLTPLCVTLKCTDLNDTNTNNSSTSENN
TNPTISGGEGMGEGEMKNCSFNVTTNIRDKVQKEYALFYKLDIIPIDNT
SYALRHCNTSVITQACPKVSEEPIPIHYCAPAGFAILQCNDKKFNGTGP
CSNVSTVQCTHGIRPVVSTQLLLNGSLAEEEIVLRSENFTNNAKTIIVQ
LNASVEINCTRLNNNTRKSIRIGPGSTFYATGAIIGDIRQAHCNISREK
WNDTLKQLVIKLGEQFGNSNIIVFKQSSGGDPEIVMHSFICGGEFFYCN
TTQLSNSTWQRSDGTWNRTGGLNETKENITLPCRIKQIINRWQEVGKAM
YAPPISGLIRCSSNITGLLLTRDGGNENNGTNGTETFRPEGGNMKDNWR
SKLYKYKVVRIEPLGIAPTRARRRVVQREKRAVTFGALELGFLGAAGST
MGCTSMTLTVQARLLLSGIVQQQNNLLRAIEAQQHLLQLTVWGIKQLQA
RVLAVERYLRDQQLLGIWGCSGKLICTTNVPWNTSWSNKSENEIWDNMT
WMEWDREINNYTNLIYDLLEKSQNQQEKNEQELLELDKWASL

According to some embodiments, at least one conserved protein of a Retroviridae virus is a human immunodeficiency virus conserved envelope protein (env), or fragment thereof, comprising an amino acid sequence at least 85% identical to SEQ ID NO: 10. According to some embodiments, at least one conserved protein of a Retroviridae virus is a human immunodeficiency virus conserved envelope protein (env), or fragment thereof, comprising an amino acid sequence at least 90% identical to SEQ ID NO: 10. According to some embodiments, at least one conserved protein of a Retroviridae virus is a human immunodeficiency virus conserved envelope protein (env), or fragment thereof, comprising an amino acid sequence at least 95% identical to SEQ ID NO: 10. According to some embodiments, at least one conserved protein of a Retroviridae virus is a human immunodeficiency virus conserved envelope protein (env), or fragment thereof, comprising an amino acid sequence at least 98% identical to SEQ ID NO: 10. According to some embodiments, at least one conserved protein of a Retroviridae virus is a human immunodeficiency virus conserved envelope protein (env), or fragment thereof, comprising an amino acid sequence at least 99% identical to SEQ ID NO: 10. According to some embodiments, at least one conserved protein of a Retroviridae virus is a human immunodeficiency virus conserved envelope protein (env), that consists of SEQ ID NO: 10.

According to some embodiments, the human immunodeficiency virus conserved polymerase protein (pol) comprises SEQ ID NO: 11, shown below.

FFRENLALLQGEARELSSEQARANSPTCRELQVRGRDSSPLLEAGAEGK
GAISLSFPQITLWQRPLVTVRIGEQLIEALLDTGADDTVLEDINLPGKW
KPKMIGGIGGFIKVRQYDQIHIEICGKKAIGTVLVGPTPVNIIGRNMLT
QIGCTLNLPISPIETVPVKLKPGMDGPRVKQWPLTEEKIKALTEICTEM
EKEGKISKIGPENPYNTPIFAIKKKDSTKWRKLVDFRELNKRTQDFWEV
QLGIPHPAGLKKKKSVTILDVGDAYFSVPLDKEFRKYTAFTIPSINNET
PGTRYQYNVLPQGWKGSPAIFQSSMTRILEPFRKQNPEIVIYQYMDDLY
VGSDLEIGQHRVKIEELREHLLKWGFTTPDKKHQKEPPFLWMGYELHPD
KWTVQPIELPDKESWTVNDIQKLVGKLNWASQIYPGIKVRQLCKLLRGA
RALTDIVPLTAEAELELAENREILKEPVHGVYYDPSKDLIAEIQKQGHG
QWTYQIYQEPHKNLKTGKYAKIKSAHTNDVKQLTEAVQKIAMESIVVWG
KTPKERLPIQKETWETWWTEYWQATWIPDWEEVNTPPLVKLWYQLETEP
IVGAETFYVDGAANRETKKGKAGYVTDRGRQRVITLSETTNQKTELQAI
NLALQDSGSEVNIVTDSQYALGIIQAQPDKSESELVNQIIEQLIKKEKV
YLSWVPAHKGIGGNEQVDKLVSSGIRKVLELDGIDKAQEEHEKYHNNWR
AMANDFNLPPIVAKEIVASCDKCQLKGEAMHGQVDCSPGIWQIDCTHLE
GKIIIVAVHVASGYIEAEVIPAETGQETAYFILKLAGRWPVKVIHTDNG
SNFTSAAVKAACWWANVTQEFGIPYNPQSQGVVESMNKELKKIIGQVRD
QAEHLKTAVQMAVEIHNFKRKGGIGGYSAGERIIDIIASDIQTKELQKQ
IIKIQNFRVYYRDSRDPIWKGPAKLLWKGEGAVVIQDNSEIKVVPRRKA
KIIRDYGKQMAGDDCVAGRQDED

According to some embodiments, at least one conserved protein of a Retroviridae virus is a human immunodeficiency virus conserved polymerase protein (pol), or fragment thereof, comprising an amino acid sequence at least 85% identical to SEQ ID NO: 11. According to some embodiments, at least one conserved protein of a Retroviridae virus is a human immunodeficiency virus conserved polymerase protein (pol), or fragment thereof, comprising an amino acid sequence at least 90% identical to SEQ ID NO: 11. According to some embodiments, at least one conserved protein of a Retroviridae virus is a human immunodeficiency virus conserved polymerase protein (pol), or fragment thereof, comprising an amino acid sequence at least 95% identical to SEQ ID NO: 11. According to some embodiments, at least one conserved protein of a Retroviridae virus is a human immunodeficiency virus conserved polymerase protein (pol), or fragment thereof, comprising an amino acid sequence at least 98% identical to SEQ ID NO: 11. According to some embodiments, at least one conserved protein of a Retroviridae virus is a human immunodeficiency virus conserved polymerase protein (pol), or fragment thereof, comprising an amino acid sequence at least 99% identical to SEQ ID NO: 11. According to some embodiments, at least one conserved protein of a Retroviridae virus is a human immunodeficiency virus conserved polymerase protein (pol), that consists of SEQ ID NO: 11.

According to some embodiments, the human immunodeficiency virus conserved protease protein (pro) comprises SEQ ID NO: 12, shown below.

PQITLWQRPLVTIKVEGQVKEALLDTGADDTVLEDMNLPGRWKPKMIGG
IGGFIKVRQYDQITLEICGKKAIGTVLVGPTPVNIIGRNMLTQLGCTLN
F

According to some embodiments, at least one conserved protein of a Retroviridae virus is a human immunodeficiency virus conserved protease protein (pro), or fragment thereof, comprising an amino acid sequence at least 85% identical to SEQ ID NO: 12. According to some embodiments, at least one conserved protein of a Retroviridae virus is a human immunodeficiency virus conserved protease protein (pro), or fragment thereof, comprising an amino acid sequence at least 90% identical to SEQ ID NO: 12. According to some embodiments, at least one conserved protein of a Retroviridae virus is a human immunodeficiency virus conserved protease protein (pro), or fragment thereof, comprising an amino acid sequence at least 95% identical to SEQ ID NO: 12. According to some embodiments, at least one conserved protein of a Retroviridae virus is a human immunodeficiency virus conserved protease protein (pro), or fragment thereof, comprising an amino acid sequence at least 98% identical to SEQ ID NO: 12. According to some embodiments, at least one conserved protein of a Retroviridae virus is a human immunodeficiency virus conserved protease protein (pro), or fragment thereof, comprising an amino acid sequence at least 99% identical to SEQ ID NO: 12. According to some embodiments, at least one conserved protein of a Retroviridae virus is a human immunodeficiency virus conserved protease protein (pro), that consists of SEQ ID NO: 12.

The HIV sequence database (www.hiv.lanl.gov/content/sequence/NEWALIGN/align.html) provides consensus sequences for the M group subtypes A (including A, A1, and A2), B, C, D, F (including F1 and F2), and G; the circulating recombinant forms CRF01 and CRF02; and group O. We also provide a Consensus M-group, which is a consensus of consensus sequences for subtypes A, B, C, D, F, G, H.

TABLE 1B

| | Reference Genome RefSeq No. | Example Conserved Proteins RefSeq |
|---|---|---|
| Pathogen | (Variant(s) Accession No(s).) | No(s). (location) |
| human | NC_001802.1 | Env: NC_001802.1 (5771 . . . 8341); |
| immunodeficiency | (MN124512.1, MN187303.1, | Gag: NC_001802.1 (336 . . . 1838); |
| virus 1 (HIV-1) | MN187302.1, MN18730.1, | Gag-Pol: NC_001802.1 (336 . . . 4642); |
| | MN187300.1, MN202472.1, | Nef: NC_001802.1 (8343 . . . 8963); |
| | MN202471.1, MK984160.1, | Asp: NC_001802.1 (6919 . . . 7488); |
| | MK984159.1, MN090935.1, . . . ) | Vpu: NC_001802.1 (5608 . . . 5856); |
| | | Rev: NC_001802.1 (5516 . . . 8199); |
| | | Tat: NC_001802.1 (5377 . . . 7970); |
| | | Vpr: NC_001802.1 (5105 . . . 5396); |
| | | Vif: NC_001802.1 (4587 . . . 5165) |

According to some embodiments, the immunogen comprises one or more epitopes. According to some embodiments, the immunogen comprises a string of multiple epitopes that are linked together without intervening sequences so that unnecessary nucleic acid and/or amino acid material is avoided. According to some embodiments, the immunogen is a CD8+ T-cell epitope. According to some embodiments, in addition to a CD8+ T-cell epitope, it may be preferable to include one or more epitopes recognized by T helper cells, to augment the immune response generated by the one or more epitopes. Particularly suitable T helper cell epitopes are ones which are active in individuals of different HLA types, for example T helper epitopes from tetanus (against which most individuals will already be primed). According to some embodiments, one or more B cell epitopes are included for stimulating B cell responses and antibody production.

Viral immunogens can be physically derived from a SARS virus or an HIV virus, or produced recombinantly or synthetically.

According to some embodiments, the viral immunogens consensus sequences as utilized in the invention of the present disclosure are generated through multiple sequence alignments of pathogen conserved proteins derived from different strains, variants, groups, clades, serotypes, subtypes, species, genera, and/or families.

### T Cell Epitope Prediction

T cells recognize an epitope comprised of a self portion (Class I MHC) and a foreign portion (a short peptide). It is known that the foreign peptide part of T cell epitopes consists of peptides of between 7 and 14 residues in length, inclusive, e.g., between 7 and 13 residues in length, inclusive, between 7 and 12 residues in length, inclusive, between 7 and 11 residues in length, inclusive, between 7 and 10 residues in length, inclusive, between 7 and 9 residues in length, inclusive; between 8 and 14 residues in length, inclusive, between 8 and 13 residues in length, inclusive, between 8 and 12 residues in length, inclusive, between 8 and 11 residues in length, inclusive, between 9 and 14 residues in length, inclusive, between 9 and 13 residues in length, inclusive, between 9 and 12 residues in length, inclusive, between 9 and 11 residues in length, inclusive; between 10 and 14 residues in length, inclusive, between 10 and 13 residues in length, inclusive; between 10 and 12 residues in length, inclusive; between 11 and 14 residues in length, inclusive, or between 11 and 13 residues in length, inclusive. Briefly, T cell epitopes can be derived from either breaking down a sample of the pathogen to test for T-cell activation against various peptides, or through bioinformatics tools that predict T cell epitopes for pathogens.

### Bioinformatics Tools

As described supra and as known in the art (for example in Khan et al., “A systematic bioinformatics approach for selection of epitope based vaccine targets.” Cell Immunol. (2006) December; 244(2): 141-147; and Soriera-Guerra, et al., (2015) “An overview of bioinformatics tools for epitope prediction: Implications on vaccine development.” Journal of Biomedical Informatics 53: 405-414; the entireties of which are incorporated herein by reference) T-cell immune responses are triggered by the recognition of foreign peptide antigens bound to cell membrane-expressed MHC molecules. Because T-cell recognition is limited to those peptides presented by MHC molecules, prediction of peptides that can bind to MHC molecules is the basis for the anticipation of T-cell epitopes binding to MHC molecules that must fit into a specific chemical and physical environment conditioned by polymorphic residues in the MHC molecule. Consequently, distinct MHC molecules have distinct peptide-binding specificities. In addition, the peptides that bind to the same MHC molecule are related by sequence similarity. Sequence patterns reflecting amino acid preferences in peptide-MHC binders (anchor residues) are routinely used for defining peptide-MHC binding motifs and prediction of peptide-MHC binding. This data is known in the art and has been collected into databases that contain MHC peptide motifs, MHC ligands, T-cell epitopes, as well as, amino acid sequences of MHC molecules.

Databases can be used as prediction tools that predict the outcome of experiments, such as MHC class I or class II binding, and MHC class I processing and immunogenicity. T cell processing predictions combine MHC binding with other parts of the MHC class I cellular pathway, namely proteasomal cleavage and TAP transport, and are generated from independent experimental datasets. There are also predictors trained on eluted MHC ligands that provide an overlay of the signals from MHC binding and MHC processing presentation pathway. The processing prediction tools offer a relatively small but statistically significant increase in accuracy compared to using the MHC binding prediction alone.

Binding prediction methods facilitate the selection of potential epitopes. The methods available in the art were developed using experimental peptide binding data for different MHC alleles to train machine learning algorithms that in turn can be used to predict the binding likelihood or binding affinity for any arbitrary peptide.

The prediction and measure of affinity binding for a given peptide sequence can analyzed through for example, the calculation of a scoring matrix. In general, matrices are constructed using amino acid frequencies at different position of known binders or quantitative MHC-binding data. The former indicates the binding likelihood of a peptide sequence to the MHC molecule, while the latter provides means of quantifying the peptide binding affinity. In a binding affinity scoring matrix, the binding affinity for the sequence is computed based on the amino acid and its position in the binding groove. The values for each residue in the sequence are summed to yield the overall binding for the entire sequence. A position-specific scoring matrix is derived by varying the values of the matrix until the sums for known, measured peptides approximate the measured affinities. Consensus scores are obtained by summing, multiplying or averaging the matrix coefficients and compared against a predetermined threshold. Thresholds are predetermined and calibrated into the prediction tools to enable machine learning.

### MHC Class I Binding Prediction Generation

Not every possible antigenic peptide will be processed and presented on the cell surface by major histocompatibility complex (MHC) molecules and not all MHC-presented peptides are immunogenic T-cell epitopes. Immunogenicity is dependent on several factors, including protein expression, antigen processing and transport, peptide—MHC binding affinity and stability of the resulting complex, peptide competition for MHC binding, as well as the T-cell receptor (TCR) repertoire. Several methods have been developed to identify and to predict T-cell epitopes from a given source protein. Based on in vitro assays, such as competitive binding assays and ELISpot assays, MHC-binding affinity and immunogenicity of peptides can be experimentally assessed. However, given the large variety of antigens and MHC allotypes, in vitro testing of all possible candidates is not feasible. For this reason, various in silico algorithms have been developed to predict the peptides resulting from the different steps involved in epitope presentation. The prediction of the binding likelihood of a given peptide to a specific MHC molecule is based on identified binding preferences and anchor motifs of MHC molecules.

Peptide binding datasets can be searched with either single or multiple different prediction algorithms, including a number of publicly available prediction websites, such as SYFPEITHI (found at http://syfpeithi.de/BMI-AInfos.html) and BIMAS (found at http://www-bimas.cit.nih.gov/molbio/hla_bind/), where the correlation between the measured binding affinity score and each algorithm's predicted score (heuristic score for SYFPEITHI or a half-life of binding score for BIMAS) is computed. The binding affinity score is the affinity between an MHC and isolated peptides, usually expressed as IC50 concentration with low IC50 value implying a high affinity binder. Generally, the half maximal inhibitory concentration (IC50) is a measure of the effectiveness of a substance in inhibiting a specific biological or biochemical function and is a measure of potency, (i.e., amount required to produce an inhibitory effect of given intensity, compared to a reference standard).

Mass spectrometry (MS) is used to analyze the repertoire of peptides presented on the cell surface by detecting ligands eluted from MHC complexes. In contrast to MHC-binding prediction, MS identification of MHC ligands naturally includes the potentially selective steps of antigen processing and transport.

### Binding Affinity Threshold Calibration

The threshold will allow for the computation of the number of true negatives, true positives, false negatives, and false positives. By systematically varying the predicted score threshold from low to high, the rate of true positives and false positives will be calculated as a function of the threshold to derive a ROC (receiver operating characteristic) curve. The area under this ROC curve is the AUC value. The AUC value is independent of the predicted scale because it compares the rank of the matrices and it is independent of the composition of the dataset, such as having different proportions of binders and non-binders. The AUC value is essentially capturing the probability that given two peptides, one a binder and the other a non-binder, the predicted score will be higher for the binder compared to the non-binder. An AUC value of 0.5 is equivalent to a random prediction and a value of 1.0 is equivalent to a perfect prediction.

### Epitope Selection

There are three main strategies for selecting potential binders. The first involves selecting all peptides with IC50 value less than 500 nM, a threshold previously associated with immunogenicity. A second strategy is to pick the top 1% of peptides for each allele/length combination. The third strategy is to pick peptides with percentile ranks below 1%.

### B Cell Antigens

According to some embodiments, the immunogen comprises a B cell antigen, wherein the B cell antigen is at least one conserved protein of a Coronaviridae virus or an immunogenic fragment thereof. According to some embodiments, the B cell antigen comprises at least one SARS-CoV conserved spike protein (S), membrane protein (M), envelope protein (E), or nucleocapsid protein (N), or an immunogenic fragment thereof. According to some embodiments, the B cell antigen comprises at least one SARS-CoV-2 conserved spike protein (S), membrane protein (M), envelope protein (E), or nucleocapsid protein (N), or an immunogenic fragment thereof. According to some embodiments, the B cell antigen comprises SEQ ID NO: 1 or SEQ ID NO: 2. According to some embodiments, the B cell antigen comprises SEQ ID NO: 3 or SEQ ID NO: 4. According to some embodiments, the B cell antigen comprises SEQ ID NO: 5 or SEQ ID NO: 6. According to some embodiments, the B cell antigen comprises SEQ ID NO: 7 or SEQ ID NO: 8. According to some embodiments, the B cell antigen consists of SEQ ID NO: 1 or SEQ ID NO: 2. According to some embodiments, the B cell antigen consists of SEQ ID NO: 3 or SEQ ID NO: 4. According to some embodiments, the B cell antigen consists of SEQ ID NO: 5 or SEQ ID NO: 6. According to some embodiments, the B cell antigen consists of SEQ ID NO: 7 or SEQ ID NO: 8. According to some embodiments, the B cell antigen contains overlapping peptides of at least 30 amino acids in length that together cover the full length sequence of a Coronavirus viral protein.

According to some embodiments, the immunogen comprises a B cell antigen, wherein the B cell antigen is at least one conserved protein of a Retroviridae virus or an immunogenic fragment thereof. According to some embodiments, the B cell antigen comprises at least one at least one human immunodeficiency virus conserved capsid protein (gag), envelope protein (env), polymerase protein (pol), or protease protein (pro), or an immunogenic fragment thereof. According to some embodiments, the B cell antigen comprises SEQ ID NO: 9. According to some embodiments, the B cell antigen comprises SEQ ID NO: 10. According to some embodiments, the B cell antigen comprises SEQ ID NO: 11. According to some embodiments, the B cell antigen comprises SEQ ID NO: 12. According to some embodiments, the B cell antigen consists of SEQ ID NO: 9. According to some embodiments, the B cell antigen consists of SEQ ID NO: 10. According to some embodiments, the B cell antigen consists of SEQ ID NO: 11. According to some embodiments, the B cell antigen consists of SEQ ID NO: 12. According to some embodiments, the B cell antigen contains overlapping peptides of at least 30 amino acids in length that together cover the full length sequence of a Retroviridae viral protein.

According to some embodiments, the B cell antigen is given as part of a prime-boost regimen.

According to some embodiments, various assays can be utilized to determine whether an immune response is stimulated in a B cell or group of B cells. In some embodiments, stimulation of the immune response in B cells includes antibody titer, antibody affinity, antibody performance in neutralization assays, class switch recombination, affinity maturation of antigen-specific antibodies, and memory B cell development.

According to some embodiments, antibody binding assays such as enzyme-linked immunosorbent assay (ELISA) and surface plasmon resonance (SPR)—based real-time kinetics assays, which are described in the art and incorporated by reference in their entireties herein (S. Khurana et al., NPJ Vaccines 3, 40 (2018); S. Khurana et al., Sci. Transl. Med. 3, 85ra48 (2011); J. L. Halliley et al., J Infect Dis 212, 1270-1278 (2015) may be used. According to further embodiments, an in vitro SARS-CoV-2 pseudovirion or wild-type virus neutralization assay as described by Ravichandran et al., (Science Translational Medicine 1 Jul. 2020: Vol. 12, Issue 550; incorporated by reference in its entirety herein) can be used to measure the quality and function of the antibodies elicited by the different SARS-CoV-2 antigens.

### Vaccines

According to some aspects, the disclosure provides vaccines generated by providing proteins and genetic constructs that encode proteins with conserved, internal CD8+ T cell epitopes that make them particularly effective as immunogens against which immune responses against a broad range of viruses can be induced. According to some embodiments, the vaccines can further comprise B-cell antigens as described herein. According to some embodiments, the vaccines can be provided to induce a therapeutic or prophylactic immune response. According to some embodiments, the vaccines stimulate humoral and cellular immune responses at systemic levels. According to some embodiments, the vaccines stimulate humoral and cellular immune responses at mucosal levels. According to some embodiments, the vaccines stimulate humoral and cellular immune responses at systemic levels and mucosal levels.

According to some embodiments, the means to deliver the immunogen is a naked nucleic acid (e.g., DNA or RNA), a DNA vaccine, a recombinant vaccine, a protein subunit vaccine, a composition comprising the immunogen, an attenuated vaccine or a killed vaccine. According to some embodiments, the vaccine comprises a combination selected from one or more DNA vaccines, one or more recombinant vaccines, one or more protein subunit vaccines, one or more compositions comprising the immunogen, one or more attenuated vaccines and one or more killed vaccines.

According to some embodiments, a vaccine according to the disclosure can be delivered to an individual to modulate the activity of the individual's immune system and thereby enhance the immune response against a viral, bacterial, fungal, or protozoan infection. According to some embodiments, when a nucleic acid molecule that encodes the protein is taken up by cells of the individual the nucleotide sequence is expressed in the cells and the protein are thereby delivered to the individual's cells.

According to some embodiments, the recombinant vaccine is constructed by using a plurality of different vaccine vectors with the above-mentioned immunogens, and each immunization is sequentially vaccinated with different recombinant vector vaccines. Each recombinant vaccine is inoculated at least once, and the vaccination program includes at least one respiratory immunization and one systemic immunization. The combination of the recombinant vector vaccine and the inoculation method may achieve high levels of T cell immune responses in the respiratory and systemic systems, thereby enabling vaccine recipients to obtain immunity against different subtypes of virus.

According to some embodiments, compositions and methods are provided which prophylactically and/or therapeutically immunize an individual against a virus from the Coronaviridae virus family.

According to some embodiments, compositions and methods are provided which prophylactically and/or therapeutically immunize an individual against a virus from the Retroviridae virus family.

### Viral Vector Vaccines

According to some embodiments, attenuated, replication-deficient viruses (such as vaccinia viruses) are used to deliver an exogenous viral immunogen as described herein, in an amount effective to elicit or stimulate a cell mediated immune response. Any one or more immunogens of interest, as described herein, can be inserted into a viral vector as described herein.

### Poxvirus Vectors

Because poxviruses have a large genome, they can readily be used to deliver a wide range of genetic material including multiple genes (i.e., act as a multivalent vector). The sizes of the poxvirus genome range from about 130 kbp to about 300 kbp, inclusive, with up to 300 genes, depending on the strain of the virus. Therefore, it is possible to insert large fragments of foreign DNA into these viruses and yet maintain stability of the viral genome.

Poxviruses are useful vectors for a range of uses, for example, for vaccines to generate immune responses, for the development of new vaccines, for delivery of desired proteins and for gene therapy. The advantages of poxvirus vectors include: (i) ease of generation and production, (ii) the large size of the genome permitting insertion of multiple genes (i.e., as a multivalent vector), (iii) efficient delivery of genes to multiple cell types, including antigen-presenting cells, (iv) high levels of protein expression, (v) optimal presentation of antigens to the immune system, (vi) the ability to elicit cell-mediated immune responses as well as antibody responses, (vii) the ability to use combinations of poxviruses from different genera, as they are not immunologically cross-reactive and (viii) the long-term experience gained with using this vector in humans as a smallpox vaccine.

Poxviruses are well known cytoplasmic viruses. The genetic material expressed by such viral vectors typically remains in the cytoplasm and does not have the potential for inadvertent integration of the genetic material into host cell genes, unless specific steps are taken. As a result of the non-integrative cytoplasmic nature of the poxvirus, the poxvirus vector system will not result in having long term persistence in other cells. Thus, the vector and the transformed cells will not adversely affect cells in the host animal at locations distant from the target cell. Further, the large genome of poxviruses enables large genes to be inserted into pox-based vectors.

Poxviruses can be genetically engineered to contain and express heterologous DNA with or without impairing the ability of the virus to replicate. According to some embodiments, the DNA encodes one or more immunogens determined using the methods described herein.

According to some embodiments, such heterologous DNA can encode a wide range of proteins, such as antigens that induce protection against one or more infectious agents, immune modulating proteins such as co-stimulatory molecules, or enzymatic proteins.

According to some embodiments, vaccine compositions described herein are based on poxvirus vectors.

A number of poxviruses have been developed as live viral vectors for the expression of heterologous proteins, e.g. attenuated vaccinia virus strains Modified Vaccinia Ankara (MVA) and Wyeth (Cepko et al., (1984) Cell 37:1053 1062; Morin et al. (1987) Proc. Natl. Acad. Sci. USA 84:4626 4630; Lowe et al., (1987) Proc. Natl. Acad. Sci. USA, 84:3896 3900; Panicali & Paoletti, (1982) Proc. Natl. Acad. Sci. USA, 79:4927 4931; Mackett et al., (1982) Proc. Natl. Acad. Sci. USA, 79:7415 7419). Other attenuated vaccinia virus strains include WR strain, NYCBH strain, ACAM2000, Lister strain, LC16 m8, Elstree-BNm, Copenhagen strain, and Tiantan strain.

Vaccinia virus is the prototype of the genus Orthopoxvirus. It is a double-stranded DNA (deoxyribonucleic acid) virus that has a broad host range under experimental conditions (Fenner et al. 1989 Orthopoxviruses. San Diego, Calif.: Academic Press, Inc.; Damaso et al., (2000) Virology 277:439-49). Modified vaccinia virus Ankara (MVA) or derivatives thereof have been generated by long-term serial passage of the parental chorioallantois vaccinia virus Ankara (CVA) strain of vaccinia virus on chicken embryo fibroblasts (for review see Mayr, A., et al., (1975) Infection, 3:6-14. The MVA virus itself may be obtained from a number of public repository sources. For example, MVA was deposited in compliance with the requirements of the Budapest Treaty at CNCM (Institut Pasteur, Collection Nationale de Cultures Microorganisms, 25, rue du Docteur Roux, 75724 Paris Cedex 15) on Dec. 15, 1987 under Depositary No. 1-721 (U.S. Pat. No. 5,185,146); MVA virus was deposited in compliance with the Budapest Treaty at the European Collection of Cell Cultures (ECACC) (CAMR, Porton Down, Salisbury, SP4 OJG, UK) on Jan. 27, 1994, under Depository No. V94012707) (U.S. Pat. No. 6,440,422 and United States patent publication number 2003/0013190). Also, United States patent publication number 2003/0013190 further discloses particular MVA strains deposited at the ECACC under Depository No. 99101431, and ECACC provisional accession number 01021411. Commercially available vectors include THERION-MVA, THERION PRIFREE vectors and THERION M-SERIES vectors (Therion Biologics Corporation, MA).

MVA was generated by 516 serial passages on chicken embryo fibroblasts of the Ankara strain of vaccinia virus (CVA) (Mayr, A., et al. [1975] Infection 3, 6-14). As a consequence of these long-term passages, about 31 kilobases of the genomic sequence were deleted from the virus (deletion I, II, III, IV, V, and VI) and, therefore, the resulting MVA virus was described as being highly host cell restricted to avian cells (Meyer, H. et al., [1991] J. Gen. Virol. 72, 1031-1038). It was shown in a variety of animal models that the resulting MVA was significantly avirulent (Mayr, A. & Danner, K. [1978] Dev. Biol. Stand. 41: 225-34). Additionally, this MVA strain has been tested in clinical trials as a vaccine to immunize against the human smallpox disease (Mayr et al., [1987] Zbl. Bakt. Hyg. I, Abt. Org. B 167, 375-390, Stickl et al., [1974] Dtsch. med. Wschr. 99, 2386-2392). These studies involved over 120,000 humans, including high-risk patients, and proved that compared to vaccinia based vaccines, MVA had diminished virulence or infectiousness while it induced a good specific immune response. Generally, a virus strain is regarded as attenuated if it has lost its capacity or only has reduced capacity to reproductively replicate in host cells.

Because the genomes of both wild type VV and MVA have been sequenced, it is possible to clone viruses that bear some resemblance to MVA with regard to replication properties, but that are genetically distinct from MVA. These may serve the same purpose, or may be more immunogenic than MVA while being just as safe by virtue of their replication deficiency.

According to some embodiments, the virus has a replication capability 5% or less, or 1% or less compared to wild-type virus. Non-replicating viruses are 100% replication deficient in normal primary human cells.

Viral replication assays are known in the art, and can be performed for vaccinia viruses on e.g. primary keratinocytes, and are described in Liu et al. J. Virol. 2005, 79:12, 7363-70. Viruses which are non-replicating or replication-impaired may have become so naturally (i.e. they may be isolated as such from nature) or artificially e.g. by breeding in vitro or by genetic manipulation, for example deletion of a gene which is critical for replication. There will generally be one or a few cell types in which the viruses can be grown, such as CEF cells for MVA.

According to some embodiments, changes in the virus include, for example, alterations in the gene expression profile of the virus. According to some embodiments, the modified virus may express genes or portions of genes that encode peptides or polypeptides that are foreign to the poxvirus, i.e. they would not be found in a wild-type virus. These foreign, heterologous or exogenous peptides or polypeptides can include sequences that are immunogenic such as, for example, bacterial, viral, fungal, and protozoal antigens, or antigenic sequences derived from viruses other than the viral vector. The genetic material may be inserted at an appropriate site within the virus genome for the recombinant virus to remain viable, i.e. the genetic material may be inserted at a site in the viral DNA (e.g., non-essential site in the viral DNA) to ensure that the recombinant virus retains the ability to infect foreign cells and to express DNA, while maintaining the desired immunogenicity and diminished virulence. For example, as described above, MVA contains 6 natural deletion sites which have been demonstrated to serve as insertion sites. See, for example, U.S. Pat. Nos. 5,185,146, and 6,440,422, incorporated by reference herein. According to some embodiments, genes that code for desired antigens are inserted into the genome of a poxvirus in such a manner as to allow them to be expressed by that virus along with the expression of the normal complement of parent virus proteins.

The modified poxviruses have a low replicative efficiency in the target cell, which prevents sustained replication and infection of other cells. According to some embodiments, the modified poxvirus may also have altered characteristics concerning aspects of the viral life cycle, such as target cell specificity, route of infection, rate of infection, rate of replication, rate of virion assembly and/or rate of viral spreading.

According to some embodiments, the inserted gene(s) encoding immunogens determined using the methods described herein may be operably linked to a promoter to express the inserted gene. Promoters are well known in the art and can readily be selected depending on the host and the cell type one wishes to target. For example in poxviruses, poxviral promoters may be used, such as the vaccinia 7.5K, 40K, fowlpox. In certain embodiments, enhancer elements can also be used in combination to increase the level of expression. In certain embodiments, inducible promoters, which are also well known in the art, may be used. Representative poxvirus promoters include an entomopox promoter, an avipox promoter, or an orthopox promoter such as a vaccinia promoter, e.g., HH, 11K or Pi. For example, the Pi promoter, from the Ava I H region of vaccinia, is described in Wachsman et al., J. of Inf. Dis. 155, 1188-1197 (1987). This promoter is derived from the Ava I H (Xho I G) fragment of the L-variant WR vaccinia strain, in which the promoter directs transcription from right to left. The map location of the promoter is approximately 1.3 Kbp (kilobase pair) from the 5′ end of Ava IH, approximately 12.5 Kbp from the 5′ end of the vaccinia genome, and about 8.5 Kbp 5′ of the Hind III C/N junction. The Hind III H promoter (also “HH” and “H6” herein) sequence is an up-stream of open reading frame H6 by Rosel et al., (1986) J. Virol. 60, 436-449. The 11K promoter is as described by Wittek, (1984) J. Virol. 49, 371-378) and Bertholet, C. et al., (1985) Proc. Natl. Acad. Sci. USA 82, 2096-2100). One can take advantage of whether the promoter is an early or late promoter to time expression of particular genes.

According to some embodiments, vaccine compositions described herein are based on vaccinia virus vectors. According to some embodiments, the vaccinia virus vector is a vaccinia virus Tiantan strain (vaccine virus TianTan strain, VTT). The vaccinia virus Tiantan strain was widely inoculated in China as a smallpox vaccine since it was isolated by Chinese scientists in the 1920s, and eventually destroyed smallpox in China. The number of inoculations of Tiantan strains reached more than one billion, and its safety has been long-term and fully verified in practice. The incidence of side effects of the Tiantan strain vaccine is significantly lower than that of other smallpox vaccine strains used internationally, including the New York strain (NYBPH) used in bioterrorism in the United States in recent years. Tiantan vaccinia vector has a wide host range, high reproductive titer, induced immune response is very long-lasting, and the capacity of inserting foreign genes is extremely large, theoretically up to 25-50 kb. The Tiantan strain carrier has high safety and good immunogenicity, and can induce strong humoral immunity and cellular immunity in vivo, and the duration of the immune reaction is much longer than that of the non-replicating vector.

According to some embodiments, the promoter is modulated by an external factor or cue, allowing control of the level of polypeptide being produced by the vectors by activating that external factor or cue. For example, heat shock proteins are proteins encoded by genes in which the promoter is regulated by temperature. The promoter of the gene which encodes the metal-containing protein metallothionine is responsive to Cd+ ions. Incorporation of this promoter or another promoter influenced by external cues also makes it possible to regulate the production of the polypeptides comprising antigen.

According to some embodiments, the nucleic acid encoding at least one gene of interest encoding, e.g. an immunogen as described herein, is operably linked to an “inducible” promoter. Inducible systems allow careful regulation of gene expression. See, Miller and Whelan, Human Gene Therapy, 8:803-815 (1997). The phrase “inducible promoter” or “inducible system” as used herein includes systems wherein promoter activity can be regulated using an externally delivered agent. Such systems include, for example, systems using the lac repressor from E. coli as a transcription modulator to regulate transcription from lac operator-bearing mammalian cell promoters (Brown et al. Cell, 49:603-612, 1987); systems using the tetracycline repressor (tetR)(Gossen and Bujard, 1992 Proc. Natl. Acad. Sci. USA 89: 5547-5551; Yao et al., 1998 Human Gene Ther. 9:1939-1950; Shokelt et al., 1995 Proc. Natl. Acad. Sci. USA 92.6522-6526). Other such systems include FK506 dimer, VP16 or p65 using castradiol, RU486/mifepristone, diphenol muristerone or rapamycin. Another example is an ecdysone inducible system (see, e.g. Karns et al, 2001 MBC Biotechnology 1:11). Inducible systems are available, e.g., from Invitrogen, Clontech, and Ariad. Systems using a repressor with the operon are preferred. These promoters may be adapted by substituting portions of pox promoters for the mammalian promoter.

According to some embodiments, a “transcriptional regulatory element” or “TRE” is introduced for regulation of the gene of interest. A TRE is a polynucleotide sequence, preferably a DNA sequence, that regulates transcription of an operably-linked polynucleotide sequence by an RNA polymerase to form RNA. A TRE increases transcription of an operably linked polynucleotide sequence in a host cell that allows the TRE to function. The TRE comprises an enhancer element and/or viral promoter element, which may or may not be derived from the same gene. The promoter and enhancer components of a TRE may be in any orientation and/or distance from the coding sequence of interest, and comprise multimers of the foregoing, as long as the desired, transcriptional activity is obtained.

According to some embodiments, an “enhancer” for regulation of the gene of interest is provided. An enhancer is a polynucleotide sequence derived from a gene which increases transcription of a gene which is operably-linked to a promoter to an extent which is greater than the transcription activation effected by the promoter itself when operably-linked to the gene, i.e. it increases transcription from the promoter.

The activity of a regulatory element such as a TRE or an enhancer generally depends upon the presence of transcriptional regulatory factors and/or the absence of transcriptional regulatory inhibitors. Transcriptional activation can be measured in a number of ways known in the art, but is generally measured by detection and/or quantification of mRNA or the protein product of the coding sequence under control of (i.e., operatively linked to) the regulatory element. The regulatory element can be of varying lengths, and of varying sequence composition. By transcriptional activation, it is intended that transcription will be increased above basal levels in the target cell by at least about 2-fold, preferably at least about 5-fold, preferably at least about 10-fold, more preferably at least about 20-fold. More preferably at least about 50-fold, more preferably at least about 100-fold, even more preferably at least about 200-fold, even more preferably at least about 400- to about 500-fold, even more preferably, at least about 1000-fold. Basal levels are generally the level of activity, if any, in a non-target cell, or the level of activity (if any) of a reporter construct lacking the TRE or enhancer of interest as tested in a target cell type.

Certain point mutations within sequences of TREs decrease transcription factor binding and gene activation. One of skill in the art would recognize that some alterations of bases in and around known the transcription factor binding sites are more likely to negatively affect gene activation and cell-specificity, while alterations in bases which are not involved in transcription factor binding are not as likely to have such effects. Certain mutations also increase TRE activity. Testing of the effects of altering bases may be performed in vitro or in vivo by any method known in the art, such as mobility shift assays, or transfecting vectors containing these alterations in TRE functional and TRE non-functional cells. Additionally, one of skill in the art would recognize that point mutations and deletions can be made to a TRE sequence without altering the ability of the sequence to regulate transcription.

### Adenoviral Vectors

Adeno-associated virus (AAV) is a replication-deficient parvovirus, the single-stranded DNA genome of which is about 4.7 kb in length including 145 nucleotide inverted terminal repeats (ITRs). The nucleotide sequence of the AAV serotype 2 (AAV2) genome is presented in Srivastava et al., (1983) J. Virol., 45: 555-564 as corrected by Ruffing et al., (1994) J. Gen. Virol., 75: 3385-3392. Cis-acting sequences directing viral DNA replication (rep), encapsidation/packaging and host cell chromosome integration are contained within the ITRs. Three AAV promoters, p5, p19, and p40 (named for their relative map locations), drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (Rep 78, Rep 68, Rep 52, and Rep 40) from the rep gene. Rep 78 and Rep 68, are respectively expressed from unspliced and spliced transcripts initiating at the p5 promoter, while Rep 52 and Rep 40, are respectively expressed from unspliced and spliced transcripts initiating at the p19 promoter. Rep proteins possess multiple enzymatic properties which are ultimately responsible for replicating the viral genome. Rep 78 and 68 appear to be involved in AAV DNA replication and in regulating AAV promoters, while Rep 52 and 40 appear to be involved in formation of single-stranded AAV DNA. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, (1992) Current Topics in Microbiology and Immunology, 158: 97-129.

When wild type AAV infects a human cell, the viral genome can integrate into chromosome 19 resulting in latent infection of the cell. Production of infectious virus does not occur unless the cell is infected with a helper virus (for example, adenovirus or herpesvirus). In the case of adenovirus, genes E1A, E1B, E2A, E4 and VA provide helper functions. Upon infection with a helper virus, the AAV provirus is rescued and amplified, and both AAV and adenovirus are produced.

AAV possesses unique features that make it attractive for delivering DNA to cells in a clinical application, for example, as a gene therapy vector or an immunization vector. AAV infection of cells in culture is noncytopathic, and natural infection of humans and other animals is silent and asymptomatic. Moreover, AAV infects many mammalian cells allowing the possibility of targeting many different tissues in vivo. The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV-vectors less critical. AAV may even be lyophilized. Finally, AAV-infected cells are not resistant to superinfection.

Production of rAAV requires the AAV rep78/68 and rep52/40 genes and expression of their gene products, a DNA of interest flanked by AAV ITRs, helper functions provided by an AAV helper virus, and a cell line comprising these components that is permissive for AAV replication. Examples of helper virus functions are adenovirus genes E1, E2A, E4 and VA (Carter, Adeno-associated virus helper functions. (1989) In “Handbook of Parvoviruses” Vol I (P. Tjissen, ed.) CRC Press, Boca Raton, pp 255-282). Wild type AAV (wt AAV) has one of the largest burst sizes of any virus following infection of cells with AAV and adenovirus. This may be well in excess of 100,000 particles per cell (Aitken et al., 2001 Hum Gene Therapy, 12:1907-1916), while some rAAV production systems have been reported to achieve 10e3 or 10e4 particles per cell. Rep proteins are absolutely required for both wt AAV and rAAV replication and assembly of intact infectious particles, as summarized in Carter et al., AAV vectors for gene therapy. (2004) In “Gene and Cell Therapy: Therapeutic Mechanisms and Strategies”, Second Edition (Ed. N. Templeton-Smith), pp 53-101, Marcel Dekker, New York). Expression of the rep proteins during the replicative phase of AAV production is both autoregulated and highly coordinated at the transcription level exhibiting both positive and negative regulatory activities. The relative ratio of the rep proteins necessary to achieve rAAV vector production levels equivalent to WT AAV has not been fully understood. See Li et al., (1997) J Virol., 71:5236-5243; Xiao et al., (1998) J Virol, 72:2224-2232; Matushita et al., (1998) Gene Therapy, 5:938-945; and Carter et al., AAV vectors for gene therapy, in “Gene and Cell Therapy: Therapeutic Mechanisms and Strategies”, Second Edition (Ed. N. Templeton-Smith), pp 53-101, Marcel Dekker, New York. Numerous vector production methods have been described which have altered the relative ratios of rep 52/40 and rep 78/68 by decoupling regulation of their respective promoters. See, e.g., Natsoulis, U.S. Pat. No. 5,622,856; Natsoulis et al., U.S. Pat. No. 6,365,403; Allen et al., U.S. Pat. No. 6,541,258; Trempe et al., U.S. Pat. No. 5,837,484; Flotte et al., U.S. Pat. No. 5,658,776; Wilson et al. U.S. Pat. No. 6,475,769; Fan and Dong, (1997) Human Gene Therapy, 8:87-98; and Vincent et al., (1997) J Virol, 71:1897-1905. This decoupling of the large and small rep proteins at the transcriptional level has been achieved by a number of methods including, replacing the native p5, p19, and p40 native AAV promoters either completely or in some combination with heterologous promoters, inducible promoters; or by physical means of either placing the components on separate genetic elements including without limitation separate plasmids; or by utilizing separate genetic constructs for transducing or transfecting the permissive cell line including carrier viruses such as adenovirus or herpes virus; inserting additional spacer elements, or physically rearranging the rep gene or its regulatory sequences within a single genetic construct. These strategies have been employed both for transient production systems where one or more of the components are introduced to the permissive cell line via plasmid transfection; hybrid viral infection such as recombinant adenoviruses, herpes virus, or baculovirus; or in stable cell line approaches utilizing production from transformed cancerous cells permissive for AAV production such as HELA and 293 cells.

According to some embodiments, vaccine compositions described herein are based on adenovirus (Ad) vectors.

According to some embodiments, vaccine compositions described herein are based on Ad serotype 5 (AdHu5). AdHu5-based vectors, have been extensively studied in laboratories and clinical trials over the past decades (Alonso-Padilla et al. 2016 Mol Ther.; 24: 6-16). It has been demonstrated that the AdHu5 vector can elicit potent antigen-specific immune responses in both preclinical and clinical studies (Alonso-Padilla 2016 Mol Ther.; 24: 6-16; Abbink et al. 2007 J Virol.; 81:4654-4663). However, although the AdHu5 vector has been shown to be very efficient, AdHu5 infection is endemic in humans. Thus, adenoviral vectors isolated from different species have been tested. In particular, chimpanzee adenoviruses (AdCs) are attractive for use because they can be cultured in human cell lines such as human embryonic kidney 293 cells (HEK293), and have a low seroprevalence in the human population as they rarely circulate in humans. Moreover, some AdCs can induce T- and B-cell immune responses comparable to those of commonly used human Ad serotypes like AdHu5 (Quinn et al. 2013 J Immunol.; 190:2720-2735; Ledgerwood et al. 2017 N Engl J Med.; 376:928-938).

According to some embodiments, vaccine compositions described herein are based on adenovirus (Ad) vectors that are derived from a chimpanzee. According to some embodiments, the adenoviral vector is based on a chimpanzee adenoviral isolate Y25 (Hillis et al. (1969) J Immunol 103: 1089-109). According to some embodiments, the adenoviral vector is based on AdC68 (also called Sad-V25; Farina S F, et al. (2001) Journal of Virology 75: 11603-11613, incorporated by reference in its entirety herein).

The chimpanzee adenovirus isolate Y25 was first described by Hillis et al. ((1969) J Immunol 103: 1089-1095). The viral genome is represented in GenBank accession no. JN254802, incorporated by reference in its entirety herein. The genome sequence data has confirmed early serological indications that this adenovirus is related to the Human adenovirus E virus, HAdV-4 (Wigand R, et al. (1989) Intervirology 30: 1-9). Simian adenoviruses (SAdV) isolated from great apes are not phylogenetically distinct from human adenoviruses (HAdV), and group together into the same viral species (Human adenovirus B, C and E) (Roy S, et al. (2009) PLoS pathogens 5: e1000503). Although HAdV-4 is the sole representative of Human adenovirus E derived from humans, many of the chimpanzee adenoviruses group phylogenetically within species E, including vaccine vector candidates ChAd63, AdC68 (SAdV-25), AdC7 (SAdV-24) and AdC6 (SAdV-23) (Farina S F, et al. (2001) Journal of virology 75: 11603-11613; Roy S. et al. (2004) Virology 324: 361-372; Roy S., et al. (2004) Human gene therapy 15: 519-530; Reyes-Sandoval et al. (2010) Infection and immunity 78: 145-153; the contents of all the foregoing are incorporated by reference in their entireties herein). Phylogenetic analysis indicates that chimpanzee adenovirus Y25 also groups with Human adenovirus E viruses.

Exemplary sequences from the Y25 genome are set forth below by GenBank Accession Number:

ChAd3 CS479276; ChAd63 CS479277; HAdV-5 AC 000008; SAdV-22 AY530876; SAdV-25 AF394196, HAdV-1 AF534906; HAdV-2 J01917; HAdV-4 AY458656; HAdV-6 FJ349096; HAdV-12 X73487; HAdV-16 AY601636; HAdV-30 DQ149628; HAdV-10 AB369368, AB330091; HAdV-17 HQ910407; HAdV-9 AJ854486; HAdV-37 AB448776; HAdV-8 AB448767; HAdV-7 AB243119, AB243118; HAdV-11 AC 000015; HAdV-21 AY601633; HAdV-34 AY737797; HAdV-35 AC 000019; HAdV-40 NC 001454; HAdV-41 DQ315364; SAdV-21 AC 000010; SAdV-23 AY530877; SAdV-24 AY530878; HAdV-3 DQ086466; HAdV-18 GU191019; HAdV-31 AM749299; HAdV-19 AB448774; SAdV-25.2 FJ025918; SAdV-30 FJ025920; SAdV-26 FJ025923; SAdV-38 FJ025922; SAdV-39 FJ025924; SAdV-36 FJ025917; SAdV-37.1 FJ025921; HAdV-14 AY803294; SAdV-27.1 FJ025909; SAdV-28.1 FJ025914; SAdV-33 FJ025908; SAdV-35.1 FJ025912; SAdV-31.1 FJ025906; SAdV-34 FJ025905; SAdV-40.1 FJ025907; SAdV-3 NC 006144; Y25 JN254802

Very few antibodies exist in the human body against chimpanzee adenovirus type 68, and this adenovirus can infect both dividing cells and non-dividing cells, including lung cells, liver cells, bone cells, cells in blood vessels, muscles, brain and central nervous, etc. Moreover, it has good gene stability and excellent ability to express foreign genes. It can be produced by HEK293 cells and has been widely used in research on vaccines such as AIDS, Ebola, influenza, malaria, and hepatitis C.

Production, purification and quality control procedures for Ad vectors are well established (Tatsis & Ertl, 2004 Mol Ther 10: 616-29). Ad vectors induce innate immune responses ameliorating the need for addition of adjuvants. They also induce very potent B and CD8 T cell responses, which, due to low-level persistence of the vectors, are remarkably sustained (Tatsis et al., 2007, Blood 110: 1916-23). Pre-existing neutralizing antibodies to common human serotypes of Ad viruses such as serotype 5, which impact vaccine efficacy, can readily be avoided by the use of by serotypes from other species such as chimpanzees, which typically neither circulate in humans nor cross-react with human serotypes (Xiang et al., 2006 Emerg Infect Dis 12: 1596-99). In cases where prime-boost regimens are needed to achieve immune responses of sufficient potency, vectors based on distinct Ad serotypes are available (Tatsis & Ertl, 2004 Mol Ther 10: 616-29). Ad viruses and Ad vectors have been used extensively in the clinic where they were well tolerated. They can be applied through a variety of routes including mucosal routes such as the airways (Xiang et al., 2003 J Virol 77: 10780-89) or even orally upon encapsidation as was shown with vaccine to Ad viruses 4 and 7 used by the US military (Lyons et al., 2008 Vaccine 26: 2890-98).

### Herpes Simplex Virus Vectors

According to some embodiments, vaccine compositions described herein are based on herpes simplex virus (HSV) vectors.

HSV can display a broad host cell range, and its cellular receptors, heparan sulfate (HS), herpesvirus entry mediator (HVEM), and nectin-1 and -2, are widely expressed on the cell surface of numerous cell types. Also, it is possible to transduce 70% cells in vitro at a low multiplicity of infection (1.0), with a replication-defective vector. Almost half of the 84 known viral genes are nonessential for growth in tissue culture and may be deleted to create genomic space for exogenous transgenes and to delete functions essential for virulence and toxicity in vivo. [Patel, D H, Misra, A. Chapter 5, “Gene Delivery Using Viral Vectors” in “Challenges in Delivery of Therapeutic Genomics and Proteomics (2011), Elsevier, Inc. pp. 207-70].

For the production of an HSV vector strain, combinations of essential and non-essential genes can be removed from the genome so that the virus is nonpathogenic and minimally cytotoxic. Vector viruses are often produced by the deletion of one or other or both of the two essential immediate early genes ICP4 and ICP27. These require growth on cell lines expressing the deleted genes. Further deletions can be made to reduce cytotoxicity. For the production of viruses which allow gene expression during latency, promoters must be designed which allow gene expression to continue during this time, and this has proved to be a considerable challenge in the field of HSV vector development. However, a number of different promoter systems, each incorporating different elements of the HSV latency associated transcript (LAT) region, do give gene expression during latency to various levels of efficiency. These either use one or other of the LAT promoters (LAP1 or LAP2; Goins et al., 1994. J of Virology. 68(4): 2239-2252) to drive directly gene expression during latency, or DNA fragments derived from the LAT region to confer a long term activity on individual or pairs of promoters. This element, referred to herein as LAT P2 (including LAP2 and other upstream sequences; (nts 118866-112019-GenBank HE1CG)), has been shown subsequently to act not as a true promoter but instead to confer long term activity on heterologous promoters placed near to it, these promoters not being active during latency when used on their own.

According to some embodiments, the herpes simplex viruses of the disclosure may be derived from, for example, HSV1 or HSV2 strains, or derivatives thereof, such as HSV1. Derivatives include inter-type recombinants containing DNA from HSV1 and HSV2 strains. Derivatives for example have at least 70% sequence homology to either the HSV1 or HSV2 genomes, for example at least 80%, for example at least 90 or 95%. Other derivatives which may be used to obtain the viruses of the present disclosure include strains that already have mutations in either ICP4 and/or ICP27, for example strain d120 which has a deletion in ICP4 (DeLuca et al., 1985 J. Virol. 56 (2): 558-70). HSV strains have also been produced with deletions in ICP27, for example Reef Hardy and Sandri-Goldin, 1994 J. Virol. 68(12): 7790-99 and Rice and Knipe, 1990 J. Virol. 64(4): 1704-15 (strain d27-1). Strains with deletions in both ICP4 and ICP27 are described in U.S. Pat. No. 5,658,724, and Samaniego et al. 1995 J. Virol. 69: 5705-15 (strain d92).

### Cytomegalovirus Vectors

According to some embodiments, vaccine compositions described herein are based on cytomegalovirus virus (CMV) vectors.

CMV is a member of the beta subclass of the Herpesvirus family. It is a large (containing a 230 kilobase genome), double stranded DNA virus that establishes life-long latent or persistent infection. In developed countries such as the United States, approximately 70% of the population is infected by CMV. In contrast to gamma herpesviruses such as Epstein-Barr Virus and Kaposi's Sarcoma-associated Herpesvirus, CMV is non-transforming and non-oncogenic.

The ability of live, recombinant CMV to generate immune responses against recombinant antigens has been demonstrated in several reports (Hansen et al, 2009 Nat. Med. 15:293-299; Karrer et al, 2004 J. Virol. 78:2255-2264). Moreover, it has been demonstrated that a recombinant, replication-competent CMV that is engineered to express a self protein will generate long-lasting, CD8+ T cell-based immunity against cells expressing the self protein (Lloyd et al, 2003 Biol. Reprod. 68:2024-2032). Hanson et al. used recombinant rhesus CMV expressing SIV antigens to immunize rhesus macaques against SIV (Hansen et al., 2009 Nat. Med. 15:293-299). The immunization induced large numbers of activated effector memory CD8+ T cells specific for SIV in peripheral tissues, which persisted for the entire multi-year duration of the study. The immunized monkeys were substantially protected from SIV challenge, which was attributed to the presence of activated effector-memory T cells. The study also demonstrated that pre-existing immunity to CMV did not prevent the ability of recombinant CMV to induce a new immune response.

According to some embodiments, vaccine compositions disclosed herein comprise a recombinant, replication-deficient cytomegalovirus comprising a heterologous nucleic acid encoding the immunogens of the present disclosure. According to some embodiments, viral latency is established in the subject, which latency results in the repeatedly stimulated immune response against the antigen. In particular embodiments, the repeatedly stimulated immune response comprises a CD8+ T cell immune response. According to some embodiments, the heterologous immunogen comprises a viral or tumor-derived polypeptide.

According to some embodiments, the recombinant replication-deficient cytomegalovirus comprises an inactivated gB, gD, gH, or gL glycoprotein gene, such as a gL glycoprotein that is inactivated by a knock out mutation. According to some embodiments, the nucleic acid encoding the immunogen is operably linked to a constitutive promoter. According to some embodiments, the nucleic acid encoding the immunogen is operably linked to an inducible promoter. According to some embodiments, the recombinant replication-deficient cytomegalovirus is a murine cytomegalovirus. In other embodiments, the recombinant replication-deficient cytomegalovirus is a human cytomegalovirus, such as an AD 169, Davis, Toledo or Towne strain of CMV.

### Virus-Like Particle (VLP) Vectors

According to some embodiments, the present disclosure provides virus-like particles (VLPs) from the plasma membrane of eukaryotic cells, which VLPs carry on their surfaces immunogenic viral proteins, as described herein. The VLPs, alone or in combination with one or more additional VLPs and/or adjuvants, stimulate an immune response that protects against viral infection.

According to some embodiments, the VLP comprises viral proteins produced from naturally occurring and/or mutated nucleic acid sequences of genes coding for matrix protein M (also known as M1) and, optionally, M2 protein. The matrix protein M is a universal component for the formation of all possible polyvalent sub-viral structure vaccine combinations. The M1 and M2 proteins may be derived from any virus. The M1 and/or M2 proteins may be modified (mutated), for example as disclosed herein or in U.S. Patent Publications 2008/0031895 and 2009/0022762, incorporated by reference in their entireties herein.

VLPs can be prepared using standard recombinant techniques. Polynucleotides encoding the VLP-forming protein(s) are introduced into a host cell and, when the proteins are expressed in the cell, they assemble into VLPs. Polynucleotide sequences coding for molecules (structural and/or antigen polypeptides, including modified antigenic polypeptides) that form and/or are incorporated into the VLPs can be obtained using recombinant methods, such as by screening cDNA and genomic libraries from cells expressing the gene, or by deriving the gene from a vector known to include the same. For example, plasmids which contain sequences that encode naturally occurring or altered cellular products may be obtained from a depository such as the A.T.C.C., or from commercial sources. Plasmids containing the nucleotide sequences of interest can be digested with appropriate restriction enzymes, and DNA fragments containing the nucleotide sequences can be inserted into a gene transfer vector using standard molecular biology techniques.

Alternatively, cDNA sequences may be obtained from cells which express or contain the sequences, using standard techniques, such as phenol extraction and PCR of cDNA or genomic DNA. See, e.g., Sambrook et al., supra, for a description of techniques used to obtain and isolate DNA. Briefly, mRNA from a cell which expresses the gene of interest can be reverse transcribed with reverse transcriptase using oligo-dT or random primers. The single stranded cDNA may then be amplified by PCR (see U.S. Pat. Nos. 4,683,202, 4,683,195 and 4,800,159, see also PCR Technology: Principles and Applications for DNA Amplification, Erlich (ed.), Stockton Press, 1989)) using oligonucleotide primers complementary-to sequences on either side of desired sequences.

The nucleotide sequence of interest can also be produced synthetically, rather than cloned, using a DNA synthesizer (e.g., an Applied Biosystems Model 392 DNA Synthesizer, available from ABI, Foster City, Calif.). The nucleotide sequence can be designed with the appropriate codons for the expression product desired. The complete sequence is assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311.

Any of the proteins used in the VLPs described herein may be hybrid (or chimeric) proteins. It will be apparent that all or parts of the polypeptides may be replaced with sequences from other viruses and/or sequences from other influenza strains. According to some exemplary embodiment, any of the proteins of the VLP may be hybrids in that they include heterologous sequences encoding the transmembrane and/or cytoplasmic tail domains, for example domains from influenza proteins such as HA or NA. See, e.g., U.S. Patent Publication Nos. 2008/0031895 and 2009/0022762.

Any of the sequences described herein may further include additional sequences. For example, to further enhance vaccine potency, hybrid molecules are expressed and incorporated into the sub-viral structure. These hybrid molecules are generated by linking, at the DNA level, the sequences coding for the matrix protein genes with sequences coding for an adjuvant or immuno-regulatory moiety. During sub-viral structure formation, these hybrid proteins are incorporated into or onto the particle depending on whether M1 or optional M2 carries the adjuvant molecule. The incorporation of one or more polypeptide immunomodulatory polypeptides (e.g., adjuvants) into the sequences described herein to form the VLP may enhance potency and therefore reduces the amount of antigen required for stimulating a protective immune response. Alternatively, one or more additional molecules (polypeptide or small molecules) may be included in the VLP-containing compositions after production of the VLP from the sequences described herein.

These sub-viral structures do not contain infectious viral nucleic acids and they are not infectious eliminating the need for chemical inactivation. Absence of chemical treatment preserves native epitopes and protein conformations enhancing the immunogenic characteristics of the vaccine.

The sequences described herein can be operably linked to each other in any combination. For example, one or more sequences may be expressed from the same promoter and/or from different promoters. As described below, sequences may be included on one or more vectors.

### Expression Vectors

Once the constructs comprising the sequences encoding the polypeptide(s) desired to be incorporated into a vector described herein have been synthesized, they can be cloned into any suitable vector or replicon for expression. Numerous cloning vectors are known to those of skill in the art, and one having ordinary skill in the art can readily select appropriate vectors and control elements for any given host cell type in view of the teachings of the present specification and information known in the art about expression. See, generally, Ausubel et al., supra or Sambrook et al, supra.

Non-limiting examples of vectors that can be used to express sequences that assemble into VLPs as described herein include viral-based vectors (e.g., retrovirus, adenovirus, adeno-associated virus, lentivirus), baculovirus vectors (see, Examples), plasmid vectors, non-viral vectors, mammalians vectors, mammalian artificial chromosomes (e.g., liposomes, particulate carriers, etc.) and combinations thereof.

The expression vector(s) typically contain(s) coding sequences and expression control elements which allow expression of the coding regions in a suitable host. The control elements generally include a promoter, translation initiation codon, and translation and transcription termination sequences, and an insertion site for introducing the insert into the vector. Translational control elements have been reviewed by M. Kozak (e.g., Kozak, M., Mamm. Genome 7(8):563-574, 1996; Kozak, M., Biochimie 76(9):815-821, 1994; Kozak, M., J Cell Biol 108(2):229-241, 1989; Kozak, M., and Shatkin, A. J., Methods Enzymol 60:360-375, 1979).

For example, typical promoters for mammalian cell expression include the SV40 early promoter, a CMV promoter such as the CMV immediate early promoter (a CMV promoter can include intron A), RSV, HIV-LTR, the mouse mammary tumor virus LTR promoter (MMLV-LTR), FIV-LTR, the adenovirus major late promoter (Ad MLP), and the herpes simplex virus promoter, among others. Other nonviral promoters, such as a promoter derived from the murine metallothionein gene, will also find use for mammalian expression. Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook, et al., supra, as well as a bovine growth hormone terminator sequence. Introns, containing splice donor and acceptor sites, may also be designed into the constructs as described herein (Chapman et al., Nuc. Acids Res. (1991) 19:3979-3986).

According to some embodiments, the promoter is a CMV promoter. According to some embodiments, the promoter is an inducible promoter.

Enhancer elements may also be used herein to increase expression levels of the mammalian constructs. According to some embodiments, an enhancer sequence is located 5′ of the promoter sequence. According to some embodiments, the enhancer sequence is located 3′ of the promoter sequence. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence (Chapman et al., Nuc. Acids Res. (1991) 19:3979-3986). According to some embodiments, the enhancer is the enhancer region for Serpinl gene as described by Chuah, M., et al. ((2014). Liver-Specific Transcriptional Modules Identified by Genome-Wide In Silico Analysis Enable Efficient Gene Therapy in Mice and Non-Human Primates Molecular Therapy 22(9), 1605-1613, incorporated by reference in its entirety herein). According to some embodiments, the enhancer is the enhancer region for Transthyretin (TTRe) gene (TTRe).

According to some embodiments, one or more vectors may contain one or more sequences encoding proteins to be incorporated into the VLP. For example, a single vector may carry sequences encoding all the proteins found in the VLP. Alternatively, multiple vectors may be used (e.g., multiple constructs, each encoding a single polypeptide-encoding sequence or multiple constructs, each encoding one or more polypeptide-encoding sequences). In embodiments in which a single vector comprises multiple polypeptide-encoding sequences, the sequences may be operably linked to the same or different transcriptional control elements (e.g., promoters) within the same vector. Furthermore, vectors may contain additional gene expression controlling sequences including chromatin opening elements which prevent transgene silencing and confer consistent, stable and high level of gene expression, irrespective of the chromosomal integration site. These are DNA sequence motifs located in proximity of house-keeping genes, which in the vectors create a transcriptionally active open chromatin environment around the integrated transgene, maximizing transcription and protein expression, irrespective of the position of the transgene in the chromosome.

In addition, one or more sequences encoding non-viral immunogens, e.g., non-influenza proteins, may be expressed and incorporated into the VLP, including, but not limited to, sequences comprising and/or encoding immunomodulatory molecules (e.g., adjuvants described below), for example, immunomodulating oligonucleotides (e.g., CpGs), cytokines, detoxified bacterial toxins and the like.

### VLP Production

The sequences and/or vectors described herein are then used to transform an appropriate host cell. The construct(s) encoding the proteins that form the VLPs described herein provide efficient means for the production of Coronavirus or Retroviridae VLPs using a variety of different cell types, including, but not limited to, insect, fungal (yeast) and mammalian cells.

According to some embodiments, the sub-viral structure vaccines are produced in eukaryotic cells following transfection, establishment of continuous cell lines (using standard protocols) and/or infection with DNA constructs that carry the immunogenic genes of interest (e.g., influenza genes) as known to one skilled in the art. The level of expression of the proteins required for sub-viral structure formation is maximized by sequence optimization of the eukaryotic or viral promoters that drive transcription of the selected genes. The sub-viral structure vaccine is released into the culture media, from where it is purified and subsequently formulated as a vaccine. The sub-viral structures are not infectious and therefore inactivation of the VLP is not required as it is for some killed viral vaccines

The ability of the immunogenic polypeptides expressed from sequences as described herein to self-assemble into VLPs with antigenic glycoproteins presented on the surface allows these VLPs to be produced in many host cells by co-introduction of the desired sequences. The sequence(s) (e.g., in one or more expression vectors) may be stably and/or transiently integrated in various combinations into a host cell.

Suitable host cells include, but are not limited to, bacterial, mammalian, baculovirus/insect, yeast, plant and Xenopus cells.

For example, a number of mammalian cell lines are known in the art and include primary cells as well as immortalized cell lines available from the American Type Culture Collection (A.T.C.C.), such as, but not limited to, MDCK, BHK, VERO, MRC-5, WI-38, HT1080, 293, 293T, RD, COS-7, CHO, Jurkat, HUT, SUPT, C8166, MOLT4/clone8, MT-2, MT-4, H9, PM1, CEM, myeloma cells (e.g., SB20 cells) and CEMX174 (such cell lines are available, for example, from the A.T.C.C.).

Similarly, bacterial hosts such as E. coli, Bacillus subtilis, and Streptococcus spp., will find use with the present expression constructs.

Yeast hosts useful in the present disclosure include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Fungal hosts include, for example, Aspergillus.

Insect cells for use with baculovirus expression vectors include, inter alia, Aedes aegypti, Autographa californica, Bombyx mori, Drosophila melanogaster, Spodoptera frugiperda, and Trichoplusia ni. See, Latham & Galarza (2001) J. Virol. 75(13):6154-6165; Galarza et al. (2005) Viral. Immunol. 18(1):244-51; and U.S. Patent Publications 200550186621 and 20060263804.

Cell lines expressing one or more of the sequences described above can readily be generated given the disclosure provided herein by stably integrating one or more expression vector constructs encoding the proteins of the VLP. The promoter regulating expression of the stably integrated influenza sequences (s) may be constitutive or inducible. Thus, a cell line can be generated in which one or more both of the matrix proteins are stably integrated such that, upon introduction of the sequences described herein (e.g., hybrid proteins) into a host cell and expression of the proteins encoded by the polynucleotides, non-replicating viral particles that present antigenic glycoproteins are formed.

The parent cell line from which a VLP-producer cell line is derived can be selected from any cell described above, including for example, mammalian, insect, yeast, bacterial cell lines. In an exemplary embodiment, the cell line is a mammalian cell line (e.g., 293, RD, COS-7, CHO, BHK, MDCK, MDBK, MRC-5, VERO, HT1080, and myeloma cells). Production of coronavirus or Retroviridae VLPs using mammalian cells provides (i) VLP formation; (ii) correct post translation modifications (glycosylation, palmitylation) and budding; (iii) absence of non-mammalian cell contaminants and (iv) ease of purification.

In addition to creating cell lines, immunogen-encoding sequences may also be transiently expressed in host cells. Suitable recombinant expression host cell systems include, but are not limited to, bacterial, mammalian, baculovirus/insect, vaccinia, Semliki Forest virus (SFV), Alphaviruses (such as, Sindbis, Venezuelan Equine Encephalitis (VEE)), mammalian, yeast and Xenopus expression systems, well known in the art. Particularly preferred expression systems are mammalian cell lines, vaccinia, Sindbis, insect and yeast systems.

Many suitable expression systems are commercially available, including, for example, the following: baculovirus expression system (Reilly, P. R., et al., (1992) BACULOVIRUS EXPRESSION VECTORS: A LABORATORY MANUAL; Beames, et al., (1991) Biotechniques 11:378; Pharmingen; Clontech, Palo Alto, Calif.)), vaccinia expression systems (Earl, P. L., et al., “Expression of proteins in mammalian cells using vaccinia” (1991) In Current Protocols in Molecular Biology (F. M. Ausubel, et al. Eds.), Greene Publishing Associates & Wiley Interscience, New York; Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., (1992) Antonie Van Leeuwenhoek, 62(1-2):79-93; Romanos, M. A., et al., (1992) Yeast 8(6):423-488; Goeddel, D. V., (1990) Methods in Enzymology 185; Guthrie, C., and G. R. Fink, (1991) Methods in Enzymology 194), expression in mammalian cells (Clontech; Gibco-BRL, Ground Island, N.Y.; e.g., Chinese hamster ovary (CHO) cell lines (Haynes, J., et al., (1983) Nuc. AcId. Res. 1983 11:687-706; Lau, Y. F., et al., (1984) Mol. Cell. Biol. 4:1469-1475; Kaufman, R. J., “Selection and coamplification of heterologous genes in mammalian cells,” (1991) in Methods in Enzymology, vol. 185, pp 537-566. Academic Press, Inc., San Diego Calif.), and expression in plant cells (plant cloning vectors, Clontech Laboratories, Inc., Palo-Alto, Calif., and Pharmacia LKB Biotechnology, Inc., Pistcataway, N.J.; Hood, E., et al., (1986) J. Bacteriol. 168:1291-1301; Nagel, R., et al., (1990) FEMS Microbiol. Lett. 67:325; An, et al., “Binary Vectors”, and others (1988) in Plant Molecular Biology Manual A3:1-19; Miki, B. L. A., et al., pp. 249-265, and others (1987) in Plant DNA Infectious Agents (Hohn, T., et al., eds.) Springer-Verlag, Wien, Austria; 1997 Plant Molecular Biology: Essential Techniques, P. G. Jones and J. M. Sutton, New York, J. Wiley; Miglani, 1998 Gurbachan Dictionary of Plant Genetics and Molecular Biology, New York, Food Products Press; Henry, R. J., 1997 Practical Applications of Plant Molecular Biology, New York, Chapman & Hall).

Depending on the expression system and host selected, the VLPs are produced by growing host cells transformed by an expression vector under conditions whereby the particle-forming polypeptide(s) is(are) expressed and VLPs can be formed. The selection of the appropriate growth conditions is within the skill of the art. If the VLPs are formed and retained intracellularly, the cells are then disrupted, using chemical, physical or mechanical means, which lyse the cells yet keep the VLPs substantially intact. Such methods are known to those of skill in the art and are described in, e.g., Protein Purification Applications: A Practical Approach, (E. L. V. Harris and S. Angal, Eds., 1990). Alternatively, VLPs may be secreted and harvested from the surrounding culture media.

The particles are then isolated (or substantially purified) using methods that preserve the integrity thereof, such as, by density gradient centrifugation, e.g., sucrose gradients, PEG-precipitation, pelleting, and the like (see, e.g., Kirnbauer et al. (1993) J. Virol. 67:6929-6936), as well as standard purification techniques including, e.g., ion exchange and gel filtration chromatography.

### Bacterial Vector Vaccines

According to some embodiments, vaccine compositions described herein are based on bacterial vectors.

Genetic engineering techniques have made it possible to identify and delete important virulence genes, enabling the attenuation of pathogenic bacteria and creating vectors unable to revert to their virulent forms. Several mutations have been described for different serotypes of Salmonella enterica (serovars Typhi and Typhimurium, referred to as S. typhi and S. typhimurium, respectively), with the most frequently used being the aroA mutation (as well as aroC and aroD), which blocks the ability of the microorganism to synthesize aromatic compounds. This renders the bacteria unable to reproduce in the host, while retaining the capacity to invade the small intestine and to persist in infecting long enough to produce the antigen and elicit an effective immune response (Cardenas and Clements, 1992 Clin Microbiol Rev 5:328-342). Other useful mutations that can attenuate pathogenicity affect biosynthesis of the nucleotides adenine (pur) and guanine (guaBA), and outer membrane proteins C and F (ompC, ompF), as well as expression of the cAMP receptor (cya/crp), the conversion of UDP-galactose to UDP-glucose (galE), DNA recombination and repair (recA, recBC), and regulation of virulence genes (phoP, phoQ) (Mastroeni et al., 2001 Vet J 161:132-164).

Listeria monocytogenes infection (listeriosis) is a rare and preventable foodborne illness that can cause bacteremia, meningitis, fetal loss, and death, with the risk being greatest for older adults, pregnant women, and persons with immunocompromising conditions. Attenuation of Listeria monocytogenes for vaccine purposes has been achieved using auxotrophic mutants (Zhao et al., 2005 Infect Immun 73:5789-5798) or deletion of virulence factors such as the genes actA and internalin B (inlB) (Brockstedt et al., 2004 Proc Natl Acad Sci USA 101:13832-13837).

Other bacterial species that have been studied for heterologous antigen delivery include Streptococcus gordonii (Lee 2003, Curr Opin Infect Dis. 2003; 16:231-235; Oggioni et al., 1995, Vaccine 13:775-779), Vibrio cholerae (Kaper and Levine 1990, Res Microbiol. 1990; 141:901-906; Silva et al., 2008 Biotechnol Lett 30:571-579), Mycobacterium bovis (BCG) (Bastos et al., 2009 Vaccine. 2009; 27:6495-6503; Nasser Eddine and Kaufmann, 2005, Microbes Infect 7:939-946), Yersinia enterocolitica (Leibiger et al., 2008, Vaccine 26:6664-6670), and Shigella flexnery (Barry et al., 2006, Vaccine 24:3727-3734). Other species that have been investigated for use as vaccine vectors include Pseudomonas aeruginosa (Epaulard et al., 2006 Mol Ther. 2006; 14:656-661), Bacillus subtilis (Duc et al., 2003, Infect Immun 71:2810-2818; Isticato et al., 2001, J Bacteriol 183:6294-6301), and Mycobacterium smegmatis (Lü et al., 2009 Vaccine. 2009; 27:972-978). In the veterinary field, other bacteria have been used to develop a double protective immune response, against a heterologous antigen and against the vector itself; these include Erysipelothrix rhusiopathiae (Ogawa et al., 2009, Vaccine 27:4543-4550), Mycoplasma gallisepticum (Muneta et al., 2008, Vaccine. 2008; 26:5449-5454), and Corynebacterium pseudotuberculosis (Moore et al., 1999, Vaccine. 1999; 18:487-497). A number of live attenuated bacterial vaccines are licensed for veterinary use, including Lawsonia intracellularis, Streptococcus equi (deleted in the aroA gene), Chlamydophila abortus, Mycoplasma synoviae, Mycoplasma gallisepticum (temperature-sensitive mutants), and Bordetella avium. Most of the strains were selected as attenuated, but were not precisely mutated to promote the attenuation and do not carry heterologous antigens (Meeusen et al., 2007, Clin Microbiol Rev 20:489-510).

### Genetic Vaccines

According to some embodiments, the disclosure relates to compositions for delivering nucleic acid molecules that comprise a nucleotide sequence that encodes a conserved immunogenic protein as described herein operably linked to regulatory elements. Aspects of the present disclosure relate to compositions for delivering a recombinant vaccine comprising a nucleotide sequence that encodes that encodes a protein of the disclosure; a live attenuated pathogen that encodes a protein of the disclosure and/or includes a protein of the disclosure; a killed pathogen includes a protein of the disclosure; or a composition such as a liposome or subunit vaccine that comprises a protein of the disclosure. The present disclosure further relates to injectable pharmaceutical compositions.

As described herein, a vaccine according to the disclosure is delivered to an individual to modulate the activity of the individual's immune system and thereby enhance the immune response. When a nucleic acid molecule that encodes the protein is taken up by cells of the individual, the nucleotide sequence is expressed in the cells and the protein are thereby delivered to the individual. Also described herein are methods of delivering the coding sequences of the protein on nucleic acid molecules such as plasmids, as part of recombinant vaccines and as part of attenuated vaccines, as isolated proteins or proteins part of a vector.

DNA vaccines are described in U.S. Pat. Nos. 5,593,972, 5,739,118, 5,817,637, 5,830,876, 5,962,428, 5,981,505, 5,580,859, 5,703,055, 5,676,594, and the priority applications cited therein, all of which are incorporated by reference in their entireties herein. In addition to the delivery protocols described in those applications, alternative methods of delivering DNA are described in U.S. Pat. Nos. 4,945,050 and 5,036,006, incorporated by reference in their entireties herein.

Genetic immunization according to some embodiments of the present disclosure elicits an effective immune response without the use of infective agents or infective vectors. Vaccination techniques which usually do produce a CTL response do so through the use of an infective agent. A complete, broad based immune response is not generally exhibited in individuals immunized with killed, inactivated or subunit vaccines. Some embodiments of the present disclosure achieve the full complement of immune responses in a safe manner without the risks and problems associated with vaccinations that use infectious agents.

According to some embodiments of the present disclosure, DNA or RNA that encodes a conserved immunogenic protein as described herein is introduced into the cells of an individual, or subject, where it is expressed, thus producing the target protein. The DNA or RNA is linked to regulatory elements necessary for expression in the cells of the individual. Regulatory elements for DNA include a promoter and a polyadenylation signal. In addition, other elements, such as a Kozak region, may also be included in the genetic construct.

The genetic constructs of genetic vaccines comprise a nucleotide sequence that encodes a conserved immunogenic protein as described herein operably linked to regulatory elements needed for gene expression. Accordingly, incorporation of the DNA or RNA molecule into a living cell results in the expression of the DNA or RNA encoding the target protein and thus, production of the target protein.

When taken up by a cell, the genetic construct, which includes the nucleotide sequence encoding the conserved immunogenic protein as described herein operably linked to the regulatory elements, may remain present in the cell as a functioning extrachromosomal molecule or it may integrate into the cell's chromosomal DNA. DNA may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Since integration into the chromosomal DNA necessarily requires manipulation of the chromosome, it is preferred to maintain the DNA construct as a replicating or non-replicating extrachromosomal molecule. This reduces the risk of damaging the cell by splicing into the chromosome without affecting the effectiveness of the vaccine. Alternatively, RNA may be administered to the cell. It is also contemplated to provide the genetic construct as a linear minichromosome including a centromere, telomeres and an origin of replication.

The necessary elements of a genetic construct of a genetic vaccine include a nucleotide sequence that encodes a conserved immunogenic protein as described herein and the regulatory elements necessary for expression of that sequence in the cells of the vaccinated individual. The regulatory elements are operably linked to the DNA sequence that encodes the target protein to enable expression.

The molecule that encodes a conserved immunogenic protein as described herein is a protein-encoding molecule which is translated into protein. Such molecules include DNA or RNA which comprise a nucleotide sequence that encodes the target protein. These molecules may be cDNA, genomic DNA, synthesized DNA or a hybrid thereof or an RNA molecule such as mRNA. Accordingly, as used herein, the terms “DNA construct”, “genetic construct” “nucleic acid molecule”, “nucleic acid” and “nucleotide sequence” are meant to refer to both DNA and RNA molecules.

Nucleic acids (DNA or RNA) encoding the immunogens of the disclosure can be used as components of, for example, a DNA or RNA vaccine wherein the encoding sequence is administered as naked DNA or naked RNA or, for example, a minigene encoding the immunogen can be present in a viral vector construct. The encoding sequences can be expressed, for example, in mycobacterium, in a recombinant chimeric adenovirus, or in a recombinant attenuated vesicular stomatitis virus. The encoding sequence can also be present, for example, in a replicating or non-replicating adenoviral vector, an adeno-associated virus vector, an attenuated Mycobacterium tuberculosis vector, a Bacillus Calmette Guerin (BCG) vector, a vaccinia or Modified Vaccinia Ankara (MVA) vector, another pox virus vector, recombinant polio and other enteric virus vector, Salmonella species bacterial vector, Shigella species bacterial vector, Venezuelean Equine Encephalitis Virus (VEE) vector, a Semliki Forest Virus vector, or a Tobacco Mosaic Virus vector. The encoding sequence, can also be expressed as a DNA plasmid with, for example, an active promoter such as a CMV promoter. Other live vectors can also be used to express the sequences of the disclosure. Expression of the immunogen of the disclosure can be induced in a patient's own cells, by introduction into those cells of nucleic acids that encode the immunogen, preferably using codons and promoters that optimize expression in human cells. Examples of methods of making and using DNA vaccines are disclosed in U.S. Pat. Nos. 5,580,859, 5,589,466, and 5,703,055

The regulatory elements necessary for gene expression of a DNA molecule include: a promoter, an initiation codon, a stop codon, and a polyadenylation signal. In addition, enhancers are often required for gene expression. It is necessary that these elements be operable in the vaccinated individual. Moreover, it is necessary that these elements be operably linked to the nucleotide sequence that encodes the target protein such that the nucleotide sequence can be expressed in the cells of a vaccinated individual and thus the target protein can be produced.

Initiation codons and stop codons are generally considered to be part of a nucleotide sequence that encodes the target protein. However, it is necessary that these elements are functional in the vaccinated individual.

Similarly, promoters and polyadenylation signals used must be functional within the cells of the vaccinated individual.

Examples of promoters useful to practice some embodiments of the present disclosure, especially in the production of a genetic vaccine for humans, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter (CMV IE), Epstein Barr Virus (EBV), Rous Sarcoma Virus (RSV) as well as promoters from human genes such as human actin, human myosin, human hemoglobin, human muscle creatine and human metalothionein.

Examples of polyadenylation signals useful to practice some embodiments of the present disclosure, especially in the production of a genetic vaccine for humans, include but are not limited to SV40 polyadenylation signals and LTR polyadenylation signals. In particular, the SV40 polyadenylation signal which is in pCEP4 plasmid (Invitrogen, San Diego Calif.), referred to as the SV40 polyadenylation signal, can be used. Additionally, the bovine growth hormone (bgh) polyadenylation signal can serve this purpose.

In addition to the regulatory elements required for DNA expression, other elements may also be included in the DNA molecule. Such additional elements include enhancers. The enhancer may be selected from the group including but not limited to: human actin, human myosin, human hemoglobin, human muscle creatine and viral enhancers such as those from CMV, RSV and EBV, such as a CMV IE enhancer.

Genetic constructs can be provided with a mammalian origin of replication in order to maintain the construct extrachromosomally and produce multiple copies of the construct in the cell. Plasmids pCEP4 and pREP4 from Invitrogen (San Diego, Calif.) contain the Epstein Barr virus origin of replication and nuclear antigen EBNA-1 coding region which produces high copy episomal replication without integration.

An additional element may be added which serves as a target for cell destruction if it is desirable to eliminate cells receiving the genetic construct for any reason. A herpes thymidine kinase (tk) gene in an expressible form can be included in the genetic construct. When the construct is introduced into the cell, tk will be produced. The drug gangcyclovir can be administered to the individual and that drug will cause the selective killing of any cell producing tk. Thus, a system can be provided which allows for the selective destruction of vaccinated cells.

In order to be a functional genetic construct, the regulatory elements must be operably linked to the nucleotide sequence that encodes the target protein. Accordingly, it is necessary for the initiation and termination codons to be in frame with the coding sequence.

Open reading frames (ORFs) encoding the protein of interest and another or other proteins of interest may be introduced into the cell on the same vector or on different vectors. ORFs on a vector may be controlled by separate promoters or by a single promoter. In the latter arrangement, which gives rise to a polycistronic message, the ORFs will be separated by translational stop and start signals. The presence of an internal ribosome entry site (IRES) site between these ORFs permits the production of the expression product originating from the second ORF of interest, or third, etc. by internal initiation of the translation of the bicistronic or polycistronic mRNA.

When taken up by a cell, the genetic construct(s) may remain present in the cell as a functioning extrachromosomal molecule and/or integrate into the cell's chromosomal DNA. DNA may be introduced into cells where it remains as separate genetic material in the form of a plasmid or plasmids. Alternatively, linear DNA that can integrate into the chromosome may be introduced into the cell. When introducing DNA into the cell, reagents that promote DNA integration into chromosomes may be added. DNA sequences that are useful to promote integration may also be included in the DNA molecule. Alternatively, RNA may be administered to the cell. It is also contemplated to provide the genetic construct as a linear minichromosome including a centromere, telomeres and an origin of replication. Gene constructs may remain part of the genetic material in attenuated live microorganisms or recombinant microbial vectors which live in cells. Gene constructs may be part of genomes of recombinant viral vaccines where the genetic material either integrates into the chromosome of the cell or remains extrachromosomal. Genetic constructs include regulatory elements necessary for gene expression of a nucleic acid molecule. The elements include: a promoter, an initiation codon, a stop codon, and a polyadenylation signal. In addition, enhancers are often required for gene expression of the sequence that encodes the target protein or the immunomodulating protein. It is necessary that these elements be operable linked to the sequence that encodes the desired proteins and that the regulatory elements are operably in the individual to whom they are administered.

According to some embodiments, in order to maximize protein production, regulatory sequences may be selected which are well suited for gene expression in the cells into which the construct is administered. Moreover, codons may be selected which are most efficiently transcribed in the cell. One having ordinary skill in the art can produce DNA constructs that are functional in the cells.

According to some embodiments for which protein is used, for example, one having ordinary skill in the art can produce and isolate proteins of the disclosure using well known techniques. According to some embodiments for which protein is used, for example, one having ordinary skill in the art can, using well known techniques, inserts DNA molecules that encode a protein of the disclosure into a commercially available expression vector for use in well-known expression systems. For example, the commercially available plasmid pSE420 (Invitrogen, San Diego, Calif.) may be used for production of protein in E. coli. The commercially available plasmid pYES2 (Invitrogen, San Diego, Calif.) may, for example, be used for production in S. cerevisiae strains of yeast. The commercially available MAXBAC™ complete baculovirus expression system (Invitrogen, San Diego, Calif.) may, for example, be used for production in insect cells. The commercially available plasmid pcDNA I or pcDNA3 (Invitrogen, San Diego, Calif.) may, for example, be used for production in mammalian cells such as Chinese Hamster Ovary cells. One having ordinary skill in the art can use these commercial expression vectors and systems or others to produce protein by routine techniques and readily available starting materials. (See e.g., Sambrook et al., (1989) Molecular Cloning a Laboratory Manual, Second Ed. Cold Spring Harbor Press.) Thus, the desired proteins can be prepared in both prokaryotic and eukaryotic systems, resulting in a spectrum of processed forms of the protein.

One having ordinary skill in the art may use other commercially available expression vectors and systems or produce vectors using well known methods and readily available starting materials. Expression systems containing the requisite control sequences, such as promoters and polyadenylation signals, and preferably enhancers are readily available and known in the art for a variety of hosts. See e.g., Sambrook et al., Molecular Cloning a Laboratory Manual, Second Ed. Cold Spring Harbor Press (1989). Genetic constructs include the protein coding sequence operably linked to a promoter that is functional in the cell line into which the constructs are transfected. Examples of constitutive promoters include promoters from cytomegalovirus or SV40. Examples of inducible promoters include mouse mammary leukemia virus or metallothionein promoters. Those having ordinary skill in the art can readily produce genetic constructs useful for transfecting with cells with DNA that encodes protein of the disclosure from readily available starting materials. The expression vector including the DNA that encodes the protein is used to transform the compatible host which is then cultured and maintained under conditions wherein expression of the foreign DNA takes place.

The protein produced is recovered from the culture, either by lysing the cells or if secreted from the culture medium as appropriate and known to those in the art. One having ordinary skill in the art can, using well known techniques, isolate protein that is produced using such expression systems. The methods of purifying protein from natural sources using antibodies which specifically bind to a specific protein as described above may be equally applied to purifying protein produced by recombinant DNA methodology.

In addition to producing proteins by recombinant techniques, automated peptide synthesizers may also be employed to produce isolated, essentially pure protein. Such techniques are well known to those having ordinary skill in the art and are useful if derivatives which have substitutions not provided for in DNA-encoded protein production.

According to some embodiments of the disclosure, the genetic vaccine may be administered directly into the individual to be immunized or ex vivo into removed cells of the individual which are reimplanted after administration. By either route, the genetic material is introduced into cells which are present in the body of the individual.

The nucleic acid molecules may be delivered using any of several well-known technologies including DNA injection (also referred to as DNA vaccination), recombinant vectors such as recombinant adenovirus, recombinant adenovirus associated virus and recombinant vaccinia.

Routes of administration of the genetic vaccine include, but are not limited to, intramuscular, intransally, intraperitoneal, intradermal, subcutaneous, intravenous, intraarterially, intraoccularly and oral as well as topically, transdermally, by inhalation or suppository or to mucosal tissue such as by lavage to vaginal, rectal, urethral, buccal and sublingual tissue. Exemplary routes of administration include intramuscular, intraperitoneal, intradermal and subcutaneous injection. Genetic constructs may be administered by means including, but not limited to, traditional syringes, needleless injection devices, or “microprojectile bombardment gene guns”.

According to some embodiments, the nucleic acid molecule is delivered to the cells in conjunction with administration of a polynucleotide function enhancer or a genetic vaccine facilitator (“GVF”) agent. Polynucleotide function enhancers are described in U.S. Pat. Nos. 5,593,972, 5,962,428 and International Application Serial Number PCT/US94/00899 filed Jan. 26, 1994. Genetic vaccine facilitator agents are described in U.S. Ser. No. 021,579 filed Apr. 1, 1994. The co-agents that are administered in conjunction with nucleic acid molecules may be administered as a mixture with the nucleic acid molecule or administered separately simultaneously, before or after administration of nucleic acid molecules. In addition, other agents which may function transfecting agents and/or replicating agents and/or inflammatory agents and which may be co-administered with a GVF include growth factors, cytokines and lymphokines such as a-interferon, gamma-interferon, GM-CSF, platelet derived growth factor (PDGF), TNF, epidermal growth factor (EGF), IL-1, IL-2, IL-4, IL-6, IL-10, IL-12 and IL-15 as well as fibroblast growth factor, surface active agents such as immune-stimulating complexes (ISCOMS), Freunds incomplete adjuvant, LPS analog including monophosphoryl Lipid A (WL), muramyl peptides, quinone analogs and vesicles such as squalene and squalene, and hyaluronic acid may also be used administered in conjunction with the genetic construct According to some embodiments, an immunomodulating protein may be used as a GVF. According to some embodiments, the nucleic acid molecule is provided in association with a biodegradable matrix polymer, e.g., poly(DL-lactide-co-glycolide) (PLG), to enhance delivery/uptake.

The genetic vaccines according to some embodiments of the present disclosure comprise about 1 nanogram to about 1000 micrograms of DNA. According to some exemplary embodiments, the vaccines contain about 10 nanograms to about 800 micrograms, inclusive, of DNA. According to some exemplary embodiments, the vaccines contain about 0.1 to about 500 micrograms of DNA, inclusive. According to some exemplary embodiments, the vaccines contain about 1 to about 350 micrograms of DNA, inclusive. According to some exemplary embodiments, the vaccines contain about 25 to about 250 micrograms of DNA, inclusive. According to some exemplary embodiments, the vaccines contain about 100 micrograms DNA.

Genetic constructs may optionally be formulated with one or more response enhancing agents such as: compounds which enhance transfection, i.e., transfecting agents; compounds which stimulate cell division, i.e., replication agents; compounds which stimulate immune cell migration to the site of administration, i.e., inflammatory agents; compounds which enhance an immune response, i.e., adjuvants or compounds having two or more of these activities.

According to some embodiment, bupivacaine, a well known and commercially available pharmaceutical compound, is administered prior to, simultaneously with or subsequent to the genetic construct. Bupivacaine and the genetic construct may be formulated in the same composition. Bupivacaine is particularly useful as a cell stimulating agent in view of its many properties and activities when administered to tissue. Bupivacaine promotes and facilitates the uptake of genetic material by the cell. As such, it is a transfecting agent. Administration of genetic constructs in conjunction with bupivacaine facilitates entry of the genetic constructs into cells. Bupivacaine is believed to disrupt or otherwise render the cell membrane more permeable. Cell division and replication is stimulated by bupivacaine. Accordingly, bupivacaine acts as a replicating agent. Administration of bupivacaine also irritates and damages the tissue. As such, it acts as an inflammatory agent which elicits migration and chemotaxis of immune cells to the site of administration. In addition to the cells normally present at the site of administration, the cells of the immune system which migrate to the site in response to the inflammatory agent can come into contact with the administered genetic material and the bupivacaine. Bupivacaine, acting as a transfection agent, is available to promote uptake of genetic material by such cells of the immune system as well.

In addition to bupivacaine, mepivacaine, lidocaine, procains, carbocaine, methyl bupivacaine, and other similarly acting compounds may be used as response enhancing agents. Such agents act as cell stimulating agents which promote the uptake of genetic constructs into the cell and stimulate cell replication as well as initiate an inflammatory response at the site of administration.

Other contemplated response enhancing agents which may function as transfecting agents and/or replicating agents and/or inflammatory agents and which may be administered include lectins, growth factors, cytokines and lymphokines such as alpha-interferon, gamma-interferon, platelet derived growth factor (PDGF), gCSF, gMCSF, TNF, epidermal growth factor (EGF), IL-1, IL-2, IL-4, IL-6, IL-8, IL-10 and IL-12 as well as collagenase, fibroblast growth factor, estrogen, dexamethasone, saponins, surface active agents such as immune-stimulating complexes (ISCOMS), Freund's incomplete adjuvant, LPS analogs, including monophosphoryl Lipid A (MPL), muramyl peptides, quinone analogs and vesicles such as squalene and squalane, hyaluronic acid and hyaluronidase may also be administered in conjunction with the genetic construct. According to some embodiments, combinations of these agents are co-administered in conjunction with the genetic construct. In other embodiments, genes encoding these agents are included in the same or different genetic construct(s) for co-expression of the agents.

### Lipid Immunogens

The immunogens (e.g., conserved immunogens) disclosed herein may also be linked directly to, or through a spacer or linker to: an immunogenic carrier such as serum albumin, tetanus toxoid, keyhole limpet hemocyanin, dextran, or a recombinant virus particle; an immunogenic peptide known to stimulate a T helper cell type immune response; a cytokine such as interferon gamma or GMCSF; a targeting agent such as an antibody or receptor ligand; a stabilizing agent such as a lipid; or a conjugate of a plurality of epitopes to a branched lysine core structure, such as the so-called “multiple antigenic peptide” described by Posenett et. al., incorporated by reference in its entirety herein; a compound such as polyethylene glycol to increase the half-life of the peptide; or additional amino acids such as a leader or secretory sequence, or a sequence employed for the purification of the mature sequence. Spacers and linkers typically comprise relatively small, neutral molecules. In addition, such linkers need not be composed of amino acids but any oligomeric structures will do as well so long as they provide the correct spacing so as to optimize the desired level of immunogenic activity of the immunogens of the present disclosure. The immunogen may therefore take any form that is capable of eliciting a CTL response.

The skilled artisan would appreciate, once armed with the teachings provided herein, that the vaccine compositions described herein encompass numerous molecules, some either expressed under the control of a single promoter/regulatory sequence or under the control of more than one such sequence. Moreover, the disclosure encompasses administration of one or more vaccines of the disclosure where the various vaccines encode different molecules. That is, the various molecules (e.g., conserved immunogens, costimulatory ligands, cytokines, and the like) can work in cis (i.e., in the same vaccine vector and/or encoded by the same contiguous nucleic acid or on separate nucleic acid molecules within the same vaccine vector) or in trans (i.e., the various molecules are expressed by different vaccines).

### Methods for Determining Immune Response

Most immune responses associated with vaccination are controlled by specific T cells of a CD4+ helper phenotype which mediate the generation of effector antibodies, cytotoxic T lymphocytes (CTLs), or the activation of innate immune effector cells. The resulting antigen-specific T cell responses need to be of the appropriate type involving: helper T cells (T_{H} cells, expressing cytokines and co-stimulatory molecules), and/or cytotoxic T lymphocytes (CTL), and with memory and homing capacity, and should not be exhausted or anergized via negative feedback or immune checkpoints. A formulation (antigen, vehicle, adjuvants; proportions thereof) and regimen (including the number and interval between immunizations, and route of vaccination) that generates the appropriate T cell response is required. The measurement and characterization of these T cells provides useful markers of immunogenicity and efficacy, and informs on mechanisms for further vaccine development.

Methods for determining immune responses are known in the art. According to some embodiments, viral lesions can be examined to determine the occurrence of an immune response to the virus and/or the antigen. According to some embodiments, in vitro assays may be used to determine the occurrence of an immune response. Examples of such in vitro assays include ELISA assays and cytotoxic T cell (CTL) assays. According to some embodiments, the immune response is measured by detecting and/or quantifying the relative amount of an antibody, which specifically recognizes an antigen in the sera of a subject who has been treated by administering the live, modified, non-replicating or replication-impaired poxvirus comprising the antigen, relative to the amount of the antibody in an untreated subject. According to some embodiments, the immune response to vaccination is measured by immunotyping the individual before, during and after vaccination. According to some embodiments, a BCR immune repertoire, TCR immune repertoire or both (e.g., iRepertoire, Inc., Huntsville, Ala.) of the vaccinated individual is determined before, during and after vaccination.

Techniques for assaying antibodies in a sample are known in the art and include, for example, sandwich assays, ELISA and ELISpot. Polyclonal sera are relatively easily prepared by injection of a suitable laboratory animal with an effective amount of the immune effector, or antigenic part thereof, collecting serum from the animal and isolating specific sera by any of the known immunoadsorbent techniques. Antibodies produced by this method are utilizable in virtually any type of immunoassay.

The use of monoclonal antibodies in an immunoassay is preferred because of the ability to produce them in large quantities and the homogeneity of the product. The preparation of hybridoma cell lines for monoclonal antibody production derived by fusing an immortal cell line and lymphocytes sensitized against the immunogenic preparation can be achieved by techniques which are well known to those who are skilled in the art. In other embodiments, ELISA assays may be used to determine the level of isotype specific antibodies using methods known in the art.

CTL assays can be used to determine the lytic activity of CTLs, measuring specific lysis of target cells expressing a certain antigen. Immune-assays may be used to measure the activation (e.g., degree of activation) of sample immune cells. “Sample immune cells” refer to immune cells contained in samples from any source, including from a human patient, human donor, animal, or tissue cultured cell line. The immune cell sample can be derived from peripheral blood, lymph nodes, bone marrow, thymus, any other tissue source including in situ or excised tumor, or from tissue or organ cultures. The sample may be fractionated or purified to generate or enrich a particular immune cell subset before analysis. The immune cells can be separated and isolated from their source by standard techniques.

Immune cells include both non-resting and resting cells, and cells of the immune system that may be assayed, including, but not limited to, B lymphocytes, T lymphocytes, natural killer (NK) cells, invariant NKT (iNKT) cells, lymphokine-activated killer (LAK) cells, monocytes, macrophages, neutrophils, granulocytes, mast cells, platelets, Langerhans cells, stem cells, dendritic cells, and peripheral blood mononuclear cells.

Immune cell activity that may be measured includes, but is not limited to (1) cell proliferation by measuring the cell or DNA replication; (2) enhanced cytokine production, including specific measurements for cytokines, such as γ-IFN, GM-CSF, or TNF-alpha, IFN-alpha, IL-6, IL-10, IL-12; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; (9) chemokine secretion such as IP-10; (10) expression of costimulatory molecules (e.g., CD80, CD86) and maturation molecules (e.g., CD83), (12) upregulation of class II MHC expression; and (13) apoptosis, which refers to fragmentation of activated immune cells under certain circumstances, as an indication of abnormal activation.

Reporter molecules may be used for many of the immune assays described. A reporter molecule is a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules. In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-antigen complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. The fluorescent labeled antibody is allowed to bind to the first antibody-antigen complex. After washing off the unbound reagent, the remaining tertiary complex is then exposed to the light of the appropriate wavelength the fluorescence observed indicates the presence of the antigen of interest.

The ability to measure memory T cells in association with vaccination is important to establish a vaccine's immunogenicity and may be a biomarker of efficacy by having a positive association with protection from infection and/or disease. Important factors in the measurement of T cell responses include, without limitation, the methods of measurement, when to make these measurements, and in which locations. T cell responses measured in the circulating PBMCs during a vaccination regimen tend to follow the typical pattern of adaptive immune response, i.e., an initial exposure is followed by a lag phase, then a peak in the response (such as an antigen-specific IFN-γ response) at about one to two weeks, that eventually settles back down to a response raised over the naïve response. Due to memory generated by the priming, a second exposure through boosting gives a more rapid, greater response. Through effector T cell (T_{E}) attrition, this response then settles to a level higher than it was before the boost. Thus, the aim of boosting is to cause the T cells to reach a putative “protective” level. To date, few direct correlations between T cell responses and degrees of protection from infection and disease have been established for clinical use. “Quantiferon™” and ELIspot tests have been able to detect latent Mycobacterium tuberculosis infection in at-risk individuals through the detection of IFNγ secretion by peripheral T cells reactive to particular TB antigens. However, such responses following vaccination for TB have not been associated with protection from infection. Even if the PBMCs may not be the ideal location to detect the reactive T cells that need to act in specific tissues (such as the mucosa), precursors in transit (such as T_{EM} and T_{CM}) are measurable with specialized or modified techniques. Ex vivo techniques on whole blood or PBMCs involve the exposure to the vaccine antigen and the measurement of responses, which typically occur within one day. The most common of such tests involves bulk cytokine secretion from blood cells (whole blood assay/ELISA) and cytokine measurement by flow cytometry and enzyme-linked immunospot (ELISpot). This is done to identify responding cells at the single cell level. The expansion in the number of available flow cytometry parameters means that cells can be identified as secreting or expressing a multitude of molecules using single-cell mass cytometry and RNA sequencing, thus allowing their characterization within effector phenotypes, memory phenotypes, and beyond. Molecular signatures in blood that are associated with vaccination continue to implicate T cells in protection. Although it is more cumbersome, incorporating a period of culture of blood cells with antigens and other factors allows specific memory cells like T_{CM} to be revealed, as the culture promotes differentiation to T_{EM} and/or T_{E}. One approach with whole blood that was cultured together with a precise vaccine formulation (antigen in adjuvant+TLR ligand) (Hakimi J. et al., 2017 Hum Vaccin Immunother. Sep. 2; 13(9):2130-2134) revealed significantly higher T cell cytokine secretion. These results suggest that components of the whole blood interact with components of the vaccine to promote T cell reactivation in a way that might emulate in vivo events. Such an assay is capable of monitoring vaccine formulations for potency as well as testing vaccine recipients for their potential to respond to a vaccine in vivo. Being able to emulate ectopic lymphoid structures (memory depots) may provide one method of recreating and studying vaccine responses in vitro.

Other exemplary immune assays are described herein below.

Cell Proliferation Assay: Activated immune cell proliferation is intended to include increase in cell number, cell growth, cell division, or cell expansion, as measured by cell number, cell weight, or by incorporation of radiolabelled nucleic acids, amino acids, proteins, or other precursor molecules. As one example, DNA replication is measured by incorporation of radioisotope labels. According to some embodiments, cultures of stimulated immune cells can be measured by DNA synthesis by pulse-labeling the cultures with tritiated thymidine (³H-Tdr), a nucleoside precursor that is incorporated into newly synthesized DNA. Thymidine incorporation provides a quantitative measure of the rate of DNA synthesis, which is usually directly proportional to the rate of cell division. The amount of ³H-labeled thymidine incorporated into the replicating DNA of cultured cells is determined by scintillation counting in a liquid scintillation spectrophotometer. Scintillation counting yields data in counts per minute (cpm) which may then be used as a standard measure of immune cell responsiveness. The cpm in resting immune cell cultures may be either subtracted from or divided into cpm of the primed immune cells, which will yield a stimulation index ratio.

Flow cytometry can also be used to measure proliferation by measuring DNA with light scatter, Coulter volume and fluorescence, all of which are techniques that are well known in the art.

Enhanced Cytokine Production Assay: A measure of immune cell stimulation is the ability of the cells to secrete cytokines, lymphokines, or other growth factors. Cytokine production, including specific measurements for cytokines, such as γ-IFN, GM-CSF, or TNF-alpha, may be made by radioimmunoassay (RIA), enzyme-linked immunoabsorbent assay (ELISA), bioassay, or measurement of messenger RNA levels. In general, with these immunoassays, a monoclonal antibody to the cytokine to be measured is used to specifically bind to and thus identify the cytokine. Immunoassays are well known in the art and can include both competitive assays and immunometric assays, such as forward sandwich immunoassays, reverse sandwich immunoassays and simultaneous immunoassays.

In each of the above assays, the sample-containing cytokine is incubated with the cytokine-specific monoclonal antibody under conditions and for a period of time sufficient to allow the cytokines to bind to the monoclonal antibodies. In general, it is desirable to provide incubation conditions sufficient to bind as much cytokine and antibody as possible, since this will maximize the signal. Of course, the specific concentrations of antibodies, the temperature and time of incubation, as well as other such assay conditions, can be varied, depending upon various factors including the concentration of cytokine in the sample, the nature of the sample, and the like. Those skilled in the art will be able to determine operative and optimal assay conditions for each determination by employing routine experimentation.

Cell-Mediated Target Cell Lysis Assay: Another type of indicator for degree of immune cell activation is immune cell-mediated target cell lysis, which is meant to encompass any type of cell killing, including cytotoxic T lymphocyte activity, apoptosis, and the induction of target lysis by molecules secreted from non-resting immune cells stimulated to activity. Cell-mediated lympholysis techniques typically measure the ability of the stimulated immune cells to lyse ⁵¹Cr-labeled target cells. Cytotoxicity is measured as a percentage of ⁵¹Cr released in specific target cells compared to percentage of ⁵¹Cr released from control target cells. Cell killing may also be measured by counting the number of target cells, or by quantifying an inhibition of target cell growth.

Cell Differentiation Assay: Another indicator of immune cell activity is immune cell differentiation and maturation. Cell differentiation may be assessed in several different ways. One such method is by measuring cell phenotypes. The phenotypes of immune cells and any phenotypic changes can be evaluated by flow cytometry after immunofluorescent staining using monoclonal antibodies that will bind membrane proteins characteristic of various immune cell types.

A second means of assessing cell differentiation is by measuring cell function. This may be done biochemically, by measuring the expression of enzymes, mRNA's, genes, proteins, or other metabolites within the cell, or secreted from the cell. Bioassays may also be used to measure functional cell differentiation.

Immune cells express a variety of cell surface molecules which can be detected with either monoclonal antibodies or polyclonal antisera. Immune cells that have undergone differentiation or activation can also be enumerated by staining for the presence of characteristic cell surface proteins by direct immunofluorescence in fixed smears of cultured cells.

Mature B cells can be measured in immunoassays, for example, by cell surface antigens including CD19 and CD20 with monoclonal antibodies labeled with fluorochromes or enzymes. B cells that have differentiated into plasma cells can be enumerated by staining for intracellular immunoglobulins by direct immunofluorescence in fixed smears of cultured cells.

Immunoglobulin Production Assay: B cell activation results in small, but detectable, quantities of polyclonal immunoglobulins. Following several days of culture, these immunoglobulins may be measured by radioimmunoassay or by enzyme-linked immunosorbent assay (ELISA) methods.

B cells that produce immunoglobulins can also be quantified by the reversed hemolytic plaque assay. In this assay, erythrocytes are coated with goat or rabbit anti-human immunoglobulins. These immunoglobulins are mixed with the activated immunoglobulin-producing lymphocytes and semisolid agar, and complement is added. The presence of hemolytic plaques indicates that there are immunoglobulin-producing cells.

Chemotactic Factor Assay: Chemotactic factors are molecules which induce or inhibit immune cell migration into or out of blood vessels, tissues or organs, including cell migration factors. The chemotactic factors of immune cells can be assayed by flow cytometry using labeled monoclonal antibodies to the chemotactic factor or factors being assayed. Chemotactic factors may also be assayed by ELISA or other immunoassays, bioassays, messenger RNA levels, and by direct measurements, such as cell counting, of immune cell movements in specialized migration chambers.

Addback Assays: When added to fresh peripheral blood mononuclear cells, autologous ex vivo activated cells exhibit an enhanced response to a “recall” antigen, which is an antigen to which the peripheral blood mononuclear cells had previously been exposed. Primed or stimulated immune cells should enhance other immune cells response to a “recall” antigen when cultured together. These assays are termed “helper” or “addback” assays. In this assay, primed or stimulated immune cells are added to untreated, usually autologous immune cells to determine the response of the untreated cells. The added primed cells may be irradiated to prevent their proliferation, simplifying the measurement of the activity of the untreated cells. These assays may be particularly useful in evaluating cells for blood exposed to virus. The addback assays can measure proliferation, cytokine production, and target cell lysis as described herein.

The above-described methods and other additional methods to determine an immune response are well known in the art.

### Methods of Use

In accordance with further embodiments of the disclosure, methods for reducing risk of an infection with an infectious agent amenable to vaccination according to the present disclosure are provided. According to some embodiments, a method includes administering to a subject in need thereof an amount of immunogen, or protein thereof, sufficient to reduce the risk of infection.

In accordance with further embodiments of the disclosure, there are provided prophylactic methods including methods of vaccinating and immunizing a subject against a viral infection. According to some embodiments, the disclosure provides methods of protecting a subject against SARS-CoV infection to decrease or reduce the probability of SARS-CoV infection or pathology in a subject or to decrease or reduce susceptibility of a subject to SARS-CoV infection or pathology or to inhibit or prevent SARS-CoV infection in a subject or to reduce risk of spread in a susceptible population. According to some embodiments, the disclosure provides methods of protecting a subject against SARS-CoV-2 infection to decrease or reduce the probability of SARS-CoV-2 infection or pathology in a subject or to decrease or reduce susceptibility of a subject to SARS-CoV-2 infection or pathology or to inhibit or prevent SARS-CoV-2 infection in a subject or to reduce risk of spread in a susceptible population. In some embodiments, the disclosure provides methods of protecting a subject against HIV infection to decrease or reduce the probability of HIV infection or pathology in a subject or to decrease or reduce susceptibility of a subject to HIV infection or pathology or to inhibit or prevent HIV infection in a subject or to reduce risk of spread in a susceptible population.

According to another embodiment, the method includes a process for inducing a cellular immune response in vitro that is specific to viral antigens expressed by a virus infected cell, comprises contacting a CTL precursor lymphocyte with an antigen presenting cell that is expressing a polynucleotide coding for a viral polypeptide of the disclosure, wherein said polynucleotide is operably linked to a promoter. The foreign viral antigen bound to MHC molecules expressed on the stimulator APCs can serve as the activating stimulus to responding T lymphocytes comprising a population of cells that exhibit an ability to kill pathogen-infected cells, while showing resistance to such killing action. The ability to kill pathogen-infected cells may be direct, though cytolytic or cytotoxic activities, or indirect, through the immunoregulation of other cells and proteins that target pathogenic cells. There are multiple kinds of cells that can display this effector function, e.g., NK cells, NKT cells, LAK cells, CIK cells, MAIT cells, CD8+ CTLs, CD4+ CTLs (collectively “CTLs”). Proliferation of the responding T lymphocytes then can be measured.

A variety of techniques exist for assaying the activity of activated CTL, and are described infra.

After expansion of the antigen-specific CTLs, the activated CTLs are then adoptively transferred back into the patient, where they will destroy their specific target cell. Methodologies for reinfusing T cells into a patient are well known and exemplified in U.S. Pat. No. 4,844,893 to Honski, et al., and U.S. Pat. No. 4,690,915 to Rosenberg.

The peptide-specific activated CTLs can be purified from the stimulator cells prior to infusion into the patient. For example, monoclonal antibodies directed toward the cell surface protein CD8, present on CTLs, can be used in conjunction with a variety of isolation techniques such as antibody panning, flow cytometric sorting, and magnetic bead separation to purify the peptide-specific CTL away from any remaining non-peptide specific lymphocytes or from the stimulator cells.

Thus, according to some embodiments of the present disclosure, a process for reducing risk of infection with a pathogen, reducing risk of spread of infection in a population, or both comprises administering, activated CTLs produced in vitro in an amount sufficient to effect the destruction of the pathogen infected cells either directly or indirectly through the elaboration of cytokines.

Another embodiment of the present disclosure is directed to a process for treating a subject at risk for infection with a pathogen, reducing risk of spread of infection in a population, or both, where the infection is characterized by pathogen-infected cells expressing any class I MHC molecule and an internal CD8+ T cell epitope as determined using methods described herein, comprising producing activated CTLs specific for the epitope or original protein in vitro, and administering the activated CTLs in an amount sufficient to destroy the infected cells through direct lysis or to effect the destruction of the infected cells indirectly through the elaboration of cytokines.

According to some embodiments, the ex vivo generated activated CTLs can be used to identify and isolate the T cell receptor molecules specific for the peptide. The genes encoding the alpha and beta chains of the T cell receptor can be cloned into an expression vector system and transferred and expressed in naive T cells from peripheral blood, T cells from lymph nodes, or T lymphocyte progenitor cells from bone marrow. These T cells, which would then be expressing a peptide-specific T cell receptor, would then have anti-viral reactivity and could be used in adoptive therapy of infection against multiple heterologous subtypes of virus.

In addition to their use for therapeutic or prophylactic purposes, the immunogenic peptides of the present disclosure are useful as screening and diagnostic agents. Thus, the immunogenic peptides of the present disclosure, together with modern techniques of CTL screening, make it possible to screen patients for the presence of T cells specific for these peptides as a test for viral infection, exposure and immune response. The results of such screening may help determine the efficacy of proceeding with the regimen of treatment disclosed herein using the immunogens of the present disclosure.

The therapeutically effective amount of a composition containing one or more of the immunogens of this disclosure is an amount sufficient to induce an effective CTL response to prevent, cure or arrest disease progression in a population. Thus, this dose will depend, among other things, on the identity of the immunogens used, the nature of the disease condition, the severity of the disease condition, the extent of any need to prevent such a condition where it has not already been detected, the manner of administration dictated by the situation requiring such administration, the weight and state of health of individuals receiving such administration, and the sound judgment of the clinician or researcher.

### Pharmaceutical Compositions

According to some embodiments, the vaccines of the present disclosure can be prepared as injectables, in the form of aqueous solutions or suspensions. Pharmaceutical carriers, diluents and excipients can be generally added that are compatible with the active ingredients and acceptable for pharmaceutical use.

While any suitable carrier known to those of ordinary skill in the art may be employed in the vaccine compositions of this disclosure, the type of carrier will vary depending on the mode of administration. Compositions of the present disclosure may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., poly(lactic-co-glycolic acid), PLGA)) may also be employed as carriers for the pharmaceutical compositions of this disclosure. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252. Modified hepatitis B core protein carrier systems are also suitable, such as those described in WO/99 40934, and references cited therein, all incorporated herein by reference. One may also employ a carrier comprising the particulate-protein complexes described in U.S. Pat. No. 5,928,647, which are capable of inducing a class I-restricted cytotoxic T lymphocyte response in a host.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants, solutes that render the formulation isotonic, hypotonic or weakly hypertonic with the blood of a recipient, suspending agents, thickening agents and/or preservatives. Alternatively, compositions of the present disclosure may be formulated as a lyophilizate. Compounds may also be encapsulated within liposomes using well known technology.

Any of a variety of immunostimulants may optionally be employed in the vaccines of this disclosure. For example, an adjuvant may be included. According to some embodiments, the adjuvant is mucosal adjuvant MALP-2. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Exemplary adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants. Within the context of the HCMV-based and rhesus cytomegalovirus (RhCMV)-based (e.g., strain 68-1) vaccine vectors provided herein, the adjuvant composition is optional, but if included, is designed to induce an immune response predominantly of the T_{HI} type. High levels of T_{HI}-type cytokines {e.g., IFN-γ, TNFα, IL-2 and IL-12) tend to favor the induction of cell mediated immune responses to an administered antigen. In contrast, high levels of T_{H2}-type cytokines {e.g., IL-4, IL-5, IL-6 and IL-10) tend to favor the induction of humoral immune responses. Following application of a vaccine as provided herein, a patient will support an immune response that includes T_{HI}- and T_{H2}-type responses. For example, in an embodiment in which a response is predominantly T_{HI}-type, the level of T_{HI}-type cytokines will increase to a greater extent than the level of T_{H2}-type cytokines. The levels of these cytokines may be readily assessed using standard assays. For a review of the families of cytokines, see Mosmann and Coffman, Ann. Rev. Immunol. 7:145-173, 1989.

According to some embodiments, adjuvants for use in eliciting a predominantly T_{HI}-type response are employed. Such adjuvants include, for example, a combination of monophosphoryl lipid A, e.g., 3-de-O-acylated monophosphoryl lipid A (3D-MPL), together with an aluminum salt. MPL adjuvants are available from Corixa Corporation (Seattle, Wash.; see U.S. Pat. Nos. 4,436,727; 4,877,611; 4,866,034 and 4,912,094). CpG-containing oligonucleotides (in which the CpG dinucleotide is unmethylated) also induce a predominantly T_{HI} response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another exemplary adjuvant is a saponin, for example QS21 (Aquila Biopharaiaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other exemplary formulations comprise an oil-in-water emulsion and tocopherol. A particularly potent adjuvant formulation involving QS21, 3D-MPL and tocopherol in an oil-in-water emulsion is described in WO 95/17210. Other exemplary adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties. Other exemplary adjuvants comprise polyoxyethylene ethers, such as those described in WO 99/52549 A1.

Any vaccine provided herein may be prepared using well known methods that result in a combination of vector, optional immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology (see, e.g., Coombes et al., Vaccine 74:1429-1438, 1996) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), polyacrylate, latex, starch, cellulose, dextran and the like. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Regardless of the nature of the composition given, additional vaccine compositions may also accompany the immunogens of the present disclosure. Thus, for purposes of preventing or treating infection (e.g., prophylactic or therapeutic vaccine), compositions containing the immunogens disclosed herein may, in addition, contain other vaccine pharmaceuticals. The use of such compositions with multiple active ingredients is left to the discretion of the clinician.

According to some embodiments, the concentration of the immunogenic polypeptides of the disclosure in pharmaceutical formulations are subject to wide variation, including anywhere from less than 0.01% by weight to as much as 50% or more. Factors such as volume and viscosity of the resulting composition must also be considered. The solvents, or diluents, used for such compositions include water, dimethylsulfoxide, PBS (phosphate buffered saline), or saline itself, or other possible carriers or excipients.

According to some embodiments, the pharmaceutical compositions according to the present disclosure comprise about 1 nanogram to about 2000 micrograms of DNA, inclusive. According to some preferred embodiments, pharmaceutical compositions according to the present disclosure comprise about 5 nanogram to about 1000 micrograms of DNA, inclusive. According to some preferred embodiments, the pharmaceutical compositions contain about 10 nanograms to about 800 micrograms of DNA, inclusive. According to some preferred embodiments, the pharmaceutical compositions contain about 0.1 to about 500 micrograms of DNA, inclusive. According to some preferred embodiments, the pharmaceutical compositions contain about 1 to about 350 micrograms of DNA, inclusive. According to some preferred embodiments, the pharmaceutical compositions contain about 25 to about 250 micrograms of DNA, inclusive. According to some preferred embodiments, the pharmaceutical compositions contain about 100 to about 200 microgram DNA, inclusive.

The peptides and polypeptides of the disclosure can also be added to professional antigen presenting cells such as dendritic cells that have been prepared ex vivo.

### Administration

The immunogenic compositions according to the present disclosure may be used against an infectious agent by administration to an individual or to a population by a variety of routes. According to some embodiments, administration may be by inhalation. Gaseous and volatile drugs may be inhaled and absorbed through the pulmonary epithelium and mucous membranes of the respiratory tract. Access to the circulation is rapid by this route because the lung's surface area is large. In addition, solutions of drugs can be atomized and the fine droplets in air (aerosol) inhaled. Advantages are the almost instantaneous absorption of a drug into the blood, avoidance of hepatic first-pass loss, and in the case of pulmonary disease, local application of the drug at the desired site of action. According to some embodiments, drugs may be applied topically to the mucous membranes of the nasopharynx by insufflation. Absorption from this site is generally excellent anday provide advantages, because vaccination of mucosal surfaces using mucosal vaccines provides the basis for generating protective immunity in both the mucosal and systemic immune compartments. [Goodman & Gilman's The Pharmacological Basis of Therapeutics, 13^{th} Ed., Brunton, L L, et al. Ed., McGraw Hill Education (2018), at 17-18]. According to some embodiments, the composition may be administered parenterally or orally, and, if parenterally, either systemically or topically. Parenteral routes include subcutaneous, intravenous, intradermal, intramuscular, intraperitoneal, intranasal, transdermal, or buccal routes. One or more such routes may be employed. Parenteral administration can be, for example, by bolus injection or by gradual perfusion over time.

According to some embodiments, the disclosure provides a vaccine in which an immunogen of the present disclosure is delivered or administered in the form of a polynucleotide encoding a polypeptide or active fragment as disclosed herein, whereby the peptide or polypeptide or active fragment is produced in vivo. The polynucleotide may be included in a suitable expression vector and combined with a pharmaceutically acceptable carrier. A wide variety of vectors are available and apparent to those skilled in the art. Vaccinia vectors and methods useful in immunization protocols are described in U.S. Pat. No. 4,722,848, the disclosure of which is incorporated herein by reference in its entirety.

The compositions described herein can be administered prior to, concurrent with, or subsequent to delivery of other vaccines. Also, the site of administration may be the same or different as other vaccine compositions that are being administered.

Dosage treatment with the composition may be a single dose schedule or a multiple dose schedule. A multiple dose schedule is one in which a primary course of vaccination may be with 1-10 separate doses, followed by other doses given at subsequent time intervals, chosen to maintain and/or reinforce the immune response, for example at 1-4 months for a second dose, and if needed, a subsequent dose(s) after several months. The dosage regimen will also, at least impart, be determined by the potency of the modality, the vaccine delivery employed, the need of the subject and be dependent on the judgment of the practitioner.

In a specific embodiment of the disclosure, each injection contains a recombinant vaccine from a different vector source during the immunization of the vaccine described above.

### Prime-Boost

According to some embodiments of the present disclosure, in the above vaccine immunization process, the vaccine is immunized with a “prime and boost” immunization strategy, and each recombinant vaccine is inoculated at least once. According to some embodiments, the disclosure relates to “prime and boost” immunization regimes in which the immune response induced by administration of a priming composition is boosted by administration of a boosting composition. For example, effective boosting can be achieved using a subunit or protein vaccine, following priming with a genetic or DNA plasmid vaccine. Some embodiments of the present disclosure employ a subunit or protein vaccine for providing a boost to an immune response primed to antigen using the genetic or DNA plasmid vaccine.

Use of embodiments of the present disclosure allows for a subunit or protein vaccine to boost an immune response primed by a DNA vaccine. Monovalent or other multivalent vaccines can also be used.

Advantageously, a vaccination regime using intramuscular immunization for both prime and boost can be employed, constituting a general immunization regime suitable for inducing an immune response, e.g., in humans.

Some embodiments of the present disclosure in various aspects and embodiments employ a subunit or protein vaccine for boosting an immune response to the antigen primed by previous administration of the nucleic acid encoding the antigen.

According to some embodiments, the present disclosure provides for the use of a subunit or protein vaccine for boosting an immune response to an antigen.

According to some embodiments, the present disclosure provides a method of inducing an immune response to an antigen in an individual, the method comprising administering to the individual a priming DNA vaccine comprising a PLG matrix containing an unformulated (naked) plasmid DNA encoding ane antigen. For example, according to some embodiments, the DNA vaccine encodes one or more conserved protein of a human coronavirus. According to some such embodiments, the conserved protein of the human coronavirus is one or more of a human coronavirus (S) protein of amino acid sequence SEQ ID NO: 2 or an immunogenic fragment thereof; a coronavirus membrane (M) protein or an immunogenic fragment thereof of amino acid sequence SEQ ID NO: 3 or 4; a coronavirus nucleocapsid (N) protein or an immunogenic fragment thereof of aminoacid sequence SEQ ID NO: 7 or 8; or a coronavirus envelope (E) protein or an immunogenic fragment thereof of amino acid sequence SEQ ID NO: 5 or 6. The method further comprises then administering a boosting composition, wherein the boosting composition comprises a subunit or protein vaccine comprising one or more conserved protein of a human coronavirus. According to some such embodiments, the conserved protein of the human coronavirus is a human coronavirus (S) protein of amino acid sequence SEQ ID NO: 2 or an immunogenic fragment thereof; a coronavirus membrane (M) protein or an immunogenic fragment thereof of amino acid sequence SEQ ID NO: 3 or 4; a coronavirus nucleocapsid (N) protein or an immunogenic fragment thereof of aminoacid sequence SEQ ID NO: 7 or 8; or a coronavirus envelope (E) protein or an immunogenic fragment thereof of amino acid sequence SEQ ID NO: 5 or 6.

According to some embodiments, the DNA vaccine encodes one or more conserved protein of a human immunodeficiency virus (HIV). According to some such embodiments, the conserved protein of the human immunodeficiency virus is an HIV conserved capsid protein (gag) of amino acid sequence SEQ ID NO: 9 or an immunogenic fragment thereof; an HIV conserved envelope protein (env) or an immunogenic fragment thereof of amino acid sequence SEQ ID NO: 10; or an immunogenic fragment thereof; an HIV conserved polymerase protein (pol) or an immunogenic fragment thereof of amino acid sequence SEQ ID NO: 11, or an immunogenic fragment thereof; or an HIV conserved protease protein (pro) or an immunogenic fragment thereof of amino acid sequence SEQ ID NO: 12. The method further comprises then administering a boosting composition, wherein the boosting composition comprises a subunit or protein vaccine comprising one or more conserved proteins of a human immunodeficiency virus. According to some such embodiments, the conserved protein of the human immunodeficiency virus is an HIV conserved capsid protein (gag) of amino acid sequence SEQ ID NO: 9 or an immunogenic fragment thereof; an HIV conserved envelope protein (env) or an immunogenic fragment thereof of amino acid sequence SEQ ID NO: 10; or an immunogenic fragment thereof; an HIV conserved polymerase protein (pol) or an immunogenic fragment thereof of amino acid sequence SEQ ID NO: 11, or an immunogenic fragment thereof; or an HIV conserved protease protein (pro) or an immunogenic fragment thereof of amino acid sequence SEQ ID NO: 12.

According to some embodiments, the present disclosure provides for use of a genetic vaccine to prime and a subunit or protein vaccine to boost.

The priming composition may comprise naked DNA or RNA encoding the sonsensus sequence of the antigen. According to some embodiments, such DNA or RNA for example may be in the form of a circular plasmid that is not capable of replicating in mammalian cells. Such plasmids can easily be produced in bacteria and manipulated using standard recombinant DNA techniques; show very little dissemination and transfection at distant sites following delivery; can result in high levels of expression; and can be readministered multiple times into mammals (including primates) without inducing an antibody response against itself [See Hobernik, D. Bros, M. Intl J. Molecular Sci. (2018) 19: 3605; Wolff, J A, Budker, V. Advances in Genetics (2005) 54: 3-20]. Any selectable marker should not be resistant to an antibiotic used clinically, so for example kanamycin resistance is preferred to ampicillin resistance. Antigen expression should be driven by a promoter which is active in mammalian cells, for instance the cytomegalovirus immediate early (CMV IE) promoter.

According to some embodiments of the present disclosure, administration of a priming naked DNA, naked RNA or genetic vector vaccine, intramuscularly, is followed by boosting with first and second boosting compositions, intranasally or by inhalation, the first and second boosting compositions being the same or different from one another, e.g., as exemplified below. Still further, boosting compositions may be employed without departing from some embodiments of the present disclosure.

Either of the boosting compositions may include an adjuvant or cytokine, such as alpha-interferon, gamma-interferon, platelet-derived growth factor (PDGF), granulocyte macrophage-colony stimulating factor (GM-CSF) granulocyte-colony stimulating factor (GCSF), tumor necrosis factor (TNF), epidermal growth factor (EGF), IL-1, IL-2, IL-4, IL-6, IL-8, IL-10 and IL-12, or encoding nucleic acid therefor.

Administration of the boosting composition is generally weeks or months after administration of the priming composition, preferably about 2-3 weeks or 4 weeks, or 8 weeks, or 16 weeks, or 20 weeks, or 24 weeks, or 28 weeks, or 32 weeks. According to some embodiment, the boosting composition is formulated for administration about 1 week, or 2 weeks, or 3 weeks, or 4 weeks, or 5 weeks, or 6 weeks, or 7 weeks, or 8 weeks, or 9 weeks, or 16 weeks, or 20 weeks, or 24 weeks, or 28 weeks, or 32 weeks after administration of the priming composition.

Regardless of the nature of the composition given, additional vaccine compositions may also accompany the immunogens of the present disclosure. Thus, for purposes of preventing or treating viral infection (e.g., prophylactic or therapeutic vaccine), compositions containing the immunogens disclosed herein may, in addition, contain other vaccine pharmaceuticals. The use of such compositions with multiple active ingredients is left to the discretion of the clinician.

### Subjects

According to some embodiments, a subject in need of treatment is a subject having or at risk of having an infection (e.g., a subject having or at risk of contracting a viral, bacterial, fungal or protozoal infection).

Viral infection during immunosuppression is a major complication in transplantation, rheumatologic, and other immune-deficient conditions (e.g., HIV). Accordingly, subjects that may be treated according to adoptive immunity-based embodiments of the present disclosure include immunocompromised subjects.

According to some embodiments, a “subject having an infection” is a subject that has been exposed to an infectious microorganism with acute or chronic detectable levels of the microorganism in his/her body or has signs and symptoms of the infectious microorganism. Methods of assessing and detecting infections in a subject are known by those of ordinary skill in the art. A “subject at risk of an infection” is a subject that may be expected to come in contact with an infectious microorganism. Examples of such subjects are medical workers or those traveling to parts of the world where the incidence of infection is high. According to some embodiments, the subject is at an elevated risk of an infection because the subject has one or more risk factors to have an infection. Examples of risk factors to have an infection include, for example, immunosuppression, immunocompromise, age, trauma, burns (e.g., thermal burns), surgery, foreign bodies, cancer, newborns especially newborns born prematurely. The degree of risk of an infection depends on the multitude and the severity or the magnitude of the risk factors that the subject has. Risk charts and prediction algorithms are available for assessing the risk of an infection in a subject based on the presence and severity of risk factors. Other methods of assessing the risk of an infection in a subject are known by those of ordinary skill in the art. According to some embodiments, the subject who is at an elevated risk of an infection may be an apparently healthy subject. An “apparently healthy subject” is a subject who has no signs or symptoms of disease.

According to some embodiments, factors other than age associated with the target population for vaccination are considered. These factors include, but are not limited to, comorbidities, geographic factors (including microbial endemicity), nutritional status, and iatrogenic immune suppression.

### Kits

To facilitate use of the methods and compositions of the disclosure, any of the vaccine components and/or compositions, e.g., virus in various formulations, etc., and additional components, such as, buffer, cells, culture medium, useful for packaging for experimental or therapeutic vaccine purposes, can be packaged in the form of a kit. Typically, the kit contains, in addition to the above components, additional materials which can include, e.g., instructions for performing the methods of the disclosure, packaging material, and a container.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited. It is also to be understood that throughout this disclosure where the singular is used, the plural may be inferred and vice versa and use of either is not to be considered limiting.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Generation of Consensus Sequences for Conserved Proteins

### Example 1A

By way of example only, the use of consensus sequences for conserved protein(s) can be attained through performing multiple sequence alignments from a prepared Pathogeneic Library of conserved sequences, and attaining consensus sequences identified thereof.

### Pathogen Library Preparation

According to some embodiments, the Pathogen Library will contain a reference genome or conserved protein sequence, and similar genomic or conserved protein sequences.

The preparation of a DNA Library containing the reference genome or protein sequence can be generated through methods known in the art.

For example, reference genomic/protein sequences can be found as described herein.

Briefly, databases such as the National Center of Biotechnology Information (NCBI) (found at https://www.ncbi.nlm.nih.gov/) specifically, for example, NCBI's GenBank, EMBL's Nucleotide Sequence Database, Universal Protein Resource (UniProt), and Protein Data Bank (PDB), can be searched for the reference genome of the selected pathogen, and then can be filtered to specify coding gene sequences for expressed conserved proteins. Once at the NCBI database, “Genome” will be selected under the all databases drop down box. The targeted pathogen's species name will be entered into the search field and submitted. The reference genome option will be selected. Next, Gene option will be chosen under the Related Topics header. The results will then be filtered to produce Protein-coding sequences. Then, a custom filter will be created to filter for Gene records associated with gene records associated with protein sequence, with variation information, with homology data, and/or with proteins calculated to contain conserved domains. Alternatively, from the Genome Overview table, Protein Details may be selected to display conserved expressed proteins. Selection of each desired record will generate the identification number or sequence to be used in multiple sequence alignment.

Similar genomic or protein sequences can be generated through methods known in the art. For example, selection of “Other Genomes” under the Related Topics header on the reference Genome record for the pathogen under investigation will produce other related genomes for various different strains, variants, groups, clades, serotypes, and subtypes. Alternatively genomic and protein sequences for various different strains, variants, groups, clades, serotypes, and subtypes of the pathogen species can be attained from the Genome Assembly and Annotation Report from the reference Genome Record through the selection of the Organism or the Protein record.

Alternatively, a search for similar conserved protein sequences can be generated through algorithms known in the art, such as FASTA (found at https://www.ebi.ac.uk/Tools/sss/fasta/), ClustalW (found at https://www.ebi.ac.uk/Tools/msa/clustalw2/), HMMER (found at http://hmmer.org/), MMseqs2 (found at https://github.com/soedinglab/mmseqs2), and Basic Local Alignment Search Tool (BLAST; found at https://blast.ncbi.nlm.nih.gov/Blast.cgi) can be utilized to generate similar genomic or protein sequences. BLAST performs and scores sequence alignment to find similar nucleotide or protein sequences for a given query sequence. The search will be executed against a large database (typically the nr database) and the algorithm will also quantify statistical significance of the scored output matches.

The search will maximize a score that quantifies similarity between sequences, given a particular scoring matrix and gap penalty. The widely used scoring matrices are known in the art and include Point Accepted Mutation (PAM) matrices and BLOcks SUbstitution Matrices (BLOSUM). These matrices will quantify similarities between all pairs of amino acids, such that pairs of the same or similar amino acids have high scores while pairs of dissimilar amino acids are associated with low scores. The gap penalty is for gaps (openings) and will be inserted into one of the aligned sequences to maximize the similarity score. The gap penalty is larger for a new gap when compared to an extension of an already existing gap. BLAST can be parametrized to use different scoring matrices and gap penalties, resulting in different alignments for the same sequences. For example, parameters can restrict the maximal number of aligned (similar) target sequences and can set the maximal E-value. The E-value will quantify statistical significance of the similarity where lower value corresponds to more significant similarity. Parameter filters also include finding word matches between the query and database sequences where the use of smaller word sizes makes the search more sensitive. The “maximal matches in a query range” parameter limits the number of matches.

Once submitted, the search will produce similar proteins, the identification of conserved proteins can be filtered by selecting a limited identify match indicating the percent the found similar sequence matches with the reference sequence, for example by 50-100%, 70-100%, 80-100%, or 90-100%. The Pathogen Library can then be prepared by selecting the desired sequences or identification number, in plain text or in FASTA format.

### Sequence Alignment

A library containing the sequences for the genome/protein reference and similar sequences thereof, for example as shown in Table 1A and Table 1B, above, will be aligned.

As described herein, there are multiple publicly available tools that align multiple sequences. ClustalOmega will be used to create multiple sequence alignments (MSAs). This is a procedure for aligning more than two homologous nucleotide or amino acid sequences together such that the homologous residues from the different sequences line up as much as possible in columns. Sequence alignment can be of two types i.e., comparing two (pair-wise) or more sequences (multiple) for a series of characters or patterns. The sequences will be aligned step-wise (first two sequences then one by one) by the program. When a sequence is aligned to a group or when there is alignment in between the two groups of sequences, the alignment is performed that had the highest alignment score. The gap symbols in the alignment will be replaced with a neutral character, such as an asterisk.

### Consensus Sequence Identification

Consensus sequence(s) will be identified by analyzing the data and recognizing regions that are largely or entirely uniform across all of the sequences. The most conserved consensus sequences will be selected for epitope prediction.

By way of example only, consensus sequences for conserved protein(s) in the Retroviridae virus family, for example, the human immunodeficiency virus, are known in the prior art and can be found by researching databases such as GenBank®, DNA DataBank of Japan (DDBJ), or the European Nucleotide Archive (ENA) for the virus and resolving for consensus sequences from the sequences provided for each virus variant therein. For example, the genome for Human Immunodeficiency Virus and serotypes thereof have been mapped and submitted to GenBank. A search of the genome database of the National Center for Biotechnology Information (NCBI) found at https://www.ncbi.nlm.nih.gov/genome, provides representative genome information for example, for HIV-1. The main page provides the record of each mRNA, and protein sequence encoded by the genome, and significantly, recorded will be the conserved protein sequences such as those listed in Table 1A and Table 1B above.

Selection of the list for all reference or representative genomes for the species (499 at the time of this submission) or the Genome Assembly and Annotation Report displays all genome variants and a list of either a GenBank Accession Number or RefSeq Accession Number for each genome variant (Assembly column).

Selection of a first GenBank Accession Number will display the record of each mRNA, and protein sequence encoded by the first genome variant. Selection of a second GenBank Accession Number will display the record of each mRNA, and protein sequence encoded by the second genome variant. Selection of the entire genome variant or protein sequence variant can be repeated until all or all desired variants are found.

Next sequence alignment of the genome or protein sequence variants can be found by inputting all variants into any publicly available software that contains sequence alignment database with the ability to align multiple sequences, such as ClustalW2 found at https://www.ebi.ac.uk/Tools/msa/clustalw2/. Importation in the sequences with the selection of DNA as the sequence type will produce the alignment of the sequence that displays any homology across the variants with an asterisk.

Next, identification of regions that are largely or entirely uniform across all of the variants will provide consensus sequence identity. If any single nucleotide polymorphisms exist, then point mutations can be replaced with appropriate IUB Wobble base code which can be retrieved from https://www.bioinformatics.org/sms/iupac.html.

Alternatively, tools such as Galaxy may be used, found at usegalaxy.org, and upon data download and selection of appropriate filters, consensus sequences may be attained.

In another alternative, virus specific sequence databases exist, for example, for HIV-1 which can be retrieved from https://www.hiv.lanl.gov/content/sequence/HIV/CONSENSUS/Consensus.html. Consensus sequences can be attained by the selection of the appropriate download format, computer type, region, and aligned proteins options. In this case, the input alignments are the HIV Sequence Database Web Alignments retrieved from https://www.hiv.lanl.gov/content/sequence/HIV/mainpage.html. These sequences have undergone additional annotation after retrieval. Specifically, question marks in consensus sequences have been resolved, and glycosylation sites have been aligned. From the input, consensus sequences were built using the consensus website. The consensus sequences were calculated according to the default values on the consensus website except that they were computed for all subtype groups having 3 or more (rather than 4 or more) sequences in the alignment. If a column in a subtype group contained equal numbers of two different letters, the tie was resolved by looking at the same column throughout the M group and using the most common letter as the consensus. An upper case letter in a DNA consensus sequence indicates that the nucleotide is preserved unanimously in that position in all sequences used to make the consensus. In cases of nonunanimity the most common nucleotide is shown in lowercase. Regions spanned by multiple insertions and deletions are difficult to align. Accordingly, alignments are anchored in such regions on glycosylation sites, and to preserve the minimal elements which span such regions. Protein consensus sequences are always upper case letters indicating most common amino acid at that position.

Alternatively, BLASTP, which is a generic purpose protein sequence alignment program that compares a query protein sequence to sequences in a specific protein database, will be searched for consensus sequences similar to the selected conserved proteins. In the “Enter Query Sequence” box, the selected protein reference sequence(s) will be inputted (either directly in the text box or uploaded via a file). The sequence(s) will be provided either in the FASTA format or the query protein will be identified with either accession number or NCBI gi number as found in the NCBI databases. In the “Choose Search Set” box the target organism (pathogen) will be selected (databases currently available include RefSeq, PDB, and SWISS-PROT). The “Program Selection” box will be ensured to select BLASTP. The BLAST parameters will be set to the pre-selected default parameters. The default value for the maximal number of aligned target sequences will be used (value=100). The default E-value is 10, which means that about 10 of the similar sequences are expected to be found by chance. The default word size for proteins will be used (value=6). The “maximal matches in a query range” parameter will be set to the default value of 0, which means that there is no limit. The default scoring matrix and default set up of gap penalties will be used as BLOSUM62 and 11 for opening the gap and 1 for the extension of the gap. Once submitted, similar sequences or identification numbers for conserved protein(s) will be searched and generated, upon which the DNA library will be prepared.

### Sequence Alignment

Sequences to be aligned will be inputted either directly into the text box or uploaded as a file. Sequences will either be inputted in plain text or in FASTA format. ClustalW2 will be run and a graphical representation of regions that have uniformity across the sequences will be produced.

### Consensus Sequence Identification

Consensus sequence(s) will be identified by analyzing the data and recognizing regions that are largely or entirely uniform across all of the sequences. The most conserved consensus sequences will be selected for epitope prediction.

### Example 2: Epitope Prediction

Identifying which peptides are able to induce CTLs is integral for the development of vaccines and immunotherapy directed against infectious pathogens. T cell epitopes for pathogens can be predicted through the use of a number of publicly available databases. Web accessible databases for major histocompatibility complex-binding and/or epitope data include the following:

| | |
|---|---|
| AntiJen | http://wwwjenner.ac.uk/antijen |
| EPIMHC | http://immunax.dfci.harvard.edu/epimhc/ |
| HCV immunology | http://hcv.lanl.gov |
| HIV immunology | http://www.hiv.lanl.gov |
| IEDB | http://www.immuneepitope.org/ |
| MHCBN | http://www.imtech.res.in/raghava/mhcbn |
| SYFPEITHI | http://www.syfpeithi.deImmune |

### MHC Class I Binding Prediction Generation

To perform class I binding predictions, the at least one consensus sequence(s) for a given conserved protein will be inputted (sequences will be inputted in as plain text, separating the sequences with blanks, in FASTA format, or by specifying a file containing the proteins) into IEDB. Consensus method(s) of search will be chosen. MHC species as a human will be specified with the selected option of comprising multiple alleles and epitope lengths (e.g., 9, 10, 11, 12, 13, 14).

As the majority of HLA class I molecules have a preference for peptides of length 9 amino acids, the majority of binding affinities have been measured using 9mer peptides. For this reason, it has been difficult to develop reliable prediction systems for lengths other than 9, which is certainly needed because a significant part of the binding peptides have lengths of 8, 10 and 11 amino acids, and some are even longer. However, prediction systems trained on 9mer data can actually be used to fairly accurately predict the binding affinities of 8-, 10-, and 11mer peptides. This method is used in the web-accessible version of NetMHC (services.healthtech.dtu.dk/).

### Binding Affinity Threshold Calibration

To ensure the quality of the data for binding affinity, searches will be calibrated to predict peptides bind with an IC50 value less than 500 nM, an established threshold associated with immunogenicity for 80-90% of all epitopes.

Once the search is submitted, the protein sequence will be parsed into all possible peptides for the specified length and the predicted binding affinity for each will be calculated. The tool will compare the predicted affinity to that of a large set of randomly selected peptides and assigns a percentile rank (lower percentile rank corresponds to higher binding affinity). The prediction tool will produce a table of results including columns for the allele, peptide start and end positions, the peptide length, the peptide sequence, the method(s) used, and the percentile rank. Results will be presented by default sorted by predicted percentile rank, but results can also be sorted by sequence position.

### Competition-Based Peptide—HLA-Binding Assays

The binding affinity of test peptides to selected HLA class-I molecules will be assessed in competition-based cellular binding assays as previously published (Kessler et al., Curr Protoc Immunol 2004; Chapter 18:Unit 18.12; Kessler et al., Hum Immunol 2003; 64:245-55). These assays are based on the HLA class-I binding competition of a known high-affinity fluorescein-labeled reference peptide and the test peptide of interest. In brief, cells of a B-LCL with the desired HLA expression will be stripped from naturally bound peptides by citric acid buffer treatment with specific pH (pH 3.1 for HLA-A*02:01, HLA-A*11:01, HLA-A*24:02, and HLA-B*07:02; and pH 2.9 for HLA-A*03:01 and HLA-B*15:01). The cells will be suspended at a concentration of 4×105 cells/mL in the B-LCL medium containing 2 μg/mL B2-microglobulin (MP Biomedicals) to reconstitute the HLA class-I complex. The cells will be transferred to a 96-well plate, and a mixture of 150 nmol/L fluorescein-labeled reference peptide and serially diluted test peptide will be added. Each test peptide will be analyzed at eight different concentrations, ranging from 100 μmol/L to 0.78 μmol/L, in a minimum of three independent experiments for binders and a minimum of two for nonbinders. Fluorescence will be measured by flow cytometry (FACS Canto II or FACS Accuri; BD Biosciences) and interpreted with FlowJo V10 (FlowJo, LLC). Background and maximum fluorescence will be determined based on cells without peptide and cells with fluorescein-labeled reference peptide only, respectively. For every test peptide concentration, the mean percentage of reference peptide inhibition will be calculated relative to the maximum fluorescence. The test peptide concentration that inhibits 50% binding of the fluorescein-labeled reference peptide will be determined by nonlinear regression analysis based on the following equation (formula A; SigmaPlot V13.0, Systat Software). This half-maximal inhibitory concentration (IC50) indicates the binding affinity.

### Epitope Selection

The top 1% of peptides will be selected for each allele/length combination.

### Example 3. In Vivo Testing—Seroconversion

Prior to human administration a test for potency will evaluate the specific ability of the vaccine in an in vitro or in vivo test to effect a given immune response. The in vivo immunogenicity will be determined by dosing groups of 10 mice with various doses of the protein antigen. Sera will be analyzed for the presence of IgG antibodies using an ELISA. The criterion for passing will be based upon the number of vaccine treated animals that are seropositive compared to a reference standard.

Initial experiments will be performed in a transgenic mouse model that expresses the hACE2 gene under the control of the human cytokeratin 18 promoter. In certain embodiments, a prime-boost regimen will be used. Mice (K18-hACE2Prlmn/J, Jax #034860; available from Jackson Laboratories) will be immunized by priming the fully human immune system with the DNA vector, and then boosting the fully human immune system by immunizing with the AdV vector or AAV vector followed by the VV vector, or the VV vector followed by the AdV vector or AAV vector, with prime at day 0, and a boost at day 5-7, day 14 and/or day 28. Secondary endpoints will be to compare the kinetics of neutralizing vs. antibody titers and to assess the Th1/Th2 profile of the specific immune response. Proliferation and IFN-γ and IL-4 production by splenic T cell against the immunogenic composition will be assessed.

Peripheral blood and spleen cells will be collected at determined time points. Neutralizing and immunogen-specific antibody titers and isotypes will be determined by inhibition of SARS-CoV infection of Vero cells and by ELISA, respectively. Proliferation of splenic cells will be determined by ³[H]-thymidine uptake. Frequencies of splenic IFN-γ and IL-4 producing CD4+ T lymphocytes, will be determined by ELISPOT and FACS analysis.

### Example 4. Use of K18-hACE2 Mice as a Model of SARS-CoV2 Infection for Evaluation of Vaccine Candidates

The transgenic mouse model that expresses the hACE2 gene under the control of the human cytokeratin 18 promoter will be used to test the efficacy of vaccine candidates as described by Moreau, G B et al. Am. J. Trop. Med. Hyg. (2020) 103 (3): 1215-19. In brief, For testing each vaccine candidate, mice (K18-hACE2Prlmn/J, Jax #034860; available from Jackson Laboratories) will be infected with median tissue culture infected dose (TCID50) of 10⁴ plaque-forming units (PFUs) of SARSCoV-2. Vaccine candidates will be administered by the intranasal route and/or intramuscularly in groups of 5 mice. In certain embodiments, a prime-boost regimen will be use. Mice will be immunized by priming with the DNA vector, and then boosting by immunizing with the AdV vector or AAV vector followed by the VV vector, or the VV vector followed by the AdV vector or AAV vector, with prime at day 0, and a boost at day 5-7. Five mock-infected mice will receive 50 μl DMEM. Mice will be followed twice daily for clinical symptoms until day 5. Categories included in clinical scoring will include weight loss; posture and appearance of fur (piloerection), activity; eye closure, and respiratory rate.

Blood samples will be collected by standard procedures. Neutralizing and immunogen-specific antibody titers and isotypes produced by vaccinated mice in serum will be determined by measuring inhibition of SARS-CoV infection of Vero cells and by ELISA, respectively.

For histology, the tissues of euthanized mice will be fixed in formaldehyde. Histopathological scoring for lung tissue will be performed according to the guidelines of the American Thoracic Society. Statistical significance will be determined by standard methods.

Viral titers will be determined by homogenizing the left lobe of the lung in 1 mL serum-free DMEM with a disposable tissue grinder and plaque assays performed. In brief, Vero cells grown in DMEM with fetal bovine serum will be seeded into multiwall plates at a concentration of 2×10⁵ cells/well the night before the assay. Serial dilutions will be added to the wells. The plate will be incubated at 37 C, 5% CO2 for 2 hr, shaking the plates every 15 minutes. After 2 hr the plate media will be replaced with a liquid overlay of DMEM, 2.5% FBS containing 1.2% Avicel PH-101 (Sigma-Aldrich, St. Louis, Mo.) and incubated at 37 C, 5% CO2. After 3 days, the overlay will be removed, wells will be fixed with 10% formaldehyde and stained with 0.1% crystal violet to visualize plaques. Plaques will be counted, and PFUs calculated according to the following equation: average # plaques/dilution factor×volume diluted virus added to the well.

### Example 5. Use of NSG Mice Reconstituted with Human Immune System Components for Evaluation of Vaccine Candidates

NSG (NOD-scid 11.2 Rγnull) mice (from The Jackson Laboratory, jax.org/jax-mice-and-services/find-and-order-jax-mice/nsg-portfolio) will be engrafted with human PBMC as follows. Fresh whole blood from healthy adult donors collected with preservative free heparin will be diluted (1:3) with low endotoxin PBS (PBSle) (Biochrom) and the leukocyte fraction enriched using standard ficoll gradient centrifugation. The interface will be harvested and washed twice with PBSle. For a 9 week reconstitution protocol, mice will be irradiated with a sub-lethal dose of 100 cGy one day before intravenous injection of 1×10⁶ human PBMCs; a 4-week protocol will use a single intravenous injection of 10×10⁶ PBMC, without irradiation.

Mice will be vaccinated first on day 42 or day 14 after reconstitution, respectively as follows. The mice comprising the fully human functional immune system will be immunized by priming the fully human immune system with the DNA vector, and then boosting the fully human immune system by immunizing with the AdV vector or AAV vector followed by the VV vector, or the VV vector followed by the AdV vector or AAV vector.

### Example 6. Evaluation of Immune Response and Selective Expansion of Immune Cell Subtypes

The quality of the immune response attained in the reconstituted NSG mice in Example 5 will be assessed, followed by selective expansion of CTL cell subsets.

Briefly, PBMCs, splenocytes, or bone marrow cells of human or murine origins will be isolated and stained for 1 h at 4° C. in the dark with the appropriate antibody cocktail. Following washing (1% (v/v) FBS in PBS), cells will be fixed with fixation buffer (1% (v/v) FBS, 4% (w/v) PFA in PBS) for 30 min at 4° C. in the dark. Flowcytometric analysis will be performed, and flow cytometry data will be analyzed using FlowJo software (TreeStar, Ashland, Oreg.). Chimerism of all humanized mice model will be assessed prior to each experiment by quantifying the following human populations: Human CD45+, human CD45+ murine CD45−; T-cells, CD45+ CD3+; CD4+ T cells, CD45+ CD3+ CD4+; CD8+ T cells, CD45+ CD3+ CD8+; CD45+ CD16+ leukocytes; B-cells, CD45+ CD19; conventional dendritic cells, CD45+ CD11c+; NK/NKT cells, CD45+ CD56+; Monocytes, CD45+ CD14+. Mouse immune cell subsets will be gated as followed: Murine CD45+, Human CD45− Murine CD45+; Conventional dendritic cells, CD45+ CD3− CD19− NK1.1− TER119− Ly-6G/Gr1− CD11c+; Plasmacytoid dendritic cells, CD45+ CD3− CD19−NK1.1− TER119− Ly-6G/Gr1− CD317+; Monocytes, CD45+ CD3− CD19− NK1.1− TER119− Ly-6G/Gr1− CD11b+ CD11c− F4/80−; Macrophages, CD45+ CD3− CD19− NK1.1− TER119− Ly-6G/Gr1− CD11b+F4/80+. Human immune cell subsets will be gated as follows: Human CD45+, human CD45+ murine CD45−; T-cells, CD45+ CD3+; CD4+ T cells, CD45+ CD3+ CD4+; CD8+ T cells, CD45+ CD3+ CD8+; Myeloid cells, CD45+ CD3− CD19− (CD56+) CD33+; Granulocytes, CD45+ CD66b+; B cells, CD45+ CD3− CD19+; Natural Killer cells, CD45+ CD3− (CD19−) CD56+; Natural Killer T cells and γδ T cells, CD45+ CD3+(CD19−) CD56+; Conventional dendritic cells, CD45+ CD3− CD19− (CD56−) (CD33+) CD11c+ (BDCA1/3+); CD45+ CD3− CD19 CD123+, group composed of monocytes, plasmacytoid dendritic cells, basophils and myeloid precursors; Plasmacytoid dendritic cells, CD45+ CD3− CD19− (CD56−) BDCA-2+ CD123+; Monocytes, CD45+ CD3− CD19− (CD56−) CD14+; Macrophages, CD45+CD3− CD19− (CD56−) CD68+.

Flow cytometry fluorophor compensation for antibodies will be performed using AbC™ Anti-Mouse Bead Kit (Life Technologies, Invitrogen, Foster City, Calif., USA). Counting beads will be added to each sample prior to flow-cytometry analysis (AccuCheck Counting Beads, Life Technologies, Invitrogen, Foster City, Calif., USA).

The frequency of each cell fraction will be shown as a percentage of CD45+ cells, with the exception of CD4+ and CD8+ T cells, which will be shown as a percentage of CD3+ T cells. The frequencies of important myeloid subsets (CD14+ monocytes and CD11c+ dendritic cells) and CD56+NK cells will also be determined.

### IFN-γ ELISpot Assay

An exemplary ELISPOT assay protocol is as follows. Enzyme-linked immunosorbent spot (ELISpot) assays are conducted using mouse IFN-γ ELISpot kit (BD Bioscience, Cat #551083). Control or vaccinated animals are sacrificed and bronchoalveolar lavage cells and splenocytes were isolated. 2×10⁵ splenocytes are plated in triplicate in 96-well plates pre-coated with 5 μg/ml of purified anti-mouse IFN-γ and subsequently stimulated with a peptide specific for a viral immunogen at a final 5 μg/ml concentration. After 24 hours of stimulation, the cells are washed with deionized water and exposed to 100 μl biotinylated anti-mouse IFN-γ (2 μg/ml) for 2 hours at room temperature, followed by extensive washing prior to the addition of 100 μl Streptavidin-HRP. After 1 hour incubation at room temperature, the cells are washed and 100 μl of substrate solution is added to develop spots. The reaction is stopped with water and the number of spot-forming cells (SFCs) is determined using an automated ELISPOT software (Saizhi, Beijing, China).

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

### REFERENCES

[1] Russell R J, Kerry P S, Stevens D J, Steinhauer D A, Martin S R, Gamblin S J, Skehel J J. Structure of influenza hemagglutinin in complex with an inhibitor of membrane fusion. Proc Natl Acad Sci US A. 2008; 105(46): 17736-17741.
[2] Gostin L O, Phelan A, Stoto M A, Kraemer J D, Reddy K S. Virus sharing, genetic sequencing, and global health security. Science. 2014; 345(6202): 1295-1296.
[3] Horimoto T, Kawaoka Y. Influenza: lessons from past pandemics, warnings from current incidents. Nat Rev Microbiol. 2005; 3(8): 591-601.
[4] Johnson N P, Mueller J. Updating the accounts: globalmortality of the 1918-1920 “Spanish” influenza pandemic. Bull Hist Med. 2002; 76(1): 105-115
[5] Gao R, Gao B, Hu Y, Feng Z, Wang D, Hu W, et al. Human infection with a novel avian-origin influenza A (H7N9) virus. N Engl J Med. 2013; 368(20): 1888-1897.
[6] Houser K, Subbarao K. Influenza Vaccines: Challenges and Solutions. Cell Host Microbe. 2015; 17(3): 295-300.
[7] Hannoun C. The evolving history of influenza viruses and influenza vaccines. Expert Rev Vaccines. 2013; 12(9): 1085-1094.
[8] Wang Z, Wan Y, Qiu C, Quinones-Parra S, Zhu Z, Loh L, et al. Recovery from severe H7N9 disease is associated with diverse response mechanisms dominated by CDS+ T cells. Nat Commun. 2015; 6: 6833.
[9] Sridhar S, Begom S, Bermingham A, Hoschler K, Adamson W,[s} ] Carman W, et al. Cellular immune correlates of protection against symptomatic pandemic influenza. Nat Med. 2013; 19(10): 1305-12.
[10] Fan J, Liang X, Horton M S, Perry H C, Citron M P, Heidecker G J, et al. Preclinical study of influenza virus A M2 peptide conjugate vaccines in mice, ferrets, and rhesus monkeys. Vaccine. 2004; 22(23-24): 2993-3003.
[11] Valkenburg S A, Li O T, Mak P W, Mok C K, Nicholls J M, Guan Y, et al. IL-15 adjuvanted multivalent vaccinia-based universal influenza vaccine requires CD4+ T cells for heterosubtypic protection. Proc Natl Acad Sci US A. 2014; 111(15): 5676-81.
[12] Lamere M W, Moquin A, Lee F E, Misra R S, Blair P J, Haynes L, et al. Regulation of antinucleoprotein IgG by systemic vaccination and its effect on influenza virus clearance. J Virol. 2011; 85(10): 5027-5035.
[13] Pica N, Palese P. Toward a universal influenza virus vaccine: prospects and challenges. Annu Rev Med. 2013; 64: 189-202.
[14] Uddback I E, Steffensen M A, Pedersen S R, Nazerai L, Thomsen A R, Christensen J P. PB1 as a potential target for increasing the breadth of T-cell mediated immunity to Influenza A. Sci Rep. 2016; 6: 35033.
[15] Cox A, Dewhurst S. A Single Mutation at PB1 Residue 319 Dramatically Increases the Safety of PR8 Live Attenuated Influenza Vaccine in a Murine Model without Compromising Vaccine Efficacy. J Virol. 2015; 90(5): 2702-5.
[16] Jaiswal V, Chanumolu S K, Sharma P, Chauhan R S, Rout C. EpiCombFlu: exploring known influenza epitopes and their combination to design a universal influenza vaccine. Bioinformatics. 2013; 29(15):1904-7.
[17] Brown L E, Kelso A. Prospects for an influenza vaccine that induces cross-protective cytotoxic T lymphocytes. Immunol Cell Biol. 2009; 87(4):300-8.
[18] Holloway R, Rasmussen S A, Zaza S, Cox N J, Jermigan D B. Updated preparedness and response framework for influenza pandemics. MMWR Recomm Rep. 2014; 63(RR-06): 1-18.
[19] Draper S J, Heeney J L. Viruses as vaccine vectors for infectious diseases and cancer. Nat Rev Microbiol. 2010; 8(1): 62-73.
[20] Stanekova Z, Varecykova E. Conserved epitopes of influenza A virus inducing protective immunity and their prospects for universal vaccine development. Viral J. 2010; 7: 351.
[21] Wang X, Xing M, Zhang C, Yang Y, Chi Y D, Tang X Y, et al. Neutralizing antibody responses to enterovirus and adenovirus in healthy adults in China. Emerg Microbes Infec. 2014; 3(5): e30.
[22] Xing M, Wang X, Chi Y, Zhou, D. Gene therapy for colorectal cancer usmg adenovirus-mediated full-length antibody, cetuximab. Oncotarget. 2016; 7(19): 28262-72.
[23] Zhang S, Huang W, Zhou X, Zhao Q, Wang Q, Jia B. Seroprevalence of neutralizing antibodies to human adenoviruses type-5 and type-26 and chimpanzee adenovirus type-68 in healthy Chinese adults. J Med Viral. 2013; 85(6): 1077-84.
[24] Barouch D H, Alter G, Broge T, Linde C, Ackerman M E, Brown E P, et al. Protective Efficacy of Adenovirus/Protein Vaccines Against SIV Challenges in Rhesus Monkeys. Science. 2015; 349(6245): 320-324.
[25] Stanley D A, Honko A N, Asiedu C, Trefry J C, Lau-Kilby A W, Johnson J C, et al. Chimpanzee adenovirus vaccine generates acute and durable protective immunity against ebolavirus challenge. Nat Med. 2014; 20(10): 1126-9.
[26] Huang X, Xu J, Qiu C, Ren L, Liu L, Wan Y, et al. Mucosa! priming with PEI/DNA complex and systemic boosting with recombinant TianTan vaccinia stimulate vigorous mucosal and systemic immune responses. Vaccine. 2007; 25(14): 2620-9
[27] Huang X, Liu L, Ren L, Qiu C, Wan Y, Xu J. Mucosal priming with replicative Tiantan vaccinia and systemic boosting with DNA vaccine raised strong mucosa! and systemic HIV-specific immune responses. Vaccine. 2007; 25(52): 8874-84.
[28] Singh H, Raghava G P. ProPredl: prediction of promiscuous MEW Class-I binding sites. Bioinformatics. 2003; 19: 1009-1014.
[29] Moutaftsi M, Peters B, Pasquetto V, Tscharke D C, Sidney J, Bui H H, et al. A consensus epitope prediction approach identifies the breadth of murine T(CD8+)-cell responses to vaccinia virus. Nat Biotechnol. 2006; 24: 817-819.
[30] Keskin D B, Reinhold B B, Zhang G L, Ivanov A R, Karger B L, Reinherz E L. Physical detection of influenza A epitopes identifies a stealth subset on human lung epithelium evading natural CD8 immunity. Proc Natl Acad Sci US A. 2015; 112(7): 2151-2156.
[31] Halstead E S, Mueller Y M, Altman J D, Katsikis P D. In vivo stimulation of CD137 broadens primary antiviral CD8+ T cell responses. Nat Immunol. 2002; 3(6): 536-41.
[32] Lawrence C W, Ream R M, Braciale T J. Frequency, specificity, and sites of expansion of CD8+ T cells during primary pulmonary influenza virus infection. J Immunol. 2005; 174(9): 5332-40.
[33] Turner S J, La Gruta N L, Stambas J, Diaz G, Doherty P C. Differential tumor necrosis factor receptor 2-mediated editing of virus-specific CD8+ effector T cells. Proc Natl Acad Sci US A. 2004; 101(10): 3545-3550.
[34] Tussey L G, Rowland-Jones S, Zheng T S, Androlewicz M J, Cresswell P, Frelinger J A, et al. Different MHC class I alleles compete for presentation of overlapping viral epitopes. Immunity. 1995; 3(1): 65-77.
[35] Belz G T, Xie W, Doherty P C. Diversity of Epitope and Cytokine Profiles for Primary and Secondary Influenza A Virus-Specific CD8+ T Cell Responses. J Immunol. 2001; 166(7): 74627-4633.
[36] Chen W, Norbury C C, Cho Y, Yewdell J W, Bennink J R. Immunoproteasomes shape immunodominance hierarchies of antiviral CD8(+) T cells at the levels of T cell repertoire and presentation of viral antigens. J Exp Med. 2001; 193(11): 1319-26.
[37] Zhu Z, Yang Y, Feng Y, Shi B, Chen L, Zheng Y, et al. Infection of inbred BALB/c and C57B L/6 and outbred Institute of Cancer Research mice with the emerging H7N9 avian influenza virus. Emerg Microbes Infect. 2013; 2(8): e50.
[38] Masopust D, Choo D, Vezys V, Wherry E J, Duraiswamy J, Akondy R, et al. Dynamic T cell migration program provides resident memory within intestinal epithelium. J Exp Med. 201'0; 207(3): 553-64 [39] Steel J, Lowen A C, Wang T T, Yondola M, Gao Q, Haye K, et al. Influenza virus vaccine based on the conserved hemagglutinin stalk domain. mBio. 2010; 1(1).
[40] Grant E, Wu C, Chan K F, Eckle S, Bharadwaj M, Zou Q M, et al. Nucleoprotein of influenza A virus is a major target of immunodominant CD8+ T-cell responses. Immunol Cell Biol. 2013; 91(2): 184-94.
[41] Saletti G, Gerlach T, Rimmelzwaan G F. Influenza vaccines: ‘tailor-made’ or ‘one fits all’. 2018; 53: 102-110.
[42] Clemens E B, van de Sandt C, Wong S S, Wakim L M, Valkenburg S A. Harnessing the Power of T Cells: The Promising Hope for a Universal Influenza Vaccine. Vaccines (Basel). 2018; 6(2).
[43] Wu T, Hu Y, Lee Y T, Bouchard K R, Benechet A, Khanna K, et al. Lung-resident memory CD8 T cells (TRM) are indispensable for optimal cross-protection against pulmonary virus infection. J Leukoc Biol. 2014; 95(2):215-24.
[44] Zens K D, Chen J K, Farber D L. Vaccine-generated lung tissue-resident memory T cells provide heterosubtypic protection to influenza infection. JCI Insight. 2016; 1(10).
[45] Ariotti S, Hogenbirk M A, Dijkgraaf F E, Visser L L, Hoekstra M E, Song J Y, et al. T cell memory. Skin-resident memory CD8+ T cells trigger a state of tissue-wide pathogen alert. Science. 2014; 346(6205): 101-5.
[46] Schenkel J M, Fraser K A, Vezys V, Masopust D. Sensing and alarm function of resident memory CDS+ T cells. Nat Immunol. 2013; 14(5): 509-13.
[47] Sliitter B, Van Braeckel-Budimir N, Abboud G, Varga S M, Salek-Ardakani S, Harty J T. Dynamics of influenza-induced lung-resident memory T cells underlie waning heterosubtypic immunity. Sci Immunol. 2017; 2(7).
[48] Pizzolla A, Nguyen T H, Sant S, Jaffar J, Loudovaris T, Mannering S I, et al. Influenza-specific lung-resident memory T cells are proliferative and polyfunctional and maintain diverse TCR profiles. J Clin Invest. 2018; 128(2): 721-733.

What is claimed is:

1. A universal vaccine against an immunogen of an infectious pathogenic virus selected from a human Coronaviridae and a human Retroviridae virus comprising a pharmaceutical composition containing
a. at least one ribonucleic acid (RNA) polynucleotide comprising an open reading frame encoding at least one polypeptide antigen or an immunogenic fragment thereof, wherein the polypeptide antigen, or the immunogenic fragment thereof, comprises a conserved internal protein that is enriched in T cell recognition antigens,
b. optionally helper T cell (Th) epitopes comprising
i. at least one full-length protein that is immunogenic; or
ii. overlapping peptides of at least 30 amino acids in length that together cover the conserved internal viral protein
c. an optional immune response enhancer; and
d. a pharmaceutically acceptable carrier
wherein a cytotoxic T lymphocyte (CTL) epitope consists of peptides of about 7 to about 14 residues in length.

2. The universal vaccine according to claim 1, wherein the Coronaviridae virus is a human coronavirus.

3. The universal vaccine according to claim 2, the optional helper T cell (Th) epitopes comprise
(a) at least one full-length protein of a human coronavirus selected from an S protein, an M protein, an E protein, or an N protein, wherein the full length protein is immunogenic; or
(b) overlapping peptides of at least 30 amino acids in length that together cover the coronavirus S protein.

4. The universal vaccine according to claim 2, wherein the immunogen contains at least one conserved protein of a human coronavirus, wherein
a. the conserved protein is a coronavirus spike (S) protein of amino acid sequence SEQ ID NO: 1 or an immunogenic fragment thereof; or
b. the conserved protein is a coronavirus spike (S) protein of amino acid sequence SEQ ID NO: 1 or an immunogenic fragment thereof of an amino acid sequence at least 85% identical to SEQ ID NO: 1; or
c. the conserved protein is an isolated coronavirus S protein 51 subunit or an immunogenic fragment thereof of amino acid sequence SEQ ID NO: 1; or
d. the conserved protein is an isolated coronavirus S protein 51 subunit cleaved by a transmembrane serine protease (TMPRSS2) comprising a receptor binding domain (RBD) or an immunogenic fragment thereof; or
e. the conserved protein is an isolated coronavirus S protein 51 subunit cleaved by a transmembrane serine protease (TMPRSS2) comprising an RBD domain of the isolated 51 subunit enzymatically stripped of at least 5% of its covering of N-linked glycans, O-linked glycans or both by limited digestion; or
f. the conserved protein is an isolated coronavirus S protein S2 subunit or an immunogenic fragment thereof; or
g. the conserved protein is an isolated coronavirus S protein S2 subunit enzymatically stripped of at least 5% of its covering of N-linked glycans by limited digestion, or
h. the conserved protein is a coronavirus membrane (M) protein or an immunogenic fragment thereof of amino acid sequence SEQ ID NO: 3 or 4; or
i. the conserved protein is a coronavirus membrane (M) protein or an immunogenic fragment thereof; of an amino acid sequence at least 85% identical to SEQ ID NO: 3 or 4; or
j. the conserved protein is a coronavirus envelope (E) protein or an immunogenic fragment thereof of amino acid sequence SEQ ID NO: 5 or 6; or
k. the conserved protein is a coronavirus envelope (E) protein or an immunogenic fragment thereof of an amino acid sequence at least 85% identical to SEQ ID NO: 5 or SEQ ID NO: 6; or
l. the conserved protein is a coronavirus nucleocapsid (N) protein or an immunogenic fragment thereof of amino acid sequence SEQ ID NO: 7 or 8; or
m. the conserved protein is a coronavirus nucleocapsid (N) protein or an immunogenic fragment thereof of an amino acid sequence at least 85% identical to SEQ ID NO: 7 or SEQ ID NO: 8, or
n. the conserved protein or immunogenic fragment is a combination thereof.

5. The universal vaccine according to claim 1, wherein the Retroviridae virus is a human immunodeficiency virus (HIV).

6. The universal vaccine according to claim 5, wherein the immunogen contains at least one conserved protein of a human immunodeficiency virus (HIV), wherein
a. the conserved protein is an HIV conserved capsid protein (gag), or an immunogenic fragment thereof of amino acid sequence SEQ ID NO: 9; or
b. the conserved protein is an HIV conserved capsid protein (gag), or an immunogenic fragment thereof of an amino acid sequence at least 85% identical to SEQ ID NO: 9, or
c. the conserved protein is an HIV conserved envelope protein (env) or an immunogenic fragment thereof of amino acid sequence SEQ ID NO: 10; or
d. the conserved protein is an HIV conserved envelope protein (env) or an immunogenic fragment thereof of an amino acid sequence at least 85% identical to SEQ ID NO: 10, or
e. the conserved protein is an HIV conserved polymerase protein (pol) or an immunogenic fragment thereof of amino acid sequence SEQ ID NO: 11; or
f. the conserved protein is an HIV conserved polymerase protein (pol) or an immunogenic fragment thereof of an amino acid sequence at least 85% identical to SEQ ID NO: 11, or
g. the conserved protein is an HIV conserved protease protein (pro) or an immunogenic fragment thereof of amino acid sequence SEQ ID NO: 12; or
h. the conserved protein is an HIV conserved protease protein (pro) or an immunogenic fragment thereof of an amino acid sequence at least 85% identical to SEQ ID NO: 12, or
i. the conserved protein or immunogenic fragment is a combination thereof.

7. The universal vaccine according to claim 1, wherein the activated cell populations comprise activated cytotoxic T lymphocytes (CTLs).

8. The universal vaccine according to claim 7, wherein the activated CTLs comprise one or more of an NK cell population, an NKT cell population, an LAK cell population, a CIK cell population, a MAIT cell population, a CD8+ CTL population, or a CD4+ CTL population.

9. The universal vaccine according to claim 1, wherein
(a) the immune enhancer comprises an adjuvant; or
(b) the immune enhancer comprises a naked DNA vector encoding a conserved polypeptide antigen or immunogenic fragment thereof comprising about 1 nanogram to about 2000 micrograms of DNA, inclusive; or
(c) the immune enhancer comprises both an adjuvant and a naked DNA vector encoding the conserved protein antigen.

10. The universal vaccine according to claim 9, wherein the adjuvant comprises one or more of alum, aluminum salts, a saponin, an oil-in-water emulsion based on squalene, an unmethyl CpG dinucleotide; monophosphoryl lipid A (MPL) or an aminoalkyl glucosaminide-4-phosphate (AGP) mimetic thereof; 3-de-O-acylated monophosphoryl lipid A (3D-MPL), together with an aluminum salt; a monophosphoryl lipid A and saponin derivative; a polyoxyethylene ether; an anti-CD40 antibody; or GM-CSF.

11. The universal vaccine of claim 1 prepared by a process comprising:
a. identifying and selecting from a consensus amino acid sequence a highly conserved internal protein of an infectious viral pathogen or an immunogenic fragment thereof enriched in T cell recognition antigens;
b. constructing immunogen sequences of the highly conserved internal proteins in (a);
c. constructing:
i. a DNA vector comprising the immunogen sequences of (b);
ii. an adenovirus-based (AdV) vector comprising the immunogen sequences of (b);
iii. a replication-competent recombinant vaccinia virus based (VV) vector comprising the immunogen sequences of (b);
d. propagating separately each of the recombinant vectors comprising encoded immunogens in (c) for immunizing a subject in vivo in an amount effective to elicit or stimulate a therapeutic or prophylactic cell mediated immune response against an infection with the infectious pathogen by:
i. priming the fully human immune system by immunizing with the phage DNA vector of (c)(i);
ii. boosting the fully human immune system by immunizing with the AdV vector of (c)(ii) followed by the VV vector of (c)(iii), or the VV vector of (c)(iii) followed by the AdV vector of (c)(ii).

12. The universal vaccine prepared by the process according to claim 11, wherein
a) the DNA vector is selected from the group consisting of a Streptomyces phage SV1.0 DNA vector, an attenuated Mycobacterium tuberculosis vector, a Bacillus Calmette Guerin (BCG) vector, a Salmonella species bacterial vector, a Shigella species bacterial vector,
b) the AdV viral vector is selected from the group consisting of Adenovirus (Ad vectors) based on Ad serotype 5 (AdHu5), adeno-associated virus (AAV), AD26 vector chimpanzee adenoviral isolate Y25, AdC68/Sad-V25), ChAd63, AdC68 (SAdV-25), AdC7 (SAdV-24) and AdC6 (SAdV-23), and ChAdOx1;
c) the vaccinia virus viral vector is selected from the group consisting of attenuated vaccinia strains Modified Vaccinia Ankara (MVA), chorioallantois vaccinia virus Ankara (CVA) strain], live vaccinia virus strains WR strain, New York City Board of Health (NYCBH) strain, ACAM2000, Lister strain, LC16 m8, Elstree-BNm, Copenhagen strain, and Tiantan strain (VTT)

13. The universal vaccine prepared by the process according to claim 11, wherein the viral pathogen is a human coronavirus.

14. The universal vaccine prepared by the process according to claim 13, wherein
a. the conserved protein is a coronavirus spike (S) protein of amino acid sequence SEQ ID NO: 1. or
b. The conserved protein is a coronavirus spike (S) protein of an amino acid sequence at least 85% identical to SEQ ID NO: 1; or
c. the conserved protein is an isolated coronavirus S protein 51 subunit of amino acid sequence SEQ ID NO: 1; or
d. the conserved protein is an isolated coronavirus S protein 51 subunit cleaved by a transmembrane serine protease (TMPRSS2) comprising a receptor binding domain (RBD); or
e. the conserved protein is an isolated coronavirus S protein 51 subunit cleaved by a transmembrane serine protease (TMPRSS2) comprising an RBD domain of an 51 subunit enzymatically stripped of at least 5% of its covering of N-linked glycans, O-linked glycans or both by limited digestion; or
f. the conserved protein is an isolated coronavirus S protein S2 subunit subunit of amino acid sequence SEQ ID NO: 1; or
g. the conserved protein is an isolated coronavirus S protein S2 subunit enzymatically stripped of at least 5% of its covering of N-linked glycans by limited digestion, or
h. the conserved protein is a coronavirus membrane (M) protein of amino acid sequence SEQ ID NO: 3 or 4; or
i. the conserved protein is a coronavirus membrane (M) protein of an amino acid sequence at least 85% identical to SEQ ID NO: 3 or 4; or
j. the conserved protein is a coronavirus envelope (E) protein of amino acid sequence SEQ ID NO: 5 or 6; or
k. the conserved protein is a coronavirus envelope (E) protein of an amino acid sequence at least 85% identical to SEQ ID NO: 5 or SEQ ID NO: 6; or
l. the conserved protein is a coronavirus nucleocapsid (N) protein of amino acid sequence SEQ ID NO: 7 or 8; or
m. the conserved protein is a coronavirus nucleocapsid (N) protein or immunogenic fragment thereof of an amino acid sequence at least 85% identical to SEQ ID NO: 7 or SEQ ID NO: 8, or
n. the conserved protein or immunogenic fragment is a combination thereof.

15. The universal vaccine prepared by the process according to claim 11, wherein the virus is a Retroviridae virus and the Retroviridae virus is a human immunodeficiency virus (HIV).

16. The universal vaccine prepared by the process according to claim according to claim 15, wherein
a. the conserved protein is an HIV conserved capsid protein (gag) of SEQ ID NO: 9; or
b. the conserved protein is an HIV conserved capsid protein (gag) of an amino acid sequence at least 85% identical to SEQ ID NO: 9, or
c. the conserved protein is an HIV conserved envelope protein (env) of SEQ ID NO: 10; or
d. the conserved protein is an HIV conserved envelope protein (env) of an amino acid sequence at least 85% identical to SEQ ID NO: 10, or
e. the conserved protein is an HIV conserved polymerase protein (pol) of SEQ ID NO: 11; or
f. the conserved protein is an HIV conserved polymerase protein (pol) of an amino acid sequence at least 85% identical to SEQ ID NO: 11, or
g. the conserved protein is an HIV conserved protease protein (pro) of SEQ ID NO: 12; or
h. the conserved protein is an HIV conserved protease protein (pro) of an amino acid sequence at least 85% identical to SEQ ID NO: 12, or
i. the conserved protein or immunogenic fragment is a combination thereof.

17. An engineered nucleic acid encoding at least one RNA polynucleotide comprising an open reading frame encoding at least one polypeptide antigen or an immunogenic fragment thereof, wherein the polypeptide antigen, or the immunogenic fragment thereof, comprises a conserved internal protein that is enriched in T cell recognition antigens of the universal vaccine of claim 1.

18. An expression vector comprising an engineered nucleic acid encoding at least one RNA polynucleotide comprising an open reading frame encoding at least one polypeptide antigen or an immunogenic fragment thereof, wherein the polypeptide antigen, or the immunogenic fragment thereof, comprises a conserved internal protein that is enriched in T cell recognition antigens of the universal vaccine of claim 1.

19. A host cell comprising an engineered nucleic acid encoding at least one RNA polynucleotide comprising an open reading frame encoding at least one polypeptide antigen or an immunogenic fragment thereof, wherein the polypeptide antigen, or the immunogenic fragment thereof, comprises a conserved internal protein that is enriched in T cell recognition antigens of the universal vaccine of claim 1.

20. A method of inducing an immune response in a subject, the method comprising administering to the subject a universal vaccine against an immunogen of an infectious pathogenic virus selected from a human Coronaviridae or a human Retroviridae virus comprising
a. at least one ribonucleic acid (RNA) polynucleotide comprising an open reading frame encoding at least one antigenic polypeptide or an immunogenic fragment thereof, wherein the antigenic peptide, or the immunogenic fragment thereof, comprises a conserved internal protein that is enriched in T cell recognition antigens,
b. optional helper T cell (Th) epitopes comprising
i. at least one full-length protein that is immunogenic; or
ii. overlapping peptides of at least 30 amino acids in length that together cover the conserved internal viral protein
c. an optional immune response enhancer; and
d. a pharmaceutically acceptable carrier,
wherein
(1) a T cell recognition antigen consists of peptides of about 7 to about 14 residues in length, and
(2) the immune response produced in response to the vaccine comprises one or more of:
(i) activation of one or more T cell populations directed to an antigen(s) present in the vaccine; or
(ii) neutralization of infectivity of the pathogen; or
(iii) an antigen-specific response comprising destruction of the infectious pathogenic organism; lysis of cells infected with the infectious pathogenic organism, or both;
compared to a control immunized with the immunogen without the ribonucleic acid (RNA) polynucleotide comprising the open reading frame encoding the at least one polypeptide antigen or the immunogenic fragment thereof.

21. The method of claim 20, wherein
a. The immune enhancer comprises an adjuvant; or
b. The immune enhancer comprises a naked DNA vector encoding a conserved polypeptide antigen or immunogenic fragment thereof comprising about 1 nanogram to about 2000 micrograms of DNA, inclusive; or
c. The immune enhancer comprises both an adjuvant and a naked DNA vector encoding the conserved protein antigen.

22. The method of claim 21, wherein the adjuvant comprises one or more of alum, aluminum salts, a saponin, an oil-in-water emulsion based on squalene, an unmethyl CpG dinucleotide; polyinosinic-polycytidylic acid (poly(I:C); monophosphoryl lipid A (MPL) or an aminoalkyl glucosaminide-4-phosphate (AGP) mimetic thereof; 3-de-O-acylated monophosphoryl lipid A (3D-MPL), together with an aluminum salt; a monophosphoryl lipid A and saponin derivative; a polyoxyethylene ether; an anti-CD40 antibody; or GM-CSF.

23. The method of claim 20, comprising
a. priming the subject with a naked nucleic acid or DNAvector comprising a first immunogen sequence encoding a conserved internal protein that is enriched in T cell recognition antigens; and
b. then boosting the subject with a boosting composition comprising a an attenuated, replication-competent recombinant vaccinia virus based (VV) vector comprising a second immunogen sequence encoding a conserved internal protein that is enriched in CD8+ T cell recognition antigens.

24. The method of claim 23, wherein
a) the DNA vector is selected from the group consisting of a Streptomyces phage SV1.0 DNA vector, an attenuated Mycobacterium tuberculosis vector, a Bacillus Calmette Guerin (BCG) vector, a Salmonella species bacterial vector, a Shigella species bacterial vector, or
b) the AdV viral vector is selected from the group consisting of Adenovirus (Ad vectors) based on Ad serotype 5 (AdHu5), adeno-associated virus (AAV), AD26 vector chimpanzee adenoviral isolate Y25, AdC68/Sad-V25), ChAd63, AdC68 (SAdV-25), AdC7 (SAdV-24) and AdC6 (SAdV-23), and ChAdOx1; or
c) the vaccinia virus viral vector is selected from the group consisting of attenuated vaccinia strains Modified Vaccinia Ankara (MVA), chorioallantois vaccinia virus Ankara (CVA) strain], Live vaccinia virus strains WR strain, New York City Board of Health (NYCBH) strain, ACAM2000, Lister strain, LC16 m8, Elstree-BNm, Copenhagen strain, and Tiantan strain (VTT).

25. The method of claim 23, wherein
(a) the naked nucleic acid or the DNA vector encodes one or more conserved protein of a human immunodeficiency virus; and
(b) the boosting composition comprises a subunit or protein vaccine comprising one or more conserved proteins of the human immunodeficiency virus.

26. The method according to claim 23, wherein
a. the naked nucleic acid or DNA vector encodes a conserved protein of a human coronavirus; and
b. the boosting composition comprises a subunit or protein vaccine comprising one or more conserved proteins of the human coronavirus.

27. The method of claim 26, wherein the conserved protein of the human coronavirus is one or more of:
a human coronavirus (S) protein of amino acid sequence SEQ ID NO: 2 or an immunogenic fragment thereof;
a coronavirus membrane (M) protein or an immunogenic fragment thereof of amino acid sequence SEQ ID NO: 3 or 4;
a coronavirus nucleocapsid (N) protein or an immunogenic fragment thereof of aminoacid sequence SEQ ID NO: 7 or 8;
or a coronavirus envelope (E) protein or an immunogenic fragment thereof of amino acid sequence SEQ ID NO: 5 or 6.

28. The method according to claim 23, wherein
a. the naked nucleic acid or DNA vector encodes a conserved protein of a human immunodeficiency virus; and
b. the boosting composition comprises a subunit or protein vaccine comprising one or more conserved proteins of the human immunodeficiency virus.

29. The method of claim 28, wherein the conserved protein of the human immunodeficiency virus is one or more of:
an HIV conserved capsid protein (gag) of amino acid sequence SEQ ID NO: 9 or an immunogenic fragment thereof;
an HIV conserved envelope protein (env) or an immunogenic fragment thereof of amino acid sequence SEQ ID NO: 10; or an immunogenic fragment thereof;
an HIV conserved polymerase protein (pol) or an immunogenic fragment thereof of amino acid sequence SEQ ID NO: 11, or an immunogenic fragment thereof; or
an HIV conserved protease protein (pro) or an immunogenic fragment thereof of amino acid sequence SEQ ID NO: 12.

30. The method of claim 20, comprising administering the vaccine to the subject by inhalation, insufflation or by intramuscular injection.

31. The method of claim 23, wherein the mode of administration of the priming dose and the mode of administration of the booster dose are different.

32. The method according to claim 20, wherein the subject is a mouse of phenotype NOD-scid γc−/− or BALB/c Rag2−/− γc−/−.

33. The method according to claim 32, comprising reconstituting the mouse of phenotype NOD-scid γc−/− with human C34+CD133+ cord blood cells injected intracardially as newborns into the NOD-scid γc−/− mouse.

34. The method according to claim 32, comprising reconstituting the mouse of phenotype BALB/c Rag2−/− γc−/− comprises CD34+ hematopoietic progenitor cells (HPCs) isolated from human fetal liver transferred intrahepatically into newborn BALB/c Rag2−/− γc−/−.

35. A method for inducing a pan-coronavirus specific cellular immune response in vivo in an animal model comprising a fully human functional immune system comprising:
(1) Identifying and selecting from a consensus amino acid sequence a plurality of highly conserved coronavirus viral proteins enriched in T cell recognition antigens;
(2) constructing concatenated immunogen sequences of the highly conserved coronavirus viral proteins in (a);
(3) constructing:
a. a DNA vector, comprising the concatenated immunogen sequences of (b);
b. an adenovirus-based (AdV) vector comprising the concatenated immunogen sequences of (b);
c. an attenuated, replication-competent recombinant vaccinia virus based (VV) vector comprising the concatenated immunogen sequences of (b);
(4) propagating separately each of the recombinant vectors comprising encoded immunogens in (3);
(5) immunizing the animal model comprising the fully human functional immune system in vivo by:
a. priming the fully human immune system by immunizing with the DNA vector of 3(a);
b. boosting the fully human immune system by immunizing with the AdV vector of 3(b) followed by the VV vector of 3(c), or the VV vector of 3(c) followed by the AdV vector of 3(b); and
(6) after the immunizing in (5), challenging the animal model comprising the immunized fully human functional immune system with SARS-CoV-1, MERS-CoV, or SARS-CoV-2 virus.

36. The method of claim 35, wherein
a) the DNA vector is selected from the group consisting of a Streptomyces phage SV1.0 DNA vector, an attenuated Mycobacterium tuberculosis vector, a Bacillus Calmette Guerin (BCG) vector, a Salmonella species bacterial vector, a Shigella species bacterial vector,
b) the AdV viral vector is selected from the group consisting of Adenovirus (Ad vectors) based on Ad serotype 5 (AdHu5), adeno-associated virus (AAV), AD26 vector chimpanzee adenoviral isolate Y25, AdC68/Sad-V25), ChAd63, AdC68 (SAdV-25), AdC7 (SAdV-24) and AdC6 (SAdV-23), and ChAdOx1;
c) the pox virus viral vector is selected from the group consisting of attenuated vaccinia strains Modified Vaccinia Ankara (MVA), chorioallantois vaccinia virus Ankara (CVA) strain], Live vaccinia virus strains WR strain, New York City Board of Health (NYCBH) strain, ACAM2000, Lister strain, LC16 m8, Elstree-BNm, Copenhagen strain, and Tiantan strain (VTT)

37. The method of claim 35, the step of immunizing further comprising administering a pharmaceutical composition containing
a. helper T cell (Th) epitopes comprising
i. at least one full-length protein that is immunogenic; or
ii. overlapping peptides of at least 30 amino acids in length that together cover the conserved internal viral protein
b. an optional immune response enhancer; and
c. a pharmaceutically acceptable carrier

38. The method of claim 37, wherein the immune response enhancer is an adjuvant, and the adjuvant comprises one or more of alum, aluminum salts, a saponin, an oil-in-water emulsion based on squalene, an unmethyl CpG dinucleotide; polyinosinic-polycytidylic acid (poly(I:C); monophosphoryl lipid A (MPL) or an aminoalkyl glucosaminide-4-phosphate (AGP) mimetic thereof; 3-de-O-acylated monophosphoryl lipid A (3D-MPL), together with an aluminum salt; a monophosphoryl lipid A and saponin derivative; a polyoxyethylene ether; an anti-CD40 antibody; or GM-CSF.

39. The method of claim 35, comprising administering the vaccine to the subject by intradermal injection, intranasally, by insufflation, or by intramuscular injection.

40. The method of claim 35, wherein the mode of administration of the priming dose and the booster dose are different.

41. The method of claim 35, wherein the animal model is a mouse of phenotype NOD-scid γc−/− or BALB/c Rag2−/− γc−/−.

42. The method of claim 35, comprising reconstituting the mouse of phenotype NOD-scid γc−/− with human C34+CD133+ cord blood cells injected intracardially as newborns into the NOD-scid γc−/− mouse.

43. The method of claim 35, comprising reconstituting the mouse of phenotype BALB/c Rag2−/− γc−/− comprises CD34+ hematopoietic progenitor cells (HPCs) isolated from human fetal liver transferred intrahepatically into newborn BALB/c Rag2−/− γc−/−.34.

44. The method of claim 35, wherein the coronavirus specific cellular and humoral immune response in the animal model may be effective to reduce spread of infection in a population of unimmunized reconstituted mice.

45. The method according to claim 35, wherein
the conserved protein is a coronavirus spike (S) protein of amino acid sequence SEQ ID NO: 1. or
The conserved protein is a coronavirus spike (S) protein of an amino acid sequence at least 85% identical to SEQ ID NO: 1; or
the conserved protein is an isolated coronavirus S protein S1 subunit of amino acid sequence SEQ ID NO: 1; or
the conserved protein is an isolated coronavirus S protein S1 subunit cleaved by a transmembrane serine protease (TMPRSS2) comprising a receptor binding domain (RBD); or
the conserved protein is an isolated coronavirus S protein S1 subunit cleaved by a transmembrane serine protease (TMPRSS2) comprising an RBD domain of an S1 subunit enzymatically stripped of at least 5% of its covering of N-linked glycans, O-linked glycans or both by limited digestion; or
the conserved protein is an isolated coronavirus S protein S2 subunit subunit of amino acid sequence SEQ ID NO: 1; or
the conserved protein is an isolated coronavirus S protein S2 subunit enzymatically stripped of at least 5% of its covering of N-linked glycans by limited digestion, or
the conserved protein is a coronavirus membrane (M) protein of amino acid sequence SEQ ID NO: 3 or 4; or
the conserved protein is a coronavirus membrane (M) protein of an amino acid sequence at least 85% identical to SEQ ID NO: 3 or 4; or
the conserved protein is a coronavirus envelope (E) protein of amino acid sequence SEQ ID NO: 5 or 6; or
the conserved protein is a coronavirus envelope (E) protein of an amino acid sequence at least 85% identical to SEQ ID NO: 5 or SEQ ID NO: 6; or
the conserved protein is a coronavirus nucleocapsid (N) protein of amino acid sequence SEQ ID NO: 7 or 8; or
the conserved protein is a coronavirus nucleocapsid (N) protein or immunogenic fragment thereof of an amino acid sequence at least 85% identical to SEQ ID NO: 7 or SEQ ID NO: 8, or
the conserved protein or immunogenic fragment is a combination thereof.
